(19) United States
(12) Patent Application Publication
(10) Pub. No.: US 2025/0346956 A1
(43) Pub. Date: Nov. 13, 2025

(54) SYSTEMS, COMPOSITIONS, AND METHODS RELATING TO NEURODEGENERATIVE DISEASES

(71) Applicants: Seer, Inc., Redwood City, CA (US); The General Hospital Corporation, Boston, MA (US)

(72) Inventors: Mahdi ZAMANIGHOMI, Belmont, MA (US); Harendra GUTURU, Oakland, CA (US); Jian WANG, San Diego, CA (US); Ting HUANG, San Leandro, CA (US); Asim SIDDIQUI, San Francisco, CA (US); Serafim BATZOGLOU, San Francisco, CA (US); Guhan Venkataraman, Cupertino, CA (US); Steven E. Arnold, Swarthmore, PA (US); Pia Kivisakk Webb, Westwood, MA (US); Sudeshna Das, Chestnut Hill, MA (US); Bradley Theodore Hyman, Madison, NH (US)

(21) Appl. No.: 18/871,105

(22) PCT Filed: Jun. 2, 2023
(86) PCT No.: PCT/US2023/067872

§ 371 Date: Dec. 2, 2024

Related U.S. Application Data
(60) Provisional application No. 63/348,697, filed on Jun. 3, 2022.
(60) Provisional application No. 63/488,430, filed on Mar. 3, 2023.

(51) Int. Cl. C12Q 1/6883

(52) U.S. Cl. CPC C12Q 1/6883; C12Q 2600/112; C12Q 2600/156; C12Q 2600/158

(57) ABSTRACT

In some aspects, the present disclosure provides a method for determining a risk or state of a neurodegenerative disease of a subject. In some embodiments, the method compriseses detecting a presence of a biomarker in a biological sample from the subject, wherein the biomarker comprises at least one of: E7EUF1, O94812, P02549, P02730, P05019, P05154, P05546, P13497, P16157, P16452, P17936, P24593, P27918, P35858, P41218, Q12797, Q13214, Q13822, Q8NI99, Q96IY4, Q99715, Q9BXN1, Q9H0B8, or a proteoform thereof. In some embodiments, the method comprises detecting a presence of a biomarker in a biological sample from the subject, wherein the biomarker comprises at least one of: P54803, P14625, P30043, P00742, A0A0D9SG88, Q5TFM2, P54803, P54803-3, P54803-4, P04196, or a proteoform thereof. In some embodiments, the method comprises determining the risk or state of the neurodegenerative disease of the subject based on the presence of the biomarker in the biological sample.

### CROSS-REFERENCE

This application is a U.S. National Phase application of International Application No. PCT/US2023/067872, filed Jun. 2, 2023, which claims the benefit of U.S. Provisional Application No. 63/348,697, filed Jun. 3, 2022, and U.S. Provisional Application No. 63/488,430, filed Mar. 3, 2023, each of which are incorporated herein by reference in their entirety.

### STATEMENT AS TO FEDERALLY SPONSORED RESEARCH

This invention was made with government support under R44 AG065051 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on May 30, 2023, is named 53344-762_601_SL.xml and is 173,970 bytes in size.

### BACKGROUND

Few methods exist for accurate neurodegenerative diagnosis. Primary screening for neurodegeneration is typically based on cognitive assessment (e.g., Mini-Mental State Examinations and Memory Impairment Screens), and therefore typically identifies cognitive decline without providing insight into underlying causes, pathologies, and risk factors. While medical imaging (e.g., Magnetic Resonance Imaging) and tissue analysis can, in certain cases, distinguish neurological conditions, these methods may struggle with early phase detection and tracking disease progression, and may be prohibitively invasive and cost intensive for routine use.

### SUMMARY

In some aspects, the present disclosure provides a method for determining a risk or state of a neurodegenerative disease of a subject comprising: (a) detecting a presence of a biomarker in a biological sample from the subject, wherein the biomarker comprises at least one of: E7EUF1, O94812, P02549, P02730, P05019, P05154, P05546, P13497, P16157, P16452, P17936, P24593, P27918, P35858, P41218, Q12797, Q13214, Q13822, Q8NI99, Q96IY4, Q99715, Q9BXN1, Q9H0B8, or a proteoform thereof; and (b) determining the risk or state of the neurodegenerative disease of the subject based on the presence of the biomarker in the biological sample.

In some embodiments, the biomarker further comprises at least one of: P04114, P30043, P00742, or a proteoform thereof.

In some embodiments, O94812 proteoform is a proteoform selected from the group consisting of: O94812-2, O94812-3, O94812-5, O94812-6, and O94812-7.

In some embodiments, P02549 proteoform is a P02549-2 proteoform.

In some embodiments, P35858 proteoform is a P35858-2 proteoform.

In some embodiments, Q13214 proteoform is a Q13214-2 proteoform.

In some embodiments, Q13822 proteoform is a Q13822-3 proteoform.

In some embodiments, Q9H0B8 proteoform is a Q9H0B8-3 proteoform.

In some embodiments, P16157 proteoform is a P16157-12 proteoform.

In some embodiments, P16452 proteoform is a P16452-2 proteoform.

In some aspects, the present disclosure also provides a method for determining a risk or state of a neurodegenerative disease of a subject comprising: (a) detecting a presence of a biomarker in a biological sample from the subject, wherein the biomarker comprises at least one of: 0A0B4JIU7, A0A0D9SG88; Q5TFM2, A0A3B3ISR2_; B4DPQ0; F5H2DO, ASYKK6, A8TX70; A8TX70-2, H3BPZ1, O14960, O14980, O75093, O75900-2, P00748, P00751, P00915, P01042-3, P01892, P02042, P04040, P04180, P04196, P07093-2; _P07093-3, P070933, P08833, P09668, P10316, P10321-2, P10643, P11226, P13747, P14625, P16871; _P16871-2; _P16871-3; _P16871-4, P19823, P19827, P21741, P26572, P26927, P28799, P36980, P36980; _P36980-2, P40937; P40937-2, P54803, P54803; _P54803-3; P54803-4, P78539; P78539-2; _P78539-5, Q03591, Q06033; Q06033-2, Q08257-3, Q13131; Q13131-2, Q14213, Q15149-2, Q15149-4, Q15149; Q15149-2; Q15149-4; Q15149-5; Q15149-6; Q15149-7; Q15149-8; Q15149-9, Q15165; Q15165-1; Q15165-3, Q15517, Q16678, Q709C8; Q709C8-3, Q8N474, Q8N6R0; Q8N6R0-1; Q8N6R0-3, Q92896; Q92896-2; Q92896-3, Q93091, Q96PD5, Q96PD5-2, Q9H479, Q9H4F8; Q9H4F8-2, Q9NR34, Q9NUQ9, Q9P035, Q9ULI3, Q9UQN3; Q9UQN3-2, Q9Y263, Q9Y450; Q9Y450-4, or a proteoform thereof; and (b) determining the risk or state of the neurodegenerative disease of the subject based on the presence of the biomarker in the biological sample.

In some embodiments, the biomarker further comprises at least one of: P54803, P14625, P30043, P00742, A0A0D9SG88, Q5TFM2, P54803, P54803-3, P54803-4, P04196, or a proteoform thereof.

In some embodiments, the state is a diagnosis or a prognosis of the neurodegenerative disease in the subject.

In some embodiments, the state is a stratification of the neurodegenerative disease in the subject.

In some embodiments, the state is a monitoring of the neurodegenerative disease in the subject.

In some embodiments, the risk is an expected probability that the subject develops the neurodegenerative disease later in life.

In some embodiments, the neurodegenerative disease is Alzheimer's disease.

In some embodiments, the biological sample comprises plasma, serum, urine, cerebrospinal fluid, synovial fluid, tears, saliva, whole blood, a white blood cell, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

In some embodiments, the biological sample is a blood, serum, or plasma.

In some embodiments, the biological sample is plasma.

In some embodiments, the presence of the biomarker comprises a level of the biomarker.

In some embodiments, the level of the biomarker is indicative for the presence of the neurodegenerative disease in the subject when the level is higher in the biological sample compared to a reference value.

In some embodiments, the level of the biomarker is indicative for the absence of the neurodegenerative disease in the subject when the level is lower in the biological sample compared to a reference value.

In some embodiments, the biomarker comprises a plurality of biomarkers comprising at least two of: E7EUF1, O94812, P02549, P02730, P05019, P05154, P05546, P13497, P16157, P16452, P17936, P24593, P27918, P35858, P41218, Q12797, Q13214, Q13822, Q8NI99, Q96IY4, Q99715, Q9BXN1, Q9H0B8, or a proteoform thereof.

In some embodiments, the presence of the biomarker comprises a pattern of levels of the plurality biomarkers.

In some embodiments, the pattern of levels of the plurality of biomarkers is indicative for the presence of the neurodegenerative disease in the subject when the pattern of levels is substantially similar in the biological sample compared to a reference pattern of levels.

In some embodiments, the pattern of levels of the plurality of biomarkers is indicative for the presence of the neurodegenerative disease in the subject when the pattern of levels is substantially different in the biological sample compared to a reference pattern of levels.

In some embodiments, the determining comprises using a machine learning algorithm to analyze the pattern of levels.

In some embodiments, the machine learning algorithm is configured to determine if the pattern of levels of the plurality of biomolecules of the biomarker is substantially similar or substantially different in the biological sample compared to the reference pattern of levels.

In some embodiments, the detecting a presence of a biomarker in the biological sample comprises selectively enriching the one or more biomolecules.

In some embodiments, the detecting a presence of a biomarker in the biological sample comprises performing mass spectrometry with the one or more biomolecules.

In some embodiments, the subject is suspected of having early-stage Alzheimer's Disease.

In some embodiments, the subject is asymptomatic for the neurodegenerative disease.

In some embodiments, the subject exhibits or reports one or more symptoms of Alzheimer's Disease comprising: impairment in cognition, impairment in memory, impairment in language, or impairment in perception.

In some embodiments, the subject has or is suspected of having Alzheimer's Disease based on an expert evaluation.

In some embodiments, the subject has or is suspected of having Alzheimer's Disease based on a positive Alzheimer's Disease diagnosis from another diagnostic.

In some aspects, the present disclosure provides a method for determining a risk or state of a neurodegenerative disease of a subject comprising: (a) detecting a presence of a biomarker in a biological sample from the subject, wherein the biomarker is involved in a fibrin clot formation cascade; and (b) determining the risk or state of the neurodegenerative disease of the subject based on the presence of the biomarker in the biological sample.

In some embodiments, the biomarker is involved in an extrinsic pathway of the fibrin clot formation cascade.

In some embodiments, the biomarker is involved in an intrinsic pathway of the fibrin clot formation cascade.

In some embodiments, the biomarker is involved in a common pathway of the fibrin clot formation cascade.

In some embodiments, the biomarker comprises at least one of: P00451, P00734, P00740, P00742, P00748, P01008, P01023, P01042, P02671, P02675, P02679, P02776, P03951, P03952, P04070, P04275, P05154, P05155, P05160, P05546, P07093, P07204, P07225, P07359, P08709, P10646, P10720, P12259, P13224, P13726, P14770, P24158, P25116, P40197, P42785, Q07021, Q8N6Q3, Q9UNN8, or a proteoform thereof.

In some embodiments, the biomarker is involved in a complement and coagulation cascade, a IGF1-IGFBP3-ALS complex, or a regulation of insulin-like growth factor (IGF) transport and uptake by Insulin-like Growth Factor Binding Proteins (IGFBPs).

In some embodiments, the biomarker is involved in the complement and coagulation cascades.

In some embodiments, the biomarker comprises at least one of: K03901, K01320, K01314, K03902, K01313, K01328, K01323, K01321, K03900, K03899, K03907, K06557, K01344, K03914, K04235, K04236, K03917, K03906, K01300, K03903, K03904, K03905, K01324, K03898, K03915, K03916, K01315, K03909, K03911, K03912, K03913, K03908, K03982, K19821, K01343, K01348, K03985, K03984, K03983, K03910, K01335, K01334, K03990, K03994, K03995, K03996, K03997, K03998, K03999, K04000, K03986, K03987, K03988, K01330, K01331, K03991, K03992, K03993, K01332, K03989, K04009, K19822, K04011, K04012, K06461, K06464, K06462, K04010, K04004, K01333, K04001, K04006, K04007, K04002, K04003, K04008, K17252, K06251, or a proteoform thereof.

In some embodiments, the biomarker is involved in the IGF1-IGFBP3-ALS complex.

In some embodiments, biomarker comprises at least one of: P05019, P17936, P35858, or a proteoform thereof.

In some embodiments, biomarker is involved in the regulation of insulin-like growth factor (IGF) transport and uptake by Insulin-like Growth Factor Binding Proteins (IGFBPs).

In some embodiments, biomarker comprises at least one of: O00255, O00391, O00622, O14672, O14791, O15232, O15240, O43493, O43852, O76024, O76061, O95084, O95633, O95972, P00450, P00734, P00747, P01008, P01009, P01024, P01033, P01034, P01042, P01210, P01344, P02647, P02649, P02652, P02671, P02679, P02751, P02765, P02768, P02771, P02787, P03956, P04070, P04114, P05019, P05060, P05067, P05231, P05546, P06870, P07237, P07288, P07942, P08253, P08311, P08582, P08833, P09382, P09603, P0C0L4, P10451, P11047, P12259, P12644, P13521, P13611, P14314, P14625, P17936, P18065, P19022, P19823, P20151, P20718, P22692, P23327, P24592, P24593, P24821, P34741, P35555, P35858, P51654, P55268, Q02818, Q06481, Q07065, Q08431, Q12841, Q13103, Q13217, Q13219, Q13316, Q13421, Q14393, Q14515, Q14703, Q14766, Q15084, Q15293, Q16270, Q24JP5, Q5JRA6, Q6P988, Q6PCB0, Q6Q788, Q6UX39, Q86UP2, Q8IXL6, Q8N114, Q8N4F0, Q8NBJ4, Q8NBP7, Q8WXD2, Q96AD5, Q96MK3, Q99217, Q9BRK3, Q9BTY2, Q9BU40, Q9BXP8, Q9GZV9, Q9H8M9, Q9HCE9, Q9NP70, Q9NQ76, Q9NRM1, Q9UK55, Q9UKR3, Q9UM21, or a proteoform thereof.

In some embodiments, the subject is suspected of having early-stage Alzheimer's Disease.

In some embodiments, the subject is asymptomatic for the neurodegenerative disease.

In some embodiments, the subject exhibits or reports one or more symptoms of Alzheimer's Disease comprising: impairment in cognition, impairment in memory, impairment in language, or impairment in perception.

In some embodiments, the subject has or is suspected of having Alzheimer's Disease based on an expert evaluation.

In some embodiments, the subject has or is suspected of having Alzheimer's Disease based on a positive Alzheimer's Disease diagnosis from another diagnostic.

In some aspects, the present disclosure provides a method for determining a risk or state of a disease of a subject comprising: (a) detecting a presence of one or more single nucleotide polymorphisms (SNPs) in a biological sample from the subject, wherein the one or more SNPs comprise at least one of: GSA-rs1046279, GSA-rs1131364, GSA-rs1165692, GSA-rs6030, rs1001179, rs1046875, rs1046896, rs1061147, rs1061147, rs10737680, rs10801555, rs11003118, rs1106766, rs11085954, rs11613352, rs1165669, rs12067507, rs12148472, rs12493926, rs1329424, rs1329428, rs1410996, rs1520079, rs17198, rs1800450, rs1801020, rs1801689, rs1831282, rs2289702, rs2545801, rs2731672, rs2749534, rs3197999, rs34882957, rs3741414, rs3784539, rs3792366, rs380390, rs395544, rs41268617, rs4524, rs5030062, rs62623707, rs6647, rs6677604, rs6695321, rs68066031, rs68147365, rs698078, rs707998, rs710446, rs7599241, rs8099840, rs880633, rs9820435, rs9898, or a SNP in linkage disequilibrium thereof; and (b) determining the risk or state of the disease of the subject based on the presence of the one or more SNPs in the biological sample.

In some aspects, the present disclosure provides a method for determining a risk or state of a disease of a subject comprising: (a) detecting a presence of one or more single nucleotide polymorphisms (SNPs) in a biological sample from the subject, wherein the one or more SNPs comprise at least one of: chr6:29928649, chr6:29939240, exm2260367, exm2260986, exm2261461, exm2261761, exm2264447, exm2264486, exm2265845, exm2270485, exm2271590, GSA-rs1131364, GSA-rs11901661, GSA-rs11915474, GSA-rs12085181, GSA-rs13355990, GSA-rs1611704, GSA-rs1651025, GSA-rs17706123, GSA-rs2975041, GSA-rs36013739, GSA-rs4280141, GSA-rs55895668, GSA-rs6030, GSA-rs7088203, GSA-rs76221975, GSA-rs78374057, GSA-rs7872425, GSA-rs7793026, GSA-rs7754077, GSA-rs7088203, rs3923387, rs11718493, rs11764079, rs11777239, rs11783655, rs11915474, rs12085181, rs12148472, rs12258356, rs12493410, rs12493926, rs1426654, rs1611715, rs1736982, rs1800450, rs1801020, rs203850, rs218396, rs2289702, xs2345436, rs2372813, rs2523409, rs2523946, rs2545801, rs272817, rs2735046, rs2735097, rs2743941, rs2975034, rs3115627, rs35620248, rs380390, rs3856650, rs387608, rs395544, rs399419, rs41268617, rs4305381, rs4344876, rs4404487, rs440770, rs4524, rs4740, rs508406, rs6003, rs6049301, rs6512033, rs6677604, rs6783962, rs68066031, rs6890853, rs7014582, rs73466148, rs7464572, rs7493, rs7533936, rs8099840, rs812498, rs9357092, rs9380141, rs9824398, rs9898, seq-rs6993938, seq-rs1126605, or a SNP in linkage disequilibrium thereof; and (b) determining the risk or state of the disease of the subject based on the presence of the one or more SNPs in the biological sample.

In some embodiments, the disease is a neurodegenerative disease.

In some embodiments, the state is a diagnosis or a prognosis of the neurodegenerative disease in the subject.

In some embodiments, the state is a stratification of the neurodegenerative disease in the subject.

In some embodiments, the state is a monitoring of the neurodegenerative disease in the subject.

In some embodiments, the risk is an expected probability that the subject develops the neurodegenerative disease later in life.

In some embodiments, the neurodegenerative disease is Alzheimer's disease.

In some embodiments, the biological sample comprises plasma, serum, urine, cerebrospinal fluid, synovial fluid, tears, saliva, whole blood, a white blood cell, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

In some embodiments, the detecting comprises nucleic acid sequencing.

In some embodiments, the nucleic acid sequencing comprises DNA sequencing.

In some embodiments, the subject is suspected of having early-stage Alzheimer's Disease.

In some embodiments, the subject is asymptomatic for the neurodegenerative disease.

In some embodiments, the subject exhibits or reports one or more symptoms of Alzheimer's Disease comprising: impairment in cognition, impairment in memory, impairment in language, or impairment in perception.

In some embodiments, the subject has or is suspected of having Alzheimer's Disease based on an expert evaluation.

In some embodiments, the subject has or is suspected of having Alzheimer's Disease based on a positive Alzheimer's Disease diagnosis from another diagnostic.

In some aspects, the present disclosure provides a method for determining a risk or state of a neurodegenerative disease of the subject comprising: (a) providing a biological sample from a subject; (b) detecting a presence of one or more protein quantitative trait loci (pQTLs) in the biological sample, wherein the one or more pQTLs is associated with a protein level in vivo of one or more proteins comprising at least one of: C9, C8B, F5, CFH, MBL2, F12, KNG1, and SERPINA1; and (c) determining the state of the neurodegenerative disease of the subject based on the presence of the one or more pQTLs in the biological sample.

In some aspects, the present disclosure provides a method for determining a risk or state of a neurodegenerative disease of a subject comprising: (a) detecting a presence of one or more single nucleotide polymorphisms (SNPs) in a biological sample from the subject, wherein the one or more SNPs is associated with a protein level in vivo of one or more proteins that are involved in (i) a complement and coagulation cascade, (ii) a complement system, (iii) a complement system in neuronal development and plasticity, (iv) a Kinin-Kallikrein pathway, or (v) any combination thereof; and (b) determining the risk or state of the neurodegenerative disease of the subject based on the presence of the one or more protein quantitative trait loci in the biological sample.

In some embodiments, the one or more proteins are involved in the complement and coagulation cascade.

In some embodiments, the one or more proteins comprise at least one of: 109821, A2M, ADN, BDKRB1, Bradykinin, C1QA, C1QB, C1QG, C1R, C1S, C2, C3, C3AR1, C4, C5R1, C6, C7, C8G, C9, CD59A, CFH, CFI, CLU, CPB2, CR1, CR2, CRRY, Daf1, Daf2, F10, F12, F13B, F2, F2R, F3, F5, F7, F8, F9, FGB, Fibrin monomer, H2-BF, Hc, KLKB1, KNG1, MASP1, MASP2, MBL1, MCP, PLAT, PLAU, PLAUR, PLG, PROC, PROS1, SERPINA1, SERPINA5, SERPINC1, SERPIND1, SERPINE1, SERPINF2, SERPING1, TFPI, THBD, VWF, or a proteoform thereof.

In some embodiments, the one or more proteins are involved in the complement system.

In some embodiments, the one or more proteins comprise at least one of: Leishmanial protein kinase-1, A2M, ADIPOQ, ADM, ALB, APOA1, ARRB2, C1INH, C1q, C1qRp, C1r, C1s, C2, C3 (H2O), C3, C3a-desArg, C3a, C3aR1, C3bB3b, C3bB3bP, C3bBb, C3bBbP, C3b, C3c, C3d, C3f, C4-A, C4BP, C4a-desArg, C4a, C4b2b, C4b2b3b, C4b, C5, C5L2, C5a-desArg, C5a, C5aR1, C5b, C6, C7, C8, C9, CD16a, CD19, CD23, CD40, CD59, CFB, CFD, CFH, CFHR1, CFHR4, CFI, CFP, CHIPS, CK, CLEC4M, CPB2, CPN, CR1, CR2, CR3, CR4, CRIg, CRP, ChpA, ClfA, ClfB, DAF, DCN, E-LDL, ELANE, Efb, Ehp, FCN1, FCN2, FGA, FGA, FGB, FGB, FGG, FKBP13, FN, FPR1, FX, FXIII, FXIIa, FXIa, FnBPA, FnBPB, GNA15, GNA1870, GNAI2, GNAI3, Gpm1p, Gpm1p, HIV-gp41, HS, HSV-gC, Heparin, IBSP, ICAM1, ICAM2, ITGA2B, ITGA2, ITGB3, KLKB1, LAMA5, LAMB1, LAMC1, LRP2, LTA, LfhA, MASP1, MAp19, MAp44, MBL2, MCP, NS1, OMCI, OPN, OmpK36, PKA, PKC, PLAUR, PLG, PTX3, Por1A, Por1B, PrP, Protein M, Protein-A, Protein-S, RPS19, RgpA, SAP, SCIN, SCP, SELE, SELL, SELP, SELPLG, SFTPA1, SFTPA2, SIC, SPICE, SSL10, SSL11, SSL5, SSL7, Sbi, Sfb, Skp, StcE, TFECP, THBS, TLR2, TXN, VCP, VTN, WAS, beta-Glucan, cC1qR, fMLP, gC1qR, gp350, iC3b, or a proteoform thereof.

In some embodiments, the one or more proteins are involved in the complement system in neuronal development and plasticity.

In some embodiments, the one or more proteins comprise at least one of: ADP, APAF1, ATP10A, ATP11A, ATP11C, ATP8B1, ATP8B2, ATP8B3, ATP, AXL, BAK1, BAX, BID, C1QA, C1QB, C1QC, C1R, C1S, C2, C2a, C2b, C3, C3AR1, C3a, C3b, C3bi, C3c, C3dg, C3f, C4A, C4B, C4BPA, C4BPB, C4a, C4b, C5, C5AR1, C5AR2, C5a, C5b, C6, C7, C8A, C8B, C8G, C9, CAP1, CASP10, CASP3, CASP7, CASP8, CASP9, CD46, CD55, CD59, CFB, CFBa, CFBb, CFD, CFH, CFI, CFP, CLU, COLEC10, COLEC11, COLEC12, CR1, CR2, CRB1, CRB2, CSMD1, CX3CL1, CX3CR1, CYCS, Ca2+, DEDD, DIABLO, DLGAP5, FAS, FASLG, FCN1, FCN2, FCN3, GAS6, H2O, HTRA2, IFNG, ITGAM, ITGAV, ITGAX, ITGB2, ITGB3, LLGL2, MARK1, MARK2, MARK3, MASP1, MASP2, MBL1P, MBL2, MBP, MERTK, MFGE8, MPP5, PARD3, PARD6A, PARD6B, PARD6G, PATJ, PLSCR1, PLSCR3, PLSCR4, PRKCI, PRKCZ, PROS1, SCRIB, SERPING1, SUSD4, TGFB1, TGFB2, TGFB3, TYRO3, VTN, XIAP, tBID, or a proteoform thereof.

In some embodiments, the one or more proteins are involved in the Kinin-Kallikrein pathway.

In some embodiments, the one or more proteins comprise at least one of: BDKRB1, BDKRB2, Bradykinin, CPN1, Coagulation factor XII, KLKB1, Kininogen-1, SERPING1, des-arg (9) bradykinin, or a proteoform thereof.

In some embodiments, the detecting comprises nucleic acid sequencing.

In some embodiments, the subject is suspected of having early-stage Alzheimer's Disease.

In some embodiments, the subject is asymptomatic for the neurodegenerative disease.

In some embodiments, the subject exhibits or reports one or more symptoms of Alzheimer's Disease comprising: impairment in cognition, impairment in memory, impairment in language, or impairment in perception.

In some embodiments, the subject has or is suspected of having Alzheimer's Disease based on an expert evaluation.

In some embodiments, the subject has or is suspected of having Alzheimer's Disease based on a positive Alzheimer's Disease diagnosis from another diagnostic.

In some aspects, the present disclosure provides a kit for use in determining a risk or state of a neurodegenerative disease of the subject comprising: one or more surfaces capable of binding to a biomarker comprising at least one of: E7EUF1, O94812, P02549, P02730, P05019, P05154, P05546, P13497, P16157, P16452, P17936, P24593, P27918, P35858, P41218, Q12797, Q13214, Q13822, Q8NI99, Q96IY4, Q99715, Q9BXN1, Q9H0B8, or a proteoform thereof, wherein binding of the biomarker to the surface is indicative of the state of the neurodegenerative disease of the subject.

In some aspects, the present disclosures also provides a kit for use in determining a risk or state of a neurodegenerative disease of the subject comprising: one or more surfaces capable of binding to a biomarker comprising at least one of: 0A0B4JIU7, A0A0D9SG88; Q5TFM2, A0A3B3ISR2; B4DPQ0; F5H2D0, ASYKK6, A8TX70; A8TX70-2, H3BPZ1, 014960, 014980, 075093, 075900-2, P00748, P00751, P00915, P01042-3, P01892, P02042, P04040, P04180, P04196, P07093-2; P07093-3, P070933. P08833, P09668, P10316, P10321-2, P10643, P11226, P13747, P14625, P16871; P16871-2; P16871-3; P16871-4, P19823, P19827, P21741, P26572, P26927, P28799, P36980, P36980; P36980-2, P40937; P40937-2, P54803, P54803; P54803-3; P54803-4, P78539; P78539-2; P78539-5, Q03591, Q06033; Q06033-2, Q08257-3, Q13131; Q13131-2, Q14213, Q15149-2, Q15149-4, Q15149; Q15149-2; Q15149-4; Q15149-5; Q15149-6; Q15149-7; Q15149-8; Q15149-9, Q15165; Q15165-1; Q15165-3, Q15517, Q16678, Q709C8; Q709C8-3, Q8N474, Q8N6R0; Q8N6R0-1; Q8N6R0-3, Q92896; Q92896-2; Q92896-3, Q93091, Q96PD5, Q96PD5-2, Q9H479, Q9H4F8; Q9H4F8-2, Q9NR34, Q9NUQ9, Q9P035, Q9ULI3, Q9UQN3; Q9UQN3-2, Q9Y263, Q9Y450; Q9Y450-4, or a proteoform thereof, wherein binding of the biomarker to the surface is indicative of the state of the neurodegenerative disease of the subject.

In some embodiments, the surface is capable of specifically binding to the biomarker.

In some embodiments, the surface is capable of non-specifically binding to the biomarker.

In some embodiments, the surface is capable of releasing the biomarker after the binding.

In some embodiments, the kit comprises a plurality of surfaces capable of binding to the biomarker.

In some embodiments, the plurality of surfaces comprises at least 2, 3, 4, or 5 surfaces.

In some embodiments, the plurality of surfaces is capable of binding to a plurality of biomarkers.

In some embodiments, each surface in the plurality of surfaces are capable of binding to a different biomarker in the plurality of biomarkers.

In some embodiments, the one or more surfaces comprise one or more antibodies capable of binding to the biomarker.

In some embodiments, a pair of antibodies in the one or more antibodies are capable of binding to the biomarker, wherein the pair of antibodies comprises complementary single-stranded nucleic acid sequences attached thereto, such that when the pair of antibodies bind to the biomarker, the complementary nucleic acids hybridize to form a double stranded nucleic acid.

In some embodiments, the double stranded nucleic acid is configured to form a binding complex with a polymerase and a plurality of nucleotides, nucleosides, nucleotide analogs, and/or nucleoside analogs to perform an amplification reaction to produce a detectable signal.

In some embodiments, the one or more surfaces comprise one or more aptamers capable of binding to the biomarker.

In some embodiments, the one or more aptamers comprise are coupled to a surface via a cleavable linker.

In some embodiments, the surface is a particle surface.

In some embodiments, the cleavable linker is photocleavable.

In some embodiments, the kit further comprises a macromolecular competitor configured to, in a fluid composition, reduce dissociation of a complex comprising the one or more aptamers and the biomarker.

In some embodiments, the macromolecular competitor is further configured to bind to a biomolecule that is different from the biomarker.

In some embodiments, the macromolecular competitor is a polyanionic macromolecule.

In some aspects, the present disclosure provides a kit for use in determining a risk or state of a neurodegenerative disease of the subject comprising: one or more surfaces capable of binding to a biomarker comprising at least one of: P00451, P00734, P00740, P00742, P00748, P01008, P01023, P01042, P02671, P02675, P02679, P02776, P03951, P03952, P04070, P04275, P05154, P05155, P05160, P05546, P07093, P07204, P07225, P07359, P08709, P10646, P10720, P12259, P13224, P13726, P14770, P24158, P25116, P40197, P42785, Q07021, Q8N6Q3, Q9UNN8, K03901, K01320, K01314, K03902, K01313, K01328, K01323, K01321, K03900, K03899, K03907, K06557, K01344, K03914, K04235, K04236, K03917, K03906, K01300, K03903, K03904, K03905, K01324, K03898, K03915, K03916, K01315, K03909, K03911, K03912, K03913, K03908, K03982, K19821, K01343, K01348, K03985, K03984, K03983, K03910, K01335, K01334, K03990, K03994, K03995, K03996, K03997, K03998, K03999, K04000, K03986, K03987, K03988, K01330, K01331, K03991, K03992, K03993, K01332, K03989, K04009, K19822, K04011, K04012, K06461, K06464, K06462, K04010, K04004, K01333, K04001, K04006, K04007, K04002, K04003, K04008, K17252, K06251, O00255, O00391, O00622, O14672, O14791, O15232, O15240, 043493, O43852, O76024, 076061, 095084, O95633, O95972, P00450, P00734, P00747, P01008, P01009, P01024, P01033, P01034, P01042, P01210, P01344, P02647, P02649, P02652, P02671, P02679, P02751, P02765, P02768, P02771, P02787, P03956, P04070, P04114, P05019, P05060, P05067, P05231, P05546, P06870, P07237, P07288, P07942, P08253, P08311, P08582, P08833, P09382, P09603, P0C0L4, P10451, P11047, P12259, P12644, P13521, P13611, P14314, P14625, P17936, P18065, P19022, P19823, P20151, P20718, P22692, P23327, P24592, P24593, P24821, P34741, P35555, P35858, P51654, P55268, Q02818, Q06481, Q07065, Q08431, Q12841, Q13103, Q13217, Q13219, Q13316, Q13421, Q14393, Q14515, Q14703, Q14766, Q15084, Q15293, Q16270, Q24JP5, Q5JRA6, Q6P988, Q6PCB0, Q6Q788, Q6UX39, Q86UP2, Q8IXL6, Q8N114, Q8N4F0, Q8NBJ4, Q8NBP7, Q8WXD2, Q96AD5, Q96MK3, Q99217, Q9BRK3, Q9BTY2, Q9BU40, Q9BXP8, Q9GZV9, Q9H8M9, Q9HCE9, Q9NP70, Q9NQ76, Q9NRM1, Q9UK55, Q9UKR3, Q9UM21, and a proteoform thereof, wherein the binding of the biomarker to the one or more surfaces are indicative of the state of the neurodegenerative disease of the subject.

In some embodiments, the one or more surfaces are capable of specifically binding to the biomarker.

In some embodiments, the one or more surfaces are capable of non-specifically binding to the biomarker.

In some embodiments, the one or more surfaces are capable of releasing the biomarker after the binding.

In some embodiments, the one or more surfaces comprise a plurality of surfaces capable of binding to the biomarker.

In some embodiments, the plurality of surfaces comprises at least 2, 3, 4, or 5 surfaces.

In some embodiments, the plurality of surfaces is capable of binding to a plurality of biomarkers.

In some embodiments, each surface in the plurality of surfaces are capable of binding to a different biomarker in the plurality of biomarkers.

In some aspects, the present disclosure provides a kit for use in determining a risk or state of a neurodegenerative disease of the subject comprising: one or more antibodies capable of binding to a biomarker comprising at least one of: E7EUF1, O94812, P02549, P02730, P05019, P05154, P05546, P13497, P16157, P16452, P17936, P24593, P27918, P35858, P41218, Q12797, Q13214, Q13822, Q8NI99, Q96IY4, Q99715, Q9BXN1, Q9H0B8, or a proteoform thereof, wherein binding of the biomarker to the one or more antibodies is indicative of the state of the neurodegenerative disease of the subject.

In some aspects, the present disclosure provides a kit for use in determining a risk or state of a neurodegenerative disease of the subject comprising: one or more antibodies capable of binding to a biomarker comprising at least one of: 0A0B4JIU7, A0A0D9SG88; Q5TFM2, A0A3B3ISR2; B4DPQ0; FSH2DO, A5YKK6, A8TX70; ASTX70-2, H3BPZ1, 014960, 014980, 075093, 075900-2, P00748, P00751, P00915, P01042-3, P01892, P02042, P04040, P04180, P04196, P07093-2; P07093-3, P070933, P08833, P09668, P10316, P10321-2, P10643, P11226, P13747, P14625, P16871; P16871-2; P16871-3; P16871-4, P19823, P19827, P21741, P26572, P26927, P28799, P36980, P36980; P36980-2, P40937; P40937-2, P54803, P54803; P54803-3; P54803-4, P78539; P78539-2; P78539-5, Q03591, Q06033; Q06033-2, Q08257-3, Q13131; Q13131-2, Q14213, Q15149-2, Q15149-4, Q15149; Q15149-2; Q15149-4; Q15149-5; Q15149-6; Q15149-7; Q15149-8; Q15149-9, Q15165; Q15165-1; Q15165-3, Q15517, Q16678, Q709C8; Q709C8-3, Q8N474, Q8N6R0; Q8N6R0-1; Q8N6R0-3, Q92896; Q92896-2; Q92896-3, Q93091, Q96PD5, Q96PD5-2, Q9H479, Q9H4F8; Q9H4F8-2, Q9NR34, Q9NUQ9, Q9P035, Q9ULI3, Q9UQN3; Q9UQN3-2, Q9Y263, Q9Y450; Q9Y450-4, or a proteoform thereof, wherein binding of the biomarker to the one or more antibodies is indicative of the state of the neurodegenerative disease of the subject.

In some embodiments, a pair of antibodies in the one or more antibodies are capable of binding to the biomarker, wherein the pair of antibodies comprises complementary single-stranded nucleic acid sequences attached thereto, such that when the pair of antibodies bind to the biomarker, the complementary nucleic acids hybridize to form a double stranded nucleic acid.

In some embodiments, the double stranded nucleic acid is configured to form a binding complex with a polymerase and a plurality of nucleotides, nucleosides, nucleotide analogs, and/or nucleoside analogs to perform an amplification reaction to produce a detectable signal.

In some embodiments, the kit further comprises the polymerase and/or the plurality of nucleotides.

In some aspects, the present disclosure provides a kit for use in determining a risk or state of a neurodegenerative disease of the subject comprising: one or more aptamers capable of binding to a biomarker comprising at least one of: E7EUF1, O94812, P02549, P02730, P05019, P05154, P05546, P13497, P16157, P16452, P17936, P24593, P27918, P35858, P41218, Q12797, Q13214, Q13822, Q8NI99, Q96IY4, Q99715, Q9BXN1, Q9H0B8, or a proteoform thereof, wherein binding of the biomarker to the one or more antibodies is indicative of the state of the neurodegenerative disease of the subject.

In some aspects, the present disclosure provides a kit for use in determining a risk or state of a neurodegenerative disease of the subject comprising: one or more aptamers capable of binding to a biomarker comprising at least one of: 0A0B4JIU7, A0A0D9SG88; Q5TFM2, A0A3B3ISR2; B4DPQ0; F5H2DO, ASYKK6, A8TX70; ASTX70-2, H3BPZ1, O14960, 014980, 075093, 075900-2, P00748, P00751, P00915, P01042-3, P01892, P02042, P04040, P04180, P04196, P07093-2; P07093-3, P070933, P08833, P09668, P10316, P10321-2, P10643, P11226, P13747, P14625, P16871; P16871-2; P16871-3; P16871-4, P19823, P19827, P21741, P26572, P26927, P28799, P36980, P36980; P36980-2, P40937: P40937-2, P54803, P54803; P54803-3; P54803-4, P78539; P78539-2; P78539-5, Q03591, Q06033; Q06033-2, Q08257-3, Q13131; Q13131-2, Q14213, Q15149-2, Q15149-4, Q15149; Q15149-2; Q15149-4; Q15149-5; Q15149-6; Q15149-7; Q15149-8; Q15149-9, Q15165; Q15165-1; Q15165-3, Q15517, Q16678, Q709C8; Q709C8-3, Q8N474, Q8N6R0; Q8N6R0-1; Q8N6R0-3, Q92896; Q92896-2; Q92896-3, Q93091, Q96PD5, Q96PD5-2, Q9H479, Q9H4F8; Q9H4F8-2, Q9NR34, Q9NUQ9, Q9P035, Q9ULI3, Q9UQN3; Q9UQN3-2, Q9Y263, Q9Y450; Q9Y450-4, or a proteoform thereof, wherein binding of the biomarker to the one or more antibodies is indicative of the state of the neurodegenerative disease of the subject.

In some embodiments, the one or more aptamers are coupled to a surface via a cleavable linker.

In some embodiments, the surface is a particle surface.

In some embodiments, the cleavable linker is photocleavable.

In some embodiments, the kit further comprises a macromolecular competitor configured to, in a fluid composition, reduce dissociation of a complex comprising the one or more aptamers and the biomarker.

In some embodiments, the macromolecular competitor is further configured to bind to a biomolecule that is different from the biomarker.

In some embodiments, the macromolecular competitor is a polyanionic macromolecule.

In some embodiments, the one or more aptamers are coupled to a plurality of surfaces.

In some embodiments, the plurality of surfaces comprises at least 2, 3, 4, or 5 surfaces.

In some embodiments, the plurality of surfaces each comprises a different aptamer in the one or more aptamers, wherein each aptamer in the one or more aptamers is capable of binding to a different biomarker.

In some aspects, the present disclosure provides a method for treating a subject afflicted with or at a risk of being afflicted with a neurodegenerative disease, comprising: administering to the subject one or more drugs to modulate a level or an activity of at least one of: E7EUF1, O94812, P02549, P02730, P05019, P05154, P05546, P13497, P16157, P16452, P17936, P24593, P27918, P35858, P41218, Q12797, Q13214, Q13822, Q8NI99, Q96IY4, Q99715, Q9BXN1, Q9H0B8, or a proteoform thereof.

In some embodiments, the biomarker further comprises at least one of: P04114, P30043, P00742, or a proteoform thereof.

In some embodiments, O94812 proteoform is selected from the group consisting of: O94812-2, O94812-3, O94812-5, O94812-6, and O94812-7.

In some embodiments, P02549 is a P02549-2 proteoform.

In some embodiments, P35858 is a P35858-2 proteoform.

In some embodiments, Q13214 is a Q13214-2 proteoform.

In some embodiments, Q13822 is a Q13822-3 proteoform.

In some embodiments, Q9H0B8 is a Q9H0B8-3 proteoform.

In some embodiments, P16157 is a P16157-12 proteoform.

In some embodiments, P16452 is a P16452-2 proteoform.

In some aspects, the present disclosure provides a method for treating a subject afflicted with or at a risk of being afflicted with a neurodegenerative disease, comprising: administering to the subject one or more drugs to modulate a level or an activity of at least one of: GSA-rs1046279, GSA-rs1131364, GSA-rs1165692, GSA-rs6030, rs1001179, rs1046875, rs1046896, rs1061147, rs1061147, rs10737680, rs10801555, rs11003118, rs1106766, rs11085954, rs11613352, rs1165669, rs12067507, rs12148472, rs12493926, rs1329424, rs1329428, rs1410996, rs1520079, rs17198, rs1800450, rs1801020, rs1801689, rs1831282, rs2289702, rs2545801, rs2731672, rs2749534, rs3197999, rs34882957, rs3741414, rs3784539, rs3792366, rs380390, rs395544, rs41268617, rs4524, rs5030062, rs62623707, rs6647, rs6677604, rs6695321, rs68066031, rs68147365, rs698078, rs707998, rs710446, rs7599241, rs8099840, rs880633, rs9820435, and rs9898.

In some aspects, the present disclosure also provides a method for determining a risk or state of a neurodegenerative disease of a subject comprising: (a) detecting a presence of a biomarker in a biological sample from the subject, wherein the biomarker comprises at least one of the biomarkers in Table 11; and (b) determining the risk or state of the neurodegenerative disease of the subject based on the presence of the biomarker in the biological sample.

In some embodiments, the subject has or is suspected of having Alzheimer's Disease based on a positive Alzheimer's Disease diagnosis from another diagnostic.

In some aspects, the present disclosure provides a device comprising a computer-readable medium having computer-executable code encoded therein, the computer-executable code adapted to be executed to implement any one of the methods provided herein.

In some aspects, the present disclosure provides a computer program product comprising a computer-readable medium having computer-executable code encoded therein, the computer-executable code adapted to be executed to implement any one of the methods provided herein.

In some aspects, the present disclosure provides a non-transitory computer-readable storage media encoded with a computer program including instructions executable by one or more processors to implement any one of the methods provided herein.

In some aspects, the present disclosure provides a computer-implemented system comprising: a digital processing device comprising: at least one processor, an operating system configured to perform executable instructions, a memory, and a computer program including instructions executable by the digital processing device to perform any one of the methods provided herein.

In some aspects, the present disclosure provides a method for determining a risk or state of a neurodegenerative disease of a subject comprising: a. detecting a presence of a biomarker in a biological sample from the subject, wherein the biomarker comprises at least one of: P10451, P00748, Q06481, P02748, Q6Q788, P05060, P0C0L4, or a proteoform thereof; and b. determining the risk or state of the neurodegenerative disease of the subject based on the presence of the biomarker in the biological sample.

In some aspects, the present disclosure provides a kit for use in determining a risk or state of a neurodegenerative disease of the subject comprising: one or more surfaces capable of binding to a biomarker comprising at least one of: P10451, P00748, Q06481, P02748, Q6Q788, P05060, P0C0L4, or a proteoform thereof, wherein binding of the biomarker to the surface is indicative of the state of the neurodegenerative disease of the subject.

In some aspects, the present disclosure provides a kit for use in determining a risk or state of a neurodegenerative disease of the subject comprising: one or more antibodies capable of binding to a biomarker comprising at least one of: P10451, P00748, Q06481, P02748, Q6Q788, P05060, P0C0L4, or a proteoform thereof, wherein binding of the biomarker to the one or more antibodies is indicative of the state of the neurodegenerative disease of the subject.

In some aspects, the present disclosure provides a kit for use in determining a risk or state of a neurodegenerative disease of the subject comprising: one or more aptamers capable of binding to a biomarker comprising at least one of: P10451, P00748, Q06481, P02748, Q6Q788, P05060, P0C0L4, or a proteoform thereof, wherein binding of the biomarker to the one or more antibodies is indicative of the state of the neurodegenerative disease of the subject.

In some aspects, the present disclosure provides a method for treating a subject afflicted with or at a risk of being afflicted with a neurodegenerative disease, comprising: administering to the subject one or more drugs to modulate a level or an activity of at least one of: P10451, P00748, Q06481, P02748, Q6Q788, P05060, P0C0L4, or a proteoform thereof.

In some embodiments, the biomarker comprises at least one of: P00748, Q6Q788, P05060, or a proteoform thereof.

In some aspects, the present disclosure provides a method for determining a risk or state of a neurodegenerative disease of a subject comprising: a. detecting a presence of a biomarker in a biological sample from the subject, wherein the biomarker is involved at least one of: degradation of the extracellular matrix; signaling by Platelet-Derived Growth Factor (PDGF); integrin cell surface interactions; regulation of Insulin-like Growth Factor (IGF) transport and uptake by Insulin-like Growth Factor Binding Proteins (IGFBPs); runt-domain transcription factor (RUNX3) which regulates immune response and cell migration; post-translational protein phosphorylation; intrinsic pathway of fibrin clot formation; defective factor XII causing hereditary angioedema; defective SERPING1 causing hereditary angioedema; platelet degranulation; terminal pathway of complement; regulation of Complement cascade; peroxisome proliferator-activated receptor alpha (PPARA) which activates gene expression; assembly of active LPL and LIPC lipase complexes; chylomicron remodeling; initial triggering of complement; and activation of C3 and C5; and b. determining the risk or state of the neurodegenerative disease of the subject based on the presence of the biomarker in the biological sample.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1B show the number of peptides and the number of protein groups identified, respectively, using MSFRAGGER™ data dependent acquisition (DDA), in accordance with some embodiments.

FIGS. 2A-2B show the number of peptides and the number of protein groups identified, respectively, using DIA-NN™ data independent acquisition (DIA), in accordance with some embodiments.

FIGS. 3A-3B show a comparison of the number of peptides and the number of protein groups identified, respectively, using MSFRAGGER™ and DIA-NN™, in accordance with some embodiments.

FIG. 4 shows a correlation between peptide quantification by DIA and peptide quantification by DDA, in accordance with some embodiments.

FIGS. 5A-5B show estimated concentration as a function of concentration rank for the protein groups identified using DDA and DIA, respectively, in accordance with some embodiments.

FIGS. 6A-6B show the number of peptides and the number of protein groups identified, respectively, when semi-tryptic peptides are included in the analysis and when semi-tryptic peptides are not included in the analysis, in accordance with some embodiments.

FIGS. 7A-7B show volcano plots that show biomolecules that are differentially expressed as a function of sex and age, respectively, in accordance with some embodiments.

FIG. 8 shows the number of phosphopeptides identified using FRAGPIPE™ and DDA, in accordance with some embodiments.

FIG. 9 shows a heat map of biomolecules that are differentially expressed between samples, in accordance with some embodiments.

FIGS. 10A-10B show receiver operating characteristic (ROC) curves of a support vector machine for early detection of Alzheimer's disease trained on DDA and DIA proteomic data, respectively, in accordance with some embodiments.

FIGS. 11A-11B show importance ranked features of a support vector machine trained on DDA and DIA proteomic data, respectively, in accordance with some embodiments.

FIGS. 12A-12B show functional annotations of importance ranked features of a support vector machine trained on DDA proteomic data, using the KYOTO ENCYCLOPEDIA OF GENES AND GENOMES™ (KEGG) and the THE COMPREHENSIVE RESOURCE OF MAMMALIAN PROTEIN COMPLEXES™ (CORUM) databases, respectively, in accordance with some embodiments.

FIGS. 12C-12D show functional annotations of importance ranked features of a support vector machine trained on DIA proteomic data, using the KEGG and the REACTOME™ (REAC) databases, respectively, in accordance with some embodiments.

FIG. 13 schematically illustrates a calculation for pQTL association, in accordance with some embodiments.

FIG. 14 shows the number of cis-protein quantitative trait loci (cis-pQTL) identified using DDA and DIA, respectively, in accordance with some embodiments.

FIG. 15 shows functional annotations of proteins regulated by cis-pQTLs using the WIKIPATHWAYS™ (WP) database, in accordance with some embodiments.

FIG. 16 shows proteins regulated by cis-pQTLs identified in association with Alzheimer's Disease, in accordance with some embodiments.

FIG. 17 shows proteins regulated by trans-pQTLs identified in association with Alzheimer's Disease, in accordance with some embodiments.

FIG. 18A shows the number of sentinel single nucleotide polymorphism (SNP) pQTLs identified as cis-expression quantitative trait loci (cis-eQTL) or protein altering variants, in accordance with some embodiments.

FIG. 18B shows the number of sentinel SNP pQTLs identified to be in linkage disequilibrium with cis-eQTL or protein altering variants, in accordance with some embodiments.

FIGS. 19A-19B show minimal number of biological replicates as a function of desired fold change in statistical confidence for identifying pQTLs using DDA and DIA, respectively, in accordance with some embodiments.

### DETAILED DESCRIPTION

Neurodegenerative diseases that humans can be difficult to diagnosis. Often, symptoms of unrelated diseases may overlap, and it is challenging to quantitatively assay various neurodegenerative diseases based on biomarkers. Alzheimer's Disease (AD), as an example, may progress for years or decades before any symptoms become apparent. Currently, diagnosis of AD may involve a multidimensional analysis of a subject and the subject's familial medical history that includes the subject's subjective reports of symptoms, MRIs, lab work, and evaluations by a multitude of medical experts. Even still, AD is associated with a high rate of misdiagnosis (10%-20%). Some of the misdiagnosis may be attributed to different neurodegenerative or psychiatric disorders mistaken for AD.

Furthermore, AD may manifest over long preclinical and prodromal phases, that can lead to slow progression of symptoms including cognitive dysfunction, behavioral abnormalities, and impaired performance of activity of daily living. Symptoms can onset over a long duration of time such that when they are detected, the causal illness may have developed significantly into moderate or severe stages of the disease, with little expectation of amelioration. In some cases, preclinical stages of AD (before any physical symptoms may become apparent) may last for years or for decades

In some aspects, the present disclosure provides biomarkers for diagnosing and/or treating AD. Using the PROTEOGRAPH™ workflow, thousands of proteins across a dynamic range greater than 8 magnitudes were assayed from plasma samples of those afflicted with late-stage AD, early-stage AD, and healthy control subjects. Analysis of the assayed proteins identified a number of proteins and proteoforms thereof, as well as genomic sequences that are correlated with AD, that could serve as diagnostic biomarkers and/or targets for treating AD. Interestingly, biomarkers were easier to identify from individuals with early-stage AD. Without being bound to a particular theory, it is hypothesized that AD becomes more heterogeneous as the disease develops into its later stages, thereby impacting more proteins and making it more difficult to identify relevant signals in the proteins that point to a biomarker. In the converse, it is hypothesized that early-stage Alzheimer's Disease is more homogeneous, thereby allowing identification of relevant signals more easily.

In some embodiments, a subject having or suspected of having early-stage AD may be diagnosed and/or treated based on the biomarkers disclosed herein. In some embodiments, the subject may be symptom free and the diagnosis and/or treatment may be a prophylactic measure. In some embodiments, the subject may exhibit or report symptoms of early AD such as mild impairments in cognition, memory, language, or perception. In some embodiments, the subject may have a positive AD diagnosis based on an expert evaluation (e.g., a medical doctor's evaluation). In some embodiments, the subject may have a positive AD diagnosis from another diagnostic, such that a diagnostic provided herein can corroborate or falsify the positive AD diagnosis. In some cases, the subject has a clinical dimension rating of 1.0 or less, or 0.5 or less. In some cases, the subject is suspected of have AD or being at risk of AD. In some cases, the subject has not been diagnosed with AD.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Proteome Identification and Quantification in Alzheimer's Disease Study

Plasma samples from 200 subjects were obtained for this study. The 200 subject comprised two groups comprising 100 AD and non-AD controls. The two groups each comprised 50 females, 50 males, 36 individuals under the age of 70, and 64 individuals over the age of 70. The plasma samples were interrogated using the PROTEOGRAPH™.

Proteins were quantified by data-dependent acquisition (DDA) and data-independent acquisition (DIA) liquid-chromatography mass-spectrometry (LC-MS) analysis.

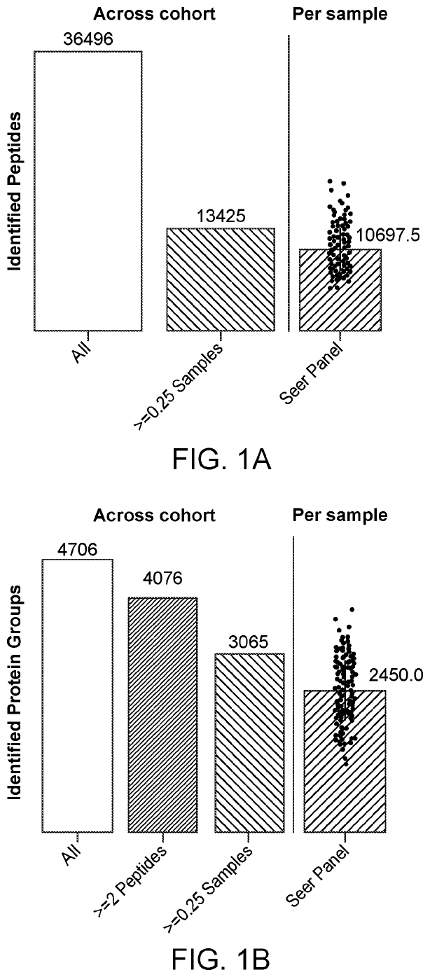

FIGS. 1A-1B show the number of peptides and the number of protein groups identified, respectively, using MSFRAGGER™ data dependent acquisition (DDA), in accordance with some embodiments. DDA detected 36,496 peptides, and 4,706 plasma proteins.

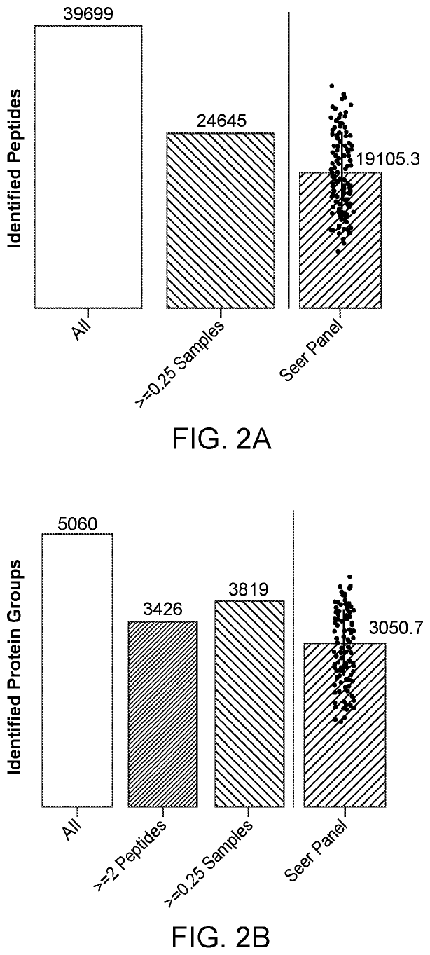

FIGS. 2A-2B show the number of peptides and the number of protein groups identified, respectively, using DIA-NN™ data independent acquisition (DIA), in accordance with some embodiments. DIA detected 39,699 peptides, and 5,060 plasma proteins.

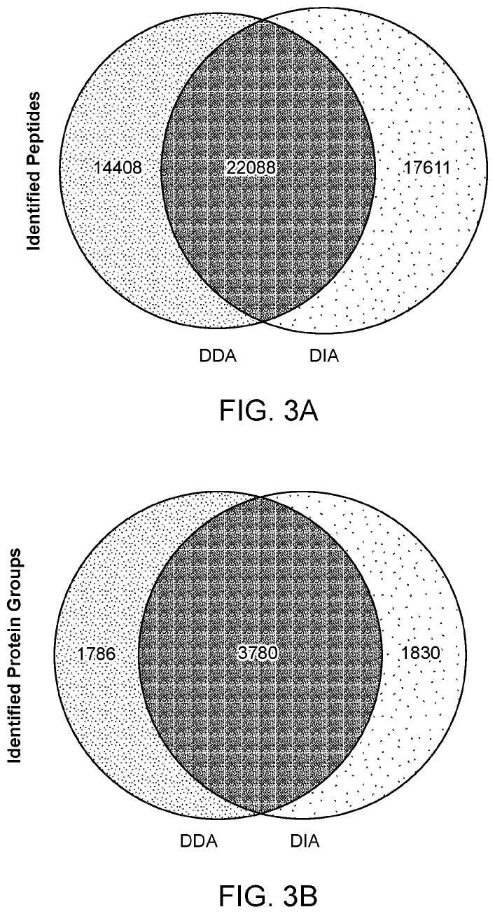

FIGS. 3A-3B show a comparison of the number of peptides and the number of protein groups identified, respectively, using MSFRAGGER™ and DIA-NN™, in accordance with some embodiments. There was a significant difference in the portion of the proteome interrogated between DDA and DIA. Exclusively DDA detected 14408 peptides and 1786 protein groups. Exclusively DIA detected 17611 peptides and 1830 protein groups.

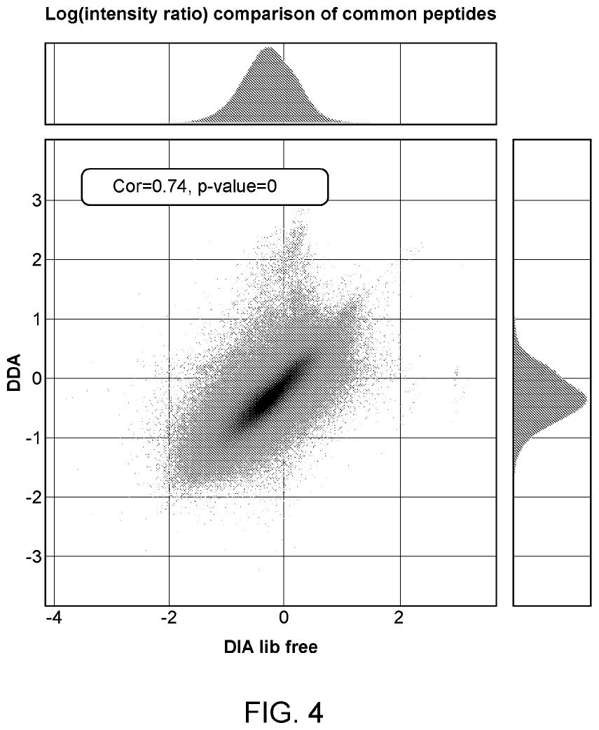

FIG. 4 shows a correlation between peptide quantification by DIA and peptide quantification by DDA, in accordance with some embodiments. Peptide quantification between DIA and DDA is well correlated.

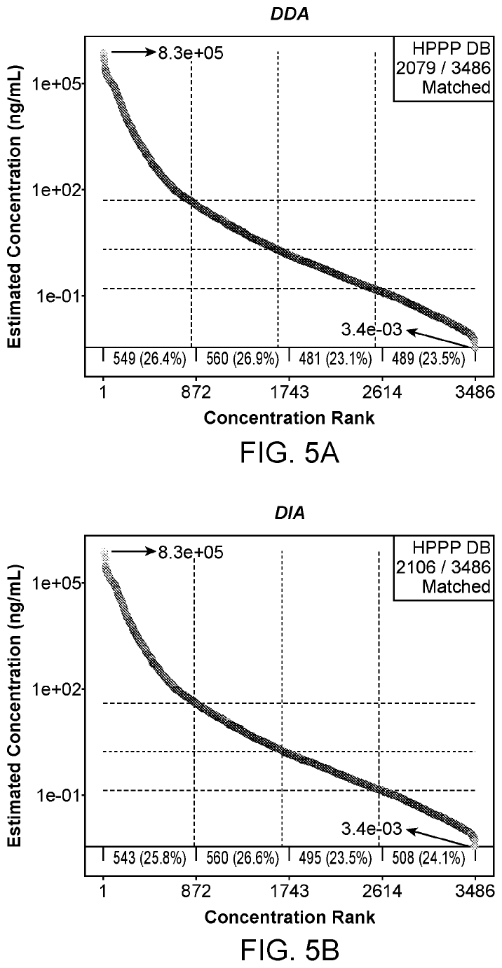

FIGS. 5A-5B show estimated concentration as a function of concentration rank for the protein groups identified using DDA and DIA, respectively, in accordance with some embodiments. The estimated concentration was obtained from a deep fractionated reference database. Proteins groups were identified at a depth of greater than 8 orders of magnitude dynamic range.

Performing a semi-tryptic peptides and phosphorylated peptides search increased the peptide identification by 41%, and the protein group identification rate by 2%, resulting in higher sequence coverage per protein.

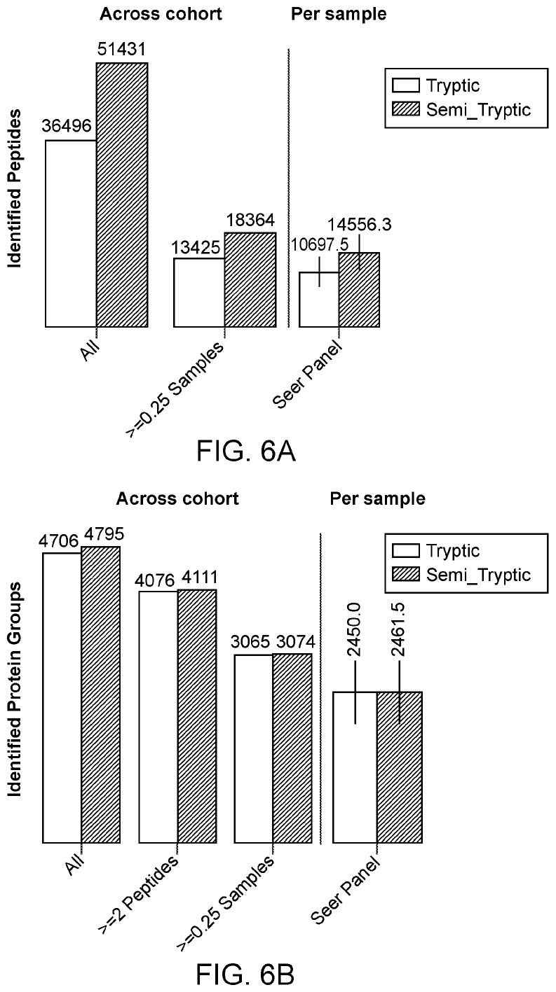

FIGS. 6A-6B show the number of peptides and the number of protein groups identified, respectively, when semi-tryptic peptides are included in the analysis and when semi-tryptic peptides are not included in the analysis, in accordance with some embodiments.

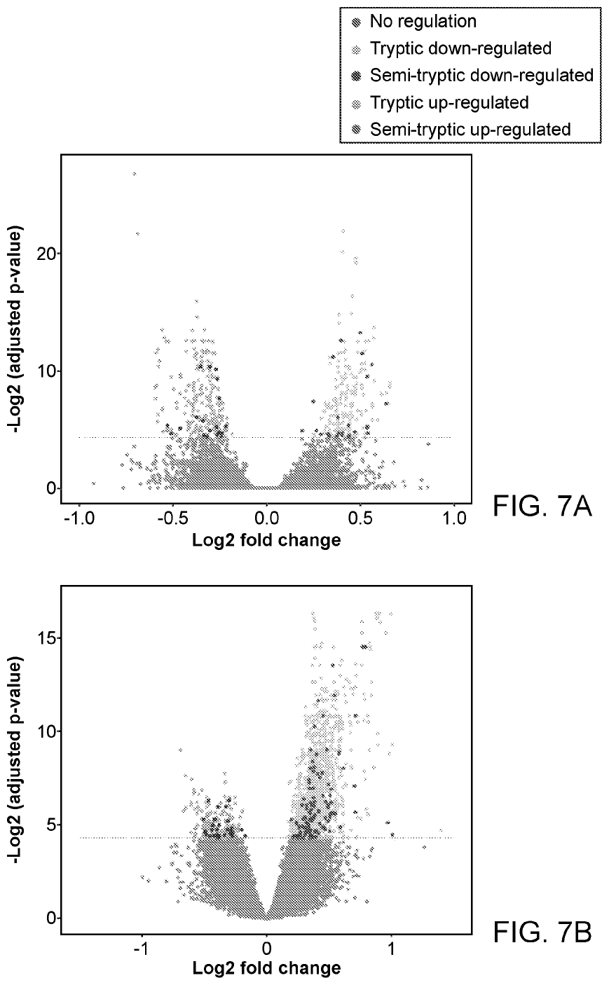

FIGS. 7A-7B show volcano plots that show biomolecules that are differentially expressed as a function of sex and age, respectively, in accordance with some embodiments. Differential expression analysis of peptide intensities from the cohort showed 788 and 608 significantly over-expressed peptides and 132 and 85 significantly under-expressed peptides for age, using DDA and DIA respectively. Differential expression analysis of peptide intensities from the cohort showed 96 and 83 significantly over-expressed peptides, and 93 and 74 significantly under-expressed peptides for sex, using DDA and DIA respectively. Semi-tryptic peptides search provided additional signals for differentiating between sex and age.

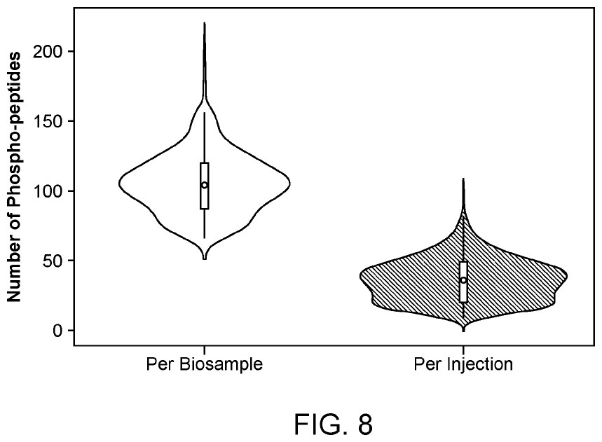

FIG. 8 shows the number of phosphopeptides identified using FRAGPIPE™ and DDA, in accordance with some embodiments. Per biosample, about 105 quantifiable phosphorylated peptides were detected, and about 35 quantifiable phosphorylated peptides per injection. 30-35% more phosphorylated peptides were detected, but were not quantified.

### Example 2: Identification of Protein Groups Biomarkers for Early-Stage Detection of Alzheimer's Disease

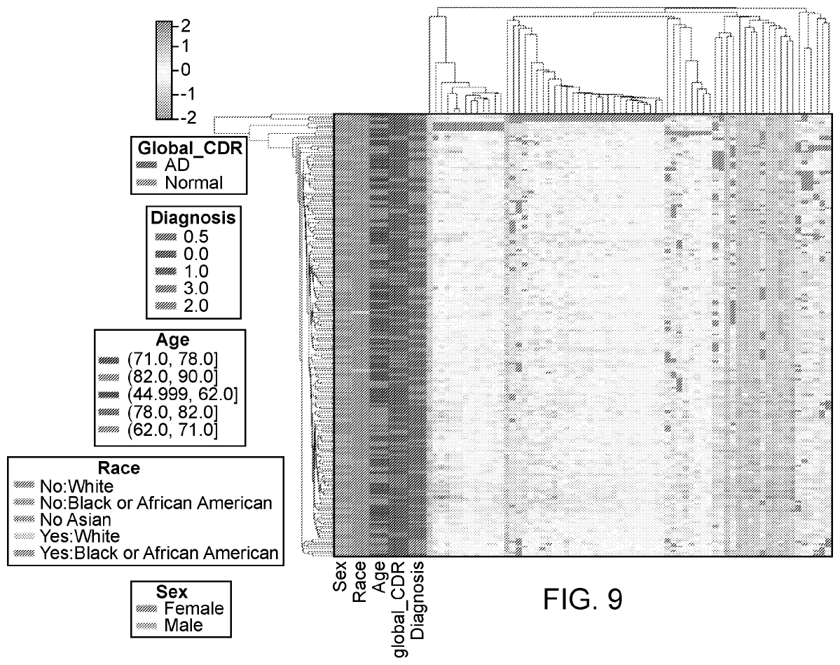

A machine learning classifier was used to select features in the protein groups that could serve as a biomarker for early-stage Alzheimer's disease. The plasma samples from subjects in the earliest stages of Alzheimer's Disease (19 samples, global clinical dementia rating=0.5) were compared against plasma samples from healthy (Normal) subjects that are 78 years of age or older (36 samples). Majority of the subjects with the earliest stages of Alzheimer's Disease were 71 years of age or older. One-sided t-test was performed to detect protein groups enriched for early-stage Alzheimer's Disease. FIG. 9 shows a heat map of biomolecules that are differentially expressed between samples, in accordance with some embodiments.

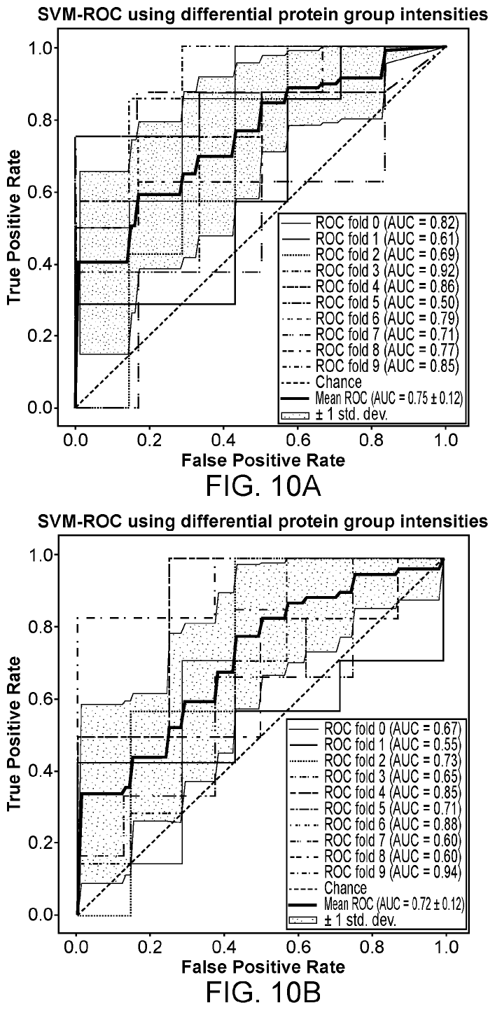

A support vector machine classifier was trained and evaluated on the differential protein groups, and 10-fold cross validation was performed. FIGS. 10A-10B show receiver operating characteristic (ROC) curves of a support vector machine for early detection of Alzheimer's disease trained on DDA and DIA proteomic data, respectively, in accordance with some embodiments. The training yielded a support vector machine classifier for detecting early-stage Alzheimer's Disease and also for identifying heterogenous late-stages of Alzheimer's Disease. The support vector machine classifier showed robustness for differentiating healthy controls from any stage of AD with an AUC-ROC of 0.75 and 0.72, using DDA and DIA data respectively. 126 and 129 protein groups, for DDA and DIA respectively, were identified that are enriched in the early stage of AD. Interestingly, relevant signals were easier to identify in samples from subjects with early-stage Alzheimer's Disease compared to samples from subjects with late-stage Alzheimer's Disease. Without being bound to a particular theory, it is hypothesized that Alzheimer's Disease becomes more heterogeneous as the disease develops into its later stages, thereby impacting more proteins. In the converse, it is hypothesized that early-stage Alzheimer's Disease is more homogeneous, thereby allowing identification of relevant signals more easily.

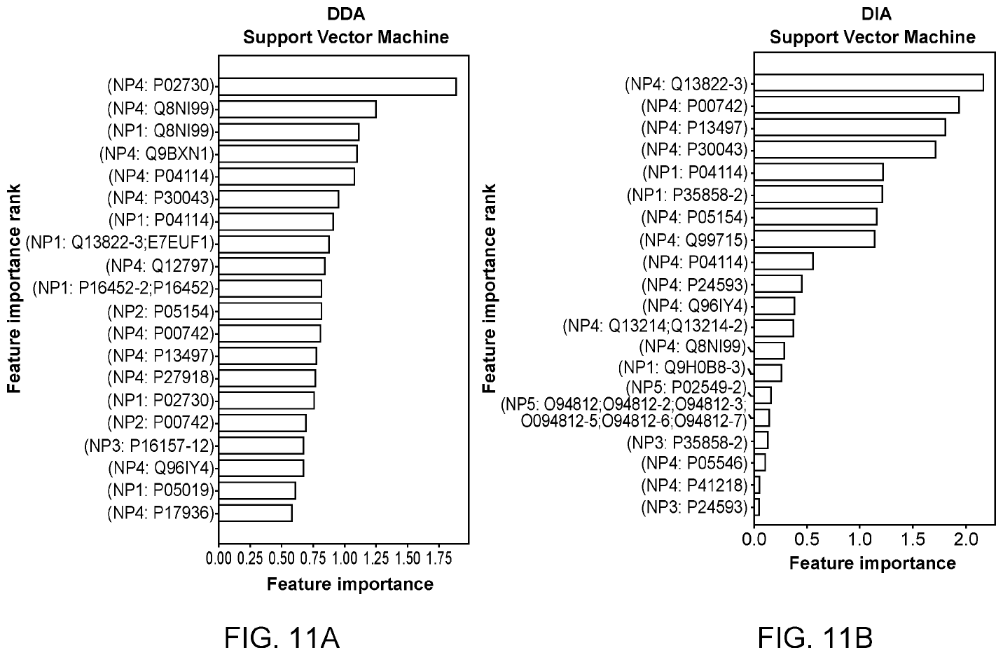

Feature importance ranking was performed on the trained support vector machines to identify biomarkers for early Alzheimer's Disease detection. FIGS. 11A-11B show importance ranked features of a support vector machine trained on DDA and DIA proteomic data, respectively, in accordance with some embodiments.

TABLE 1
Top Protein Group Features from DDA and DIA Data

| DDA (UniProtKB) | DIA (UniProtKB) |
|---|---|
| P00742 | O94812; O94812-2; O94812-3; |
| | O94812-5; O94812-6; O94812-7 |
| P02730 | P00742 |
| P04114 | P02549-2 |
| P05019 | P04114 |
| P05154 | P05154 |
| P13497 | P05546 |
| P16157-12 | P13497 |
| P16452-2; P16452 | P24593 |
| P17936 | P30043 |
| P27918 | P35858-2 |
| P30043 | P41218 |
| Q12797 | Q13214; Q13214-2 |
| Q13822-3; E7EUF1 | Q13822-3 |
| Q8NI99 | Q8NI99 |
| Q96IY4 | Q96IY4 |
| Q9BXN1 | Q99715 |
| | Q9H0B8-3 |

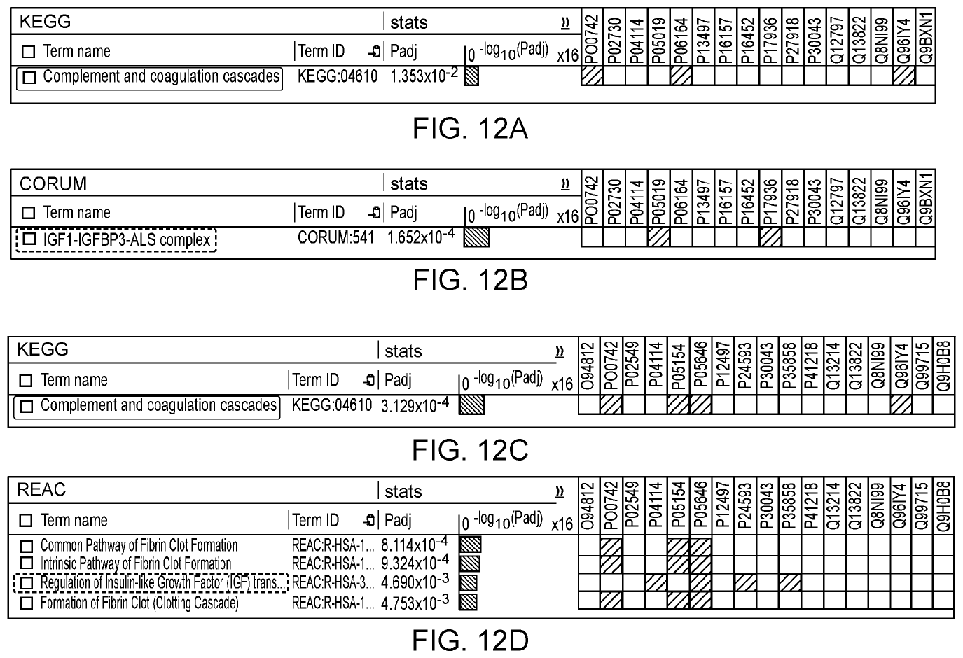

Some of the highly ranked proteins in the feature importance ranking were functionally annotated using various databases. FIGS. 12A-12B show functional annotations of importance ranked features of a support vector machine trained on DDA proteomic data, using the KYOTO ENCYCLOPEDIA OF GENES AND GENOMES™ (KEGG) and the THE COMPREHENSIVE RESOURCE OF MAMMALIAN PROTEIN COMPLEXES™ (CORUM) databases, respectively, in accordance with some embodiments. FIGS. 12C-12D show functional annotations of importance ranked features of a support vector machine trained on DIA proteomic data, using the KEGG and the REACTOME™ (REAC) databases, respectively, in accordance with some embodiments.

TABLE 2
Sequences of Top Protein Group
Features and Isoforms from DDA Data

| | Iso- | SEQ | |
|---|---|---|---|
| | form | ID | |
| UniProtKB | No. | NO. | FASTA SEQUENCE |
| P00742 | 1 | 1 | MGRPLHLVLLSASLAGLLLLGESLFIRREQ |
| | | | ANNILARVTRANSFLEEMKKGHLERECMEE |
| | | | TCSYEEAREVFEDSDKTNEFWNKYKDGDQC |
| | | | ETSPCQNQGKCKDGLGEYTCTCLEGFEGKN |
| | | | CELFTRKLCSLDNGDCDQFCHEEQNSVVCS |
| | | | CARGYTLADNGKACIPTGPYPCGKQTLERR |
| | | | KRSVAQATSSSGEAPDSITWKPYDAADLDP |
| | | | TENPFDLLDFNQTQPERGDNNLTRIVGGQE |
| | | | CKDGECPWQALLINEENEGFCGGTILSEFY |
| | | | ILTAAHCLYQAKRFKVRVGDRNTEQEEGGE |
| | | | AVHEVEVVIKHNRFTKETYDFDIAVLRLKT |
| | | | PITFRMNVAPACLPERDWAESTLMTQKTGI |
| | | | VSGFGRTHEKGRQSTRLKMLEVPYVDRNSC |
| | | | KLSSSFIITQNMFCAGYDTKQEDACQGDSG |
| | | | GPHVTRFKDTYFVTGIVSWGEGCARKGKYG |
| | | | IYTKVTAFLKWIDRSMKTRGLPKAKSHAPE |
| | | | VITSSPLK |
| P02730 | 1 | 2 | MEELQDDYEDMMEENLEQEEYEDPDIPESQ |
| | | | MEEPAAHDTEATATDYHTTSHPGTHKVYVE |
| | | | LQELVMDEKNQELRWMEAARWVQLEENLGE |
| | | | NGAWGRPHLSHLTFWSLLELRRVFTKGTVL |
| | | | LDLQETSLAGVANQLLDRFIFEDQIRPQDR |
| | | | EELLRALLLKHSHAGELEALGGVKPAVLTR |
| | | | SGDPSQPLLPQHSSLETQLFCEQGDGGTEG |
| | | | HSPSGILEKIPPDSEATLVLVGRADFLEQP |
| | | | VLGFVRLQEAAELEAVELPVPIRFLFVLLG |
| | | | PEAPHIDYTQLGRAAATLMSERVFRIDAYM |
| | | | AQSRGELLHSLEGFLDCSLVLPPTDAPSEQ |
| | | | ALLSLVPVQRELLRRRYQSSPAKPDSSFYK |
| | | | GLDLNGGPDDPLQQTGQLFGGLVRDIRRRY |
| | | | PYYLSDITDAFSPQVLAAVIFIYFAALSPA |
| | | | ITFGGLLGEKTRNQMGVSELLISTAVQGIL |
| | | | FALLGAQPLLVVGFSGPLLVFEEAFFSFCE |
| | | | TNGLEYIVGRVWIGFWLILLVVLVVAFEGS |
| | | | FLVRFISRYTQEIFSFLISLIFIYETFSKL |
| | | | IKIFQDHPLQKTYNYNVLMVPKPQGPLPNT |
| | | | ALLSLVLMAGTFFFAMMLRKFKNSSYFPGK |
| | | | LRRVIGDFGVPISILIMVLVDFFIQDTYTQ |
| | | | KLSVPDGFKVSNSSARGWVIHPLGLRSEFP |
| | | | IWMMFASALPALLVFILIFLESQITTLIVS |
| | | | KPERKMVKGSGFHLDLLLVVGMGGVAALFG |
| | | | MPWLSATTVRSVTHANALTVMGKASTPGAA |
| | | | AQIQEVKEQRISGLLVAVLVGLSILMEPIL |
| | | | SRIPLAVLFGIFLYMGVTSLSGIQLFDRIL |
| | | | LLFKPPKYHPDVPYVKRVKTWRMHLFTGIQ |
| | | | IICLAVLWVVKSTPASLALPFVLILTVPLR |
| | | | RVLLPLIFRNVELQCLDADDAKATFDEEEG |
| | | | RDEYDEVAMPV |
| P02730 | 2 | 3 | MDEKNQELRWMEAARWVQLEENLGENGAWG |
| | | | RPHLSHLTFWSLLELRRVFTKGTVLLDLQE |
| | | | TSLAGVANQLLDRFIFEDQIRPQDREELLR |
| | | | ALLLKHSHAGELEALGGVKPAVLTRSGDPS |
| | | | QPLLPQHSSLETQLFCEQGDGGTEGHSPSG |
| | | | ILEKIPPDSEATLVLVGRADFLEQPVLGFV |
| | | | RLQEAAELEAVELPVPIRFLFVLLGPEAPH |
| | | | IDYTQLGRAAATLMSERVFRIDAYMAQSRG |
| | | | ELLHSLEGFLDCSLVLPPTDAPSEQALLSL |
| | | | VPVQRELLRRRYQSSPAKPDSSFYKGLDLN |
| | | | GGPDDPLQQTGQLFGGLVRDIRRRYPYYLS |
| | | | DITDAFSPQVLAAVIFIYFAALSPAITFGG |
| | | | LLGEKTRNQMGVSELLISTAVQGILFALLG |
| | | | AQPLLVVGFSGPLLVFEEAFFSFCETNGLE |
| | | | YIVGRVWIGFWLILLVVLVVAFEGSFLVRF |
| | | | ISRYTQEIFSFLISLIFIYETFSKLIKIFQ |
| | | | DHPLQKTYNYNVLMVPKPQGPLPNTALLSL |
| | | | VLMAGTFFFAMMLRKFKNSSYFPGKLRRVI |
| | | | GDFGVPISILIMVLVDFFIQDTYTQKLSVP |
| | | | DGFKVSNSSARGWVIHPLGLRSEFPIWMMF |
| | | | ASALPALLVFILIFLESQITTLIVSKPERK |
| | | | MVKGSGFHLDLLLVVGMGGVAALFGMPWLS |
| | | | ATTVRSVTHANALTVMGKASTPGAAAQIQE |
| | | | VKEQRISGLLVAVLVGLSILMEPILSRIPL |
| | | | AVLFGIFLYMGVTSLSGIQLFDRILLLFKP |
| | | | PKYHPDVPYVKRVKTWRMHLFTGIQIICLA |
| | | | VLWVVKSTPASLALPFVLILTVPLRRVLLP |
| | | | LIFRNVELQCLDADDAKATFDEEEGRDEYD |
| | | | EVAMPV |
| P04114 | 1 | 4 | MDPPRPALLALLALPALLLLLLAGARAEEE |
| | | | MLENVSLVCPKDATRFKHLRKYTYNYEAES |
| | | | SSGVPGTADSRSATRINCKVELEVPQLCSF |
| | | | ILKTSQCTLKEVYGFNPEGKALLKKTKNSE |
| | | | EFAAAMSRYELKLAIPEGKQVFLYPEKDEP |
| | | | TYILNIKRGIISALLVPPETEEAKQVLFLD |
| | | | TVYGNCSTHFTVKTRKGNVATEISTERDLG |
| | | | QCDRFKPIRTGISPLALIKGMTRPLSTLIS |
| | | | SSQSCQYTLDAKRKHVAEAICKEQHLFLPF |
| | | | SYKNKYGMVAQVTQTLKLEDTPKINSRFFG |
| | | | EGTKKMGLAFESTKSTSPPKQAEAVLKTLQ |
| | | | ELKKLTISEQNIQRANLFNKLVTELRGLSD |
| | | | EAVTSLLPQLIEVSSPITLQALVQCGQPQC |
| | | | STHILQWLKRVHANPLLIDVVTYLVALIPE |
| | | | PSAQQLREIFNMARDQRSRATLYALSHAVN |
| | | | NYHKTNPTGTQELLDIANYLMEQIQDDCTG |
| | | | DEDYTYLILRVIGNMGQTMEQLTPELKSSI |
| | | | LKCVQSTKPSLMIQKAAIQALRKMEPKDKD |
| | | | QEVLLQTFLDDASPGDKRLAAYLMLMRSPS |
| | | | QADINKIVQILPWEQNEQVKNFVASHIANI |
| | | | LNSEELDIQDLKKLVKEALKESQLPTVMDF |
| | | | RKFSRNYQLYKSVSLPSLDPASAKIEGNLI |
| | | | FDPNNYLPKESMLKTTLTAFGFASADLIEI |
| | | | GLEGKGFEPTLEALFGKQGFFPDSVNKALY |
| | | | WVNGQVPDGVSKVLVDHFGYTKDDKHEQDM |
| | | | VNGIMLSVEKLIKDLKSKEVPEARAYLRIL |
| | | | GEELGFASLHDLQLLGKLLLMGARTLQGIP |
| | | | QMIGEVIRKGSKNDFFLHYIFMENAFELPT |
| | | | GAGLQLQISSSGVIAPGAKAGVKLEVANMQ |
| | | | AELVAKPSVSVEFVTNMGIIIPDFARSGVQ |
| | | | MNTNFFHESGLEAHVALKAGKLKFIIPSPK |
| | | | RPVKLLSGGNTLHLVSTTKTEVIPPLIENR |
| | | | QSWSVCKQVFPGLNYCTSGAYSNASSTDSA |
| | | | SYYPLTGDTRLELELRPTGEIEQYSVSATY |
| | | | ELQREDRALVDTLKFVTQAEGAKQTEATMT |
| | | | FKYNRQSMTLSSEVQIPDFDVDLGTILRVN |
| | | | DESTEGKTSYRLTLDIQNKKITEVALMGHL |
| | | | SCDTKEERKIKGVISIPRLQAEARSEILAH |
| | | | WSPAKLLLQMDSSATAYGSTVSKRVAWHYD |
| | | | EEKIEFEWNTGTNVDTKKMTSNFPVDLSDY |
| | | | PKSLHMYANRLLDHRVPQTDMTFRHVGSKL |
| | | | IVAMSSWLQKASGSLPYTQTLQDHLNSLKE |
| | | | FNLQNMGLPDFHIPENLFLKSDGRVKYTLN |
| | | | KNSLKIEIPLPFGGKSSRDLKMLETVRTPA |
| | | | LHFKSVGFHLPSREFQVPTFTIPKLYQLQV |
| | | | PLLGVLDLSTNVYSNLYNWSASYSGGNTST |
| | | | DHFSLRARYHMKADSVVDLLSYNVQGSGET |
| | | | TYDHKNTFTLSYDGSLRHKFLDSNIKFSHV |
| | | | EKLGNNPVSKGLLIFDASSSWGPQMSASVH |
| | | | LDSKKKQHLFVKEVKIDGQFRVSSFYAKGT |
| | | | YGLSCQRDPNTGRLNGESNLRFNSSYLQGT |
| | | | NQITGRYEDGTLSLTSTSDLQSGIIKNTAS |
| | | | LKYENYELTLKSDTNGKYKNFATSNKMDMT |
| | | | FSKQNALLRSEYQADYESLRFFSLLSGSLN |
| | | | SHGLELNADILGTDKINSGAHKATLRIGQD |
| | | | GISTSATTNLKCSLLVLENELNAELGLSGA |
| | | | SMKLTTNGRFREHNAKFSLDGKAALTELSL |
| | | | GSAYQAMILGVDSKNIFNFKVSQEGLKLSN |
| | | | DMMGSYAEMKFDHTNSLNIAGLSLDFSSKL |
| | | | DNIYSSDKFYKQTVNLQLQPYSLVTTLNSD |
| | | | LKYNALDLTNNGKLRLEPLKLHVAGNLKGA |
| | | | YQNNEIKHIYAISSAALSASYKADTVAKVQ |
| | | | GVEFSHRLNTDIAGLASAIDMSTNYNSDSL |
| | | | HFSNVFRSVMAPFTMTIDAHTNGNGKLALW |
| | | | GEHTGQLYSKFLLKAEPLAFTFSHDYKGST |
| | | | SHHLVSRKSISAALEHKVSALLTPAEQTGT |
| | | | WKLKTQFNNNEYSQDLDAYNTKDKIGVELT |
| | | | GRTLADLTLLDSPIKVPLLLSEPINIIDAL |
| | | | EMRDAVEKPQEFTIVAFVKYDKNQDVHSIN |
| | | | LPFFETLQEYFERNRQTIIVVLENVQRNLK |
| | | | HINIDQFVRKYRAALGKLPQQANDYLNSFN |
| | | | WERQVSHAKEKLTALTKKYRITENDIQIAL |
| | | | DDAKINFNEKLSQLQTYMIQFDQYIKDSYD |
| | | | LHDLKIAIANIIDEIIEKLKSLDEHYHIRV |
| | | | NLVKTIHDLHLFIENIDENKSGSSTASWIQ |
| | | | NVDTKYQIRIQIQEKLQQLKRHIQNIDIQH |
| | | | LAGKLKQHIEAIDVRVLLDQLGTTISFERI |
| | | | NDILEHVKHFVINLIGDFEVAEKINAFRAK |
| | | | VHELIERYEVDQQIQVLMDKLVELAHQYKL |
| | | | KETIQKLSNVLQQVKIKDYFEKLVGFIDDA |
| | | | VKKLNELSFKTFIEDVNKFLDMLIKKLKSF |
| | | | DYHQFVDETNDKIREVTQRLNGEIQALELP |
| | | | QKAEALKLFLEETKATVAVYLESLQDTKIT |
| | | | LIINWLQEALSSASLAHMKAKFRETLEDTR |
| | | | DRMYQMDIQQELQRYLSLVGQVYSTLVTYI |
| | | | SDWWTLAAKNLTDFAEQYSIQDWAKRMKAL |
| | | | VEQGFTVPEIKTILGTMPAFEVSLQALQKA |
| | | | TFQTPDFIVPLTDLRIPSVQINFKDLKNIK |
| | | | IPSRFSTPEFTILNTFHIPSFTIDFVEMKV |
| | | | KIIRTIDQMLNSELQWPVPDIYLRDLKVED |
| | | | IPLARITLPDFRLPEIAIPEFIIPTLNLND |
| | | | FQVPDLHIPEFQLPHISHTIEVPTFGKLYS |
| | | | ILKIQSPLFTLDANADIGNGTTSANEAGIA |
| | | | ASITAKGESKLEVLNFDFQANAQLSNPKIN |
| | | | PLALKESVKFSSKYLRTEHGSEMLFFGNAI |
| | | | EGKSNTVASLHTEKNTLELSNGVIVKINNQ |
| | | | LTLDSNTKYFHKLNIPKLDFSSQADLRNEI |
| | | | KTLLKAGHIAWTSSGKGSWKWACPRFSDEG |
| | | | THESQISFTIEGPLTSFGLSNKINSKHLRV |
| | | | NQNLVYESGSLNFSKLEIQSQVDSQHVGHS |
| | | | VLTAKGMALFGEGKAEFTGRHDAHLNGKVI |
| | | | GTLKNSLFFSAQPFEITASTNNEGNLKVRF |
| | | | PLRLTGKIDFLNNYALFLSPSAQQASWQVS |
| | | | ARFNQYKYNQNFSAGNNENIMEAHVGINGE |
| | | | ANLDFLNIPLTIPEMRLPYTIITTPPLKDF |
| | | | SLWEKTGLKEFLKTTKQSFDLSVKAQYKKN |
| | | | KHRHSITNPLAVLCEFISQSIKSFDRHFEK |
| | | | NRNNALDFVTKSYNETKIKFDKYKAEKSHD |
| | | | ELPRTFQIPGYTVPVVNVEVSPFTIEMSAF |
| | | | GYVFPKAVSMPSFSILGSDVRVPSYTLILP |
| | | | SLELPVLHVPRNLKLSLPDFKELCTISHIF |
| | | | IPAMGNITYDFSFKSSVITLNTNAELFNQS |
| | | | DIVAHLLSSSSSVIDALQYKLEGTTRLTRK |
| | | | RGLKLATALSLSNKFVEGSHNSTVSLTTKN |
| | | | MEVSVATTTKAQIPILRMNFKQELNGNTKS |
| | | | KPTVSSSMEFKYDFNSSMLYSTAKGAVDHK |
| | | | LSLESLTSYFSIESSTKGDVKGSVLSREYS |
| | | | GTIASEANTYLNSKSTRSSVKLQGTSKIDD |
| | | | IWNLEVKENFAGEATLQRIYSLWEHSTKNH |
| | | | LQLEGLFFTNGEHTSKATLELSPWQMSALV |
| | | | QVHASQPSSFHDFPDLGQEVALNANTKNQK |
| | | | IRWKNEVRIHSGSFQSQVELSNDQEKAHLD |
| | | | IAGSLEGHLRFLKNIILPVYDKSLWDFLKL |
| | | | DVTTSIGRRQHLRVSTAFVYTKNPNGYSFS |
| | | | IPVKVLADKFIIPGLKLNDLNSVLVMPTFH |
| | | | VPFTDLQVPSCKLDFREIQIYKKLRTSSFA |
| | | | LNLPTLPEVKFPEVDVLTKYSQPEDSLIPF |
| | | | FEITVPESQLTVSQFTLPKSVSDGIAALDL |
| | | | NAVANKIADFELPTIIVPEQTIEIPSIKFS |
| | | | VPAGIVIPSFQALTARFEVDSPVYNATWSA |
| | | | SLKNKADYVETVLDSTCSSTVQFLEYELNV |
| | | | LGTHKIEDGTLASKTKGTFAHRDFSAEYEE |
| | | | DGKYEGLQEWEGKAHLNIKSPAFTDLHLRY |
| | | | QKDKKGISTSAASPAVGTVGMDMDEDDDFS |
| | | | KWNFYYSPQSSPDKKLTIFKTELRVRESDE |
| | | | ETQIKVNWEEEAASGLLTSLKDNVPKATGV |
| | | | LYDYVNKYHWEHTGLTLREVSSKLRRNLQN |
| | | | NAEWVYQGAIRQIDDIDVRFQKAASGTTGT |
| | | | YQEWKDKAQNLYQELLTQEGQASFQGLKDN |
| | | | VFDGLVRVTQEFHMKVKHLIDSLIDFLNFP |
| | | | RFQFPGKPGIYTREELCTMFIREVGTVLSQ |
| | | | VYSKVHNGSEILFSYFQDLVITLPFELRKH |
| | | | KLIDVISMYRELLKDLSKEAQEVFKAIQSL |
| | | | KTTEVLRNLQDLLQFIFQLIEDNIKQLKEM |
| | | | KFTYLINYIQDEINTIFSDYIPYVFKLLKE |
| | | | NLCLNLHKFNEFIQNELQEASQELQQIHQY |
| | | | IMALREEYFDPSIVGWTVKYYELEEKIVSL |
| | | | IKNLLVALKDFHSEYIVSASNFTSQLSSQV |
| | | | EQFLHRNIQEYLSILTDPDGKGKEKIAELS |
| | | | ATAQEIIKSQAIATKKIISDYHQQFRYKLQ |
| | | | DFSDQLSDYYEKFIAESKRLIDLSIQNYHT |
| | | | FLIYITELLKKLQSTTVMNPYMKLAPGELT |
| | | | IIL |
| P05019 | 1 | 5 | MGKISSLPTQLFKCCFCDFLKVKMHTMSSS |
| | | | HLFYLALCLLTFTSSATAGPETLCGAELVD |
| | | | ALQFVCGDRGFYFNKPTGYGSSSRRAPQTG |
| | | | IVDECCFRSCDLRRLEMYCAPLKPAKSARS |
| | | | VRAQRHTDMPKTQKYQPPSTNKNTKSQRRK |
| | | | GWPKTHPGGEQKEGTEASLQIRGKKKEQRR |
| | | | EIGSRNAECRGKKGK |
| P05019 | 2 | 6 | MGKISSLPTQLFKCCFCDFLKVKMHTMSSS |
| | | | HLFYLALCLLTFTSSATAGPETLCGAELVD |
| | | | ALQFVCGDRGFYFNKPTGYGSSSRRAPQTG |
| | | | IVDECCFRSCDLRRLEMYCAPLKPAKSARS |
| | | | VRAQRHTDMPKTQKEVHLKNASRGSAGNKN |
| | | | YRM |
| P05019 | 3 | 7 | MITPTVKMHTMSSSHLFYLALCLLTFTSSA |
| | | | TAGPETLCGAELVDALQFVCGDRGFYFNKP |
| | | | TGYGSSSRRAPQTGIVDECCFRSCDLRRLE |
| | | | MYCAPLKPAKSARSVRAQRHTDMPKTQKEV |
| | | | HLKNASRGSAGNKNYRM |
| P05019 | 4 | 8 | MGKISSLPTQLFKCCFCDFLKVKMHTMSSS |
| | | | HLFYLALCLLTFTSSATAGPETLCGAELVD |
| | | | ALQFVCGDRGFYFNKPTGYGSSSRRAPQTG |
| | | | IVDECCFRSCDLRRLEMYCAPLKPAKSARS |
| | | | VRAQRHTDMPKTQKYQPPSTNKNTKSQRRK |
| | | | GSTFEERK |
| P05154 | 1 | 9 | MQLFLLLCLVLLSPQGASLHRHHPREMKKR |
| | | | VEDLHVGATVAPSSRRDFTFDLYRALASAA |
| | | | PSQSIFFSPVSISMSLAMLSLGAGSSTKMQ |
| | | | ILEGLGLNLQKSSEKELHRGFQQLLQELNQ |
| | | | PRDGFQLSLGNALFTDLVVDLQDTFVSAMK |
| | | | TLYLADTFPTNFRDSAGAMKQINDYVAKQT |
| | | | KGKIVDLLKNLDSNAVVIMVNYIFFKAKWE |
| | | | TSFNHKGTQEQDFYVTSETVVRVPMMSRED |
| | | | QYHYLLDRNLSCRVVGVPYQGNATALFILP |
| | | | SEGKMQQVENGLSEKTLRKWLKMFKKRQLE |
| | | | LYLPKFSIEGSYQLEKVLPSLGISNVFTSH |
| | | | ADLSGISNHSNIQVSEMVHKAVVEVDESGT |
| | | | RAAAATGTIFTFRSARLNSQRLVFNRPFLM |
| | | | FIVDNNILFLGKVNRP |
| P13497 | 1 | 10 | MPGVARLPLLLGLLLLPRPGRPLDLADYTY |
| | | | DLAEEDDSEPLNYKDPCKAAAFLGDIALDE |
| | | | EDLRAFQVQQAVDLRRHTARKSSIKAAVPG |
| | | | NTSTPSCQSTNGQPQRGACGRWRGRSRSRR |
| | | | AATSRPERVWPDGVIPFVIGGNFTGSQRAV |
| | | | FRQAMRHWEKHTCVTFLERTDEDSYIVFTY |
| | | | RPCGCCSYVGRRGGGPQAISIGKNCDKFGI |
| | | | VVHELGHVVGFWHEHTRPDRDRHVSIVREN |
| | | | IQPGQEYNFLKMEPQEVESLGETYDFDSIM |
| | | | HYARNTFSRGIFLDTIVPKYEVNGVKPPIG |
| | | | QRTRLSKGDIAQARKLYKCPACGETLQDST |
| | | | GNFSSPEYPNGYSAHMHCVWRISVTPGEKI |
| | | | ILNFTSLDLYRSRLCWYDYVEVRDGFWRKA |
| | | | PLRGRFCGSKLPEPIVSTDSRLWVEFRSSS |
| | | | NWVGKGFFAVYEAICGGDVKKDYGHIQSPN |
| | | | YPDDYRPSKVCIWRIQVSEGFHVGLTFQSF |
| | | | EIERHDSCAYDYLEVRDGHSESSTLIGRYC |
| | | | GYEKPDDIKSTSSRLWLKFVSDGSINKAGF |
| | | | AVNFFKEVDECSRPNRGGCEQRCLNTLGSY |
| | | | KCSCDPGYELAPDKRRCEAACGGFLTKLNG |
| | | | SITSPGWPKEYPPNKNCIWQLVAPTQYRIS |
| | | | LQFDFFETEGNDVCKYDFVEVRSGLTADSK |
| | | | LHGKFCGSEKPEVITSQYNNMRVEFKSDNT |
| | | | VSKKGFKAHFFSDKDECSKDNGGCQQDCVN |
| | | | TFGSYECQCRSGFVLHDNKHDCKEAGCDHK |
| | | | VTSTSGTITSPNWPDKYPSKKECTWAISST |
| | | | PGHRVKLTFMEMDIESQPECAYDHLEVFDG |
| | | | RDAKAPVLGRFCGSKKPEPVLATGSRMFLR |
| | | | FYSDNSVQRKGFQASHATECGGQVRADVKT |
| | | | KDLYSHAQFGDNNYPGGVDCEWVIVAEEGY |
| | | | GVELVFQTFEVEEETDCGYDYMELFDGYDS |
| | | | TAPRLGRYCGSGPPEEVYSAGDSVLVKFHS |
| | | | DDTITKKGFHLRYTSTKFQDTLHSRK |
| P13497 | 2 | 11 | MPGVARLPLLLGLLLLPRPGRPLDLADYTY |
| | | | DLAEEDDSEPLNYKDPCKAAAFLGDIALDE |
| | | | EDLRAFQVQQAVDLRRHTARKSSIKAAVPG |
| | | | NTSTPSCQSTNGQPQRGACGRWRGRSRSRR |
| | | | AATSRPERVWPDGVIPFVIGGNFTGSQRAV |
| | | | FRQAMRHWEKHTCVTFLERTDEDSYIVFTY |
| | | | RPCGCCSYVGRRGGGPQAISIGKNCDKFGI |
| | | | VVHELGHVVGFWHEHTRPDRDRHVSIVREN |
| | | | IQPGQEYNFLKMEPQEVESLGETYDFDSIM |
| | | | HYARNTFSRGIFLDTIVPKYEVNGVKPPIG |
| | | | QRTRLSKGDIAQARKLYKCPACGETLQDST |
| | | | GNFSSPEYPNGYSAHMHCVWRISVTPGEKI |
| | | | ILNFTSLDLYRSRLCWYDYVEVRDGFWRKA |
| | | | PLRGRFCGSKLPEPIVSTDSRLWVEFRSSS |
| | | | NWVGKGFFAVYEAICGGDVKKDYGHIQSPN |
| | | | YPDDYRPSKVCIWRIQVSEGFHVGLTFQSF |
| | | | EIERHDSCAYDYLEVRDGHSESSTLIGRYC |
| | | | GYEKPDDIKSTSSRLWLKFVSDGSINKAGF |
| | | | AVNFFKEVDECSRPNRGGCEQRCLNTLGSY |
| | | | KCSCDPGYELAPDKRRCEAACGGFLTKLNG |
| | | | SITSPGWPKEYPPNKNCIWQLVAPTQYRIS |
| | | | LQFDFFETEGNDVCKYDFVEVRSGLTADSK |
| | | | LHGKFCGSEKPEVITSQYNNMRVEFKSDNT |
| | | | VSKKGFKAHFFSEKRPALQPPRGRPHQLKF |
| | | | RVQKRNRTPQ |
| P13497 | 3 | 12 | MPGVARLPLLLGLLLLPRPGRPLDLADYTY |
| | | | DLAEEDDSEPLNYKDPCKAAAFLGDIALDE |
| | | | EDLRAFQVQQAVDLRRHTARKSSIKAAVPG |
| | | | NTSTPSCQSTNGQPQRGACGRWRGRSRSRR |
| | | | AATSRPERVWPDGVIPFVIGGNFTGSQRAV |
| | | | FRQAMRHWEKHTCVTFLERTDEDSYIVFTY |
| | | | RPCGCCSYVGRRGGGPQAISIGKNCDKFGI |
| | | | VVHELGHVVGFWHEHTRPDRDRHVSIVREN |
| | | | IQPGVLHSSLLLLSCGSRNGASFPCSLESS |
| | | | THQALCWTGLFLRPSPFPRLPLAAPRTLRA |
| | | | GV |
| P13497 | 4 | 13 | MPGVARLPLLLGLLLLPRPGRPLDLADYTY |
| | | | DLAEEDDSEPLNYKDPCKAAAFLGDIALDE |
| | | | EDLRAFQVQQAVDLRRHTARKSSIKAAVPG |
| | | | NTSTPSCQSTNGQPQRGACGRWRGRSRSRR |
| | | | AATSRPERVWPDGVIPFVIGGNFTGSQRAV |
| | | | FRQAMRHWEKHTCVTFLERTDEDSYIVFTY |
| | | | RPCGCCSYVGRRGGGPQAISIGKNCDKFGI |
| | | | VVHELGHVVGFWHEHTRPDRDRHVSIVREN |
| | | | IQPGQEYNFLKMEPQEVESLGETYDFDSIM |
| | | | HYARNTFSRGIFLDTIVPKYEVNGVKPPIG |
| | | | QRTRLSKGDIAQARKLYKCPACGETLQDST |
| | | | GNFSSPEYPNGYSAHMHCVWRISVTPGEKI |
| | | | ILNFTSLDLYRSRLCWYDYVEVRDGFWRKA |
| | | | PLRGRFCGSKLPEPIVSTDSRLWVEFRSSS |
| | | | NWVGKGFFAVYEAICGGDVKKDYGHIQSPN |
| | | | YPDDYRPSKVCIWRIQVSEGFHVGLTFQSF |
| | | | EIERHDSCAYDYLEVRDGHSESSTLIGRYC |
| | | | GYEKPDDIKSTSSRLWLKFVSDGSINKAGF |
| | | | AVNFFKEVDECSRPNRGGCEQRCLNTLGSY |
| | | | KCSCDPGYELAPDKRRCEGCYDLQVGKPLL |
| | | | WDRHCFRLSTHGPEMLGTALRG |
| P13497 | 5 | 14 | MPGVARLPLLLGLLLLPRPGRPLDLADYTY |
| | | | DLAEEDDSEPLNYKDPCKAAAFLGDIALDE |
| | | | EDLRAFQVQQAVDLRRHTARKSSIKAAVPG |
| | | | NTSTPSCQSTNGQPQRGACGRWRGRSRSRR |
| | | | AATSRPERVWPDGVIPFVIGGNFTGSQRAV |
| | | | FRQAMRHWEKHTCVTFLERTDEDSYIVFTY |
| | | | RPCGCCSYVGRRGGGPQAISIGKNCDKFGI |
| | | | VVHELGHVVGFWHEHTRPDRDRHVSIVREN |
| | | | IQPGQEYNFLKMEPQEVESLGETYDFDSIM |
| | | | HYARNTFSRGIFLDTIVPKYEVNGVKPPIG |
| | | | QRTRLSKGDIAQARKLYKCPACGETLQDST |
| | | | GNFSSPEYPNGYSAHMHCVWRISVTPGEKI |
| | | | ILNFTSLDLYRSRLCWYDYVEVRDGFWRKA |
| | | | PLRGRFCGSKLPEPIVSTDSRLWVEFRSSS |
| | | | NWVGKGFFAVYEAICGGDVKKDYGHIQSPN |
| | | | YPDDYRPSKVCIWRIQVSEGFHVGLTFQSF |
| | | | EIERHDSCAYDYLEVRDGHSESSTLIGRYC |
| | | | GYEKPDDIKSTSSRLWLKFVSDGSINKAGF |
| | | | AVNFFKEVDECSRPNRGGCEQRCLNTLGSY |
| | | | KCSCDPGYELAPDKRRCEAACGGFLTKLNG |
| | | | SITSPGWPKEYPPNKNCIWQLVAPTQYRIS |
| | | | LQFDFFETEGNDVCKYDFVEVRSGLTADSK |
| | | | LHGKFCGSEKPEVITSQYNNMRVEFKSDNT |
| | | | VSKKGFKAHFFSGGELFGLLGHPPRRP |
| P13497 | 6 | 15 | MPGVARLPLLLGLLLLPRPGRPLDLADYTY |
| | | | DLAEEDDSEPLNYKDPCKAAAFLGDIALDE |
| | | | EDLRAFQVQQAVDLRRHTARKSSIKAAVPG |
| | | | NTSTPSCQSTNGQPQRGACGRWRGRSRSRR |
| | | | AATSRPERVWPDGVIPFVIGGNFTGSQRAV |
| | | | FRQAMRHWEKHTCVTFLERTDEDSYIVFTY |
| | | | RPCGCCSYVGRRGGGPQAISIGKNCDKFGI |
| | | | VVHELGHVVGFWHEHTRPDRDRHVSIVREN |
| | | | IQPGQEYNFLKMEPQEVESLGETYDFDSIM |
| | | | HYARNTFSRGIFLDTIVPKYEVNGVKPPIG |
| | | | QRTRLSKGDIAQARKLYKCPACGETLQDST |
| | | | GNFSSPEYPNGYSAHMHCVWRISVTPGEKI |
| | | | ILNFTSLDLYRSRLCWYDYVEVRDGFWRKA |
| | | | PLRGRFCGSKLPEPIVSTDSRLWVEFRSSS |
| | | | NWVGKGFFAVYEAICGGDVKKDYGHIQSPN |
| | | | YPDDYRPSKVCIWRIQVSEGFHVGLTFQSF |
| | | | EIERHDSCAYDYLEVRDGHSESSTLIGRYC |
| | | | GYEKPDDIKSTSSRLWLKFVSDGSINKAGF |
| | | | AVNFFKEVDECSRPNRGGCEQRCLNTLGSY |
| | | | KCSCDPGYELAPDKRRCEAACGGFLTKLNG |
| | | | SITSPGWPKEYPPNKNCIWQLVAPTQYRIS |
| | | | LQFDFFETEGNDVCKYDFVEVRSGLTADSK |
| | | | LHGKFCGSEKPEVITSQYNNMRVEFKSDNT |
| | | | VSKKGFKAHFFSVLEGAGDRHSHLSGLELL |
| | | | LCPHALVDTVPAPPSALHGDTHAHTHTHVH |
| | | | THCPIAQETCRGPPLGASRLSPQGPGHLTL |
| | | | APQEGSYLDFWDTHRGDPKPRRRRKSLKTF |
| | | | SLTPATFRGIWAL |
| P13497 | 7 | 16 | UNKNOWN |
| P16157 | 1 | 17 | MPYSVGFREADAATSFLRAARSGNLDKALD |
| | | | HLRNGVDINTCNQNGLNGLHLASKEGHVKM |
| | | | VVELLHKEIILETTTKKGNTALHIAALAGQ |
| | | | DEVVRELVNYGANVNAQSQKGFTPLYMAAQ |
| | | | ENHLEVVKFLLENGANQNVATEDGFTPLAV |
| | | | ALQQGHENVVAHLINYGTKGKVRLPALHIA |
| | | | ARNDDTRTAAVLLQNDPNPDVLSKTGFTPL |
| | | | HIAAHYENLNVAQLLLNRGASVNFTPQNGI |
| | | | TPLHIASRRGNVIMVRLLLDRGAQIETKTK |
| | | | DELTPLHCAARNGHVRISEILLDHGAPIQA |
| | | | KTKNGLSPIHMAAQGDHLDCVRLLLQYDAE |
| | | | IDDITLDHLTPLHVAAHCGHHRVAKVLLDK |
| | | | GAKPNSRALNGFTPLHIACKKNHVRVMELL |
| | | | LKTGASIDAVTESGLTPLHVASFMGHLPIV |
| | | | KNLLQRGASPNVSNVKVETPLHMAARAGHT |
| | | | EVAKYLLQNKAKVNAKAKDDQTPLHCAARI |
| | | | GHTNMVKLLLENNANPNLATTAGHTPLHIA |
| | | | AREGHVETVLALLEKEASQACMTKKGFTPL |
| | | | HVAAKYGKVRVAELLLERDAHPNAAGKNGL |
| | | | TPLHVAVHHNNLDIVKLLLPRGGSPHSPAW |
| | | | NGYTPLHIAAKQNQVEVARSLLQYGGSANA |
| | | | ESVQGVTPLHLAAQEGHAEMVALLLSKQAN |
| | | | GNLGNKSGLTPLHLVAQEGHVPVADVLIKH |
| | | | GVMVDATTRMGYTPLHVASHYGNIKLVKFL |
| | | | LQHQADVNAKTKLGYSPLHQAAQQGHTDIV |
| | | | TLLLKNGASPNEVSSDGTTPLAIAKRLGYI |
| | | | SVTDVLKVVTDETSFVLVSDKHRMSFPETV |
| | | | DEILDVSEDEGEELISFKAERRDSRDVDEE |
| | | | KELLDFVPKLDQVVESPAIPRIPCAMPETV |
| | | | VIRSEEQEQASKEYDEDSLIPSSPATETSD |
| | | | NISPVASPVHTGFLVSFMVDARGGSMRGSR |
| | | | HNGLRVVIPPRTCAAPTRITCRLVKPQKLS |
| | | | TPPPLAEEEGLASRIIALGPTGAQFLSPVI |
| | | | VEIPHFASHGRGDRELVVLRSENGSVWKEH |
| | | | RSRYGESYLDQILNGMDEELGSLEELEKKR |
| | | | VCRIITTDFPLYFVIMSRLCQDYDTIGPEG |
| | | | GSLKSKLVPLVQATFPENAVTKRVKLALQA |
| | | | QPVPDELVTKLLGNQATFSPIVTVEPRRRK |
| | | | FHRPIGLRIPLPPSWTDNPRDSGEGDTTSL |
| | | | RLLCSVIGGTDQAQWEDITGTTKLVYANEC |
| | | | ANFTTNVSARFWLSDCPRTAEAVNFATLLY |
| | | | KELTAVPYMAKFVIFAKMNDPREGRLRCYC |
| | | | MTDDKVDKTLEQHENFVEVARSRDIEVLEG |
| | | | MSLFAELSGNLVPVKKAAQQRSFHFQSFRE |
| | | | NRLAMPVKVRDSSREPGGSLSFLRKAMKYE |
| | | | DTQHILCHLNITMPPCAKGSGAEDRRRTPT |
| | | | PLALRYSILSESTPGSLSGTEQAEMKMAVI |
| | | | SEHLGLSWAELARELQFSVEDINRIRVENP |
| | | | NSLLEQSVALLNLWVIREGQNANMENLYTA |
| | | | LQSIDRGEIVNMLEGSGRQSRNLKPDRRHT |
| | | | DRDYSLSPSQMNGYSSLQDELLSPASLGCA |
| | | | LSSPLRADQYWNEVAVLDAIPLAATEHDTM |
| | | | LEMSDMQVWSAGLTPSLVTAEDSSLECSKA |
| | | | EDSDATGHEWKLEGALSEEPRGPELGSLEL |
| | | | VEDDTVDSDATNGLIDLLEQEEGQRSEEKL |
| | | | PGSKRQDDATGAGQDSENEVSLVSGHQRGQ |
| | | | ARITHSPTVSQVTERSQDRLQDWDADGSIV |
| | | | SYLQDAAQGSWQEEVTQGPHSFQGTSTMTE |
| | | | GLEPGGSQEYEKVLVSVSEHTWTEQPEAES |
| | | | SQADRDRRQQGQEEQVQEAKNTFTQVVQGN |
| | | | EFQNIPGEQVTEEQFTDEQGNIVTKKIIRK |
| | | | VVRQIDLSSADAAQEHEEVTVEGPLEDPSE |
| | | | LEVDIDYFMKHSKDHTSTPNP |
| P16157 | 2 | 18 | MPYSVGFREADAATSFLRAARSGNLDKALD |
| | | | HLRNGVDINTCNQNGLNGLHLASKEGHVKM |
| | | | VVELLHKEIILETTTKKGNTALHIAALAGQ |
| | | | DEVVRELVNYGANVNAQSQKGFTPLYMAAQ |
| | | | ENHLEVVKFLLENGANQNVATEDGFTPLAV |
| | | | ALQQGHENVVAHLINYGTKGKVRLPALHIA |
| | | | ARNDDTRTAAVLLQNDPNPDVLSKTGFTPL |
| | | | HIAAHYENLNVAQLLLNRGASVNFTPQNGI |
| | | | TPLHIASRRGNVIMVRLLLDRGAQIETKTK |
| | | | DELTPLHCAARNGHVRISEILLDHGAPIQA |
| | | | KTKNGLSPIHMAAQGDHLDCVRLLLQYDAE |
| | | | IDDITLDHLTPLHVAAHCGHHRVAKVLLDK |
| | | | GAKPNSRALNGFTPLHIACKKNHVRVMELL |
| | | | LKTGASIDAVTESGLTPLHVASFMGHLPIV |
| | | | KNLLQRGASPNVSNVKVETPLHMAARAGHT |
| | | | EVAKYLLQNKAKVNAKAKDDQTPLHCAARI |
| | | | GHTNMVKLLLENNANPNLATTAGHTPLHIA |
| | | | AREGHVETVLALLEKEASQACMTKKGFTPL |
| | | | HVAAKYGKVRVAELLLERDAHPNAAGKNGL |
| | | | TPLHVAVHHNNLDIVKLLLPRGGSPHSPAW |
| | | | NGYTPLHIAAKQNQVEVARSLLQYGGSANA |
| | | | ESVQGVTPLHLAAQEGHAEMVALLLSKQAN |
| | | | GNLGNKSGLTPLHLVAQEGHVPVADVLIKH |
| | | | GVMVDATTRMGYTPLHVASHYGNIKLVKFL |
| | | | LQHQADVNAKTKLGYSPLHQAAQQGHTDIV |
| | | | TLLLKNGASPNEVSSDGTTPLAIAKRLGYI |
| | | | SVTDVLKVVTDETSFVLVSDKHRMSFPETV |
| | | | DEILDVSEDEGEELISFKAERRDSRDVDEE |
| | | | KELLDFVPKLDQVVESPAIPRIPCAMPETV |
| | | | VIRSEEQEQASKEYDEDSLIPSSPATETSD |
| | | | NISPVASPVHTGFLVSFMVDARGGSMRGSR |
| | | | HNGLRVVIPPRTCAAPTRITCRLVKPQKLS |
| | | | TPPPLAEEEGLASRIIALGPTGAQFLSPVI |
| | | | VEIPHFASHGRGDRELVVLRSENGSVWKEH |
| | | | RSRYGESYLDQILNGMDEELGSLEELEKKR |
| | | | VCRIITTDFPLYFVIMSRLCQDYDTIGPEG |
| | | | GSLKSKLVPLVQATFPENAVTKRVKLALQA |
| | | | QPVPDELVTKLLGNQATFSPIVTVEPRRRK |
| | | | FHRPIGLRIPLPPSWTDNPRDSGEGDTTSL |
| | | | RLLCSVIGGTDQAQWEDITGTTKLVYANEC |
| | | | ANFTTNVSARFWLSDCPRTAEAVNFATLLY |
| | | | KELTAVPYMAKFVIFAKMNDPREGRLRCYC |
| | | | MTDDKVDKTLEQHENFVEVARSRDIEVLEG |
| | | | MSLFAELSGNLVPVKKAAQQRSFHFQSFRE |
| | | | NRLAMPVKVRDSSREPGGSLSFLRKAMKYE |
| | | | DTQHILCHLNITMPPCAKGSGAEDRRRTPT |
| | | | PLALRYSILSESTPGSLSGTEQAEMKMAVI |
| | | | SEHLGLSWAELARELQFSVEDINRIRVENP |
| | | | NSLLEQSVALLNLWVIREGQNANMENLYTA |
| | | | LQSIDRGEIVNMLEGSGRQSRNLKPDRRHT |
| | | | DRDYSLSPSQMNDTSTPNP |
| P16157 | 3 | 19 | MPYSVGFREADAATSFLRAARSGNLDKALD |
| | | | HLRNGVDINTCNQNGLNGLHLASKEGHVKM |
| | | | VVELLHKEIILETTTKKGNTALHIAALAGQ |
| | | | DEVVRELVNYGANVNAQSQKGFTPLYMAAQ |
| | | | ENHLEVVKFLLENGANQNVATEDGFTPLAV |
| | | | ALQQGHENVVAHLINYGTKGKVRLPALHIA |
| | | | ARNDDTRTAAVLLQNDPNPDVLSKTGFTPL |
| | | | HIAAHYENLNVAQLLLNRGASVNFTPQNGI |
| | | | TPLHIASRRGNVIMVRLLLDRGAQIETKTK |
| | | | DELTPLHCAARNGHVRISEILLDHGAPIQA |
| | | | KTKNGLSPIHMAAQGDHLDCVRLLLQYDAE |
| | | | IDDITLDHLTPLHVAAHCGHHRVAKVLLDK |
| | | | GAKPNSRALNGFTPLHIACKKNHVRVMELL |
| | | | LKTGASIDAVTESGLTPLHVASFMGHLPIV |
| | | | KNLLQRGASPNVSNVKVETPLHMAARAGHT |
| | | | EVAKYLLQNKAKVNAKAKDDQTPLHCAARI |
| | | | GHTNMVKLLLENNANPNLATTAGHTPLHIA |
| | | | AREGHVETVLALLEKEASQACMTKKGFTPL |
| | | | HVAAKYGKVRVAELLLERDAHPNAAGKNGL |
| | | | TPLHVAVHHNNLDIVKLLLPRGGSPHSPAW |
| | | | NGYTPLHIAAKQNQVEVARSLLQYGGSANA |
| | | | ESVQGVTPLHLAAQEGHAEMVALLLSKQAN |
| | | | GNLGNKSGLTPLHLVAQEGHVPVADVLIKH |
| | | | GVMVDATTRMGYTPLHVASHYGNIKLVKFL |
| | | | LQHQADVNAKTKLGYSPLHQAAQQGHTDIV |
| | | | TLLLKNGASPNEVSSDGTTPLAIAKRLGYI |
| | | | SVTDVLKVVTDETSFVLVSDKHRMSFPETV |
| | | | DEILDVSEDEGEELISFKAERRDSRDVDEE |
| | | | KELLDFVPKLDQVVESPAIPRIPCAMPETV |
| | | | VIRSEEQEQASKEYDEDSLIPSSPATETSD |
| | | | NISPVASPVHTGFLVSFMVDARGGSMRGSR |
| | | | HNGLRVVIPPRTCAAPTRITCRLVKPQKLS |
| | | | TPPPLAEEEGLASRIIALGPTGAQFLSPVI |
| | | | VEIPHFASHGRGDRELVVLRSENGSVWKEH |
| | | | RSRYGESYLDQILNGMDEELGSLEELEKKR |
| | | | VCRIITTDFPLYFVIMSRLCQDYDTIGPEG |
| | | | GSLKSKLVPLVQATFPENAVTKRVKLALQA |
| | | | QPVPDELVTKLLGNQATFSPIVTVEPRRRK |
| | | | FHRPIGLRIPLPPSWTDNPRDSGEGDTTSL |
| | | | RLLCSVIGGTDQAQWEDITGTTKLVYANEC |
| | | | ANFTTNVSARFWLSDCPRTAEAVNFATLLY |
| | | | KELTAVPYMAKFVIFAKMNDPREGRLRCYC |
| | | | MTDDKVDKTLEQHENFVEVARSRDIEVLEG |
| | | | MSLFAELSGNLVPVKKAAQQRSFHFQSFRE |
| | | | NRLAMPVKVRDSSREPGGSLSFLRKAMKYE |
| | | | DTQHILCHLNITMPPCAKGSGAEDRRRTPT |
| | | | PLALRYSILSESTPGSLSGTEQAEMKMAVI |
| | | | SEHLGLSWAELARELQFSVEDINRIRVENP |
| | | | NSLLEQSVALLNLWVIREGQNANMENLYTA |
| | | | LQSIDRGEIVNMLEGSGRQSRNLKPDRRHT |
| | | | DRDYSLSPSQMNGYSSLQDELLSPASLGCA |
| | | | LSSPLRADQYWNEVAVLDAIPLAATEHDTM |
| | | | LEMSDMQVWSAGLTPSLVTAEDSSLECSKA |
| | | | EDSDATGHEWKLEGALSEEPRGPELGSLEL |
| | | | VEDDTVDSDATNGLIDLLEQEEGQRSEEKL |
| | | | PGSKRQDDATGAGQDSENEVSLVSGHQRGQ |
| | | | ARITHSPTVSQVTERSQDRLQDWDADGSIV |
| | | | SYLQDAAQGSWQEEVTQGPHSFQGTSTMTE |
| | | | GLEPGGSQEYEKVLVSVSEHTWTEQPEAES |
| | | | SQADRDRRQQGQEEQVQEAKNTFTQVVQGN |
| | | | EFQNIPGEQVTEEQFTDEQGNIVTKKIIRK |
| | | | VVRQIDLSSADAAQEHEEVELRGSGLQPDL |
| | | | IEGRKGAQIVKRASLKRGKQ |
| P16157 | 4 | 20 | MPYSVGFREADAATSFLRAARSGNLDKALD |
| | | | HLRNGVDINTCNQNGLNGLHLASKEGHVKM |
| | | | VVELLHKEIILETTTKKGNTALHIAALAGQ |
| | | | DEVVRELVNYGANVNAQSQKGFTPLYMAAQ |
| | | | ENHLEVVKFLLENGANQNVATEDGFTPLAV |
| | | | ALQQGHENVVAHLINYGTKGKVRLPALHIA |
| | | | ARNDDTRTAAVLLQNDPNPDVLSKTGFTPL |
| | | | HIAAHYENLNVAQLLLNRGASVNFTPQNGI |
| | | | TPLHIASRRGNVIMVRLLLDRGAQIETKTK |
| | | | DELTPLHCAARNGHVRISEILLDHGAPIQA |
| | | | KTKNGLSPIHMAAQGDHLDCVRLLLQYDAE |
| | | | IDDITLDHLTPLHVAAHCGHHRVAKVLLDK |
| | | | GAKPNSRALNGFTPLHIACKKNHVRVMELL |
| | | | LKTGASIDAVTESGLTPLHVASFMGHLPIV |
| | | | KNLLQRGASPNVSNVKVETPLHMAARAGHT |
| | | | EVAKYLLQNKAKVNAKAKDDQTPLHCAARI |
| | | | GHTNMVKLLLENNANPNLATTAGHTPLHIA |
| | | | AREGHVETVLALLEKEASQACMTKKGFTPL |
| | | | HVAAKYGKVRVAELLLERDAHPNAAGKNGL |
| | | | TPLHVAVHHNNLDIVKLLLPRGGSPHSPAW |
| | | | NGYTPLHIAAKQNQVEVARSLLQYGGSANA |
| | | | ESVQGVTPLHLAAQEGHAEMVALLLSKQAN |
| | | | GNLGNKSGLTPLHLVAQEGHVPVADVLIKH |
| | | | GVMVDATTRMGYTPLHVASHYGNIKLVKFL |
| | | | LQHQADVNAKTKLGYSPLHQAAQQGHTDIV |
| | | | TLLLKNGASPNEVSSDGTTPLAIAKRLGYI |
| | | | SVTDVLKVVTDETSFVLVSDKHRMSFPETV |
| | | | DEILDVSEDEGEELISFKAERRDSRDVDEE |
| | | | KELLDFVPKLDQVVESPAIPRIPCAMPETV |
| | | | VIRSEEQEQASKEYDEDSLIPSSPATETSD |
| | | | NISPVASPVHTGFLVSFMVDARGGSMRGSR |
| | | | HNGLRVVIPPRTCAAPTRITCRLVKPQKLS |
| | | | TPPPLAEEEGLASRIIALGPTGAQFLSPVI |
| | | | VEIPHFASHGRGDRELVVLRSENGSVWKEH |
| | | | RSRYGESYLDQILNGMDEELGSLEELEKKR |
| | | | VCRIITTDFPLYFVIMSRLCQDYDTIGPEG |
| | | | GSLKSKLVPLVQATFPENAVTKRVKLALQA |
| | | | QPVPDELVTKLLGNQATFSPIVTVEPRRRK |
| | | | FHRPIGLRIPLPPSWTDNPRDSGEGDTTSL |
| | | | RLLCSVIGGTDQAQWEDITGTTKLVYANEC |
| | | | ANFTTNVSARFWLSDCPRTAEAVNFATLLY |
| | | | KELTAVPYMAKFVIFAKMNDPREGRLRCYC |
| | | | MTDDKVDKTLEQHENFVEVARSRDIEVLEG |
| | | | MSLFAELSGNLVPVKKAAQQRSFHFQSFRE |
| | | | NRLAMPVKVRDSSREPGGSLSFLRKAMKYE |
| | | | DTQHILCHLNITMPPCAKGSGAEDRRRTPT |
| | | | PLALRYSILSESTPGSLSGTEQAEMKMAVI |
| | | | SEHLGLSWAELARELQFSVEDINRIRVENP |
| | | | NSLLEQSVALLNLWVIREGQNANMENLYTA |
| | | | LQSIDRGEIVNMLEGSGRQSRNLKPDRRHT |
| | | | DRDYSLSPSQMNGHQRGQARITHSPTVSQV |
| | | | TERSQDRLQDWDADGSIVSYLQDAAQGSWQ |
| | | | EEVTQGPHSFQGTSTMTEGLEPGGSQEYEK |
| | | | VLVSVSEHTWTEQPEAESSQADRDRRQQGQ |
| | | | EEQVQEAKNTFTQVVQGNEFQNIPGEQVTE |
| | | | EQFTDEQGNIVTKKIIRKVVRQIDLSSADA |
| | | | AQEHEEVTVEGPLEDPSELEVDIDYFMKHS |
| | | | KDHTSTPNP |
| P16157 | 5 | 21 | MPYSVGFREADAATSFLRAARSGNLDKALD |
| | | | HLRNGVDINTCNQNGLNGLHLASKEGHVKM |
| | | | VVELLHKEIILETTTKKGNTALHIAALAGQ |
| | | | DEVVRELVNYGANVNAQSQKGFTPLYMAAQ |
| | | | ENHLEVVKFLLENGANQNVATEDGFTPLAV |
| | | | ALQQGHENVVAHLINYGTKGKVRLPALHIA |
| | | | ARNDDTRTAAVLLQNDPNPDVLSKTGFTPL |
| | | | HIAAHYENLNVAQLLLNRGASVNFTPQNGI |
| | | | TPLHIASRRGNVIMVRLLLDRGAQIETKTK |
| | | | DELTPLHCAARNGHVRISEILLDHGAPIQA |
| | | | KTKNGLSPIHMAAQGDHLDCVRLLLQYDAE |
| | | | IDDITLDHLTPLHVAAHCGHHRVAKVLLDK |
| | | | GAKPNSRALNGFTPLHIACKKNHVRVMELL |
| | | | LKTGASIDAVTESGLTPLHVASFMGHLPIV |
| | | | KNLLQRGASPNVSNVKVETPLHMAARAGHT |
| | | | EVAKYLLQNKAKVNAKAKDDQTPLHCAARI |
| | | | GHTNMVKLLLENNANPNLATTAGHTPLHIA |
| | | | AREGHVETVLALLEKEASQACMTKKGFTPL |
| | | | HVAAKYGKVRVAELLLERDAHPNAAGKNGL |
| | | | TPLHVAVHHNNLDIVKLLLPRGGSPHSPAW |
| | | | NGYTPLHIAAKQNQVEVARSLLQYGGSANA |
| | | | ESVQGVTPLHLAAQEGHAEMVALLLSKQAN |
| | | | GNLGNKSGLTPLHLVAQEGHVPVADVLIKH |
| | | | GVMVDATTRMGYTPLHVASHYGNIKLVKFL |
| | | | LQHQADVNAKTKLGYSPLHQAAQQGHTDIV |
| | | | TLLLKNGASPNEVSSDGTTPLAIAKRLGYI |
| | | | SVTDVLKVVTDETSFVLVSDKHRMSFPETV |
| | | | DEILDVSEDEGEELISFKAERRDSRDVDEE |
| | | | KELLDFVPKLDQVVESPAIPRIPCAMPETV |
| | | | VIRSEEQEQASKEYDEDSLIPSSPATETSD |
| | | | NISPVASPVHTGFLVSFMVDARGGSMRGSR |
| | | | HNGLRVVIPPRTCAAPTRITCRLVKPQKLS |
| | | | TPPPLAEEEGLASRIIALGPTGAQFLSPVI |
| | | | VEIPHFASHGRGDRELVVLRSENGSVWKEH |
| | | | RSRYGESYLDQILNGMDEELGSLEELEKKR |
| | | | VCRIITTDFPLYFVIMSRLCQDYDTIGPEG |
| | | | GSLKSKLVPLVQATFPENAVTKRVKLALQA |
| | | | QPVPDELVTKLLGNQATFSPIVTVEPRRRK |
| | | | FHRPIGLRIPLPPSWTDNPRDSGEGDTTSL |
| | | | RLLCSVIGGTDQAQWEDITGTTKLVYANEC |
| | | | ANFTTNVSARFWLSDCPRTAEAVNFATLLY |
| | | | KELTAVPYMAKFVIFAKMNDPREGRLRCYC |
| | | | MTDDKVDKTLEQHENFVEVARSRDIEVLEG |
| | | | MSLFAELSGNLVPVKKAAQQRSFHFQSFRE |
| | | | NRLAMPVKVRDSSREPGGSLSFLRKAMKYE |
| | | | DTQHILCHLNITMPPCAKGSGAEDRRRTPT |
| | | | PLALRYSILSESTPGSLSGTEQAEMKMAVI |
| | | | SEHLGLSWAELARELQFSVEDINRIRVENP |
| | | | NSLLEQSVALLNLWVIREGQNANMENLYTA |
| | | | LQSIDRGEIVNMLEGSGRQSRNLKPDRRHT |
| | | | DRDYSLSPSQMNGYSSLQDELLSPASLGCA |
| | | | LSSPLRADQYWNEVAVLDAIPLAATEHDTM |
| | | | LEMSDMQVWSAGLTPSLVTAEDSSLECSKA |
| | | | EDSDATGHEWKLEGALSEEPRGPELGSLEL |
| | | | VEDDTVDSDATNGLIDLLEQEEGQRSEEKL |
| | | | PGSKRQDDATGAGQDSENEVSLVSGHQRGQ |
| | | | ARITHSPTVSQVTERSQDRLQDWDADGSIV |
| | | | SYLQDAAQGSWQEEVTQGPHSFQGTSTMTE |
| | | | GLEPGGSQEYEKVLVSVSEHTWTEQPEAES |
| | | | SQADRDRRQQGQEEQVQEAKNTFTQVVQGN |
| | | | EFQNIPGEQVTEEQFTDEQGNIVTKKIIRK |
| | | | VVRQIDLSSADAAQEHEEDHTSTPNP |
| P16157 | 6 | 22 | MPYSVGFREADAATSFLRAARSGNLDKALD |
| | | | HLRNGVDINTCNQNGLNGLHLASKEGHVKM |
| | | | VVELLHKEIILETTTKKGNTALHIAALAGQ |
| | | | DEVVRELVNYGANVNAQSQKGFTPLYMAAQ |
| | | | ENHLEVVKFLLENGANQNVATEDGFTPLAV |
| | | | ALQQGHENVVAHLINYGTKGKVRLPALHIA |
| | | | ARNDDTRTAAVLLQNDPNPDVLSKTGFTPL |
| | | | HIAAHYENLNVAQLLLNRGASVNFTPQNGI |
| | | | TPLHIASRRGNVIMVRLLLDRGAQIETKTK |
| | | | DELTPLHCAARNGHVRISEILLDHGAPIQA |
| | | | KTKNGLSPIHMAAQGDHLDCVRLLLQYDAE |
| | | | IDDITLDHLTPLHVAAHCGHHRVAKVLLDK |
| | | | GAKPNSRALNGFTPLHIACKKNHVRVMELL |
| | | | LKTGASIDAVTESGLTPLHVASFMGHLPIV |
| | | | KNLLQRGASPNVSNVKVETPLHMAARAGHT |
| | | | EVAKYLLQNKAKVNAKAKDDQTPLHCAARI |
| | | | GHTNMVKLLLENNANPNLATTAGHTPLHIA |
| | | | AREGHVETVLALLEKEASQACMTKKGFTPL |
| | | | HVAAKYGKVRVAELLLERDAHPNAAGKNGL |
| | | | TPLHVAVHHNNLDIVKLLLPRGGSPHSPAW |
| | | | NGYTPLHIAAKQNQVEVARSLLQYGGSANA |
| | | | ESVQGVTPLHLAAQEGHAEMVALLLSKQAN |
| | | | GNLGNKSGLTPLHLVAQEGHVPVADVLIKH |
| | | | GVMVDATTRMGYTPLHVASHYGNIKLVKFL |
| | | | LQHQADVNAKTKLGYSPLHQAAQQGHTDIV |
| | | | TLLLKNGASPNEVSSDGTTPLAIAKRLGYI |
| | | | SVTDVLKVVTDETSFVLVSDKHRMSFPETV |
| | | | DEILDVSEDEGEELISFKAERRDSRDVDEE |
| | | | KELLDFVPKLDQVVESPAIPRIPCAMPETV |
| | | | VIRSEEQEQASKEYDEDSLIPSSPATETSD |
| | | | NISPVASPVHTGFLVSFMVDARGGSMRGSR |
| | | | HNGLRVVIPPRTCAAPTRITCRLVKPQKLS |
| | | | TPPPLAEEEGLASRIIALGPTGAQFLSPVI |
| | | | VEIPHFASHGRGDRELVVLRSENGSVWKEH |
| | | | RSRYGESYLDQILNGMDEELGSLEELEKKR |
| | | | VCRIITTDFPLYFVIMSRLCQDYDTIGPEG |
| | | | GSLKSKLVPLVQATFPENAVTKRVKLALQA |
| | | | QPVPDELVTKLLGNQATFSPIVTVEPRRRK |
| | | | FHRPIGLRIPLPPSWTDNPRDSGEGDTTSL |
| | | | RLLCSVIGGTDQAQWEDITGTTKLVYANEC |
| | | | ANFTTNVSARFWLSDCPRTAEAVNFATLLY |
| | | | KELTAVPYMAKFVIFAKMNDPREGRLRCYC |
| | | | MTDDKVDKTLEQHENFVEVARSRDIEVLEG |
| | | | MSLFAELSGNLVPVKKAAQQRSFHFQSFRE |
| | | | NRLAMPVKVRDSSREPGGSLSFLRKAMKYE |
| | | | DTQHILCHLNITMPPCAKGSGAEDRRRTPT |
| | | | PLALRYSILSESTPGSLSGTEQAEMKMAVI |
| | | | SEHLGLSWAELARELQFSVEDINRIRVENP |
| | | | NSLLEQSVALLNLWVIREGQNANMENLYTA |
| | | | LQSIDRGEIVNMLEGSGRQSRNLKPDRRHT |
| | | | DRDYSLSPSQMNGHQRGQARITHSPTVSQV |
| | | | TERSQDRLQDWDADGSIVSYLQDAAQGSWQ |
| | | | EEVTQGPHSFQGTSTMTEGLEPGGSQEYEK |
| | | | VLVSVSEHTWTEQPEAESSQADRDRRQQGQ |
| | | | EEQVQEAKNTFTQVVQGNEFQNIPGEQVTE |
| | | | EQFTDEQGNIVTKKIIRKVVRQIDLSSADA |
| | | | AQEHEEDHTSTPNP |
| P16157 | 7 | 23 | MPYSVGFREADAATSFLRAARSGNLDKALD |
| | | | HLRNGVDINTCNQNGLNGLHLASKEGHVKM |
| | | | VVELLHKEIILETTTKKGNTALHIAALAGQ |
| | | | DEVVRELVNYGANVNAQSQKGFTPLYMAAQ |
| | | | ENHLEVVKFLLENGANQNVATEDGFTPLAV |
| | | | ALQQGHENVVAHLINYGTKGKVRLPALHIA |
| | | | ARNDDTRTAAVLLQNDPNPDVLSKTGFTPL |
| | | | HIAAHYENLNVAQLLLNRGASVNFTPQNGI |
| | | | TPLHIASRRGNVIMVRLLLDRGAQIETKTK |
| | | | DELTPLHCAARNGHVRISEILLDHGAPIQA |
| | | | KTKNGLSPIHMAAQGDHLDCVRLLLQYDAE |
| | | | IDDITLDHLTPLHVAAHCGHHRVAKVLLDK |
| | | | GAKPNSRALNGFTPLHIACKKNHVRVMELL |
| | | | LKTGASIDAVTESGLTPLHVASFMGHLPIV |
| | | | KNLLQRGASPNVSNVKVETPLHMAARAGHT |
| | | | EVAKYLLQNKAKVNAKAKDDQTPLHCAARI |
| | | | GHTNMVKLLLENNANPNLATTAGHTPLHIA |
| | | | AREGHVETVLALLEKEASQACMTKKGFTPL |
| | | | HVAAKYGKVRVAELLLERDAHPNAAGKNGL |
| | | | TPLHVAVHHNNLDIVKLLLPRGGSPHSPAW |
| | | | NGYTPLHIAAKQNQVEVARSLLQYGGSANA |
| | | | ESVQGVTPLHLAAQEGHAEMVALLLSKQAN |
| | | | GNLGNKSGLTPLHLVAQEGHVPVADVLIKH |
| | | | GVMVDATTRMGYTPLHVASHYGNIKLVKFL |
| | | | LQHQADVNAKTKLGYSPLHQAAQQGHTDIV |
| | | | TLLLKNGASPNEVSSDGTTPLAIAKRLGYI |
| | | | SVTDVLKVVTDETSFVLVSDKHRMSFPETV |
| | | | DEILDVSEDEGEELISFKAERRDSRDVDEE |
| | | | KELLDFVPKLDQVVESPAIPRIPCAMPETV |
| | | | VIRSEEQEQASKEYDEDSLIPSSPATETSD |
| | | | NISPVASPVHTGFLVSFMVDARGGSMRGSR |
| | | | HNGLRVVIPPRTCAAPTRITCRLVKPQKLS |
| | | | TPPPLAEEEGLASRIIALGPTGAQFLSPVI |
| | | | VEIPHFASHGRGDRELVVLRSENGSVWKEH |
| | | | RSRYGESYLDQILNGMDEELGSLEELEKKR |
| | | | VCRIITTDFPLYFVIMSRLCQDYDTIGPEG |
| | | | GSLKSKLVPLVQATFPENAVTKRVKLALQA |
| | | | QPVPDELVTKLLGNQATFSPIVTVEPRRRK |
| | | | FHRPIGLRIPLPPSWTDNPRDSGEGDTTSL |
| | | | RLLCSVIGGTDQAQWEDITGTTKLVYANEC |
| | | | ANFTTNVSARFWLSDCPRTAEAVNFATLLY |
| | | | KELTAVPYMAKFVIFAKMNDPREGRLRCYC |
| | | | MTDDKVDKTLEQHENFVEVARSRDIEVLEG |
| | | | MSLFAELSGNLVPVKKAAQQRSFHFQSFRE |
| | | | NRLAMPVKVRDSSREPGGSLSFLRKAMKYE |
| | | | DTQHILCHLNITMPPCAKGSGAEDRRRTPT |
| | | | PLALRYSILSESTPGSLSGTEQAEMKMAVI |
| | | | SEHLGLSWAELARELQFSVEDINRIRVENP |
| | | | NSLLEQSVALLNLWVIREGQNANMENLYTA |
| | | | LQSIDRGEIVNMLEGSGRQSRNLKPDRRHT |
| | | | DRDYSLSPSQMNGHQRGQARITHSPTVSQV |
| | | | TERSQDRLQDWDADGSIVSYLQDAAQGSWQ |
| | | | EEVTQGPHSFQGTSTMTEGLEPGGSQEYEK |
| | | | VLVSVSEHTWTEQPEAESSQADRDRRQQGQ |
| | | | EEQVQEAKNTFTQVVQGNEFQNIPGEQVTE |
| | | | EQFTDEQGNIVTKKIIRKVVRQIDLSSADA |
| | | | AQEHEEVELRGSGLQPDLIEGRKGAQIVKR |
| | | | ASLKRGKQ |
| P16157 | 8 | 24 | MPYSVGFREADAATSFLRAARSGNLDKALD |
| | | | HLRNGVDINTCNQNGLNGLHLASKEGHVKM |
| | | | VVELLHKEIILETTTKKGNTALHIAALAGQ |
| | | | DEVVRELVNYGANVNAQSQKGFTPLYMAAQ |
| | | | ENHLEVVKFLLENGANQNVATEDGFTPLAV |
| | | | ALQQGHENVVAHLINYGTKGKVRLPALHIA |
| | | | ARNDDTRTAAVLLQNDPNPDVLSKTGFTPL |
| | | | HIAAHYENLNVAQLLLNRGASVNFTPQNGI |
| | | | TPLHIASRRGNVIMVRLLLDRGAQIETKTK |
| | | | DELTPLHCAARNGHVRISEILLDHGAPIQA |
| | | | KTKNGLSPIHMAAQGDHLDCVRLLLQYDAE |
| | | | IDDITLDHLTPLHVAAHCGHHRVAKVLLDK |
| | | | GAKPNSRALNGFTPLHIACKKNHVRVMELL |
| | | | LKTGASIDAVTESGLTPLHVASFMGHLPIV |
| | | | KNLLQRGASPNVSNVKVETPLHMAARAGHT |
| | | | EVAKYLLQNKAKVNAKAKDDQTPLHCAARI |
| | | | GHTNMVKLLLENNANPNLATTAGHTPLHIA |
| | | | AREGHVETVLALLEKEASQACMTKKGFTPL |
| | | | HVAAKYGKVRVAELLLERDAHPNAAGKNGL |
| | | | TPLHVAVHHNNLDIVKLLLPRGGSPHSPAW |
| | | | NGYTPLHIAAKQNQVEVARSLLQYGGSANA |
| | | | ESVQGVTPLHLAAQEGHAEMVALLLSKQAN |
| | | | GNLGNKSGLTPLHLVAQEGHVPVADVLIKH |
| | | | GVMVDATTRMGYTPLHVASHYGNIKLVKFL |
| | | | LQHQADVNAKTKLGYSPLHQAAQQGHTDIV |
| | | | TLLLKNGASPNEVSSDGTTPLAIAKRLGYI |
| | | | SVTDVLKVVTDETSFVLVSDKHRMSFPETV |
| | | | DEILDVSEDEGEELISFKAERRDSRDVDEE |
| | | | KELLDFVPKLDQVVESPAIPRIPCAMPETV |
| | | | VIRSEEQEQASKEYDEDSLIPSSPATETSD |
| | | | NISPVASPVHTGFLVSFMVDARGGSMRGSR |
| | | | HNGLRVVIPPRTCAAPTRITCRLVKPQKLS |
| | | | TPPPLAEEEGLASRIIALGPTGAQFLSPVI |
| | | | VEIPHFASHGRGDRELVVLRSENGSVWKEH |
| | | | RSRYGESYLDQILNGMDEELGSLEELEKKR |
| | | | VCRIITTDFPLYFVIMSRLCQDYDTIGPEG |
| | | | GSLKSKLVPLVQATFPENAVTKRVKLALQA |
| | | | QPVPDELVTKLLGNQATFSPIVTVEPRRRK |
| | | | FHRPIGLRIPLPPSWTDNPRDSGEGDTTSL |
| | | | RLLCSVIGGTDQAQWEDITGTTKLVYANEC |
| | | | ANFTTNVSARFWLSDCPRTAEAVNFATLLY |
| | | | KELTAVPYMAKFVIFAKMNDPREGRLRCYC |
| | | | MTDDKVDKTLEQHENFVEVARSRDIEVLEG |
| | | | MSLFAELSGNLVPVKKAAQQRSFHFQSFRE |
| | | | NRLAMPVKVRDSSREPGGSLSFLRKAMKYE |
| | | | DTQHILCHLNITMPPCAKGSGAEDRRRTPT |
| | | | PLALRYSILSESTPGSLSGTEQAEMKMAVI |
| | | | SEHLGLSWAELARELQFSVEDINRIRVENP |
| | | | NSLLEQSVALLNLWVIREGQNANMENLYTA |
| | | | LQSIDRGEIVNMLEGSGRQSRNLKPDRRHT |
| | | | DRDYSLSPSQMNGYSSLQDELLSPASLGCA |
| | | | LSSPLRADQYWNEVAVLDAIPLAATEHDTM |
| | | | LEMSDMQVWSAGLTPSLVTAEDSSLECSKA |
| | | | EDSDATGHEWKLEGALSEEPRGPELGSLEL |
| | | | VEDDTVDSDATNGLIDLLEQEEGQRSEEKL |
| | | | PGSKRQDDATGAGQDSENEVSLVSGHQRGQ |
| | | | ARITHSPTVSQVTERSQDRLQDWDADGSIV |
| | | | SYLQDAAQGSWQEEVTQGPHSFQGTSTMTE |
| | | | GLEPGGSQEYEKVLVSVSEHTWTEQPEAES |
| | | | SQADRDRRQQGQEEQVQEAKNTFTQVVQGN |
| | | | EFQNIPGEQVTEEQFTDEQGNIVTKKDHTS |
| | | | TPNP |
| P16157 | 9 | 25 | MPYSVGFREADAATSFLRAARSGNLDKALD |
| | | | HLRNGVDINTCNQNGLNGLHLASKEGHVKM |
| | | | VVELLHKEIILETTTKKGNTALHIAALAGQ |
| | | | DEVVRELVNYGANVNAQSQKGFTPLYMAAQ |
| | | | ENHLEVVKFLLENGANQNVATEDGFTPLAV |
| | | | ALQQGHENVVAHLINYGTKGKVRLPALHIA |
| | | | ARNDDTRTAAVLLQNDPNPDVLSKTGFTPL |
| | | | HIAAHYENLNVAQLLLNRGASVNFTPQNGI |
| | | | TPLHIASRRGNVIMVRLLLDRGAQIETKTK |
| | | | DELTPLHCAARNGHVRISEILLDHGAPIQA |
| | | | KTKNGLSPIHMAAQGDHLDCVRLLLQYDAE |
| | | | IDDITLDHLTPLHVAAHCGHHRVAKVLLDK |
| | | | GAKPNSRALNGFTPLHIACKKNHVRVMELL |
| | | | LKTGASIDAVTESGLTPLHVASFMGHLPIV |
| | | | KNLLQRGASPNVSNVKVETPLHMAARAGHT |
| | | | EVAKYLLQNKAKVNAKAKDDQTPLHCAARI |
| | | | GHTNMVKLLLENNANPNLATTAGHTPLHIA |
| | | | AREGHVETVLALLEKEASQACMTKKGFTPL |
| | | | HVAAKYGKVRVAELLLERDAHPNAAGKNGL |
| | | | TPLHVAVHHNNLDIVKLLLPRGGSPHSPAW |
| | | | NGYTPLHIAAKQNQVEVARSLLQYGGSANA |
| | | | ESVQGVTPLHLAAQEGHAEMVALLLSKQAN |
| | | | GNLGNKSGLTPLHLVAQEGHVPVADVLIKH |
| | | | GVMVDATTRMGYTPLHVASHYGNIKLVKFL |
| | | | LQHQADVNAKTKLGYSPLHQAAQQGHTDIV |
| | | | TLLLKNGASPNEVSSDGTTPLAIAKRLGYI |
| | | | SVTDVLKVVTDETSFVLVSDKHRMSFPETV |
| | | | DEILDVSEDEGEELISFKAERRDSRDVDEE |
| | | | KELLDFVPKLDQVVESPAIPRIPCAMPETV |
| | | | VIRSEEQEQASKEYDEDSLIPSSPATETSD |
| | | | NISPVASPVHTGFLVSFMVDARGGSMRGSR |
| | | | HNGLRVVIPPRTCAAPTRITCRLVKPQKLS |
| | | | TPPPLAEEEGLASRIIALGPTGAQFLSPVI |
| | | | VEIPHFASHGRGDRELVVLRSENGSVWKEH |
| | | | RSRYGESYLDQILNGMDEELGSLEELEKKR |
| | | | VCRIITTDFPLYFVIMSRLCQDYDTIGPEG |
| | | | GSLKSKLVPLVQATFPENAVTKRVKLALQA |
| | | | QPVPDELVTKLLGNQATFSPIVTVEPRRRK |
| | | | FHRPIGLRIPLPPSWTDNPRDSGEGDTTSL |
| | | | RLLCSVIGGTDQAQWEDITGTTKLVYANEC |
| | | | ANFTTNVSARFWLSDCPRTAEAVNFATLLY |
| | | | KELTAVPYMAKFVIFAKMNDPREGRLRCYC |
| | | | MTDDKVDKTLEQHENFVEVARSRDIEVLEG |
| | | | MSLFAELSGNLVPVKKAAQQRSFHFQSFRE |
| | | | NRLAMPVKVRDSSREPGGSLSFLRKAMKYE |
| | | | DTQHILCHLNITMPPCAKGSGAEDRRRTPT |
| | | | PLALRYSILSESTPGSLSGTEQAEMKMAVI |
| | | | SEHLGLSWAELARELQFSVEDINRIRVENP |
| | | | NSLLEQSVALLNLWVIREGQNANMENLYTA |
| | | | LQSIDRGEIVNMLEGSGRQSRNLKPDRRHT |
| | | | DRDYSLSPSQMNGHQRGQARITHSPTVSQV |
| | | | TERSQDRLQDWDADGSIVSYLQDAAQGSWQ |
| | | | EEVTQGPHSFQGTSTMTEGLEPGGSQEYEK |
| | | | VLVSVSEHTWTEQPEAESSQADRDRRQQGQ |
| | | | EEQVQEAKNTFTQVVQGNEFQNIPGEQVTE |
| | | | EQFTDEQGNIVTKKDHTSTPNP |
| P16157 | 10 | 26 | MPYSVGFREADAATSFLRAARSGNLDKALD |
| | | | HLRNGVDINTCNQNGLNGLHLASKEGHVKM |
| | | | VVELLHKEIILETTTKKGNTALHIAALAGQ |
| | | | DEVVRELVNYGANVNAQSQKGFTPLYMAAQ |
| | | | ENHLEVVKFLLENGANQNVATEDGFTPLAV |
| | | | ALQQGHENVVAHLINYGTKGKVRLPALHIA |
| | | | ARNDDTRTAAVLLQNDPNPDVLSKTGFTPL |
| | | | HIAAHYENLNVAQLLLNRGASVNFTPQNGI |
| | | | TPLHIASRRGNVIMVRLLLDRGAQIETKTK |
| | | | DELTPLHCAARNGHVRISEILLDHGAPIQA |
| | | | KTKNGLSPIHMAAQGDHLDCVRLLLQYDAE |
| | | | IDDITLDHLTPLHVAAHCGHHRVAKVLLDK |
| | | | GAKPNSRALNGFTPLHIACKKNHVRVMELL |
| | | | LKTGASIDAVTESGLTPLHVASFMGHLPIV |
| | | | KNLLQRGASPNVSNVKVETPLHMAARAGHT |
| | | | EVAKYLLQNKAKVNAKAKDDQTPLHCAARI |
| | | | GHTNMVKLLLENNANPNLATTAGHTPLHIA |
| | | | AREGHVETVLALLEKEASQACMTKKGFTPL |
| | | | HVAAKYGKVRVAELLLERDAHPNAAGKNGL |
| | | | TPLHVAVHHNNLDIVKLLLPRGGSPHSPAW |
| | | | NGYTPLHIAAKQNQVEVARSLLQYGGSANA |
| | | | ESVQGVTPLHLAAQEGHAEMVALLLSKQAN |
| | | | GNLGNKSGLTPLHLVAQEGHVPVADVLIKH |
| | | | GVMVDATTRMGYTPLHVASHYGNIKLVKFL |
| | | | LQHQADVNAKTKLGYSPLHQAAQQGHTDIV |
| | | | TLLLKNGASPNEVSSDGTTPLAIAKRLGYI |
| | | | SVTDVLKVVTDETSFVLVSDKHRMSFPETV |
| | | | DEILDVSEDEGEELISFKAERRDSRDVDEE |
| | | | KELLDFVPKLDQVVESPAIPRIPCAMPETV |
| | | | VIRSEEQEQASKEYDEDSLIPSSPATETSD |
| | | | NISPVASPVHTGFLVSFMVDARGGSMRGSR |
| | | | HNGLRVVIPPRTCAAPTRITCRLVKPQKLS |
| | | | TPPPLAEEEGLASRIIALGPTGAQFLSPVI |
| | | | VEIPHFASHGRGDRELVVLRSENGSVWKEH |
| | | | RSRYGESYLDQILNGMDEELGSLEELEKKR |
| | | | VCRIITTDFPLYFVIMSRLCQDYDTIGPEG |
| | | | GSLKSKLVPLVQATFPENAVTKRVKLALQA |
| | | | QPVPDELVTKLLGNQATFSPIVTVEPRRRK |
| | | | FHRPIGLRIPLPPSWTDNPRDSGEGDTTSL |
| | | | RLLCSVIGGTDQAQWEDITGTTKLVYANEC |
| | | | ANFTTNVSARFWLSDCPRTAEAVNFATLLY |
| | | | KELTAVPYMAKFVIFAKMNDPREGRLRCYC |
| | | | MTDDKVDKTLEQHENFVEVARSRDIEVLEG |
| | | | MSLFAELSGNLVPVKKAAQQRSFHFQSFRE |
| | | | NRLAMPVKVRDSSREPGGSLSFLRKAMKYE |
| | | | DTQHILCHLNITMPPCAKGSGAEDRRRTPT |
| | | | PLALRYSILSESTPGSLSGTEQAEMKMAVI |
| | | | SEHLGLSWAELARELQFSVEDINRIRVENP |
| | | | NSLLEQSVALLNLWVIREGQNANMENLYTA |
| | | | LQSIDRGEIVNMLEGSGRQSRNLKPDRRHT |
| | | | DRDYSLSPSQMNGYSSLQDELLSPASLGCA |
| | | | LSSPLRADQYWNEVAVLDAIPLAATEHDTM |
| | | | LEMSDMQVWSAGLTPSLVTAEDSSLECSKA |
| | | | EDSDATGHEWKLEGALSEEPRGPELGSLEL |
| | | | VEDDTVDSDATNGLIDLLEQEEGQRSEEKL |
| | | | PGSKRQDDATGAGQDSENEVSLVSGHQRGQ |
| | | | ARITHSPTVSQVTERSQDRLQDWDADGSIV |
| | | | SYLQDAAQGSWQEEVTQGPHSFQGTSTMTE |
| | | | GLEPGGSQEYEKVLVSVSEHTWTEQPEAES |
| | | | SQADRDRRQQGQEEQVQEAKNTFTQVVQDH |
| | | | TSTPNP |
| P16157 | 11 | 27 | MPYSVGFREADAATSFLRAARSGNLDKALD |
| | | | HLRNGVDINTCNQNGLNGLHLASKEGHVKM |
| | | | VVELLHKEIILETTTKKGNTALHIAALAGQ |
| | | | DEVVRELVNYGANVNAQSQKGFTPLYMAAQ |
| | | | ENHLEVVKFLLENGANQNVATEDGFTPLAV |
| | | | ALQQGHENVVAHLINYGTKGKVRLPALHIA |
| | | | ARNDDTRTAAVLLQNDPNPDVLSKTGFTPL |
| | | | HIAAHYENLNVAQLLLNRGASVNFTPQNGI |
| | | | TPLHIASRRGNVIMVRLLLDRGAQIETKTK |
| | | | DELTPLHCAARNGHVRISEILLDHGAPIQA |
| | | | KTKNGLSPIHMAAQGDHLDCVRLLLQYDAE |
| | | | IDDITLDHLTPLHVAAHCGHHRVAKVLLDK |
| | | | GAKPNSRALNGFTPLHIACKKNHVRVMELL |
| | | | LKTGASIDAVTESGLTPLHVASFMGHLPIV |
| | | | KNLLQRGASPNVSNVKVETPLHMAARAGHT |
| | | | EVAKYLLQNKAKVNAKAKDDQTPLHCAARI |
| | | | GHTNMVKLLLENNANPNLATTAGHTPLHIA |
| | | | AREGHVETVLALLEKEASQACMTKKGFTPL |
| | | | HVAAKYGKVRVAELLLERDAHPNAAGKNGL |
| | | | TPLHVAVHHNNLDIVKLLLPRGGSPHSPAW |
| | | | NGYTPLHIAAKQNQVEVARSLLQYGGSANA |
| | | | ESVQGVTPLHLAAQEGHAEMVALLLSKQAN |
| | | | GNLGNKSGLTPLHLVAQEGHVPVADVLIKH |
| | | | GVMVDATTRMGYTPLHVASHYGNIKLVKFL |
| | | | LQHQADVNAKTKLGYSPLHQAAQQGHTDIV |
| | | | TLLLKNGASPNEVSSDGTTPLAIAKRLGYI |
| | | | SVTDVLKVVTDETSFVLVSDKHRMSFPETV |
| | | | DEILDVSEDEGEELISFKAERRDSRDVDEE |
| | | | KELLDFVPKLDQVVESPAIPRIPCAMPETV |
| | | | VIRSEEQEQASKEYDEDSLIPSSPATETSD |
| | | | NISPVASPVHTGFLVSFMVDARGGSMRGSR |
| | | | HNGLRVVIPPRTCAAPTRITCRLVKPQKLS |
| | | | TPPPLAEEEGLASRIIALGPTGAQFLSPVI |
| | | | VEIPHFASHGRGDRELVVLRSENGSVWKEH |
| | | | RSRYGESYLDQILNGMDEELGSLEELEKKR |
| | | | VCRIITTDFPLYFVIMSRLCQDYDTIGPEG |
| | | | GSLKSKLVPLVQATFPENAVTKRVKLALQA |
| | | | QPVPDELVTKLLGNQATFSPIVTVEPRRRK |
| | | | FHRPIGLRIPLPPSWTDNPRDSGEGDTTSL |
| | | | RLLCSVIGGTDQAQWEDITGTTKLVYANEC |
| | | | ANFTTNVSARFWLSDCPRTAEAVNFATLLY |
| | | | KELTAVPYMAKFVIFAKMNDPREGRLRCYC |
| | | | MTDDKVDKTLEQHENFVEVARSRDIEVLEG |
| | | | MSLFAELSGNLVPVKKAAQQRSFHFQSFRE |
| | | | NRLAMPVKVRDSSREPGGSLSFLRKAMKYE |
| | | | DTQHILCHLNITMPPCAKGSGAEDRRRTPT |
| | | | PLALRYSILSESTPGSLSGTEQAEMKMAVI |
| | | | SEHLGLSWAELARELQFSVEDINRIRVENP |
| | | | NSLLEQSVALLNLWVIREGQNANMENLYTA |
| | | | LQSIDRGEIVNMLEGSGRQSRNLKPDRRHT |
| | | | DRDYSLSPSQMNGHQRGQARITHSPTVSQV |
| | | | TERSQDRLQDWDADGSIVSYLQDAAQGSWQ |
| | | | EEVTQGPHSFQGTSTMTEGLEPGGSQEYEK |
| | | | VLVSVSEHTWTEQPEAESSQADRDRRQQGQ |
| | | | EEQVQEAKNTFTQVVQDHTSTPNP |
| P16157 | 12 | 28 | MPYSVGFREADAATSFLRAARSGNLDKALD |
| | | | HLRNGVDINTCNQNGLNGLHLASKEGHVKM |
| | | | VVELLHKEIILETTTKKGNTALHIAALAGQ |
| | | | DEVVRELVNYGANVNAQSQKGFTPLYMAAQ |
| | | | ENHLEVVKFLLENGANQNVATEDGFTPLAV |
| | | | ALQQGHENVVAHLINYGTKGKVRLPALHIA |
| | | | ARNDDTRTAAVLLQNDPNPDVLSKTGFTPL |
| | | | HIAAHYENLNVAQLLLNRGASVNFTPQNGI |
| | | | TPLHIASRRGNVIMVRLLLDRGAQIETKTK |
| | | | DELTPLHCAARNGHVRISEILLDHGAPIQA |
| | | | KTKNGLSPIHMAAQGDHLDCVRLLLQYDAE |
| | | | IDDITLDHLTPLHVAAHCGHHRVAKVLLDK |
| | | | GAKPNSRALNGFTPLHIACKKNHVRVMELL |
| | | | LKTGASIDAVTESGLTPLHVASFMGHLPIV |
| | | | KNLLQRGASPNVSNVKVETPLHMAARAGHT |
| | | | EVAKYLLQNKAKVNAKAKDDQTPLHCAARI |
| | | | GHTNMVKLLLENNANPNLATTAGHTPLHIA |
| | | | AREGHVETVLALLEKEASQACMTKKGFTPL |
| | | | HVAAKYGKVRVAELLLERDAHPNAAGKNGL |
| | | | TPLHVAVHHNNLDIVKLLLPRGGSPHSPAW |
| | | | NGYTPLHIAAKQNQVEVARSLLQYGGSANA |
| | | | ESVQGVTPLHLAAQEGHAEMVALLLSKQAN |
| | | | GNLGNKSGLTPLHLVAQEGHVPVADVLIKH |
| | | | GVMVDATTRMGYTPLHVASHYGNIKLVKFL |
| | | | LQHQADVNAKTKLGYSPLHQAAQQGHTDIV |
| | | | TLLLKNGASPNEVSSDGTTPLAIAKRLGYI |
| | | | SVTDVLKVVTDETSFVLVSDKHRMSFPETV |
| | | | DEILDVSEDEGEELISFKAERRDSRDVDEE |
| | | | KELLDFVPKLDQVVESPAIPRIPCAMPETV |
| | | | VIRSEEQEQASKEYDEDSLIPSSPATETSD |
| | | | NISPVASPVHTGFLVSFMVDARGGSMRGSR |
| | | | HNGLRVVIPPRTCAAPTRITCRLVKPQKLS |
| | | | TPPPLAEEEGLASRIIALGPTGAQFLSPVI |
| | | | VEIPHFASHGRGDRELVVLRSENGSVWKEH |
| | | | RSRYGESYLDQILNGMDEELGSLEELEKKR |
| | | | VCRIITTDFPLYFVIMSRLCQDYDTIGPEG |
| | | | GSLKSKLVPLVQATFPENAVTKRVKLALQA |
| | | | QPVPDELVTKLLGNQATFSPIVTVEPRRRK |
| | | | FHRPIGLRIPLPPSWTDNPRDSGEGDTTSL |
| | | | RLLCSVIGGTDQAQWEDITGTTKLVYANEC |
| | | | ANFTTNVSARFWLSDCPRTAEAVNFATLLY |
| | | | KELTAVPYMAKFVIFAKMNDPREGRLRCYC |
| | | | MTDDKVDKTLEQHENFVEVARSRDIEVLEG |
| | | | MSLFAELSGNLVPVKKAAQQRSFHFQSFRE |
| | | | NRLAMPVKVRDSSREPGGSLSFLRKAMKYE |
| | | | DTQHILCHLNITMPPCAKGSGAEDRRRTPT |
| | | | PLALRYSILSESTPGSLSGTEQAEMKMAVI |
| | | | SEHLGLSWAELARELQFSVEDINRIRVENP |
| | | | NSLLEQSVALLNLWVIREGQNANMENLYTA |
| | | | LQSIDRGEIVNMLEGSGRQSRNLKPDRRHT |
| | | | DRDYSLSPSQMNGYSSLQDELLSPASLGCA |
| | | | LSSPLRADQYWNEVAVLDAIPLAATEHDTM |
| | | | LEMSDMQVWSAGLTPSLVTAEDSSLECSKA |
| | | | EDSDATGHEWKLEGALSEEPRGPELGSLEL |
| | | | VEDDTVDSDATNGLIDLLEQEEGQRSEEKL |
| | | | PGSKRQDDATGAGQDSENEVSLVSGHQRGQ |
| | | | ARITHSPTVSQVTERSQDRLQDWDADGSIV |
| | | | SYLQDAAQGSWQEEVTQGPHSFQGTSTMTE |
| | | | GLEPGGSQEYEKVLVSVSEHTWTEQPEAES |
| | | | SQADRDRRQQGQEEQVQEAKNTFTQVVQGN |
| | | | EFQNIPGEQVTEEQFTDEQGNIVTKKIIRK |
| | | | VVRQIDLSSADAAQEHEEVTVEGPLEDPSE |
| | | | LEVDIDYFMKHSKVELRGSGLQPDLIEGRK |
| | | | GAQIVKRASLKRGKQ |
| P16157 | 13 | 29 | MPYSVGFREADAATSFLRAARSGNLDKALD |
| | | | HLRNGVDINTCNQNGLNGLHLASKEGHVKM |
| | | | VVELLHKEIILETTTKKGNTALHIAALAGQ |
| | | | DEVVRELVNYGANVNAQSQKGFTPLYMAAQ |
| | | | ENHLEVVKFLLENGANQNVATEDGFTPLAV |
| | | | ALQQGHENVVAHLINYGTKGKVRLPALHIA |
| | | | ARNDDTRTAAVLLQNDPNPDVLSKTGFTPL |
| | | | HIAAHYENLNVAQLLLNRGASVNFTPQNGI |
| | | | TPLHIASRRGNVIMVRLLLDRGAQIETKTK |
| | | | DELTPLHCAARNGHVRISEILLDHGAPIQA |
| | | | KTKNGLSPIHMAAQGDHLDCVRLLLQYDAE |
| | | | IDDITLDHLTPLHVAAHCGHHRVAKVLLDK |
| | | | GAKPNSRALNGFTPLHIACKKNHVRVMELL |
| | | | LKTGASIDAVTESGLTPLHVASFMGHLPIV |
| | | | KNLLQRGASPNVSNVKVETPLHMAARAGHT |
| | | | EVAKYLLQNKAKVNAKAKDDQTPLHCAARI |
| | | | GHTNMVKLLLENNANPNLATTAGHTPLHIA |
| | | | AREGHVETVLALLEKEASQACMTKKGFTPL |
| | | | HVAAKYGKVRVAELLLERDAHPNAAGKNGL |
| | | | TPLHVAVHHNNLDIVKLLLPRGGSPHSPAW |
| | | | NGYTPLHIAAKQNQVEVARSLLQYGGSANA |
| | | | ESVQGVTPLHLAAQEGHAEMVALLLSKQAN |
| | | | GNLGNKSGLTPLHLVAQEGHVPVADVLIKH |
| | | | GVMVDATTRMGYTPLHVASHYGNIKLVKFL |
| | | | LQHQADVNAKTKLGYSPLHQAAQQGHTDIV |
| | | | TLLLKNGASPNEVSSDGTTPLAIAKRLGYI |
| | | | SVTDVLKVVTDETSFVLVSDKHRMSFPETV |
| | | | DEILDVSEDEGEELISFKAERRDSRDVDEE |
| | | | KELLDFVPKLDQVVESPAIPRIPCAMPETV |
| | | | VIRSEEQEQASKEYDEDSLIPSSPATETSD |
| | | | NISPVASPVHTGFLVSFMVDARGGSMRGSR |
| | | | HNGLRVVIPPRTCAAPTRITCRLVKPQKLS |
| | | | TPPPLAEEEGLASRIIALGPTGAQFLSPVI |
| | | | VEIPHFASHGRGDRELVVLRSENGSVWKEH |
| | | | RSRYGESYLDQILNGMDEELGSLEELEKKR |
| | | | VCRIITTDFPLYFVIMSRLCQDYDTIGPEG |
| | | | GSLKSKLVPLVQATFPENAVTKRVKLALQA |
| | | | QPVPDELVTKLLGNQATFSPIVTVEPRRRK |
| | | | FHRPIGLRIPLPPSWTDNPRDSGEGDTTSL |
| | | | RLLCSVIGGTDQAQWEDITGTTKLVYANEC |
| | | | ANFTTNVSARFWLSDCPRTAEAVNFATLLY |
| | | | KELTAVPYMAKFVIFAKMNDPREGRLRCYC |
| | | | MTDDKVDKTLEQHENFVEVARSRDIEVLEG |
| | | | MSLFAELSGNLVPVKKAAQQRSFHFQSFRE |
| | | | NRLAMPVKVRDSSREPGGSLSFLRKAMKYE |
| | | | DTQHILCHLNITMPPCAKGSGAEDRRRTPT |
| | | | PLALRYSILSESTPGSLSGTEQAEMKMAVI |
| | | | SEHLGLSWAELARELQFSVEDINRIRVENP |
| | | | NSLLEQSVALLNLWVIREGQNANMENLYTA |
| | | | LQSIDRGEIVNMLEGSGRQSRNLKPDRRHT |
| | | | DRDYSLSPSQMNGHQRGQARITHSPTVSQV |
| | | | TERSQDRLQDWDADGSIVSYLQDAAQGSWQ |
| | | | EEVTQGPHSFQGTSTMTEGLEPGGSQEYEK |
| | | | VLVSVSEHTWTEQPEAESSQADRDRRQQGQ |
| | | | EEQVQEAKNTFTQVVQGNEFQNIPGEQVTE |
| | | | EQFTDEQGNIVTKKIIRKVVRQIDLSSADA |
| | | | AQEHEEVTVEGPLEDPSELEVDIDYFMKHS |
| | | | KVELRGSGLQPDLIEGRKGAQIVKRASLKR |
| | | | GKQ |
| P16157 | 14 | 30 | MPYSVGFREADAATSFLRAARSGNLDKALD |
| | | | HLRNGVDINTCNQNGLNGLHLASKEGHVKM |
| | | | VVELLHKEIILETTTKKGNTALHIAALAGQ |
| | | | DEVVRELVNYGANVNAQSQKGFTPLYMAAQ |
| | | | ENHLEVVKFLLENGANQNVATEDGFTPLAV |
| | | | ALQQGHENVVAHLINYGTKGKVRLPALHIA |
| | | | ARNDDTRTAAVLLQNDPNPDVLSKTGFTPL |
| | | | HIAAHYENLNVAQLLLNRGASVNFTPQNGI |
| | | | TPLHIASRRGNVIMVRLLLDRGAQIETKTK |
| | | | DELTPLHCAARNGHVRISEILLDHGAPIQA |
| | | | KTKNGLSPIHMAAQGDHLDCVRLLLQYDAE |
| | | | IDDITLDHLTPLHVAAHCGHHRVAKVLLDK |
| | | | GAKPNSRALNGFTPLHIACKKNHVRVMELL |
| | | | LKTGASIDAVTESGLTPLHVASFMGHLPIV |
| | | | KNLLQRGASPNVSNVKVETPLHMAARAGHT |
| | | | EVAKYLLQNKAKVNAKAKDDQTPLHCAARI |
| | | | GHTNMVKLLLENNANPNLATTAGHTPLHIA |
| | | | AREGHVETVLALLEKEASQACMTKKGFTPL |
| | | | HVAAKYGKVRVAELLLERDAHPNAAGKNGL |
| | | | TPLHVAVHHNNLDIVKLLLPRGGSPHSPAW |
| | | | NGYTPLHIAAKQNQVEVARSLLQYGGSANA |
| | | | ESVQGVTPLHLAAQEGHAEMVALLLSKQAN |
| | | | GNLGNKSGLTPLHLVAQEGHVPVADVLIKH |
| | | | GVMVDATTRMGYTPLHVASHYGNIKLVKFL |
| | | | LQHQADVNAKTKLGYSPLHQAAQQGHTDIV |
| | | | TLLLKNGASPNEVSSDGTTPLAIAKRLGYI |
| | | | SVTDVLKVVTDETSFVLVSDKHRMSFPETV |
| | | | DEILDVSEDEGEELISFKAERRDSRDVDEE |
| | | | KELLDFVPKLDQVVESPAIPRIPCAMPETV |
| | | | VIRSEEQEQASKEYDEDSLIPSSPATETSD |
| | | | NISPVASPVHTGFLVSFMVDARGGSMRGSR |
| | | | HNGLRVVIPPRTCAAPTRITCRLVKPQKLS |
| | | | TPPPLAEEEGLASRIIALGPTGAQFLSPVI |
| | | | VEIPHFASHGRGDRELVVLRSENGSVWKEH |
| | | | RSRYGESYLDQILNGMDEELGSLEELEKKR |
| | | | VCRIITTDFPLYFVIMSRLCQDYDTIGPEG |
| | | | GSLKSKLVPLVQATFPENAVTKRVKLALQA |
| | | | QPVPDELVTKLLGNQATFSPIVTVEPRRRK |
| | | | FHRPIGLRIPLPPSWTDNPRDSGEGDTTSL |
| | | | RLLCSVIGGTDQAQWEDITGTTKLVYANEC |
| | | | ANFTTNVSARFWLSDCPRTAEAVNFATLLY |
| | | | KELTAVPYMAKFVIFAKMNDPREGRLRCYC |
| | | | MTDDKVDKTLEQHENFVEVARSRDIEVLEG |
| | | | MSLFAELSGNLVPVKKAAQQRSFHFQSFRE |
| | | | NRLAMPVKVRDSSREPGGSLSFLRKAMKYE |
| | | | DTQHILCHLNITMPPCAKGSGAEDRRRTPT |
| | | | PLALRYSILSESTPGSLSGTEQAEMKMAVI |
| | | | SEHLGLSWAELARELQFSVEDINRIRVENP |
| | | | NSLLEQSVALLNLWVIREGQNANMENLYTA |
| | | | LQSIDRGEIVNMLEGSGRQSRNLKPDRRHT |
| | | | DRDYSLSPSQMNGYSSLQDELLSPASLGCA |
| | | | LSSPLRADQYWNEVAVLDAIPLAATEHDTM |
| | | | LEMSDMQVWSAGLTPSLVTAEDSSLECSKA |
| | | | EDSDATGHEWKLEGALSEEPRGPELGSLEL |
| | | | VEDDTVDSDATNGLIDLLEQEEGQRSEEKL |
| | | | PGSKRQDDATGAGQDSENEVSLVSGHQRGQ |
| | | | ARITHSPTVSQVTERSQDRLQDWDADGSIV |
| | | | SYLQDAAQGSWQEEVTQGPHSFQGTSTMTE |
| | | | GLEPGGSQEYEKVLVSVSEHTWTEQPEAES |
| | | | SQADRDRRQQGQEEQVQEAKNTFTQVVQGN |
| | | | EFQNIPGEQVTEEQFTDEQGNIVTKKIIRK |
| | | | VVRQIDLSSADAAQEHEEVTVEGPLEDPSE |
| | | | LEVDIDYFMKHSKVLRRPRPWGTQRHHCCL |
| | | | ALPGRLHDTSLHSPLYELSLQSLFSLVGSV |
| | | | SAPPCRSFRSSACVLPVFAICPAFCLCCCL |
| | | | QVELRGSGLQPDLIEGRKGAQIVKRASLKR |
| | | | GKQ |
| P16157 | 15 | 31 | MPYSVGFREADAATSFLRAARSGNLDKALD |
| | | | HLRNGVDINTCNQNGLNGLHLASKEGHVKM |
| | | | VVELLHKEIILETTTKKGNTALHIAALAGQ |
| | | | DEVVRELVNYGANVNAQSQKGFTPLYMAAQ |
| | | | ENHLEVVKFLLENGANQNVATEDGFTPLAV |
| | | | ALQQGHENVVAHLINYGTKGKVRLPALHIA |
| | | | ARNDDTRTAAVLLQNDPNPDVLSKTGFTPL |
| | | | HIAAHYENLNVAQLLLNRGASVNFTPQNGI |
| | | | TPLHIASRRGNVIMVRLLLDRGAQIETKTK |
| | | | DELTPLHCAARNGHVRISEILLDHGAPIQA |
| | | | KTKNGLSPIHMAAQGDHLDCVRLLLQYDAE |
| | | | IDDITLDHLTPLHVAAHCGHHRVAKVLLDK |
| | | | GAKPNSRALNGFTPLHIACKKNHVRVMELL |
| | | | LKTGASIDAVTESGLTPLHVASFMGHLPIV |
| | | | KNLLQRGASPNVSNVKVETPLHMAARAGHT |
| | | | EVAKYLLQNKAKVNAKAKDDQTPLHCAARI |
| | | | GHTNMVKLLLENNANPNLATTAGHTPLHIA |
| | | | AREGHVETVLALLEKEASQACMTKKGFTPL |
| | | | HVAAKYGKVRVAELLLERDAHPNAAGKNGL |
| | | | TPLHVAVHHNNLDIVKLLLPRGGSPHSPAW |
| | | | NGYTPLHIAAKQNQVEVARSLLQYGGSANA |
| | | | ESVQGVTPLHLAAQEGHAEMVALLLSKQAN |
| | | | GNLGNKSGLTPLHLVAQEGHVPVADVLIKH |
| | | | GVMVDATTRMGYTPLHVASHYGNIKLVKFL |
| | | | LQHQADVNAKTKLGYSPLHQAAQQGHTDIV |
| | | | TLLLKNGASPNEVSSDGTTPLAIAKRLGYI |
| | | | SVTDVLKVVTDETSFVLVSDKHRMSFPETV |
| | | | DEILDVSEDEGEELISFKAERRDSRDVDEE |
| | | | KELLDFVPKLDQVVESPAIPRIPCAMPETV |
| | | | VIRSEEQEQASKEYDEDSLIPSSPATETSD |
| | | | NISPVASPVHTGFLVSFMVDARGGSMRGSR |
| | | | HNGLRVVIPPRTCAAPTRITCRLVKPQKLS |
| | | | TPPPLAEEEGLASRIIALGPTGAQFLSPVI |
| | | | VEIPHFASHGRGDRELVVLRSENGSVWKEH |
| | | | RSRYGESYLDQILNGMDEELGSLEELEKKR |
| | | | VCRIITTDFPLYFVIMSRLCQDYDTIGPEG |
| | | | GSLKSKLVPLVQATFPENAVTKRVKLALQA |
| | | | QPVPDELVTKLLGNQATFSPIVTVEPRRRK |
| | | | FHRPIGLRIPLPPSWTDNPRDSGEGDTTSL |
| | | | RLLCSVIGGTDQAQWEDITGTTKLVYANEC |
| | | | ANFTTNVSARFWLSDCPRTAEAVNFATLLY |
| | | | KELTAVPYMAKFVIFAKMNDPREGRLRCYC |
| | | | MTDDKVDKTLEQHENFVEVARSRDIEVLEG |
| | | | MSLFAELSGNLVPVKKAAQQRSFHFQSFRE |
| | | | NRLAMPVKVRDSSREPGGSLSFLRKAMKYE |
| | | | DTQHILCHLNITMPPCAKGSGAEDRRRTPT |
| | | | PLALRYSILSESTPGSLSGTEQAEMKMAVI |
| | | | SEHLGLSWAELARELQFSVEDINRIRVENP |
| | | | NSLLEQSVALLNLWVIREGQNANMENLYTA |
| | | | LQSIDRGEIVNMLEGSGRQSRNLKPDRRHT |
| | | | DRDYSLSPSQMNGHQRGQARITHSPTVSQV |
| | | | TERSQDRLQDWDADGSIVSYLQDAAQGSWQ |
| | | | EEVTQGPHSFQGTSTMTEGLEPGGSQEYEK |
| | | | VLVSVSEHTWTEQPEAESSQADRDRRQQGQ |
| | | | EEQVQEAKNTFTQVVQGNEFQNIPGEQVTE |
| | | | EQFTDEQGNIVTKKIIRKVVRQIDLSSADA |
| | | | AQEHEEVTVEGPLEDPSELEVDIDYFMKHS |
| | | | KVLRRPRPWGTQRHHCCLALPGRLHDTSLH |
| | | | SPLYELSLQSLFSLVGSVSAPPCRSFRSSA |
| | | | CVLPVFAICPAFCLCCCLQVELRGSGLQPD |
| | | | LIEGRKGAQIVKRASLKRGKQ |
| P16157 | 16 | 32 | MPYSVGFREADAATSFLRAARSGNLDKALD |
| | | | HLRNGVDINTCNQNGLNGLHLASKEGHVKM |
| | | | VVELLHKEIILETTTKKGNTALHIAALAGQ |
| | | | DEVVRELVNYGANVNAQSQKGFTPLYMAAQ |
| | | | ENHLEVVKFLLENGANQNVATEDGFTPLAV |
| | | | ALQQGHENVVAHLINYGTKGKVRLPALHIA |
| | | | ARNDDTRTAAVLLQNDPNPDVLSKTGFTPL |
| | | | HIAAHYENLNVAQLLLNRGASVNFTPQNGI |
| | | | TPLHIASRRGNVIMVRLLLDRGAQIETKTK |
| | | | DELTPLHCAARNGHVRISEILLDHGAPIQA |
| | | | KTKNGLSPIHMAAQGDHLDCVRLLLQYDAE |
| | | | IDDITLDHLTPLHVAAHCGHHRVAKVLLDK |
| | | | GAKPNSRALNGFTPLHIACKKNHVRVMELL |
| | | | LKTGASIDAVTESGLTPLHVASFMGHLPIV |
| | | | KNLLQRGASPNVSNVKVETPLHMAARAGHT |
| | | | EVAKYLLQNKAKVNAKAKDDQTPLHCAARI |
| | | | GHTNMVKLLLENNANPNLATTAGHTPLHIA |
| | | | AREGHVETVLALLEKEASQACMTKKGFTPL |
| | | | HVAAKYGKVRVAELLLERDAHPNAAGKNGL |
| | | | TPLHVAVHHNNLDIVKLLLPRGGSPHSPAW |
| | | | NGYTPLHIAAKQNQVEVARSLLQYGGSANA |
| | | | ESVQGVTPLHLAAQEGHAEMVALLLSKQAN |
| | | | GNLGNKSGLTPLHLVAQEGHVPVADVLIKH |
| | | | GVMVDATTRMGYTPLHVASHYGNIKLVKFL |
| | | | LQHQADVNAKTKLGYSPLHQAAQQGHTDIV |
| | | | TLLLKNGASPNEVSSDGTTPLAIAKRLGYI |
| | | | SVTDVLKVVTDETSFVLVSDKHRMSFPETV |
| | | | DEILDVSEDEGEELISFKAERRDSRDVDEE |
| | | | KELLDFVPKLDQVVESPAIPRIPCAMPETV |
| | | | VIRSEEQEQASKEYDEDSLIPSSPATETSD |
| | | | NISPVASPVHTGFLVSFMVDARGGSMRGSR |
| | | | HNGLRVVIPPRTCAAPTRITCRLVKPQKLS |
| | | | TPPPLAEEEGLASRIIALGPTGAQFLSPVI |
| | | | VEIPHFASHGRGDRELVVLRSENGSVWKEH |
| | | | RSRYGESYLDQILNGMDEELGSLEELEKKR |
| | | | VCRIITTDFPLYFVIMSRLCQDYDTIGPEG |
| | | | GSLKSKLVPLVQATFPENAVTKRVKLALQA |
| | | | QPVPDELVTKLLGNQATFSPIVTVEPRRRK |
| | | | FHRPIGLRIPLPPSWTDNPRDSGEGDTTSL |
| | | | RLLCSVIGGTDQAQWEDITGTTKLVYANEC |
| | | | ANFTTNVSARFWLSDCPRTAEAVNFATLLY |
| | | | KELTAVPYMAKFVIFAKMNDPREGRLRCYC |
| | | | MTDDKVDKTLEQHENFVEVARSRDIEVLEG |
| | | | MSLFAELSGNLVPVKKAAQQRSFHFQSFRE |
| | | | NRLAMPVKVRDSSREPGGSLSFLRKAMKYE |
| | | | DTQHILCHLNITMPPCAKGSGAEDRRRTPT |
| | | | PLALRYSILSESTPGSLSGTEQAEMKMAVI |
| | | | SEHLGLSWAELARELQFSVEDINRIRVENP |
| | | | NSLLEQSVALLNLWVIREGQNANMENLYTA |
| | | | LQSIDRGEIVNMLEGSGRQSRNLKPDRRHT |
| | | | DRDYSLSPSQMNGYSSLQDELLSPASLGCA |
| | | | LSSPLRADQYWNEVAVLDAIPLAATEHDTM |
| | | | LEMSDMQVWSAGLTPSLVTAEDSSLECSKA |
| | | | EDSDATGHEWKLEGALSEEPRGPELGSLEL |
| | | | VEDDTVDSDATNGLIDLLEQEEGQRSEEKL |
| | | | PGSKRQDDATGAGQDSENEVSLVSGHQRGQ |
| | | | ARITHSPTVSQVTERSQDRLQDWDADGSIV |
| | | | SYLQDAAQGSWQEEVTQGPHSFQGTSTMTE |
| | | | GLEPGGSQEYEKVLVSVSEHTWTEQPEAES |
| | | | SQADRDRRQQGQEEQVQEAKNTFTQVVQGN |
| | | | EFQNIPGEQVTEEQFTDEQGNIVTKKVELR |
| | | | GSGLQPDLIEGRKGAQIVKRASLKRGKQ |
| P16157 | 17 | 33 | MWTFVTQLLVTLVLLSFFLVSCQNVMHIVR |
| | | | GSLCFVLKHIHQELDKELGESEGLSDDEET |
| | | | ISTRVVRRRVFLKGNEFQNIPGEQVTEEQF |
| | | | TDEQGNIVTKKIIRKVVRQIDLSSADAAQE |
| | | | HEEVELRGSGLQPDLIEGRKGAQIVKRASL |
| | | | KRGKQ |
| P16157 | 18 | 34 | MWTFVTQLLVTLVLLSFFLVSCQNVMHIVR |
| | | | GSLCFVLKHIHQELDKELGESEGLSDDEET |
| | | | ISTRVVRRRVFLKGNEFQNIPGEQVTEEQF |
| | | | TDEQGNIVTKKVELRGSGLQPDLIEGRKGA |
| | | | QIVKRASLKRGKQ |
| P16157 | 19 | 35 | MWTFVTQLLVTLVLLSFFLVSCQNVMHIVR |
| | | | GSLCFVLKHIHQELDKELGESEGLSDDEET |
| | | | ISTRVVRRRVFLKDHTSTPNP |
| P16157 | 20 | 36 | MWTFVTQLLVTLVLLSFFLVSCQNVMHIVR |
| | | | GSLCFVLKHIHQVELRGSGLQPDLIEGRKG |
| | | | AQIVKRASLKRGKQ |
| P16157 | 21 | 37 | MAQAAKQLKKIKDIEAQALQEQKEKEESNR |
| | | | KRRNRSRDRKKKADAATSFLRAARSGNLDK |
| | | | ALDHLRNGVDINTCNQNGLNGLHLASKEGH |
| | | | VKMVVELLHKEIILETTTKKGNTALHIAAL |
| | | | AGQDEVVRELVNYGANVNAQSQKGFTPLYM |
| | | | AAQENHLEVVKFLLENGANQNVATEDGFTP |
| | | | LAVALQQGHENVVAHLINYGTKGKVRLPAL |
| | | | HIAARNDDTRTAAVLLQNDPNPDVLSKTGF |
| | | | TPLHIAAHYENLNVAQLLLNRGASVNFTPQ |
| | | | NGITPLHIASRRGNVIMVRLLLDRGAQIET |
| | | | KTKDELTPLHCAARNGHVRISEILLDHGAP |
| | | | IQAKTKNGLSPIHMAAQGDHLDCVRLLLQY |
| | | | DAEIDDITLDHLTPLHVAAHCGHHRVAKVL |
| | | | LDKGAKPNSRALNGFTPLHIACKKNHVRVM |
| | | | ELLLKTGASIDAVTESGLTPLHVASFMGHL |
| | | | PIVKNLLQRGASPNVSNVKVETPLHMAARA |
| | | | GHTEVAKYLLQNKAKVNAKAKDDQTPLHCA |
| | | | ARIGHTNMVKLLLENNANPNLATTAGHTPL |
| | | | HIAAREGHVETVLALLEKEASQACMTKKGF |
| | | | TPLHVAAKYGKVRVAELLLERDAHPNAAGK |
| | | | NGLTPLHVAVHHNNLDIVKLLLPRGGSPHS |
| | | | PAWNGYTPLHIAAKQNQVEVARSLLQYGGS |
| | | | ANAESVQGVTPLHLAAQEGHAEMVALLLSK |
| | | | QANGNLGNKSGLTPLHLVAQEGHVPVADVL |
| | | | IKHGVMVDATTRMGYTPLHVASHYGNIKLV |
| | | | KFLLQHQADVNAKTKLGYSPLHQAAQQGHT |
| | | | DIVTLLLKNGASPNEVSSDGTTPLAIAKRL |
| | | | GYISVTDVLKVVTDETSFVLVSDKHRMSFP |
| | | | ETVDEILDVSEDEGTAHITIMGEELISFKA |
| | | | ERRDSRDVDEEKELLDFVPKLDQVVESPAI |
| | | | PRIPCAMPETVVIRSEEQEQASKEYDEDSL |
| | | | IPSSPATETSDNISPVASPVHTGFLVSFMV |
| | | | DARGGSMRGSRHNGLRVVIPPRTCAAPTRI |
| | | | TCRLVKPQKLSTPPPLAEEEGLASRIIALG |
| | | | PTGAQFLSPVIVEIPHFASHGRGDRELVVL |
| | | | RSENGSVWKEHRSRYGESYLDQILNGMDEE |
| | | | LGSLEELEKKRVCRIITTDFPLYFVIMSRL |
| | | | CQDYDTIGPEGGSLKSKLVPLVQATFPENA |
| | | | VTKRVKLALQAQPVPDELVTKLLGNQATFS |
| | | | PIVTVEPRRRKFHRPIGLRIPLPPSWTDNP |
| | | | RDSGEGDTTSLRLLCSVIGGTDQAQWEDIT |
| | | | GTTKLVYANECANFTTNVSARFWLSDCPRT |
| | | | AEAVNFATLLYKELTAVPYMAKFVIFAKMN |
| | | | DPREGRLRCYCMTDDKVDKTLEQHENFVEV |
| | | | ARSRDIEVLEGMSLFAELSGNLVPVKKAAQ |
| | | | QRSFHFQSFRENRLAMPVKVRDSSREPGGS |
| | | | LSFLRKAMKYEDTQHILCHLNITMPPCAKG |
| | | | SGAEDRRRTPTPLALRYSILSESTPGSLSG |
| | | | TEQAEMKMAVISEHLGLSWAELARELQFSV |
| | | | EDINRIRVENPNSLLEQSVALLNLWVIREG |
| | | | QNANMENLYTALQSIDRGEIVNMLEGSGRQ |
| | | | SRNLKPDRRHTDRDYSLSPSQMNGYSSLQD |
| | | | ELLSPASLGCALSSPLRADQYWNEVAVLDA |
| | | | IPLAATEHDTMLEMSDMQVWSAGLTPSLVT |
| | | | AEDSSLECSKAEDSDATGHEWKLEGALSEE |
| | | | PRGPELGSLELVEDDTVDSDATNGLIDLLE |
| | | | QEEGQRSEEKLPGSKRQDDATGAGQDSENE |
| | | | VSLVSGHQRGQARITHSPTVSQVTERSQDR |
| | | | LQDWDADGSIVSYLQDAAQGSWQEEVTQGP |
| | | | HSFQGTSTMTEGLEPGGSQEYEKVLVSVSE |
| | | | HTWTEQPEAESSQADRDRRQQGQEEQVQEA |
| | | | KNTFTQVVQGNEFQNIPGEQVTEEQFTDEQ |
| | | | GNIVTKKIIRKVVRQIDLSSADAAQEHEED |
| | | | HTSTPNP |
| P16157 | 22 | 38 | MWTFVTQLLVTLVLLSFFLVSCQNVMHIVR |
| | | | GSLCFVLKHIHQELDKELGESEGLSDDEET |
| | | | ISTRVVRRRVFLKGNEFQNIPGEQVTEEQF |
| | | | TDEQGNIVTKKDHTSTPNP |
| P16157 | 23 | 39 | MWTFVTQLLVTLVLLSFFLVSCQNVMHIVR |
| | | | GSLCFVLKHIHQELDKELGESEGLSDDEET |
| | | | ISTRVVRRRVFLKGNEFQNIPGEQVTEEQF |
| | | | TDEQGNIVTKKIIRKVVRQIDLSSADAAQE |
| | | | HEEVTVEGPLEDPSELEVDIDYFMKHSKDH |
| | | | TSTPNP |
| P16452 | 1 | 40 | MGQALGIKSCDFQAARNNEEHHTKALSSRR |
| | | | LFVRRGQPFTIILYFRAPVRAFLPALKKVA |
| | | | LTAQTGEQPSKINRTQATFPISSLGDRKWW |
| | | | SAVVEERDAQSWTISVTTPADAVIGHYSLL |
| | | | LQVSGRKQLLLGQFTLLFNPWNREDAVFLK |
| | | | NEAQRMEYLLNQNGLIYLGTADCIQAESWD |
| | | | FGQFEGDVIDLSLRLLSKDKQVEKWSQPVH |
| | | | VARVLGALLHFLKEQRVLPTPQTQATQEGA |
| | | | LLNKRRGSVPILRQWLTGRGRPVYDGQAWV |
| | | | LAAVACTVLRCLGIPARVVTTFASAQGTGG |
| | | | RLLIDEYYNEEGLQNGEGQRGRIWIFQTST |
| | | | ECWMTRPALPQGYDGWQILHPSAPNGGGVL |
| | | | GSCDLVPVRAVKEGTLGLTPAVSDLFAAIN |
| | | | ASCVVWKCCEDGTLELTDSNTKYVGNNIST |
| | | | KGVGSDRCEDITQNYKYPEGSLQEKEVLER |
| | | | VEKEKMEREKDNGIRPPSLETASPLYLLLK |
| | | | APSSLPLRGDAQISVTLVNHSEQEKAVQLA |
| | | | IGVQAVHYNGVLAAKLWRKKLHLTLSANLE |
| | | | KIITIGLFFSNFERNPPENTFLRLTAMATH |
| | | | SESNLSCFAQEDIAICRPHLAIKMPEKAEQ |
| | | | YQPLTASVSLQNSLDAPMEDCVISILGRGL |
| | | | IHRERSYRFRSVWPENTMCAKFQFTPTHVG |
| | | | LQRLTVEVDCNMFQNLTNYKSVTVVAPELS |
| | | | A |
| P16452 | 2 | 41 | MGQGEPSQRSTGLAGLYAAPAASPVFIKGS |
| | | | GMDALGIKSCDFQAARNNEEHHTKALSSRR |
| | | | LFVRRGQPFTIILYFRAPVRAFLPALKKVA |
| | | | LTAQTGEQPSKINRTQATFPISSLGDRKWW |
| | | | SAVVEERDAQSWTISVTTPADAVIGHYSLL |
| | | | LQVSGRKQLLLGQFTLLENPWNREDAVFLK |
| | | | NEAQRMEYLLNQNGLIYLGTADCIQAESWD |
| | | | FGQFEGDVIDLSLRLLSKDKQVEKWSQPVH |
| | | | VARVLGALLHFLKEQRVLPTPQTQATQEGA |
| | | | LLNKRRGSVPILRQWLTGRGRPVYDGQAWV |
| | | | LAAVACTVLRCLGIPARVVTTFASAQGTGG |
| | | | RLLIDEYYNEEGLQNGEGQRGRIWIFQTST |
| | | | ECWMTRPALPQGYDGWQILHPSAPNGGGVL |
| | | | GSCDLVPVRAVKEGTLGLTPAVSDLFAAIN |
| | | | ASCVVWKCCEDGTLELTDSNTKYVGNNIST |
| | | | KGVGSDRCEDITQNYKYPEGSLQEKEVLER |
| | | | VEKEKMEREKDNGIRPPSLETASPLYLLLK |
| | | | APSSLPLRGDAQISVTLVNHSEQEKAVQLA |
| | | | IGVQAVHYNGVLAAKLWRKKLHLTLSANLE |
| | | | KIITIGLFFSNFERNPPENTFLRLTAMATH |
| | | | SESNLSCFAQEDIAICRPHLAIKMPEKAEQ |
| | | | YQPLTASVSLQNSLDAPMEDCVISILGRGL |
| | | | IHRERSYRFRSVWPENTMCAKFQFTPTHVG |
| | | | LQRLTVEVDCNMFQNLTNYKSVTVVAPELS |
| | | | A |
| P16452 | 3 | 42 | MGQALGIKSCDFQAARNNEEHHTKALSSRR |
| | | | LFVRRGQPFTIILYFRAPVRAFLPALKKVA |
| | | | LTAQTGEQPSKINRTQATFPISSLGDRKWW |
| | | | SAVVEERDAQSWTISVTTPADAVIGHYSLL |
| | | | LQVSGRKQLLLGQFTLLENPWNREDAVFLK |
| | | | NEAQRMEYLLNQNGLIYLGTADCIQAESWD |
| | | | FGQFEGDVIDLSLRLLSKDKQVEKWSQPVH |
| | | | VARVLGALLHFLKEQRVLPTPQTQATQEGA |
| | | | LLNKRRGSVPILRQWLTGRGRPVYDGQAWV |
| | | | LAAVACTVLRCLGIPARVVTTFASAQGTGG |
| | | | RLLIDEYYNEEGLQNGEGQRGRIWKCCEDG |
| | | | TLELTDSNTKYVGNNISTKGVGSDRCEDIT |
| | | | QNYKYPEGSLQEKEVLERVEKEKMEREKDN |
| | | | GIRPPSLETASPLYLLLKAPSSLPLRGDAQ |
| | | | ISVTLVNHSEQEKAVQLAIGVQAVHYNGVL |
| | | | AAKLWRKKLHLTLSANLEKIITIGLFFSNF |
| | | | ERNPPENTFLRLTAMATHSESNLSCFAQED |
| | | | IAICRPHLAIKMPEKAEQYQPLTASVSLQN |
| | | | SLDAPMEDCVISILGRGLIHRERSYRFRSV |
| | | | WPENTMCAKFQFTPTHVGLQRLTVEVDCNM |
| | | | FQNLTNYKSVTVVAPELSA |
| P17936 | 1 | 43 | MQRARPTLWAAALTLLVLLRGPPVARAGAS |
| | | | SAGLGPVVRCEPCDARALAQCAPPPAVCAE |
| | | | LVREPGCGCCLTCALSEGQPCGIYTERCGS |
| | | | GLRCQPSPDEARPLQALLDGRGLCVNASAV |
| | | | SRLRAYLLPAPPAPGNASESEEDRSAGSVE |
| | | | SPSVSSTHRVSDPKFHPLHSKIIIIKKGHA |
| | | | KDSQRYKVDYESQSTDTQNFSSESKRETEY |
| | | | GPCRREMEDTLNHLKFLNVLSPRGVHIPNC |
| | | | DKKGFYKKKQCRPSKGRKRGFCWCVDKYGQ |
| | | | PLPGYTTKGKEDVHCYSMQSK |
| P17936 | 2 | 44 | MQRARPTLWAAALTLLVLLRGPPVARAGAS |
| | | | SAGLGPVVRCEPCDARALAQCAPPPAVCAE |
| | | | LVREPGCGCCLTCALSEGQPCGIYTERCGS |
| | | | GLRCQPSPDEARPLQALLDGRGLCVNASAV |
| | | | SRLRAYLLPAPPAPGEPPAPGNASESEEDR |
| | | | SAGSVESPSVSSTHRVSDPKFHPLHSKIII |
| | | | IKKGHAKDSQRYKVDYESQSTDTQNFSSES |
| | | | KRETEYGPCRREMEDTLNHLKFLNVLSPRG |
| | | | VHIPNCDKKGFYKKKQCRPSKGRKRGFCWC |
| | | | VDKYGQPLPGYTTKGKEDVHCYSMQSK |
| P27918 | 1 | 45 | MITEGAQAPRLLLPPLLLLLTLPATGSDPV |
| | | | LCFTQYEESSGKCKGLLGGGVSVEDCCLNT |
| | | | AFAYQKRSGGLCQPCRSPRWSLWSTWAPCS |
| | | | VTCSEGSQLRYRRCVGWNGQCSGKVAPGTL |
| | | | EWQLQACEDQQCCPEMGGWSGWGPWEPCSV |
| | | | TCSKGTRTRRRACNHPAPKCGGHCPGQAQE |
| | | | SEACDTQQVCPTHGAWATWGPWTPCSASCH |
| | | | GGPHEPKETRSRKCSAPEPSQKPPGKPCPG |
| | | | LAYEQRRCTGLPPCPVAGGWGPWGPVSPCP |
| | | | VTCGLGQTMEQRTCNHPVPQHGGPFCAGDA |
| | | | TRTHICNTAVPCPVDGEWDSWGEWSPCIRR |
| | | | NMKSISCQEIPGQQSRGRTCRGRKFDGHRC |
| | | | AGQQQDIRHCYSIQHCPLKGSWSEWSTWGL |
| | | | CMPPCGPNPTRARQRLCTPLLPKYPPTVSM |
| | | | VEGQGEKNVTFWGRPLPRCEELQGQKLVVE |
| | | | EKRPCLHVPACKDPEEEEL |
| P30043 | 1 | 46 | MAVKKIAIFGATGQTGLTTLAQAVQAGYEV |
| | | | TVLVRDSSRLPSEGPRPAHVVVGDVLQAAD |
| | | | VDKTVAGQDAVIVLLGTRNDLSPTTVMSEG |
| | | | ARNIVAAMKAHGVDKVVACTSAFLLWDPTK |
| | | | VPPRLQAVTDDHIRMHKVLRESGLKYVAVM |
| | | | PPHIGDQPLTGAYTVTLDGRGPSRVISKHD |
| | | | LGHFMLRCLTTDEYDGHSTYPSHQYQ |
| Q12797 | 1 | 47 | MAQRKNAKSSGNSSSSGSGSGSTSAGSSSP |
| | | | GARRETKHGGHKNGRKGGLSGTSFFTWFMV |
| | | | IALLGVWTSVAVVWFDLVDYEEVLGKLGIY |
| | | | DADGDGDFDVDDAKVLLGLKERSTSEPAVP |
| | | | PEEAEPHTEPEEQVPVEAEPQNIEDEAKEQ |
| | | | IQSLLHEMVHAEHVEGEDLQQEDGPTGEPQ |
| | | | QEDDEFLMATDVDDRFETLEPEVSHEETEH |
| | | | SYHVEETVSQDCNQDMEEMMSEQENPDSSE |
| | | | PVVEDERLHHDTDDVTYQVYEEQAVYEPLE |
| | | | NEGIEITEVTAPPEDNPVEDSQVIVEEVSI |
| | | | FPVEEQQEVPPETNRKTDDPEQKAKVKKKK |
| | | | PKLLNKFDKTIKAELDAAEKLRKRGKIEEA |
| | | | VNAFKELVRKYPQSPRARYGKAQCEDDLAE |
| | | | KRRSNEVLRGAIETYQEVASLPDVPADLLK |
| | | | LSLKRRSDRQQFLGHMRGSLLTLQRLVQLF |
| | | | PNDTSLKNDLGVGYLLIGDNDNAKKVYEEV |
| | | | LSVTPNDGFAKVHYGFILKAQNKIAESIPY |
| | | | LKEGIESGDPGTDDGRFYFHLGDAMQRVGN |
| | | | KEAYKWYELGHKRGHFASVWQRSLYNVNGL |
| | | | KAQPWWTPKETGYTELVKSLERNWKLIRDE |
| | | | GLAVMDKAKGLFLPEDENLREKGDWSQFTL |
| | | | WQQGRRNENACKGAPKTCTLLEKFPETTGC |
| | | | RRGQIKYSIMHPGTHVWPHTGPTNCRLRMH |
| | | | LGLVIPKEGCKIRCANETKTWEEGKVLIFD |
| | | | DSFEHEVWQDASSFRLIFIVDVWHPELTPQ |
| | | | QRRSLPAI |
| Q12797 | 2 | 48 | MAQRKNAKSSGNSSSSGSGSGSTSAGSSSP |
| | | | GARRETKHGGHKNGRKGGLSGTSFFTWFMV |
| | | | IALLGVWTSVAVVWFDLVDYEEVLGKLGIY |
| | | | DADGDGDFDVDDAKVLLGLKERSTSEPAVP |
| | | | PEEAEPHTEPEEQVPVEAEPQNIEDEAKEQ |
| | | | IQSLLHEMVHAEHVEGEDLQQEDGPTGEPQ |
| | | | QEDDEFLMATDVDDRFETLEPEVSHEETEH |
| | | | SYHVEETVSQDCNQDMEEMMSEQENPDSSE |
| | | | PVVEDERLHHDTDDVTYQVYEEQAVYEPLE |
| | | | NEGIEITEVTAPPEDNPVEDSQVIVEEVSI |
| | | | FPVEEQQEVPPDT |
| Q12797 | 3 | 49 | MAEDKETKHGGHKNGRKGGLSGTSFFTWFM |
| | | | VIALLGVWTSVAVVWFDLVDYEEVLAKAKD |
| | | | FRYNLSEVLQGKLGIYDADGDGDFDVDDAK |
| | | | VLLEGPSGVAKRKTKAKVKELTKEELKKEK |
| | | | EKPESRKESKNEERKKGKKEDVRKDKKIAD |
| | | | ADLSRKESPKGKKDREKEKVDLEKSAKTKE |
| | | | NRKKSTNMKDVSSKMASRDKDDRKESRSST |
| | | | RYAHLTKGNTQKRNG |
| Q12797 | 4 | 50 | MAEDKETKHGGHKNGRKGGLSGTSFFTWFM |
| | | | VIALLGVWTSVAVVWFDLVDYEEVLGKLGI |
| | | | YDADGDGDFDVDDAKVLLEGPSGVAKRKTK |
| | | | AKVKELTKEELKKEKEKPESRKESKNEERK |
| | | | KGKKEDVRKDKKIADADLSRKESPKGKKDR |
| | | | EKEKVDLEKSAKTKENRKKSTNMKDVSSKM |
| | | | ASRDKDDRKESRSSTRYAHLTKGNTQKRNG |
| Q12797 | 5 | 51 | MAEDKETKHGGHKNGRKGGLSGTSFFTWFM |
| | | | VIALLGVWTSVAVVWFDLVDYEEVLAKAKD |
| | | | FRYNLSEVLQGKLGIYDADGDGDFDVDDAK |
| | | | VLLGLKERSTSEPAVPPEEAEPHTEPEEQV |
| | | | PVEAEPQNIEDEAKEQIQSLLHEMVHAEHV |
| | | | EGEDLQQEDGPTGEPQQEDDEFLMATDVDD |
| | | | RFETLEPEVSHEETEHSYHVEETVSQDCNQ |
| | | | DMEEMMSEQENPDSSEPVVEDERLHHDTDD |
| | | | VTYQVYEEQVYEPLENEGIEITEVTAPPED |
| | | | NPVEDSQVIVEEVSIFPVEEQQEVPPDT |
| Q12797 | 6 | 52 | MAQRKNAKSSGNSSSSGSGSGSTSAGSSSP |
| | | | GARRETKHGGHKNGRKGGLSGTSFFTWFMV |
| | | | IALLGVWTSVAVVWFDLVDYEEVLGKLGIY |
| | | | DADGDGDFDVDDAKVLLGLKERSTSEPAVP |
| | | | PEEAEPHTEPEEQVPVEAEPQNIEDEAKEQ |
| | | | IQSLLHEMVHAEHETEHSYHVEETVSQDCN |
| | | | QDMEEMMSEQENPDSSEPVVEDERLHHDTD |
| | | | DVTYQVYEEQAVYEPLENEGIEITEVTAPP |
| | | | EDNPVEDSQVIVEEVSIFPVEEQQEVPPDT |
| Q12797 | 7 | 53 | MAQRKNAKSSGNSSSSGSGSGSTSAGSSSP |
| | | | GARRETKHGGHKNGRKGGLSGTSFFTWFMV |
| | | | IALLGVWTSVAVVWFDLVDYEEVLAKAKDF |
| | | | RYNLSEVLQGKLGIYDADGDGDFDVDDAKV |
| | | | LLGLTKDGSNENIDSLEEVLNILAEESSDW |
| | | | FYGFLSFLYDIMTPFEMLEEEEEESETADG |
| | | | VDGTSQNEGVQGKTCVILDLHNQ |
| Q12797 | 8 | 54 | MAEDKETKHGGHKNGRKGGLSGTSFFTWFM |
| | | | VIALLGVWTSVAVVWFDLVDYEEVLAKAKD |
| | | | FRYNLSEVLQGKLGIYDADGDGDFDVDDAK |
| | | | VLLGLKERSTSEPAVPPEEAEPHTEPEEQV |
| | | | PVEAEPQNIEDEAKEQIQSLLHEMVHAEHV |
| | | | EGEDLQQEDGPTGEPQQEDDEFLMATDVDD |
| | | | RFETLEPEVSHEETEHSYHVEETVSQDCNQ |
| | | | DMEEMMSEQENPDSSEPVVEDERLHHDTDD |
| | | | VTYQVYEEQAVYEPLENEGIEITEVTAPPE |
| | | | DNPVEDSQVIVEEVSIFPVEEQQEVPPDT |
| Q12797 | 9 | 55 | MAEDKETKHGGHKNGRKGGLSGTSFFTWFM |
| | | | VIALLGVWTSVAVVWFDLVDYEEVLAKAKD |
| | | | FRYNLSEVLQGKLGIYDADGDGDFDVDDAK |
| | | | VLLGLKERSTSEPAVPPEEAEPHTEPEEQV |
| | | | PVEAEPQNIEDEAKEQIQSLLHEMVHAEHE |
| | | | TEHSYHVEETVSQDCNQDMEEMMSEQENPD |
| | | | SSEPVVEDERLHHDTDDVTYQVYEEQAVYE |
| | | | PLENEGIEITEVTAPPEDNPVEDSQVIVEE |
| | | | VSIFPVEEQQEVPPDT |
| Q12797 | 10 | 56 | MAEDKETKHGGHKNGRKGGLSGTSFFTWFM |
| | | | VIALLGVWTSVAVVWFDLVDYEEVLGKLGI |
| | | | YDADGDGDFDVDDAKVLLGLKERSTSEPAV |
| | | | PPEEAEPHTEPEEQVPVEAEPQNIEDEAKE |
| | | | QIQSLLHEMVHAEHVEGEDLQQEDGPTGEP |
| | | | QQEDDEFLMATDVDDRFETLEPEVSHEETE |
| | | | HSYHVEETVSQDCNQDMEEMMSEQENPDSS |
| | | | EPVVEDERLHHDTDDVTYQVYEEQAVYEPL |
| | | | ENEGIEITEVTAPPEDNPVEDSQVIVEEVS |
| | | | IFPVEEQQEVPPETNRKTDDPEQKAKVKKK |
| | | | KPKLLNKFDKTIKAELDAAEKLRKRGKIEE |
| | | | AVNAFKELVRKYPQSPRARYGKAQCEDDLA |
| | | | EKRRSNEVLRGAIETYQEVASLPDVPADLL |
| | | | KLSLKRRSDRQQFLGHMRGSLLTLQRLVQL |
| | | | FPNDTSLKNDLGVGYLLIGDNDNAKKVYEE |
| | | | VLSVTPNDGFAKVHYGFILKAQNKIAESIP |
| | | | YLKEGIESGDPGTDDGRFYFHLGDAMQRVG |
| | | | NKEAYKWYELGHKRGHFASVWQRSLYNVNG |
| | | | LKAQPWWTPKETGYTELVKSLERNWKLIRD |
| | | | EGLAVMDKAKGLFLPEDENLREKGDWSQFT |
| | | | LWQQGRRNENACKGAPKTCTLLEKFPETTG |
| | | | CRRGQIKYSIMHPGTHVWPHTGPTNCRLRM |
| | | | HLGLVIPKEGCKIRCANETKTWEEGKVLIF |
| | | | DDSFEHEVWQDASSFRLIFIVDVWHPELTP |
| | | | QQRRSLPAI |
| Q12797 | 11 | 57 | MAQRKNAKSSGNSSSSGSGSGSTSAGSSSP |
| | | | GARRETKHGGHKNGRKGGLSGTSFFTWFMV |
| | | | IALLGVWTSVAVVWFDLVDYEEVLGKLGIY |
| | | | DADGDGDFDVDDAKVLLGLKERSTSEPAVP |
| | | | PEEAEPHTEPEEQVPVEAEPQNIEDEAKEQ |
| | | | IQSLLHEMVHAEHVEGEDLQQEDGPTGEPQ |
| | | | QEDDEFLMATDVDDRFETLEPEVSHEETEH |
| | | | SYHVEETDSSEPVVEDERLHHDTDDVTYQV |
| | | | YEEQAVYEPLENEGIEITEVTAPPEDNPVE |
| | | | DSQVIVEEVSIFPVEEQQEVPPDT |
| Q13822 | 1 | 58 | MARRSSFQSCQIISLFTFAVGVNICLGFTA |
| | | | HRIKRAEGWEEGPPTVLSDSPWTNISGSCK |
| | | | GRCFELQEAGPPDCRCDNLCKSYTSCCHDF |
| | | | DELCLKTARGWECTKDRCGEVRNEENACHC |
| | | | SEDCLARGDCCTNYQVVCKGESHWVDDDCE |
| | | | EIKAAECPAGFVRPPLIIFSVDGFRASYMK |
| | | | KGSKVMPNIEKLRSCGTHSPYMRPVYPTKT |
| | | | FPNLYTLATGLYPESHGIVGNSMYDPVFDA |
| | | | TFHLRGREKFNHRWWGGQPLWITATKQGVK |
| | | | AGTFFWSVVIPHERRILTILQWLTLPDHER |
| | | | PSVYAFYSEQPDFSGHKYGPFGPEMTNPLR |
| | | | EIDKIVGQLMDGLKQLKLHRCVNVIFVGDH |
| | | | GMEDVTCDRTEFLSNYLTNVDDITLVPGTL |
| | | | GRIRSKFSNNAKYDPKAIIANLTCKKPDQH |
| | | | FKPYLKQHLPKRLHYANNRRIEDIHLLVER |
| | | | RWHVARKPLDVYKKPSGKCFFQGDHGFDNK |
| | | | VNSMQTVFVGYGSTFKYKTKVPPFENIELY |
| | | | NVMCDLLGLKPAPNNGTHGSLNHLLRTNTF |
| | | | RPTMPEEVTRPNYPGIMYLQSDFDLGCTCD |
| | | | DKVEPKNKLDELNKRLHTKGSTEERHLLYG |
| | | | RPAVLYRTRYDILYHTDFESGYSEIFLMPL |
| | | | WTSYTVSKQAEVSSVPDHLTSCVRPDVRVS |
| | | | PSFSQNCLAYKNDKQMSYGFLFPPYLSSSP |
| | | | EAKYDAFLVTNMVPMYPAFKRVWNYFQRVL |
| | | | VKKYASERNGVNVISGPIFDYDYDGLHDTE |
| | | | DKIKQYVEGSSIPVPTHYYSIITSCLDFTQ |
| | | | PADKCDGPLSVSSFILPHRPDNEESCNSSE |
| | | | DESKWVEELMKMHTARVRDIEHLTSLDFFR |
| | | | KTSRSYPEILTLKTYLHTYESEI |
| Q13822 | 2 | 59 | MARRSSFQSCQIISLFTFAVGVNICLGFTA |
| | | | HRIKRAEGWEEGPPTVLSDSPWTNISGSCK |
| | | | GRCFELQEAGPPDCRCDNLCKSYTSCCHDF |
| | | | DELCLKTARGWECTKDRCGEVRNEENACHC |
| | | | SEDCLARGDCCTNYQVVCKGESHWVDDDCE |
| | | | EIKAAECPAGFVRPPLIIFSVDGFRASYMK |
| | | | KGSKVMPNIEKLRSCGTHSPYMRPVYPTKT |
| | | | FPNLYTLATGLYPESHGIVGNSMYDPVFDA |
| | | | TFHLRGREKFNHRWWGGQPLWITATKQGVK |
| | | | AGTFFWSVVIPHERRILTILQWLTLPDHER |
| | | | PSVYAFYSEQPDFSGHKYGPFGPEESSYGS |
| | | | PFTPAKRPKRKVAPKRRQERPVAPPKKRRR |
| | | | KIHRMDHYAAETRQDKMTNPLREIDKIVGQ |
| | | | LMDGLKQLKLHRCVNVIFVGDHGMEDVTCD |
| | | | RTEFLSNYLTNVDDITLVPGTLGRIRSKFS |
| | | | NNAKYDPKAIIANLTCKKPDQHFKPYLKQH |
| | | | LPKRLHYANNRRIEDIHLLVERRWHVARKP |
| | | | LDVYKKPSGKCFFQGDHGFDNKVNSMQTVF |
| | | | VGYGSTFKYKTKVPPFENIELYNVMCDLLG |
| | | | LKPAPNNGTHGSLNHLLRTNTFRPTMPEEV |
| | | | TRPNYPGIMYLQSDFDLGCTCDDKVEPKNK |
| | | | LDELNKRLHTKGSTEERHLLYGRPAVLYRT |
| | | | RYDILYHTDFESGYSEIFLMPLWTSYTVSK |
| | | | QAEVSSVPDHLTSCVRPDVRVSPSFSQNCL |
| | | | AYKNDKQMSYGFLFPPYLSSSPEAKYDAFL |
| | | | VTNMVPMYPAFKRVWNYFQRVLVKKYASER |
| | | | NGVNVISGPIFDYDYDGLHDTEDKIKQYVE |
| | | | GSSIPVPTHYYSIITSCLDFTQPADKCDGP |
| | | | LSVSSFILPHRPDNEESCNSSEDESKWVEE |
| | | | LMKMHTARVRDIEHLTSLDFFRKTSRSYPE |
| | | | ILTLKTYLHTYESEI |
| Q13822 | 3 | 60 | MARRSSFQSCQIISLFTFAVGVNICLGFTA |
| | | | HRIKRAEGWEEGPPTVLSDSPWTNISGSCK |
| | | | GRCFELQEAGPPDCRCDNLCKSYTSCCHDF |
| | | | DELCLKTARGWECTKDRCGEVRNEENACHC |
| | | | SEDCLARGDCCTNYQVVCKGESHWVDDDCE |
| | | | EIKAAECPAGFVRPPLIIFSVDGFRASYMK |
| | | | KGSKVMPNIEKLRSCGTHSPYMRPVYPTKT |
| | | | FPNLYTLATGLYPESHGIVGNSMYDPVFDA |
| | | | TFHLRGREKFNHRWWGGQPLWITATKQGVK |
| | | | AGTFFWSVVIPHERRILTILQWLTLPDHER |
| | | | PSVYAFYSEQPDFSGHKYGPFGPEMTNPLR |
| | | | EIDKIVGQLMDGLKQLKLHRCVNVIFVGDH |
| | | | GMEDVTCDRTEFLSNYLTNVDDITLVPGTL |
| | | | GRIRSKFSNNAKYDPKAIIANLTCKKPDQH |
| | | | FKPYLKQHLPKRLHYANNRRIEDIHLLVER |
| | | | RWHVARKPLDVYKKPSGKCFFQGDHGFDNK |
| | | | VNSMQTVFVGYGSTFKYKTKVPPFENIELY |
| | | | NVMCDLLGLKPAPNNGTHGSLNHLLRTNTF |
| | | | RPTMPEEVTRPNYPGIMYLQSDFDLGCTCD |
| | | | DKVEPKNKLDELNKRLHTKGSTEAETRKFR |
| | | | GSRNENKENINGNFEPRKERHLLYGRPAVL |
| | | | YRTRYDILYHTDFESGYSEIFLMPLWTSYT |
| | | | VSKQAEVSSVPDHLTSCVRPDVRVSPSFSQ |
| | | | NCLAYKNDKQMSYGFLFPPYLSSSPEAKYD |
| | | | AFLVTNMVPMYPAFKRVWNYFQRVLVKKYA |
| | | | SERNGVNVISGPIFDYDYDGLHDTEDKIKQ |
| | | | YVEGSSIPVPTHYYSIITSCLDFTQPADKC |
| | | | DGPLSVSSFILPHRPDNEESCNSSEDESKW |
| | | | VEELMKMHTARVRDIEHLTSLDFFRKTSRS |
| | | | YPEILTLKTYLHTYESEI |
| E7EUF1 | 1 | 61 | MTRHADRIISLFTFAVGVNICLGFTAHRIK |
| | | | RAEGWEEGPPTVLSDSPWTNISGSCKGRCF |
| | | | ELQEAGPPDCRCDNLCKSYTSCCHDFDELC |
| | | | LKTARGWECTKDRCGEVRNEENACHCSEDC |
| | | | LARGDCCTNYQVVCKGESHWVDDDCEEIKA |
| | | | AECPAGFVRPPLIIFSVDGFRASYMKKGSK |
| | | | VMPNIEKLRSCGTHSPYMRPVYPTKTFPNL |
| | | | YTLATGLYPESHGIVGNSMYDPVFDATFHL |
| | | | RGREKFNHRWWGGQPLWITATKQGVKAGTF |
| | | | FWSVVIPHERRILTILQWLTLPDHERPSVY |
| | | | AFYSEQPDFSGHKYGPFGPEMTNPLREIDK |
| | | | IVGQLMDGLKQLKLHRCVNVIFVGDHGMED |
| | | | VTCDRTEFLSNYLTNVDDITLVPGTLGRIR |
| | | | SKFSNNAKYDPKAIIANLTCKKPDQHFKPY |
| | | | LKQHLPKRLHYANNRRIEDIHLLVERRWHV |
| | | | ARKPLDVYKKPSGKCFFQGDHGFDNKVNSM |
| | | | QTVFVGYGSTFKYKTKVPPFENIELYNVMC |
| | | | DLLGLKPAPNNGTHGSLNHLLRTNTFRPTM |
| | | | PEEVTRPNYPGIMYLQSDFDLGCTCDDKVE |
| | | | PKNKLDELNKRLHTKGSTEAETRKFRGSRN |
| | | | ENKENINGNFEPRKERHLLYGRPAVLYRTR |
| | | | YDILYHTDFESGYSEIFLMPLWTSYTVSKQ |
| | | | AEVSSVPDHLTSCVRPDVRVSPSFSQNCLA |
| | | | YKNDKQMSYGFLFPPYLSSSPEAKYDAFLV |
| | | | TNMVPMYPAFKRVWNYFQRVLVKKYASERN |
| | | | GVNVISGPIFDYDYDGLHDTEDKIKQYVEG |
| | | | SSIPVPTHYYSIITSCLDFTQPADKCDGPL |
| | | | SVSSFILPHRPDNEESCNSSEDESKWVEEL |
| | | | MKMHTARVRDIEHLTSLDFFRKTSRSYPEI |
| | | | LTLKTYLHTYESEI |
| Q8NI99 | 1 | 62 | MGKPWLRALQLLLLLGASWARAGAPRCTYT |
| | | | FVLPPQKFTGAVCWSGPASTRATPEAANAS |
| | | | ELAALRMRVGRHEELLRELQRLAAADGAVA |
| | | | GEVRALRKESRGLSARLGQLRAQLQHEAGP |
| | | | GAGPGADLGAEPAAALALLGERVLNASAEA |
| | | | QRAAARFHQLDVKFRELAQLVTQQSSLIAR |
| | | | LERLCPGGAGGQQQVLPPPPLVPVVPVRLV |
| | | | GSTSDTSRMLDPAPEPQRDQTQRQQEPMAS |
| | | | PMPAGHPAVPTKPVGPWQDCAEARQAGHEQ |
| | | | SGVYELRVGRHVVSVWCEQQLEGGGWTVIQ |
| | | | RRQDGSVNFFTTWQHYKAGFGRPDGEYWLG |
| | | | LEPVYQLTSRGDHELLVLLEDWGGRGARAH |
| | | | YDGFSLEPESDHYRLRLGQYHGDAGDSLSW |
| | | | HNDKPFSTVDRDRDSYSGNCALYQRGGWWY |
| | | | HACAHSNLNGVWHHGGHYRSRYQDGVYWAE |
| | | | FRGGAYSLRKAAMLIRPLKL |
| Q96IY4 | 1 | 63 | MKLCSLAVLVPIVLFCEQHVFAFQSGQVLA |
| | | | ALPRTSRQVQVLQNLTTTYEIVLWQPVTAD |
| | | | LIVKKKQVHFFVNASDVDNVKAHLNVSGIP |
| | | | CSVLLADVEDLIQQQISNDTVSPRASASYY |
| | | | EQYHSLNEIYSWIEFITERHPDMLTKIHIG |
| | | | SSFEKYPLYVLKVSGKEQAAKNAIWIDCGI |
| | | | HAREWISPAFCLWFIGHITQFYGIIGQYTN |
| | | | LLRLVDFYVMPVVNVDGYDYSWKKNRMWRK |
| | | | NRSFYANNHCIGTDLNRNFASKHWCEEGAS |
| | | | SSSCSETYCGLYPESEPEVKAVASFLRRNI |
| | | | NQIKAYISMHSYSQHIVFPYSYTRSKSKDH |
| | | | EELSLVASEAVRAIEKISKNTRYTHGHGSE |
| | | | TLYLAPGGGDDWIYDLGIKYSFTIELRDTG |
| | | | TYGFLLPERYIKPTCREAFAAVSKIAWHVI |
| | | | RNV |
| Q96IY4 | 2 | 64 | MKLCSLAVLVPIVLFCEQHVFAFQSGQVLA |
| | | | ALPRTSRQVQVLQNLTTTYEIVLWQPVTAD |
| | | | LIVKKKQVHFFVNASDVDNVKAHLNVSGIP |
| | | | CSVLLADVEDLIQQQISNDTVSPRASASYY |
| | | | EQYHSLNEIYSWIEFITERHPDMLTKIHIG |
| | | | SSFEKYPLYVLKVSGKEQAAKNAIWIDCGI |
| | | | HAREWISPAFCLWFIGHNRMWRKNRSFYAN |
| | | | NHCIGTDLNRNFASKHWCEEGASSSSCSET |
| | | | YCGLYPESEPEVKAVASFLRRNINQIKAYI |
| | | | SMHSYSQHIVFPYSYTRSKSKDHEELSLVA |
| | | | SEAVRAIEKISKNTRYTHGHGSETLYLAPG |
| | | | GGDDWIYDLGIKYSFTSNPPVEKLLPLSLK |
| Q9BXN1 | 1 | 65 | MKEYVLLLFLALCSAKPFFSPSHIALKNMM |
| | | | LKDMEDTDDDDDDDDDDDDDDEDNSLFPTR |
| | | | EPRSHFFPFDLFPMCPFGCQCYSRVVHCSD |
| | | | LGLTSVPTNIPFDTRMLDLQNNKIKEIKEN |
| | | | DFKGLTSLYGLILNNNKLTKIHPKAFLTTK |
| | | | KLRRLYLSHNQLSEIPLNLPKSLAELRIHE |
| | | | NKVKKIQKDTFKGMNALHVLEMSANPLDNN |
| | | | GIEPGAFEGVTVFHIRIAEAKLTSVPKGLP |
| | | | PTLLELHLDYNKISTVELEDFKRYKELQRL |
| | | | GLGNNKITDIENGSLANIPRVREIHLENNK |
| | | | LKKIPSGLPELKYLQIIFLHSNSIARVGVN |
| | | | DFCPTVPKMKKSLYSAISLFNNPVKYWEMQ |
| | | | PATFRCVLSRMSVQLGNFGM |

TABLE 3
Sequences of Top Protein Group Features and Isoforms from DIA Data

| | Isoform | SEQ ID | |
|---|---|---|---|
| UniProtKB | No. | NO. | SEQUENCE |
| O94812 | 1 | 66 | MRPRGAAFAAGPPGDLHLGTAIGFAGAIWR |
| | | | SRSPAMSTLLDIKSSVLRQVQVCPSFRRRT |
| | | | EQDPGSASADPQEPATGAWKPGDGVEFFAH |
| | | | MRLMLKKGEGRQGLPCLEVPLRSGSPAPPE |
| | | | PVDPSLGLRALAPEEVEMLYEEALYTVLYR |
| | | | AGTMGPDQVDDEEALLSYLQQVFGTSLEEH |
| | | | TEAIERVRKAKAPTYALKVSVMRAKNLLAK |
| | | | DPNGFSDPYCMLGILPASDATREPRAQKEQ |
| | | | RFGFRKGSKRGGPLPAKCIQVTEVKSSTLN |
| | | | PVWKEHFLFEIEDVSTDQLHLDIWDHDDDV |
| | | | SLVEACRKLNEVIGLKGMGRYFKQIVKSAR |
| | | | ANGTAGPTEDHTDDFLGCLNIPVREVPVAG |
| | | | VDRWFKLEPRSSASRVQGHCHLVLKLITTQ |
| | | | RDTAMSQRGRSGFLSHLLLLSHLLRLEHSA |
| | | | EEPNSSSWRGELSTPAATILCLHGAQSNLS |
| | | | PLQLAVLHWQVSSRHHQTCTLDYSYLLGLL |
| | | | EDMQAHWEEAPSLPQEQEESLADSLSAFSE |
| | | | FGLQLLRQLRDYFPATNSTAVHRLELLLKC |
| | | | LGKLQLFQPSFEICPFESELNMDIAAALKR |
| | | | GNREWYDRILNDKSPREQPGPQRLPGLVVL |
| | | | ADAVYDDLQFCYSVYASLFHSILNVDVFTL |
| | | | TFRQLERLVAEEAWVLTEELSPKMTLEVAS |
| | | | GLFELYLTLADLQRFWDSIPGRDSRSLALA |
| | | | GIHAPFLPAVKLWFQVLRDQAKWRLQGAVD |
| | | | MDTLEPVDASSRHSSSAATAGLCLSHIQEL |
| | | | WVRLAWPDPAQAQGLGTQLGQDVCEATLFY |
| | | | TELLRKKVDTQPGAAGEAVSEALCVVLNNV |
| | | | ELVRKAAGQALKGLAWPEGATGPEGVLPRP |
| | | | LLSCTQALDDDLQREAHTVTAHLTSKMVGD |
| | | | IRKYVQHISLSPDSIQNDEAVAPLMKYLDE |
| | | | KLALLNASLVKGNLSRVLEALWELLLQAIL |
| | | | QALGANRDVSADFYSRFHFTLEALVSFFHA |
| | | | EGQGLPLESLRDGSYKRLKEELRLHKCSTR |
| | | | ECIEQFYLDKLKQRTLEQNRFGRLSVRCHY |
| | | | EAAEQRLAVEVLHAADLLPLDANGLSDPFV |
| | | | IVELGPPHLFPLVRSQRTQVKTRTLHPVYD |
| | | | ELFYFSVPAEACRRRAACVLFTVMDHDWLS |
| | | | TNDFAGEAALGLGGVTGVARPQVGGGARAG |
| | | | QPVTLHLCRPRAQVRSALRRLEGRTSKEAQ |
| | | | EFVKKLKELEKCMEADP |
| O94812 | 2 | 67 | MSTLLDIKSSVLRQVQVCPSFRRRTEQDPG |
| | | | SASADPQEPATGAWKPGDGVEFFAHMRLML |
| | | | KKGEGRQGLPCLEAWGSPCRQSPHPGPHTQ |
| | | | VPLRSGSPAPPEPVDPSLGLRALAPEEVEM |
| | | | LYEEALYTVLYRAGTMGPDQVDDEEALLSY |
| | | | LQQVFGTSLEEHTEAIERVRKAKAPTYALK |
| | | | VSVMRAKNLLAKDPNGFSDPYCMLGILPAS |
| | | | DATREPRAQKEQRFGFRKGSKRGGPLPAKC |
| | | | IQVTEVKSSTLNPVWKEHFLFEIEDVSTDQ |
| | | | LHLDIWDHDDDVSLVEACRKLNEVIGLKGM |
| | | | GRYFKQIVKSARANGTAGPTEDHTDDFLGC |
| | | | LNIPVREVPVAGVDRWFKLEPRSSASRVQG |
| | | | HCHLVLKLITTQRDTAMSQRGRSGFLSHLL |
| | | | LLSHLLRLEHSAEEPNSSSWRGELSTPAAT |
| | | | ILCLHGAQSNLSPLQLAVLHWQVSSRHHQT |
| | | | CTLDYSYLLGLLEDMQAHWEEAPSLPQEQE |
| | | | ESLADSLSAFSEFGLQLLRQLRDYFPATNS |
| | | | TAVHRLELLLKCLGKLQLFQPSFEICPFES |
| | | | ELNMDIAAALKRGNREWYDRILNDKSPREQ |
| | | | PGPQRLPGLVVLADAVYDDLQFCYSVYASL |
| | | | FHSILNVDVFTLTFRQLERLVAEEAWVLTE |
| | | | ELSPKMTLEVASGLFELYLTLADLQRFWDS |
| | | | IPGRDSRSLALAGIHAPFLPAVKLWFQVLR |
| | | | DQAKWRLQGAVDMDTLEPVDASSRHSSSAA |
| | | | TAGLCLSHIQELWVRLAWPDPAQAQGLGTQ |
| | | | LGQDVCEATLFYTELLRKKVDTQPGAAGEA |
| | | | VSEALCVVLNNVELVRKAAGQALKGLAWPE |
| | | | GATGPEGVLPRPLLSCTQALDDDLQREAHT |
| | | | VTAHLTSKMVGDIRKYVQHISLSPDSIQND |
| | | | EAVAPLMKYLDEKLALLNASLVKGNLSRVL |
| | | | EALWELLLQAILQALGANRDVSADFYSRFH |
| | | | FTLEALVSFFHAEGQGLPLESLRDGSYKRL |
| | | | KEELRLHKCSTRECIEQFYLDKLKQRTLEQ |
| | | | NRFGRLSVRCHYEAAEQRLAVEVLHAADLL |
| | | | PLDANGLSDPFVIVELGPPHLFPLVRSQRT |
| | | | QVKTRTLHPVYDELFYFSVPAEACRRRAAC |
| | | | VLFTVMDHDWLSTNDFAGEAALGLGGVTGV |
| | | | ARPQVGGGARAGQPVTLHLCRPRAQVRSAL |
| | | | RRLEGRTSKEAQEFVKKLKELEKCMEADP |
| O94812 | 3 | 68 | MSTLLDIKSSVLRQVQVCPSFRRRTEQDPG |
| | | | SASADPQEPATGAWKPGDGVEFFAHMRLML |
| | | | KKGEGRQGLPCLEVPLRSGSPAPPEPVDPS |
| | | | LGLRALAPEEVDDEEALLSYLQQVFGTSLE |
| | | | EHTEAIERVRKAKAPTYALKVSVMRAKNLL |
| | | | AKDPNGFSDPYCMLGILPASDATREPRAQK |
| | | | EQRFGFRKGSKRGGPLPAKCIQVTEVKSST |
| | | | LNPVWKEHFLFEIEDVSTDQLHLDIWDHDD |
| | | | DVSLVEACRKLNEVIGLKGMGRYFKQIVKS |
| | | | ARANGTAGPTEDHTDDFLGCLNIPVREVPV |
| | | | AGVDRWFKLEPRSSASRVQGHCHLVLKLIT |
| | | | TQRDTAMSQRGRSGFLSHLLLLSHLLRLEH |
| | | | SAEEPNSSSWRGELSTPAATILCLHGAQSN |
| | | | LSPLQLAVLHWQVSSRHHQTCTLDYSYLLG |
| | | | LLEDMQAHWEEAPSLPQEQEESLADSLSAF |
| | | | SEFGLQLLRQLRDYFPATNSTAVHRLELLL |
| | | | KCLGKLQLFQPSFEICPFESELNMDIAAAL |
| | | | KRGNREWYDRILNDKSPREQPGPQRLPGLV |
| | | | VLADAVYDDLQFCYSVYASLFHSILNVDVF |
| | | | TLTFRQLERLVAEEAWVLTEELSPKMTLEV |
| | | | ASGLFELYLTLADLQRFWDSIPGRDSRSLA |
| | | | LAGIHAPFLPAVKLWFQVLRDQAKWRLQGA |
| | | | VDMDTLEPVDASSRHSSSAATAGLCLSHIQ |
| | | | ELWVRLAWPDPAQAQGLGTQLGQDVCEATL |
| | | | FYTELLRKKVDTQPGAAGEAVSEALCVVLN |
| | | | NVELVRKAAGQALKGLAWPEGATGPEGVLP |
| | | | RPLLSCTQALDDDLQREAHTVTAHLTSKMV |
| | | | GDIRKYVQHISLSPDSIQNDEAVAPLMKYL |
| | | | DEKLALLNASLVKGNLSRVLEALWELLLQA |
| | | | ILQALGANRDVSADFYSRFHFTLEALVSFF |
| | | | HAEGQGLPLESLRDGSYKRLKEELRLHKCS |
| | | | TRECIEQFYLDKLKQRTLEQNRFGRLSVRC |
| | | | HYEAAEQRLAVEVLHAADLLPLDANGLSDP |
| | | | FVIVELGPPHLFPLVRSQRTQVKTRTLHPV |
| | | | YDELFYFSVPAEACRRRAACVLFTVMDHDW |
| | | | LSTNDFAGEAALGLGGVTGVARPQVGGGAR |
| | | | AGQPVTLHLCRPRAQVRSALRRLEGRTSKE |
| | | | AQEFVKKLKELEKCMEADP |
| O94812 | 5 | 69 | MSTLLDIKSSVLRQVQVCPSFRRRTEQDPG |
| | | | SASADPQEPATGAWKPGDGVEFFAHMRLML |
| | | | KKGEGRQGLPCLEVPLRSGSPAPPEPVDPS |
| | | | LGLRALAPEEALLSYLQQVFGTSLEEHTEA |
| | | | IERVRKAKAPTYALKVSVMRAKNLLAKDPN |
| | | | GFSDPYCMLGILPASDATREPRAQKEQRFG |
| | | | FRKGSKRGGPLPAKCIQVTEVKSSTLNPVW |
| | | | KEHFLFEIEDVSTDQLHLDIWDHDDDVSLV |
| | | | EACRKLNEVIGLKGMGRYFKQIVKSARANG |
| | | | TAGPTEDHTDDFLGCLNIPVREVPVAGVDR |
| | | | WFKLEPRSSASRVQGHCHLVLKLITTQRDT |
| | | | AMSQRGRSGFLSHLLLLSHLLRLEHSAEEP |
| | | | NSSSWRGELSTPAATILCLHGAQSNLSPLQ |
| | | | LAVLHWQVSSRHHQTCTLDYSYLLGLLEDM |
| | | | QAHWEEAPSLPQEQEESLADSLSAFSEFGL |
| | | | QLLRQLRDYFPATNSTAVHRLELLLKCLGK |
| | | | LQLFQPSFEICPFESELNMDIAAALKRGNR |
| | | | EWYDRILNDKSPREQPGPQRLPGLVVLADA |
| | | | VYDDLQFCYSVYASLFHSILNVDVFTLTFR |
| | | | QLERLVAEEAWVLTEELSPKMTLEVASGLF |
| | | | ELYLTLADLQRFWDSIPGRDSRSLALAGIH |
| | | | APFLPAVKLWFQVLRDQAKWRLQGAVDMDT |
| | | | LEPVDASSRHSSSAATAGLCLSHIQELWVR |
| | | | LAWPDPAQAQGLGTQLGQDVCEATLFYTEL |
| | | | LRKKVDTQPGAAGEAVSEALCVVLNNVELV |
| | | | RKAAGQALKGLAWPEGATGPEGVLPRPLLS |
| | | | CTQALDDDLQREAHTVTAHLTSKMVGDIRK |
| | | | YVQHISLSPDSIQNDEAVAPLMKYLDEKLA |
| | | | LLNASLVKGNLSRVLEALWELLLQAILQAL |
| | | | GANRDVSADFYSRFHFTLEALVSFFHAEGQ |
| | | | GLPLESLRDGSYKRLKEELRLHKCSTRECI |
| | | | EQFYLDKLKQRTLEQNRFGRLSVRCHYEAA |
| | | | EQRLAVEVLHAADLLPLDANGLSDPFVIVE |
| | | | LGPPHLFPLVRSQRTQVKTRTLHPVYDELF |
| | | | YFSVPAEACRRRAACVLFTVMDHDWLSTND |
| | | | FAGEAALGLGGVTGVARPQVGGGARAGQPV |
| | | | TLHLCRPRAQVRSALRRLEGRTSKEAQEFV |
| | | | KKLKELEKCMEADP |
| O94812 | 6 | 70 | MSTLLDIKSSVLRQVQVCPSFRRRTEQDPG |
| | | | SASADPQEPATGAWKPGDGVEFFAHMRLML |
| | | | KKGEGRQGLPCLEVPLRSGSPAPPEPVDPS |
| | | | LGLRALAPEEVEMLYEEALYTVLYRAGTMG |
| | | | PDQVDDEEALLSYLQQVFGTSLEEHTEAIE |
| | | | RVRKAKAPTYALKVSVMRAKNLLAKDPNGF |
| | | | SDPYCMLGILPASDATREPRAQKEQRFGFR |
| | | | KGSKRGGPLPAKCIQVTEVKSSTLNPVWKE |
| | | | HFLFEIEDVSTDQLHLDIWDHDDDVSLVEA |
| | | | CRKLNEVIGLKGMGRYFKQIVKSARANGTA |
| | | | GPTEDHTDDFLGCLNIPVREVPVAGVDRWF |
| | | | KLEPRSSASRVQGHCHLVLKLITTQRDTAM |
| | | | SQRGRSGFLSHLLLLSHLLRLEHSAEEPNS |
| | | | SSWRGELSTPAATILCLHGAQSNLSPLQLA |
| | | | VLHWQVSSRHHQTCTLDYSYLLGLLEDMQA |
| | | | HWEEAPSLPQEQEESLADSLSAFSEFGLQL |
| | | | LRQLRDYFPATNSTAVHRLELLLKCLGKLQ |
| | | | LFQPSFEICPFESELNMDIAAALKRGNREW |
| | | | YDRILNDKSPREQPGPQRLPGLVVLADAVY |
| | | | DDLQFCYSVYASLFHSILNVDVFTLTFRQL |
| | | | ERLVAEEAWVLTEELSPKMTLEVASGLFEL |
| | | | YLTLADLQRFWDSIPGRDSRSLALAGIHAP |
| | | | FLPAVKLWFQVLRDQAKWRLQGAVDMDTLE |
| | | | PVDASSRHSSSAATAGLCLSHIQELWVRLA |
| | | | WPDPAQAQGLGTQLGQDVCEATLFYTELLR |
| | | | KKVDTQPGAAGEAVSEALCVVLNNVELVRK |
| | | | AAGQALKGLAWPEGATGPEGVLPRPLLSCT |
| | | | QALDDDLQREAHTVTAHLTSKMVGDIRKYV |
| | | | QHISLSPDSIQNDEAVAPLMKYLDEKLALL |
| | | | NASLVKGNLSRVLEALWELLLQAILQALGA |
| | | | NRDVSADFYSRFHFTLEALVSFFHAEGQGL |
| | | | PLESLRDGSYKRLKEELRLHKCSTRECIEQ |
| | | | FYLDKLKQRTLEQNRFGRLSVRCHYEAAEQ |
| | | | RLAVEVLHAADLLPLDANGLSDPFVIVELG |
| | | | PPHLFPLVRSQRTQVKTRTLHPVYDELFYF |
| | | | SVPAEACRRRAACVLFTVMDHDWLSTNDFA |
| | | | GEAALGLGGVTGVARPQVGGGARAGQPVTL |
| | | | HLCRPRAQVRSALRRLEGRTSKEAQEFVKK |
| | | | LKELEKCMEADP |
| O94812 | 7 | 71 | MSTLLDIKSSVLRQVQVCPSFRRRTEQDPG |
| | | | SASADPQEPATGAWKPGDGVEFFAHMRLML |
| | | | KKGEGRQGLPCLEVPLRSGSPAPPEPVDPS |
| | | | LGLRALAPEEVEMLYEEALYTVLYRAGTMG |
| | | | PDQVDDEEALLSYLQQVFGTSLEEHTEAIE |
| | | | RVRKAKAPTYALKVSVMRAKNLLAKDPNGF |
| | | | SDPYCMLGILPASDATREPRAQKEQRFGFR |
| | | | KGSKRGGPLPAKCIQVTEVKSSTLNPVWKE |
| | | | HFLFEIEDVSTDQLHLDIWDHDDDVSLVEA |
| | | | CRKLNEVIGLKGMGRYFKQIVKSARANGTA |
| | | | GPTEDHTDDFLGCLNIPVRRDTAMSQRGRS |
| | | | GFLSHLLLLSHLLRLEHSAEEPNSSSWRGE |
| | | | LSTPAATILCLHGAQSNLSPLQLAVLHWQV |
| | | | SSRHHQTCTLDYSYLLGLLEDMQAHWEEAP |
| | | | SLPQEQEESLADSLSAFSEFGLQLLRQLRD |
| | | | YFPATNSTAVHRLELLLKCLGKLQLFQPSF |
| | | | EICPFESELNMDIAAALKRGNREWYDRILN |
| | | | DKSPREQPGPQRLPGLVVLADAVYDDLQFC |
| | | | YSVYASLFHSILNVDVFTLTFRQLERLVAE |
| | | | EAWVLTEELSPKMTLEVASGLFELYLTLAD |
| | | | LQRFWDSIPGRDSRSLALAGIHAPFLPAVK |
| | | | LWFQVLRDQAKWRLQGAVDMDTLEPVDASS |
| | | | RHSSSAATAGLCLSHIQELWVRLAWPDPAQ |
| | | | AQGLGTQLGQDVCEATLFYTELLRKKVDTQ |
| | | | PGAAGEAVSEALCVVLNNVELVRKAAGQAL |
| | | | KGLAWPEGATGPEGVLPRPLLSCTQALDDD |
| | | | LQREAHTVTAHLTSKMVGDIRKYVQHISLS |
| | | | PDSIQNDEAVAPLMKYLDEKLALLNASLVK |
| | | | GNLSRVLEALWELLLQAILQALGANRDVSA |
| | | | DFYSRFHFTLEALVSFFHAEGQGLPLESLR |
| | | | DGSYKRLKEELRLHKCSTRECIEQFYLDKL |
| | | | KQRTLEQNRFGRLSVRCHYEAAEQRLAVEV |
| | | | LHAADLLPLDANGLSDPFVIVELGPPHLFP |
| | | | LVRSQRTQVKTRTLHPVYDELFYFSVPAEA |
| | | | CRRRAACVLFTVMDHDWLSTNDFAGEAALG |
| | | | LGGVTGVARPQVGGGARAGQPVTLHLCRPR |
| | | | AQVRSALRRLEGRTSKEAQEFVKKLKELEK |
| | | | CMEADP |
| P00742 | 1 | 72 | MGRPLHLVLLSASLAGLLLLGESLFIRREQ |
| | | | ANNILARVTRANSFLEEMKKGHLERECMEE |
| | | | TCSYEEAREVFEDSDKTNEFWNKYKDGDQC |
| | | | ETSPCQNQGKCKDGLGEYTCTCLEGFEGKN |
| | | | CELFTRKLCSLDNGDCDQFCHEEQNSVVCS |
| | | | CARGYTLADNGKACIPTGPYPCGKQTLERR |
| | | | KRSVAQATSSSGEAPDSITWKPYDAADLDP |
| | | | TENPFDLLDFNQTQPERGDNNLTRIVGGQE |
| | | | CKDGECPWQALLINEENEGFCGGTILSEFY |
| | | | ILTAAHCLYQAKRFKVRVGDRNTEQEEGGE |
| | | | AVHEVEVVIKHNRFTKETYDFDIAVLRLKT |
| | | | PITFRMNVAPACLPERDWAESTLMTQKTGI |
| | | | VSGFGRTHEKGRQSTRLKMLEVPYVDRNSC |
| | | | KLSSSFIITQNMFCAGYDTKQEDACQGDSG |
| | | | GPHVTRFKDTYFVTGIVSWGEGCARKGKYG |
| | | | IYTKVTAFLKWIDRSMKTRGLPKAKSHAPE |
| | | | VITSSPLK |
| P02549 | 1 | 73 | MEQFPKETVVESSGPKVLETAEEIQERRQE |
| | | | VLTRYQSFKERVAERGQKLEDSYHLQVFKR |
| | | | DADDLGKWIMEKVNILTDKSYEDPTNIQGK |
| | | | YQKHQSLEAEVQTKSRLMSELEKTREERFT |
| | | | MGHSAHEETKAHIEELRHLWDLLLELTLEK |
| | | | GDQLLRALKFQQYVQECADILEWIGDKEAI |
| | | | ATSVELGEDWERTEVLHKKFEDFQVELVAK |
| | | | EGRVVEVNQYANECAEENHPDLPLIQSKQN |
| | | | EVNAAWERLRGLALQRQKALSNAANLQRFK |
| | | | RDVTEAIQWIKEKEPVLTSEDYGKDLVASE |
| | | | GLFHSHKGLERNLAVMSDKVKELCAKAEKL |
| | | | TLSHPSDAPQIQEMKEDLVSSWEHIRALAT |
| | | | SRYEKLQATYWYHRFSSDFDELSGWMNEKT |
| | | | AAINADELPTDVAGGEVLLDRHQQHKHEID |
| | | | SYDDRFQSADETGQDLVNANHEASDEVREK |
| | | | MEILDNNWTALLELWDERHRQYEQCLDFHL |
| | | | FYRDSEQVDSWMSRQEAFLENEDLGNSLGS |
| | | | AEALLQKHEDFEEAFTAQEEKIITVDKTAT |
| | | | KLIGDDHYDSENIKAIRDGLLARRDALREK |
| | | | AATRRRLLKESLLLQKLYEDSDDLKNWINK |
| | | | KKKLADDEDYKDIQNLKSRVQKQQVFEKEL |
| | | | AVNKTQLENIQKTGQEMIEGGHYASDNVTT |
| | | | RLSEVASLWEELLEATKQKGTQLHEANQQL |
| | | | QFENNAEDLQRWLEDVEWQVTSEDYGKGLA |
| | | | EVQNRLRKHGLLESAVAARQDQVDILTDLA |
| | | | AYFEEIGHPDSKDIRARQESLVCRFEALKE |
| | | | PLATRKKKLLDLLHLQLICRDTEDEEAWIQ |
| | | | ETEPSATSTYLGKDLIASKKLLNRHRVILE |
| | | | NIASHEPRIQEITERGNKMVEEGHFAAEDV |
| | | | ASRVKSLNQNMESLRARAARRQNDLEANVQ |
| | | | FQQYLADLHEAETWIREKEPIVDNTNYGAD |
| | | | EEAAGALLKKHEAFLLDLNSFGDSMKALRN |
| | | | QANACQQQQAAPVEGVAGEQRVMALYDFQA |
| | | | RSPREVTMKKGDVLTLLSSINKDWWKVEAA |
| | | | DHQGIVPAVYVRRLAHDEFPMLPQRRREEP |
| | | | GNITQRQEQIENQYRSLLDRAEERRRRLLQ |
| | | | RYNEFLLAYEAGDMLEWIQEKKAENTGVEL |
| | | | DDVWELQKKFDEFQKDLNTNEPRLRDINKV |
| | | | ADDLLFEGLLTPEGAQIRQELNSRWGSLQR |
| | | | LADEQRQLLGSAHAVEVFHREADDTKEQIE |
| | | | KKCQALSAADPGSDLFSVQALQRRHEGFER |
| | | | DLVPLGDKVTILGETAERLSESHPDATEDL |
| | | | QRQKMELNEAWEDLQGRTKDRKESLNEAQK |
| | | | FYLFLSKARDLQNWISSIGGMVSSQELAED |
| | | | LTGIEILLERHQEHRADMEAEAPTFQALED |
| | | | FSAELIDSGHHASPEIEKKLQAVKLERDDL |
| | | | EKAWEKRKKILDQCLELQMFQGNCDQVESW |
| | | | MVARENSLRSDDKSSLDSLEALMKKRDDLD |
| | | | KAITAQEGKITDLEHFAESLIADEHYAKEE |
| | | | IATRLQRVLDRWKALKAQLIDERTKLGDYA |
| | | | NLKQFYRDLEELEEWISEMLPTACDESYKD |
| | | | ATNIQRKYLKHQTFAHEVDGRSEQVHGVIN |
| | | | LGNSLIECSACDGNEEAMKEQLEQLKEHWD |
| | | | HLLERTNDKGKKLNEASRQQRENTSIRDFE |
| | | | FWLSEAETLLAMKDQARDLASAGNLLKKHQ |
| | | | LLEREMLAREDALKDLNTLAEDLLSSGTEN |
| | | | VDQIVKKKDNVNKRFLNVQELAAAHHEKLK |
| | | | EAYALFQFFQDLDDEESWIEEKLIRVSSQD |
| | | | YGRDLQGVQNLLKKHKRLEGELVAHEPAIQ |
| | | | NVLDMAEKLKDKAAVGQEEIQLRLAQFVEH |
| | | | WEKLKELAKARGLKLEESLEYLQFMQNAEE |
| | | | EEAWINEKNALAVRGDCGDTLAATQSLLMK |
| | | | HEALENDFAVHETRVQNVCAQGEDILNKVL |
| | | | QEESQNKEISSKIEALNEKTPSLAKAIAAW |
| | | | KLQLEDDYAFQEFNWKADVVEAWIADKETS |
| | | | LKTNGNGADLGDFLTLLAKQDTLDASLQSF |
| | | | QQERLPEITDLKDKLISAQHNQSKAIEERY |
| | | | AALLKRWEQLLEASAVHRQKLLEKQLPLQK |
| | | | AEDLFVEFAHKASALNNWCEKMEENLSEPV |
| | | | HCVSLNEIRQLQKDHEDFLASLARAQADFK |
| | | | CLLELDQQIKALGVPSSPYTWLTVEVLERT |
| | | | WKHLSDIIEEREQELQKEEARQVKNFEMCQ |
| | | | EFEQNASTFLQWILETRAYFLDGSLLKETG |
| | | | TLESQLEANKRKQKEIQAMKRQLTKIVDLG |
| | | | DNLEDALILDIKYSTIGLAQQWDQLYQLGL |
| | | | RMQHNLEQQIQAKDIKGVSEETLKEFSTIY |
| | | | KHFDENLTGRLTHKEFRSCLRGLNYYLPMV |
| | | | EEDEHEPKFEKFLDAVDPGRKGYVSLEDYT |
| | | | AFLIDKESENIKSSDEIENAFQALAEGKSY |
| | | | ITKEDMKQALTPEQVSFCATHMQQYMDPRG |
| | | | RSHLSGYDYVGFTNSYFGN |
| P02549 | 2 | 74 | MEQFPKETVVESSGPKVLETAEEIQERRQE |
| | | | VLTRYQSFKERVAERGQKLEDSYHLQVFKR |
| | | | DADDLGKWIMEKVNILTDKSYEDPTNIQGK |
| | | | YQKHQSLEAEVQTKSRLMSELEKTREERFT |
| | | | MGHSAHEETKAHIEELRHLWDLLLELTLEK |
| | | | GDQLLRALKFQQYVQECADILEWIGDKEAI |
| | | | ATSVELGEDWERTEVLHKKFEDFQVELVAK |
| | | | EGRVVEVNQYANECAEENHPDLPLIQSKQN |
| | | | EVNAAWERLRGLALQRQKALSNAANLQRFK |
| | | | RDVTEAIQWIKEKEPVLTSEDYGKDLVASE |
| | | | GLFHSHKGLERNLAVMSDKVKELCAKAEKL |
| | | | TLSHPSDAPQIQEMKEDLVSSWEHIRALAT |
| | | | SRYEKLQATYWYHRFSSDFDELSGWMNEKT |
| | | | AAINADELPTDVAGGEVLLDRHQQHKHEID |
| | | | SYDDRFQSADETGQDLVNANHEASDEVREK |
| | | | MEILDNNWTALLELWDERHRQYEQCLDFHL |
| | | | FYRDSEQVDSWMSRQEAFLENEDLGNSLGS |
| | | | AEALLQKHEDFEEAFTAQEEKIITVDKTAT |
| | | | KLIGDDHYDSENIKAIRDGLLARRDALREK |
| | | | AATRRRLLKESLLLQKLYEDSDDLKNWINK |
| | | | KKKLADDEDYKDIQNLKSRVQKQQVFEKEL |
| | | | AVNKTQLENIQKTGQEMIEGGHYASDNVTT |
| | | | RLSEVASLWEELLEATKQKGTQLHEANQQL |
| | | | QFENNAEDLQRWLEDVEWQVTSEDYGKGLA |
| | | | EVQNRLRKHGLLESAVAARQDQVDILTDLA |
| | | | AYFEEIGHPDSKDIRARQESLVCRFEALKE |
| | | | PLATRKKKLLDLLHLQLICRDTEDEEAWIQ |
| | | | ETEPSATSTYLGKDLIASKKLLNRHRVILE |
| | | | NIASHEPRIQEITERGNKMVEEGHFAAEDV |
| | | | ASRVKSLNQNMESLRARAARRQNDLEANVQ |
| | | | FQQYLADLHEAETWIREKEPIVDNTNYGAD |
| | | | EEAAGALLKKHEAFLLDLNSFGDSMKALRN |
| | | | QANACQQQQAAPVEGVAGEQRVMALYDFQA |
| | | | RSPREVTMKKGDVLTLLSSINKDWWKVEAA |
| | | | DHQGIVPAVYVRRLAHDEFPMLPQRRREEP |
| | | | GNITQRQEQIENQYRSLLDRAEERRRRLLQ |
| | | | RYNEFLLAYEAGDMLEWIQEKKAENTGVEL |
| | | | DDVWELQKKFDEFQKDLNTNEPRLRDINKV |
| | | | ADDLLFEGLLTPEGAQIRQELNSRWGSLQR |
| | | | LADEQRQLLGSAHAVEVFHREADDTKEQIE |
| | | | KKCQALSAADPGSDLFSVQALQRRHEGFER |
| | | | DLVPLGDKVTILGETAERLSESHPDATEDL |
| | | | QRQKMELNEAWEDLQGRTKDRKESLNEAQK |
| | | | FYLFLSKARDLQNWISSIGGMVSSQELAED |
| | | | LTGIEILLERHQEHRADMEAEAPTFQALED |
| | | | FSAELIDSGHHASPEIEKKLQAVKLERDDL |
| | | | EKAWEKRKKILDQCLELQMFQGNCDQVESW |
| | | | MVARENSLRSDDKSSLDSLEALMKKRDDLD |
| | | | KAITAQEGKITDLEHFAESLIADEHYAKEE |
| | | | IATRLQRVLDRWKALKAQLIDERTKLGDYA |
| | | | NLKQFYRDLEELEEWISEMLPTACDESYKD |
| | | | ATNIQRKYLKHQTFAHEVDGRSEQVHGVIN |
| | | | LGNSLIECSACDGNEEAMKEQLEQLKEHWD |
| | | | HLLERTNDKGKKLNEASRQQRENTSIRDFE |
| | | | FWLSEAETLLAMKDQARDLASAGNLLKKHQ |
| | | | LLEREMLAREDALKDLNTLAEDLLSSGTFN |
| | | | VDQIVKKKDNVNKRFLNVQELAAAHHEKLK |
| | | | EAYALFQFFQDLDDEESWIEEKLIRVSSQD |
| | | | YGRDLQGVQNLLKKHKRLEGELVAHEPAIQ |
| | | | NVLDMAEKLKDKAAVGQEEIQLRLAQFVEH |
| | | | WEKLKELAKARGLKLEESLEYLQFMQNAEE |
| | | | EEAWINEKNALAVRGDCGDTLAATQSLLMK |
| | | | HEALENDFAVHETRVQNVCAQGEDILNKEE |
| | | | SQNKEISSKIEALNEKTPSLAKAIAAWKLQ |
| | | | LEDDYAFQEFNWKADVVEAWIADKETSLKT |
| | | | NGNGADLGDFLTLLAKQDTLDASLQSFQQE |
| | | | RLPEITDLKDKLISAQHNQSKAIEERYAAL |
| | | | LKRWEQLLEASAVHRQKLLEKQLPLQKAED |
| | | | LFVEFAHKASALNNWCEKMEENLSEPVHCV |
| | | | SLNEIRQLQKDHEDFLASLARAQADFKCLL |
| | | | ELDQQIKALGVPSSPYTWLTVEVLERTWKH |
| | | | LSDIIEEREQELQKEEARQVKNFEMCQEFE |
| | | | QNASTFLQWILETRAYFLDGSLLKETGTLE |
| | | | SQLEANKRKQKEIQAMKRQLTKIVDLGDNL |
| | | | EDALILDIKYSTIGLAQQWDQLYQLGLRMQ |
| | | | HNLEQQIQAKDIKGVSEETLKEFSTIYKHF |
| | | | DENLTGRLTHKEFRSCLRGLNYYLPMVEED |
| | | | EHEPKFEKFLDAVDPGRKGYVSLEDYTAFL |
| | | | IDKESENIKSSDEIENAFQALAEGKSYITK |
| | | | EDMKQALTPEQVSFCATHMQQYMDPRGRSH |
| | | | LSGYDYVGFTNSYFGN |
| P04114 | 1 | 75 | MDPPRPALLALLALPALLLLLLAGARAEEE |
| | | | MLENVSLVCPKDATRFKHLRKYTYNYEAES |
| | | | SSGVPGTADSRSATRINCKVELEVPQLCSF |
| | | | ILKTSQCTLKEVYGFNPEGKALLKKTKNSE |
| | | | EFAAAMSRYELKLAIPEGKQVFLYPEKDEP |
| | | | TYILNIKRGIISALLVPPETEEAKQVLFLD |
| | | | TVYGNCSTHFTVKTRKGNVATEISTERDLG |
| | | | QCDRFKPIRTGISPLALIKGMTRPLSTLIS |
| | | | SSQSCQYTLDAKRKHVAEAICKEQHLFLPF |
| | | | SYKNKYGMVAQVTQTLKLEDTPKINSRFFG |
| | | | EGTKKMGLAFESTKSTSPPKQAEAVLKTLQ |
| | | | ELKKLTISEQNIQRANLFNKLVTELRGLSD |
| | | | EAVTSLLPQLIEVSSPITLQALVQCGQPQC |
| | | | STHILQWLKRVHANPLLIDVVTYLVALIPE |
| | | | PSAQQLREIFNMARDQRSRATLYALSHAVN |
| | | | NYHKTNPTGTQELLDIANYLMEQIQDDCTG |
| | | | DEDYTYLILRVIGNMGQTMEQLTPELKSSI |
| | | | LKCVQSTKPSLMIQKAAIQALRKMEPKDKD |
| | | | QEVLLQTFLDDASPGDKRLAAYLMLMRSPS |
| | | | QADINKIVQILPWEQNEQVKNFVASHIANI |
| | | | LNSEELDIQDLKKLVKEALKESQLPTVMDF |
| | | | RKFSRNYQLYKSVSLPSLDPASAKIEGNLI |
| | | | FDPNNYLPKESMLKTTLTAFGFASADLIEI |
| | | | GLEGKGFEPTLEALFGKQGFFPDSVNKALY |
| | | | WVNGQVPDGVSKVLVDHFGYTKDDKHEQDM |
| | | | VNGIMLSVEKLIKDLKSKEVPEARAYLRIL |
| | | | GEELGFASLHDLQLLGKLLLMGARTLQGIP |
| | | | QMIGEVIRKGSKNDFFLHYIFMENAFELPT |
| | | | GAGLQLQISSSGVIAPGAKAGVKLEVANMQ |
| | | | AELVAKPSVSVEFVTNMGIIIPDFARSGVQ |
| | | | MNTNFFHESGLEAHVALKAGKLKFIIPSPK |
| | | | RPVKLLSGGNTLHLVSTTKTEVIPPLIENR |
| | | | QSWSVCKQVFPGLNYCTSGAYSNASSTDSA |
| | | | SYYPLTGDTRLELELRPTGEIEQYSVSATY |
| | | | ELQREDRALVDTLKFVTQAEGAKQTEATMT |
| | | | FKYNRQSMTLSSEVQIPDFDVDLGTILRVN |
| | | | DESTEGKTSYRLTLDIQNKKITEVALMGHL |
| | | | SCDTKEERKIKGVISIPRLQAEARSEILAH |
| | | | WSPAKLLLQMDSSATAYGSTVSKRVAWHYD |
| | | | EEKIEFEWNTGTNVDTKKMTSNFPVDLSDY |
| | | | PKSLHMYANRLLDHRVPQTDMTFRHVGSKL |
| | | | IVAMSSWLQKASGSLPYTQTLQDHLNSLKE |
| | | | FNLQNMGLPDFHIPENLFLKSDGRVKYTLN |
| | | | KNSLKIEIPLPFGGKSSRDLKMLETVRTPA |
| | | | LHFKSVGFHLPSREFQVPTFTIPKLYQLQV |
| | | | PLLGVLDLSTNVYSNLYNWSASYSGGNTST |
| | | | DHFSLRARYHMKADSVVDLLSYNVQGSGET |
| | | | TYDHKNTFTLSYDGSLRHKFLDSNIKFSHV |
| | | | EKLGNNPVSKGLLIFDASSSWGPQMSASVH |
| | | | LDSKKKQHLFVKEVKIDGQFRVSSFYAKGT |
| | | | YGLSCQRDPNTGRLNGESNLRFNSSYLQGT |
| | | | NQITGRYEDGTLSLTSTSDLQSGIIKNTAS |
| | | | LKYENYELTLKSDTNGKYKNFATSNKMDMT |
| | | | FSKQNALLRSEYQADYESLRFFSLLSGSLN |
| | | | SHGLELNADILGTDKINSGAHKATLRIGQD |
| | | | GISTSATTNLKCSLLVLENELNAELGLSGA |
| | | | SMKLTTNGRFREHNAKFSLDGKAALTELSL |
| | | | GSAYQAMILGVDSKNIFNFKVSQEGLKLSN |
| | | | DMMGSYAEMKFDHTNSLNIAGLSLDFSSKL |
| | | | DNIYSSDKFYKQTVNLQLQPYSLVTTLNSD |
| | | | LKYNALDLTNNGKLRLEPLKLHVAGNLKGA |
| | | | YQNNEIKHIYAISSAALSASYKADTVAKVQ |
| | | | GVEFSHRLNTDIAGLASAIDMSTNYNSDSL |
| | | | HFSNVFRSVMAPFTMTIDAHTNGNGKLALW |
| | | | GEHTGQLYSKFLLKAEPLAFTFSHDYKGST |
| | | | SHHLVSRKSISAALEHKVSALLTPAEQTGT |
| | | | WKLKTQFNNNEYSQDLDAYNTKDKIGVELT |
| | | | GRTLADLTLLDSPIKVPLLLSEPINIIDAL |
| | | | EMRDAVEKPQEFTIVAFVKYDKNQDVHSIN |
| | | | LPFFETLQEYFERNRQTIIVVLENVQRNLK |
| | | | HINIDQFVRKYRAALGKLPQQANDYLNSFN |
| | | | WERQVSHAKEKLTALTKKYRITENDIQIAL |
| | | | DDAKINFNEKLSQLQTYMIQFDQYIKDSYD |
| | | | LHDLKIAIANIIDEIIEKLKSLDEHYHIRV |
| | | | NLVKTIHDLHLFIENIDENKSGSSTASWIQ |
| | | | NVDTKYQIRIQIQEKLQQLKRHIQNIDIQH |
| | | | LAGKLKQHIEAIDVRVLLDQLGTTISFERI |
| | | | NDILEHVKHFVINLIGDFEVAEKINAFRAK |
| | | | VHELIERYEVDQQIQVLMDKLVELAHQYKL |
| | | | KETIQKLSNVLQQVKIKDYFEKLVGFIDDA |
| | | | VKKLNELSFKTFIEDVNKFLDMLIKKLKSF |
| | | | DYHQFVDETNDKIREVTQRLNGEIQALELP |
| | | | QKAEALKLFLEETKATVAVYLESLQDTKIT |
| | | | LIINWLQEALSSASLAHMKAKFRETLEDTR |
| | | | DRMYQMDIQQELQRYLSLVGQVYSTLVTYI |
| | | | SDWWTLAAKNLTDFAEQYSIQDWAKRMKAL |
| | | | VEQGFTVPEIKTILGTMPAFEVSLQALQKA |
| | | | TFQTPDFIVPLTDLRIPSVQINFKDLKNIK |
| | | | IPSRFSTPEFTILNTFHIPSFTIDFVEMKV |
| | | | KIIRTIDQMLNSELQWPVPDIYLRDLKVED |
| | | | IPLARITLPDFRLPEIAIPEFIIPTLNLND |
| | | | FQVPDLHIPEFQLPHISHTIEVPTFGKLYS |
| | | | ILKIQSPLFTLDANADIGNGTTSANEAGIA |
| | | | ASITAKGESKLEVLNFDFQANAQLSNPKIN |
| | | | PLALKESVKFSSKYLRTEHGSEMLFFGNAI |
| | | | EGKSNTVASLHTEKNTLELSNGVIVKINNQ |
| | | | LTLDSNTKYFHKLNIPKLDFSSQADLRNEI |
| | | | KTLLKAGHIAWTSSGKGSWKWACPRFSDEG |
| | | | THESQISFTIEGPLTSFGLSNKINSKHLRV |
| | | | NQNLVYESGSLNFSKLEIQSQVDSQHVGHS |
| | | | VLTAKGMALFGEGKAEFTGRHDAHLNGKVI |
| | | | GTLKNSLFFSAQPFEITASTNNEGNLKVRF |
| | | | PLRLTGKIDFLNNYALFLSPSAQQASWQVS |
| | | | ARFNQYKYNQNFSAGNNENIMEAHVGINGE |
| | | | ANLDFLNIPLTIPEMRLPYTIITTPPLKDF |
| | | | SLWEKTGLKEFLKTTKQSFDLSVKAQYKKN |
| | | | KHRHSITNPLAVLCEFISQSIKSFDRHFEK |
| | | | NRNNALDFVTKSYNETKIKFDKYKAEKSHD |
| | | | ELPRTFQIPGYTVPVVNVEVSPFTIEMSAF |
| | | | GYVFPKAVSMPSFSILGSDVRVPSYTLILP |
| | | | SLELPVLHVPRNLKLSLPDFKELCTISHIF |
| | | | IPAMGNITYDFSFKSSVITLNTNAELFNQS |
| | | | DIVAHLLSSSSSVIDALQYKLEGTTRLTRK |
| | | | RGLKLATALSLSNKFVEGSHNSTVSLTTKN |
| | | | MEVSVATTTKAQIPILRMNFKQELNGNTKS |
| | | | KPTVSSSMEFKYDFNSSMLYSTAKGAVDHK |
| | | | LSLESLTSYFSIESSTKGDVKGSVLSREYS |
| | | | GTIASEANTYLNSKSTRSSVKLQGTSKIDD |
| | | | IWNLEVKENFAGEATLQRIYSLWEHSTKNH |
| | | | LQLEGLFFTNGEHTSKATLELSPWQMSALV |
| | | | QVHASQPSSFHDFPDLGQEVALNANTKNQK |
| | | | IRWKNEVRIHSGSFQSQVELSNDQEKAHLD |
| | | | IAGSLEGHLRFLKNIILPVYDKSLWDFLKL |
| | | | DVTTSIGRRQHLRVSTAFVYTKNPNGYSFS |
| | | | IPVKVLADKFIIPGLKLNDLNSVLVMPTFH |
| | | | VPFTDLQVPSCKLDFREIQIYKKLRTSSFA |
| | | | LNLPTLPEVKFPEVDVLTKYSQPEDSLIPF |
| | | | FEITVPESQLTVSQFTLPKSVSDGIAALDL |
| | | | NAVANKIADFELPTIIVPEQTIEIPSIKFS |
| | | | VPAGIVIPSFQALTARFEVDSPVYNATWSA |
| | | | SLKNKADYVETVLDSTCSSTVQFLEYELNV |
| | | | LGTHKIEDGTLASKTKGTFAHRDFSAEYEE |
| | | | DGKYEGLQEWEGKAHLNIKSPAFTDLHLRY |
| | | | QKDKKGISTSAASPAVGTVGMDMDEDDDFS |
| | | | KWNFYYSPQSSPDKKLTIFKTELRVRESDE |
| | | | ETQIKVNWEEEAASGLLTSLKDNVPKATGV |
| | | | LYDYVNKYHWEHTGLTLREVSSKLRRNLQN |
| | | | NAEWVYQGAIRQIDDIDVRFQKAASGTTGT |
| | | | YQEWKDKAQNLYQELLTQEGQASFQGLKDN |
| | | | VFDGLVRVTQEFHMKVKHLIDSLIDFLNFP |
| | | | RFQFPGKPGIYTREELCTMFIREVGTVLSQ |
| | | | VYSKVHNGSEILFSYFQDLVITLPFELRKH |
| | | | KLIDVISMYRELLKDLSKEAQEVFKAIQSL |
| | | | KTTEVLRNLQDLLQFIFQLIEDNIKQLKEM |
| | | | KFTYLINYIQDEINTIFSDYIPYVFKLLKE |
| | | | NLCLNLHKFNEFIQNELQEASQELQQIHQY |
| | | | IMALREEYFDPSIVGWTVKYYELEEKIVSL |
| | | | IKNLLVALKDFHSEYIVSASNFTSQLSSQV |
| | | | EQFLHRNIQEYLSILTDPDGKGKEKIAELS |
| | | | ATAQEIIKSQAIATKKIISDYHQQFRYKLQ |
| | | | DFSDQLSDYYEKFIAESKRLIDLSIQNYHT |
| | | | FLIYITELLKKLQSTTVMNPYMKLAPGELT |
| | | | IIL |
| P05154 | 1 | 76 | MQLFLLLCLVLLSPQGASLHRHHPREMKKR |
| | | | VEDLHVGATVAPSSRRDFTFDLYRALASAA |
| | | | PSQSIFFSPVSISMSLAMLSLGAGSSTKMQ |
| | | | ILEGLGLNLQKSSEKELHRGFQQLLQELNQ |
| | | | PRDGFQLSLGNALFTDLVVDLQDTFVSAMK |
| | | | TLYLADTFPTNFRDSAGAMKQINDYVAKQT |
| | | | KGKIVDLLKNLDSNAVVIMVNYIFFKAKWE |
| | | | TSFNHKGTQEQDFYVTSETVVRVPMMSRED |
| | | | QYHYLLDRNLSCRVVGVPYQGNATALFILP |
| | | | SEGKMQQVENGLSEKTLRKWLKMFKKRQLE |
| | | | LYLPKFSIEGSYQLEKVLPSLGISNVFTSH |
| | | | ADLSGISNHSNIQVSEMVHKAVVEVDESGT |
| | | | RAAAATGTIFTFRSARLNSQRLVFNRPFLM |
| | | | FIVDNNILFLGKVNRP |
| P05546 | 1 | 77 | MKHSLNALLIFLIITSAWGGSKGPLDQLEK |
| | | | GGETAQSADPQWEQLNNKNLSMPLLPADFH |
| | | | KENTVTNDWIPEGEEDDDYLDLEKIFSEDD |
| | | | DYIDIVDSLSVSPTDSDVSAGNILQLFHGK |
| | | | SRIQRLNILNAKFAFNLYRVLKDQVNTFDN |
| | | | IFIAPVGISTAMGMISLGLKGETHEQVHSI |
| | | | LHFKDFVNASSKYEITTIHNLFRKLTHRLF |
| | | | RRNFGYTLRSVNDLYIQKQFPILLDFKTKV |
| | | | REYYFAEAQIADFSDPAFISKTNNHIMKLT |
| | | | KGLIKDALENIDPATQMMILNCIYFKGSWV |
| | | | NKFPVEMTHNHNFRLNEREVVKVSMMQTKG |
| | | | NFLAANDQELDCDILQLEYVGGISMLIVVP |
| | | | HKMSGMKTLEAQLTPRVVERWQKSMTNRTR |
| | | | EVLLPKFKLEKNYNLVESLKLMGIRMLFDK |
| | | | NGNMAGISDQRIAIDLFKHQGTITVNEEGT |
| | | | QATTVTTVGFMPLSTQVRFTVDRPFLFLIY |
| | | | EHRTSCLLFMGRVANPSRS |
| P13497 | 1 | 10 | MPGVARLPLLLGLLLLPRPGRPLDLADYTY |
| | | | DLAEEDDSEPLNYKDPCKAAAFLGDIALDE |
| | | | EDLRAFQVQQAVDLRRHTARKSSIKAAVPG |
| | | | NTSTPSCQSTNGQPQRGACGRWRGRSRSRR |
| | | | AATSRPERVWPDGVIPFVIGGNFTGSQRAV |
| | | | FRQAMRHWEKHTCVTFLERTDEDSYIVFTY |
| | | | RPCGCCSYVGRRGGGPQAISIGKNCDKFGI |
| | | | VVHELGHVVGFWHEHTRPDRDRHVSIVREN |
| | | | IQPGQEYNFLKMEPQEVESLGETYDFDSIM |
| | | | HYARNTFSRGIFLDTIVPKYEVNGVKPPIG |
| | | | QRTRLSKGDIAQARKLYKCPACGETLQDST |
| | | | GNFSSPEYPNGYSAHMHCVWRISVTPGEKI |
| | | | ILNFTSLDLYRSRLCWYDYVEVRDGFWRKA |
| | | | PLRGRFCGSKLPEPIVSTDSRLWVEFRSSS |
| | | | NWVGKGFFAVYEAICGGDVKKDYGHIQSPN |
| | | | YPDDYRPSKVCIWRIQVSEGFHVGLTFQSF |
| | | | EIERHDSCAYDYLEVRDGHSESSTLIGRYC |
| | | | GYEKPDDIKSTSSRLWLKFVSDGSINKAGF |
| | | | AVNFFKEVDECSRPNRGGCEQRCLNTLGSY |
| | | | KCSCDPGYELAPDKRRCEAACGGFLTKLNG |
| | | | SITSPGWPKEYPPNKNCIWQLVAPTQYRIS |
| | | | LQFDFFETEGNDVCKYDFVEVRSGLTADSK |
| | | | LHGKFCGSEKPEVITSQYNNMRVEFKSDNT |
| | | | VSKKGFKAHFFSDKDECSKDNGGCQQDCVN |
| | | | TFGSYECQCRSGFVLHDNKHDCKEAGCDHK |
| | | | VTSTSGTITSPNWPDKYPSKKECTWAISST |
| | | | PGHRVKLTFMEMDIESQPECAYDHLEVFDG |
| | | | RDAKAPVLGRFCGSKKPEPVLATGSRMFLR |
| | | | FYSDNSVQRKGFQASHATECGGQVRADVKT |
| | | | KDLYSHAQFGDNNYPGGVDCEWVIVAEEGY |
| | | | GVELVFQTFEVEEETDCGYDYMELFDGYDS |
| | | | TAPRLGRYCGSGPPEEVYSAGDSVLVKFHS |
| | | | DDTITKKGFHLRYTSTKFQDTLHSRK |
| P13497 | 2 | 11 | MPGVARLPLLLGLLLLPRPGRPLDLADYTY |
| | | | DLAEEDDSEPLNYKDPCKAAAFLGDIALDE |
| | | | EDLRAFQVQQAVDLRRHTARKSSIKAAVPG |
| | | | NTSTPSCQSTNGQPQRGACGRWRGRSRSRR |
| | | | AATSRPERVWPDGVIPFVIGGNFTGSQRAV |
| | | | FRQAMRHWEKHTCVTFLERTDEDSYIVFTY |
| | | | RPCGCCSYVGRRGGGPQAISIGKNCDKFGI |
| | | | VVHELGHVVGFWHEHTRPDRDRHVSIVREN |
| | | | IQPGQEYNFLKMEPQEVESLGETYDFDSIM |
| | | | HYARNTFSRGIFLDTIVPKYEVNGVKPPIG |
| | | | QRTRLSKGDIAQARKLYKCPACGETLQDST |
| | | | GNFSSPEYPNGYSAHMHCVWRISVTPGEKI |
| | | | ILNFTSLDLYRSRLCWYDYVEVRDGFWRKA |
| | | | PLRGRFCGSKLPEPIVSTDSRLWVEFRSSS |
| | | | NWVGKGFFAVYEAICGGDVKKDYGHIQSPN |
| | | | YPDDYRPSKVCIWRIQVSEGFHVGLTFQSF |
| | | | EIERHDSCAYDYLEVRDGHSESSTLIGRYC |
| | | | GYEKPDDIKSTSSRLWLKFVSDGSINKAGF |
| | | | AVNFFKEVDECSRPNRGGCEQRCLNTLGSY |
| | | | KCSCDPGYELAPDKRRCEAACGGFLTKLNG |
| | | | SITSPGWPKEYPPNKNCIWQLVAPTQYRIS |
| | | | LQFDFFETEGNDVCKYDFVEVRSGLTADSK |
| | | | LHGKFCGSEKPEVITSQYNNMRVEFKSDNT |
| | | | VSKKGFKAHFFSEKRPALQPPRGRPHQLKF |
| | | | RVQKRNRTPQ |
| P13497 | 3 | 12 | MPGVARLPLLLGLLLLPRPGRPLDLADYTY |
| | | | DLAEEDDSEPLNYKDPCKAAAFLGDIALDE |
| | | | EDLRAFQVQQAVDLRRHTARKSSIKAAVPG |
| | | | NTSTPSCQSTNGQPQRGACGRWRGRSRSRR |
| | | | AATSRPERVWPDGVIPFVIGGNFTGSQRAV |
| | | | FRQAMRHWEKHTCVTFLERTDEDSYIVFTY |
| | | | RPCGCCSYVGRRGGGPQAISIGKNCDKFGI |
| | | | VVHELGHVVGFWHEHTRPDRDRHVSIVREN |
| | | | IQPGVLHSSLLLLSCGSRNGASFPCSLESS |
| | | | THQALCWTGLFLRPSPFPRLPLAAPRTLRA |
| | | | GV |
| P13497 | 4 | 13 | MPGVARLPLLLGLLLLPRPGRPLDLADYTY |
| | | | DLAEEDDSEPLNYKDPCKAAAFLGDIALDE |
| | | | EDLRAFQVQQAVDLRRHTARKSSIKAAVPG |
| | | | NTSTPSCQSTNGQPQRGACGRWRGRSRSRR |
| | | | AATSRPERVWPDGVIPFVIGGNFTGSQRAV |
| | | | FRQAMRHWEKHTCVTFLERTDEDSYIVFTY |
| | | | RPCGCCSYVGRRGGGPQAISIGKNCDKFGI |
| | | | VVHELGHVVGFWHEHTRPDRDRHVSIVREN |
| | | | IQPGQEYNFLKMEPQEVESLGETYDFDSIM |
| | | | HYARNTFSRGIFLDTIVPKYEVNGVKPPIG |
| | | | QRTRLSKGDIAQARKLYKCPACGETLQDST |
| | | | GNFSSPEYPNGYSAHMHCVWRISVTPGEKI |
| | | | ILNFTSLDLYRSRLCWYDYVEVRDGFWRKA |
| | | | PLRGRFCGSKLPEPIVSTDSRLWVEFRSSS |
| | | | NWVGKGFFAVYEAICGGDVKKDYGHIQSPN |
| | | | YPDDYRPSKVCIWRIQVSEGFHVGLTFQSF |
| | | | EIERHDSCAYDYLEVRDGHSESSTLIGRYC |
| | | | GYEKPDDIKSTSSRLWLKFVSDGSINKAGF |
| | | | AVNFFKEVDECSRPNRGGCEQRCLNTLGSY |
| | | | KCSCDPGYELAPDKRRCEGCYDLQVGKPLL |
| | | | WDRHCFRLSTHGPEMLGTALRG |
| P13497 | 5 | 14 | MPGVARLPLLLGLLLLPRPGRPLDLADYTY |
| | | | DLAEEDDSEPLNYKDPCKAAAFLGDIALDE |
| | | | EDLRAFQVQQAVDLRRHTARKSSIKAAVPG |
| | | | NTSTPSCQSTNGQPQRGACGRWRGRSRSRR |
| | | | AATSRPERVWPDGVIPFVIGGNFTGSQRAV |
| | | | FRQAMRHWEKHTCVTFLERTDEDSYIVFTY |
| | | | RPCGCCSYVGRRGGGPQAISIGKNCDKFGI |
| | | | VVHELGHVVGFWHEHTRPDRDRHVSIVREN |
| | | | IQPGQEYNFLKMEPQEVESLGETYDFDSIM |
| | | | HYARNTFSRGIFLDTIVPKYEVNGVKPPIG |
| | | | QRTRLSKGDIAQARKLYKCPACGETLQDST |
| | | | GNFSSPEYPNGYSAHMHCVWRISVTPGEKI |
| | | | ILNFTSLDLYRSRLCWYDYVEVRDGFWRKA |
| | | | PLRGRFCGSKLPEPIVSTDSRLWVEFRSSS |
| | | | NWVGKGFFAVYEAICGGDVKKDYGHIQSPN |
| | | | YPDDYRPSKVCIWRIQVSEGFHVGLTFQSF |
| | | | EIERHDSCAYDYLEVRDGHSESSTLIGRYC |
| | | | GYEKPDDIKSTSSRLWLKFVSDGSINKAGF |
| | | | AVNFFKEVDECSRPNRGGCEQRCLNTLGSY |
| | | | KCSCDPGYELAPDKRRCEAACGGFLTKLNG |
| | | | SITSPGWPKEYPPNKNCIWQLVAPTQYRIS |
| | | | LQFDFFETEGNDVCKYDFVEVRSGLTADSK |
| | | | LHGKFCGSEKPEVITSQYNNMRVEFKSDNT |
| | | | VSKKGFKAHFFSGGELFGLLGHPPRRP |
| P13497 | 6 | 15 | MPGVARLPLLLGLLLLPRPGRPLDLADYTY |
| | | | DLAEEDDSEPLNYKDPCKAAAFLGDIALDE |
| | | | EDLRAFQVQQAVDLRRHTARKSSIKAAVPG |
| | | | NTSTPSCQSTNGQPQRGACGRWRGRSRSRR |
| | | | AATSRPERVWPDGVIPFVIGGNFTGSQRAV |
| | | | FRQAMRHWEKHTCVTFLERTDEDSYIVFTY |
| | | | RPCGCCSYVGRRGGGPQAISIGKNCDKFGI |
| | | | VVHELGHVVGFWHEHTRPDRDRHVSIVREN |
| | | | IQPGQEYNFLKMEPQEVESLGETYDFDSIM |
| | | | HYARNTFSRGIFLDTIVPKYEVNGVKPPIG |
| | | | QRTRLSKGDIAQARKLYKCPACGETLQDST |
| | | | GNFSSPEYPNGYSAHMHCVWRISVTPGEKI |
| | | | ILNFTSLDLYRSRLCWYDYVEVRDGFWRKA |
| | | | PLRGRFCGSKLPEPIVSTDSRLWVEFRSSS |
| | | | NWVGKGFFAVYEAICGGDVKKDYGHIQSPN |
| | | | YPDDYRPSKVCIWRIQVSEGFHVGLTFQSF |
| | | | EIERHDSCAYDYLEVRDGHSESSTLIGRYC |
| | | | GYEKPDDIKSTSSRLWLKFVSDGSINKAGF |
| | | | AVNFFKEVDECSRPNRGGCEQRCLNTLGSY |
| | | | KCSCDPGYELAPDKRRCEAACGGFLTKLNG |
| | | | SITSPGWPKEYPPNKNCIWQLVAPTQYRIS |
| | | | LQFDFFETEGNDVCKYDFVEVRSGLTADSK |
| | | | LHGKFCGSEKPEVITSQYNNMRVEFKSDNT |
| | | | VSKKGFKAHFFSVLEGAGDRHSHLSGLELL |
| | | | LCPHALVDTVPAPPSALHGDTHAHTHTHVH |
| | | | THCPIAQETCRGPPLGASRLSPQGPGHLTL |
| | | | APQEGSYLDFWDTHRGDPKPRRRRKSLKTF |
| | | | SLTPATFRGIWAL |
| P13497 | 7 | 16 | UNKNOWN |
| P24593 | 1 | 78 | MVLLTAVLLLLAAYAGPAQSLGSFVHCEPC |
| | | | DEKALSMCPPSPLGCELVKEPGCGCCMTCA |
| | | | LAEGQSCGVYTERCAQGLRCLPRQDEEKPL |
| | | | HALLHGRGVCLNEKSYREQVKIERDSREHE |
| | | | EPTTSEMAEETYSPKIFRPKHTRISELKAE |
| | | | AVKKDRRKKLTQSKFVGGAENTAHPRIISA |
| | | | PEMRQESEQGPCRRHMEASLQELKASPRMV |
| | | | PRAVYLPNCDRKGFYKRKQCKPSRGRKRGI |
| | | | CWCVDKYGMKLPGMEYVDGDFQCHTFDSSN |
| | | | VE |
| P30043 | 1 | 79 | MAVKKIAIFGATGQTGLTTLAQAVQAGYEV |
| | | | TVLVRDSSRLPSEGPRPAHVVVGDVLQAAD |
| | | | VDKTVAGQDAVIVLLGTRNDLSPTTVMSEG |
| | | | ARNIVAAMKAHGVDKVVACTSAFLLWDPTK |
| | | | VPPRLQAVTDDHIRMHKVLRESGLKYVAVM |
| | | | PPHIGDQPLTGAYTVTLDGRGPSRVISKHD |
| | | | LGHFMLRCLTTDEYDGHSTYPSHQYQ |
| P35858 | 1 | 80 | MALRKGGLALALLLLSWVALGPRSLEGADP |
| | | | GTPGEAEGPACPAACVCSYDDDADELSVFC |
| | | | SSRNLTRLPDGVPGGTQALWLDGNNLSSVP |
| | | | PAAFQNLSSLGFLNLQGGQLGSLEPQALLG |
| | | | LENLCHLHLERNQLRSLALGTFAHTPALAS |
| | | | LGLSNNRLSRLEDGLFEGLGSLWDLNLGWN |
| | | | SLAVLPDAAFRGLGSLRELVLAGNRLAYLQ |
| | | | PALFSGLAELRELDLSRNALRAIKANVFVQ |
| | | | LPRLQKLYLDRNLIAAVAPGAFLGLKALRW |
| | | | LDLSHNRVAGLLEDTFPGLLGLRVLRLSHN |
| | | | AIASLRPRTFKDLHFLEELQLGHNRIRQLA |
| | | | ERSFEGLGQLEVLTLDHNQLQEVKAGAFLG |
| | | | LTNVAVMNLSGNCLRNLPEQVFRGLGKLHS |
| | | | LHLEGSCLGRIRPHTFTGLSGLRRLFLKDN |
| | | | GLVGIEEQSLWGLAELLELDLTSNQLTHLP |
| | | | HRLFQGLGKLEYLLLSRNRLAELPADALGP |
| | | | LQRAFWLDVSHNRLEALPNSLLAPLGRLRY |
| | | | LSLRNNSLRTFTPQPPGLERLWLEGNPWDC |
| | | | GCPLKALRDFALQNPSAVPRFVQAICEGDD |
| | | | CQPPAYTYNNITCASPPEVVGLDLRDLSEA |
| | | | HFAPC |
| P35858 | 2 | 81 | MALRKAGDLEPQFTPERRFRLCWYQAHSGR |
| | | | ALLGPPPQASPPAGGLALALLLLSWVALGP |
| | | | RSLEGADPGTPGEAEGPACPAACVCSYDDD |
| | | | ADELSVFCSSRNLTRLPDGVPGGTQALWLD |
| | | | GNNLSSVPPAAFQNLSSLGFLNLQGGQLGS |
| | | | LEPQALLGLENLCHLHLERNQLRSLALGTF |
| | | | AHTPALASLGLSNNRLSRLEDGLFEGLGSL |
| | | | WDLNLGWNSLAVLPDAAFRGLGSLRELVLA |
| | | | GNRLAYLQPALFSGLAELRELDLSRNALRA |
| | | | IKANVFVQLPRLQKLYLDRNLIAAVAPGAF |
| | | | LGLKALRWLDLSHNRVAGLLEDTFPGLLGL |
| | | | RVLRLSHNAIASLRPRTFKDLHFLEELQLG |
| | | | HNRIRQLAERSFEGLGQLEVLTLDHNQLQE |
| | | | VKAGAFLGLTNVAVMNLSGNCLRNLPEQVF |
| | | | RGLGKLHSLHLEGSCLGRIRPHTFTGLSGL |
| | | | RRLFLKDNGLVGIEEQSLWGLAELLELDLT |
| | | | SNQLTHLPHRLFQGLGKLEYLLLSRNRLAE |
| | | | LPADALGPLQRAFWLDVSHNRLEALPNSLL |
| | | | APLGRLRYLSLRNNSLRTFTPQPPGLERLW |
| | | | LEGNPWDCGCPLKALRDFALQNPSAVPRFV |
| | | | QAICEGDDCQPPAYTYNNITCASPPEVVGL |
| | | | DLRDLSEAHFAPC |
| P41218 | 1 | 82 | MVNEYKKILLLKGFELMDDYHFTSIKSLLA |
| | | | YDLGLTTKMQEEYNRIKITDLMEKKFQGVA |
| | | | CLDKLIELAKDMPSLKNLVNNLRKEKSKVA |
| | | | KKIKTQEKAPVKKINQEEVGLAAPAPTARN |
| | | | KLTSEARGRIPVAQKRKTPNKEKTEAKRNK |
| | | | VSQEQSKPPGPSGASTSAAVDHPPLPQTSS |
| | | | STPSNTSFTPNQETQAQRQVDARRNVPQND |
| | | | PVTVVVLKATAPFKYESPENGKSTMFHATV |
| | | | ASKTQYFHVKVFDINLKEKFVRKKVITISD |
| | | | YSECKGVMEIKEASSVSDFNQNFEVPNRII |
| | | | EIANKTPKISQLYKQASGTMVYGLFMLQKK |
| | | | SVHKKNTIYEIQDNTGSMDVVGSGKWHNIK |
| | | | CEKGDKLRLFCLQLRTVDRKLKLVCGSHSF |
| | | | IKVIKAKKNKEGPMNVN |
| Q13214 | 1 | 83 | MGRAGAAAVIPGLALLWAVGLGSAAPSPPR |
| | | | LRLSFQELQAWHGLQTFSLERTCCYQALLV |
| | | | DEERGRLFVGAENHVASLNLDNISKRAKKL |
| | | | AWPAPVEWREECNWAGKDIGTECMNFVKLL |
| | | | HAYNRTHLLACGTGAFHPTCAFVEVGHRAE |
| | | | EPVLRLDPGRIEDGKGKSPYDPRHRAASVL |
| | | | VGEELYSGVAADLMGRDFTIFRSLGQRPSL |
| | | | RTEPHDSRWLNEPKFVKVFWIPESENPDDD |
| | | | KIYFFFRETAVEAAPALGRLSVSRVGQICR |
| | | | NDVGGQRSLVNKWTTFLKARLVCSVPGVEG |
| | | | DTHFDQLQDVFLLSSRDHRTPLLYAVFSTS |
| | | | SSIFQGSAVCVYSMNDVRRAFLGPFAHKEG |
| | | | PMHQWVSYQGRVPYPRPGMCPSKTFGTFSS |
| | | | TKDFPDDVIQFARNHPLMYNSVLPTGGRPL |
| | | | FLQVGANYTFTQIAADRVAAADGHYDVLFI |
| | | | GTDVGTVLKVISVPKGSRPSAEGLLLEELH |
| | | | VFEDSAAVTSMQISSKRHQLYVASRSAVAQ |
| | | | IALHRCAAHGRVCTECCLARDPYCAWDGVA |
| | | | CTRFQPSAKRRFRRQDVRNGDPSTLCSGDS |
| | | | SRPALLEHKVFGVEGSSAFLECEPRSLQAR |
| | | | VEWTFQRAGVTAHTQVLAEERTERTARGLL |
| | | | LRRLRRRDSGVYLCAAVEQGFTQPLRRLSL |
| | | | HVLSATQAERLARAEEAAPAAPPGPKLWYR |
| | | | DFLQLVEPGGGGSANSLRMCRPQPALQSLP |
| | | | LESRRKGRNRRTHAPEPRAERGPRSATHW |
| Q13214 | 2 | 84 | MGRAGAAAVIPGLALLWAVGLGSAAPSPPR |
| | | | LRLSFQELQAWHGLQTFSLERTCCYQALLV |
| | | | DEERGRLFVGAENHVASLNLDNISKRAKKL |
| | | | AWPAPVEWREECNWAGKDIGTECMNFVKLL |
| | | | HAYNRTHLLACGTGAFHPTCAFVEVGHRAE |
| | | | EPVLRLDPGRIEDGKGKSPYDPRHRAASVL |
| | | | VGEELYSGVAADLMGRDFTIFRSLGQRPSL |
| | | | RTEPHDSRWLNEPKFVKVFWIPESENPDDD |
| | | | KIYFFFRETAVEAAPALGRLSVSRVGQICR |
| | | | NDVGGQRSLVNKWTTFLKARLVCSVPGVEG |
| | | | DTHFDQLQDVFLLSSRDHRTPLLYAVESTS |
| | | | SIFQGSAVCVYSMNDVRRAFLGPFAHKEGP |
| | | | MHQWVSYQGRVPYPRPGMCPSKTFGTFSST |
| | | | KDFPDDVIQFARNHPLMYNSVLPTGGRPLF |
| | | | LQVGANYTFTQIAADRVAAADGHYDVLFIG |
| | | | TDVGTVLKVISVPKGSRPSAEGLLLEELHV |
| | | | FEDSAAVTSMQISSKRHQLYVASRSAVAQI |
| | | | ALHRCAAHGRVCTECCLARDPYCAWDGVAC |
| | | | TRFQPSAKRRFRRQDVRNGDPSTLCSGDSS |
| | | | RPALLEHKVFGVEGSSAFLECEPRSLQARV |
| | | | EWTFQRAGVTAHTQVLAEERTERTARGLLL |
| | | | RRLRRRDSGVYLCAAVEQGFTQPLRRLSLH |
| | | | VLSATQAERLARAEEAAPAAPPGPKLWYRD |
| | | | FLQLVEPGGGGSANSLRMCRPQPALQSLPL |
| | | | ESRRKGRNRRTHAPEPRAERGPRSATHW |
| Q13822 | 1 | 85 | MARRSSFQSCQIISLFTFAVGVNICLGFTA |
| | | | HRIKRAEGWEEGPPTVLSDSPWTNISGSCK |
| | | | GRCFELQEAGPPDCRCDNLCKSYTSCCHDF |
| | | | DELCLKTARGWECTKDRCGEVRNEENACHC |
| | | | SEDCLARGDCCTNYQVVCKGESHWVDDDCE |
| | | | EIKAAECPAGFVRPPLIIFSVDGFRASYMK |
| | | | KGSKVMPNIEKLRSCGTHSPYMRPVYPTKT |
| | | | FPNLYTLATGLYPESHGIVGNSMYDPVFDA |
| | | | TFHLRGREKFNHRWWGGQPLWITATKQGVK |
| | | | AGTFFWSVVIPHERRILTILQWLTLPDHER |
| | | | PSVYAFYSEQPDFSGHKYGPFGPEMTNPLR |
| | | | EIDKIVGQLMDGLKQLKLHRCVNVIFVGDH |
| | | | GMEDVTCDRTEFLSNYLTNVDDITLVPGTL |
| | | | GRIRSKFSNNAKYDPKAIIANLTCKKPDQH |
| | | | FKPYLKQHLPKRLHYANNRRIEDIHLLVER |
| | | | RWHVARKPLDVYKKPSGKCFFQGDHGFDNK |
| | | | VNSMQTVFVGYGSTFKYKTKVPPFENIELY |
| | | | NVMCDLLGLKPAPNNGTHGSLNHLLRTNTF |
| | | | RPTMPEEVTRPNYPGIMYLQSDFDLGCTCD |
| | | | DKVEPKNKLDELNKRLHTKGSTEERHLLYG |
| | | | RPAVLYRTRYDILYHTDFESGYSEIFLMPL |
| | | | WTSYTVSKQAEVSSVPDHLTSCVRPDVRVS |
| | | | PSFSQNCLAYKNDKQMSYGFLFPPYLSSSP |
| | | | EAKYDAFLVTNMVPMYPAFKRVWNYFQRVL |
| | | | VKKYASERNGVNVISGPIFDYDYDGLHDTE |
| | | | DKIKQYVEGSSIPVPTHYYSIITSCLDFTQ |
| | | | PADKCDGPLSVSSFILPHRPDNEESCNSSE |
| | | | DESKWVEELMKMHTARVRDIEHLTSLDFFR |
| | | | KTSRSYPEILTLKTYLHTYESEI |
| Q13822 | 2 | 86 | MARRSSFQSCQIISLFTFAVGVNICLGFTA |
| | | | HRIKRAEGWEEGPPTVLSDSPWTNISGSCK |
| | | | GRCFELQEAGPPDCRCDNLCKSYTSCCHDF |
| | | | DELCLKTARGWECTKDRCGEVRNEENACHC |
| | | | SEDCLARGDCCTNYQVVCKGESHWVDDDCE |
| | | | EIKAAECPAGFVRPPLIIFSVDGFRASYMK |
| | | | KGSKVMPNIEKLRSCGTHSPYMRPVYPTKT |
| | | | FPNLYTLATGLYPESHGIVGNSMYDPVFDA |
| | | | TFHLRGREKFNHRWWGGQPLWITATKQGVK |
| | | | AGTFFWSVVIPHERRILTILQWLTLPDHER |
| | | | PSVYAFYSEQPDFSGHKYGPFGPEESSYGS |
| | | | PFTPAKRPKRKVAPKRRQERPVAPPKKRRR |
| | | | KIHRMDHYAAETRQDKMTNPLREIDKIVGQ |
| | | | LMDGLKQLKLHRCVNVIFVGDHGMEDVTCD |
| | | | RTEFLSNYLTNVDDITLVPGTLGRIRSKFS |
| | | | NNAKYDPKAIIANLTCKKPDQHFKPYLKQH |
| | | | LPKRLHYANNRRIEDIHLLVERRWHVARKP |
| | | | LDVYKKPSGKCFFQGDHGFDNKVNSMQTVF |
| | | | VGYGSTFKYKTKVPPFENIELYNVMCDLLG |
| | | | LKPAPNNGTHGSLNHLLRTNTFRPTMPEEV |
| | | | TRPNYPGIMYLQSDFDLGCTCDDKVEPKNK |
| | | | LDELNKRLHTKGSTEERHLLYGRPAVLYRT |
| | | | RYDILYHTDFESGYSEIFLMPLWTSYTVSK |
| | | | QAEVSSVPDHLTSCVRPDVRVSPSFSQNCL |
| | | | AYKNDKQMSYGFLFPPYLSSSPEAKYDAFL |
| | | | VTNMVPMYPAFKRVWNYFQRVLVKKYASER |
| | | | NGVNVISGPIFDYDYDGLHDTEDKIKQYVE |
| | | | GSSIPVPTHYYSIITSCLDFTQPADKCDGP |
| | | | LSVSSFILPHRPDNEESCNSSEDESKWVEE |
| | | | LMKMHTARVRDIEHLTSLDFFRKTSRSYPE |
| | | | ILTLKTYLHTYESEI |
| Q13822 | 3 | 87 | MARRSSFQSCQIISLFTFAVGVNICLGFTA |
| | | | HRIKRAEGWEEGPPTVLSDSPWTNISGSCK |
| | | | GRCFELQEAGPPDCRCDNLCKSYTSCCHDF |
| | | | DELCLKTARGWECTKDRCGEVRNEENACHC |
| | | | SEDCLARGDCCTNYQVVCKGESHWVDDDCE |
| | | | EIKAAECPAGFVRPPLIIFSVDGFRASYMK |
| | | | KGSKVMPNIEKLRSCGTHSPYMRPVYPTKT |
| | | | FPNLYTLATGLYPESHGIVGNSMYDPVFDA |
| | | | TFHLRGREKFNHRWWGGQPLWITATKQGVK |
| | | | AGTFFWSVVIPHERRILTILQWLTLPDHER |
| | | | PSVYAFYSEQPDFSGHKYGPFGPEMTNPLR |
| | | | EIDKIVGQLMDGLKQLKLHRCVNVIFVGDH |
| | | | GMEDVTCDRTEFLSNYLTNVDDITLVPGTL |
| | | | GRIRSKFSNNAKYDPKAIIANLTCKKPDQH |
| | | | FKPYLKQHLPKRLHYANNRRIEDIHLLVER |
| | | | RWHVARKPLDVYKKPSGKCFFQGDHGFDNK |
| | | | VNSMQTVFVGYGSTFKYKTKVPPFENIELY |
| | | | NVMCDLLGLKPAPNNGTHGSLNHLLRTNTF |
| | | | RPTMPEEVTRPNYPGIMYLQSDFDLGCTCD |
| | | | DKVEPKNKLDELNKRLHTKGSTEAETRKFR |
| | | | GSRNENKENINGNFEPRKERHLLYGRPAVL |
| | | | YRTRYDILYHTDFESGYSEIFLMPLWTSYT |
| | | | VSKQAEVSSVPDHLTSCVRPDVRVSPSFSQ |
| | | | NCLAYKNDKQMSYGFLFPPYLSSSPEAKYD |
| | | | AFLVTNMVPMYPAFKRVWNYFQRVLVKKYA |
| | | | SERNGVNVISGPIFDYDYDGLHDTEDKIKQ |
| | | | YVEGSSIPVPTHYYSIITSCLDFTQPADKC |
| | | | DGPLSVSSFILPHRPDNEESCNSSEDESKW |
| | | | VEELMKMHTARVRDIEHLTSLDFFRKTSRS |
| | | | YPEILTLKTYLHTYESEI |
| Q8NI99 | 1 | 88 | MGKPWLRALQLLLLLGASWARAGAPRCTYT |
| | | | FVLPPQKFTGAVCWSGPASTRATPEAANAS |
| | | | ELAALRMRVGRHEELLRELQRLAAADGAVA |
| | | | GEVRALRKESRGLSARLGQLRAQLQHEAGP |
| | | | GAGPGADLGAEPAAALALLGERVLNASAEA |
| | | | QRAAARFHQLDVKFRELAQLVTQQSSLIAR |
| | | | LERLCPGGAGGQQQVLPPPPLVPVVPVRLV |
| | | | GSTSDTSRMLDPAPEPQRDQTQRQQEPMAS |
| | | | PMPAGHPAVPTKPVGPWQDCAEARQAGHEQ |
| | | | SGVYELRVGRHVVSVWCEQQLEGGGWTVIQ |
| | | | RRQDGSVNFFTTWQHYKAGFGRPDGEYWLG |
| | | | LEPVYQLTSRGDHELLVLLEDWGGRGARAH |
| | | | YDGFSLEPESDHYRLRLGQYHGDAGDSLSW |
| | | | HNDKPFSTVDRDRDSYSGNCALYQRGGWWY |
| | | | HACAHSNLNGVWHHGGHYRSRYQDGVYWAE |
| | | | FRGGAYSLRKAAMLIRPLKL |
| Q96IY4 | 1 | 89 | MKLCSLAVLVPIVLFCEQHVFAFQSGQVLA |
| | | | ALPRTSRQVQVLQNLTTTYEIVLWQPVTAD |
| | | | LIVKKKQVHFFVNASDVDNVKAHLNVSGIP |
| | | | CSVLLADVEDLIQQQISNDTVSPRASASYY |
| | | | EQYHSLNEIYSWIEFITERHPDMLTKIHIG |
| | | | SSFEKYPLYVLKVSGKEQAAKNAIWIDCGI |
| | | | HAREWISPAFCLWFIGHITQFYGIIGQYTN |
| | | | LLRLVDFYVMPVVNVDGYDYSWKKNRMWRK |
| | | | NRSFYANNHCIGTDLNRNFASKHWCEEGAS |
| | | | SSSCSETYCGLYPESEPEVKAVASFLRRNI |
| | | | NQIKAYISMHSYSQHIVFPYSYTRSKSKDH |
| | | | EELSLVASEAVRAIEKISKNTRYTHGHGSE |
| | | | TLYLAPGGGDDWIYDLGIKYSFTIELRDTG |
| | | | TYGFLLPERYIKPTCREAFAAVSKIAWHVI |
| | | | RNV |
| Q96IY4 | 2 | 90 | MKLCSLAVLVPIVLFCEQHVFAFQSGQVLA |
| | | | ALPRTSRQVQVLQNLTTTYEIVLWQPVTAD |
| | | | LIVKKKQVHFFVNASDVDNVKAHLNVSGIP |
| | | | CSVLLADVEDLIQQQISNDTVSPRASASYY |
| | | | EQYHSLNEIYSWIEFITERHPDMLTKIHIG |
| | | | SSFEKYPLYVLKVSGKEQAAKNAIWIDCGI |
| | | | HAREWISPAFCLWFIGHNRMWRKNRSFYAN |
| | | | NHCIGTDLNRNFASKHWCEEGASSSSCSET |
| | | | YCGLYPESEPEVKAVASFLRRNINQIKAYI |
| | | | SMHSYSQHIVFPYSYTRSKSKDHEELSLVA |
| | | | SEAVRAIEKISKNTRYTHGHGSETLYLAPG |
| | | | GGDDWIYDLGIKYSFTSNPPVEKLLPLSLK |
| Q99715 | 1 | 91 | MRSRLPPALAALGAALLLSSIEAEVDPPSD |
| | | | LNFKIIDENTVHMSWAKPVDPIVGYRITVD |
| | | | PTTDGPTKEFTLSASTTETLLSELVPETEY |
| | | | VVTITSYDEVEESVPVIGQLTIQTGSSTKP |
| | | | VEKKPGKTEIQKCSVSAWTDLVFLVDGSWS |
| | | | VGRNNFKYILDFIAALVSAFDIGEEKTRVG |
| | | | VVQYSSDTRTEFNLNQYYQRDELLAAIKKI |
| | | | PYKGGNTMTGDAIDYLVKNTFTESAGARVG |
| | | | FPKVAIIITDGKSQDEVEIPARELRNVGVE |
| | | | VFSLGIKAADAKELKQIASTPSLNHVFNVA |
| | | | NFDAIVDIQNEIISQVCSGVDEQLGELVSG |
| | | | EEVVEPPSNLIAMEVSSKYVKLNWNPSPSP |
| | | | VTGYKVILTPMTAGSRQHALSVGPQTTTLS |
| | | | VRDLSADTEYQISVSAMKGMTSSEPISIME |
| | | | KTQPMKVQVECSRGVDIKADIVFLVDGSYS |
| | | | IGIANFVKVRAFLEVLVKSFEISPNRVQIS |
| | | | LVQYSRDPHTEFTLKKFTKVEDIIEAINTF |
| | | | PYRGGSTNTGKAMTYVREKIFVPSKGSRSN |
| | | | VPKVMILITDGKSSDAFRDPAIKLRNSDVE |
| | | | IFAVGVKDAVRSELEAIASPPAETHVFTVE |
| | | | DFDAFQRISFELTQSICLRIEQELAAIKKK |
| | | | AYVPPKDLSFSEVTSYGFKTNWSPAGENVF |
| | | | SYHITYKEAAGDDEVTVVEPASSTSVVLSS |
| | | | LKPETLYLVNVTAEYEDGFSIPLAGEETTE |
| | | | EVKGAPRNLKVTDETTDSFKITWTQAPGRV |
| | | | LRYRIIYRPVAGGESREVTTPPNQRRRTLE |
| | | | NLIPDTKYEVSVIPEYFSGPGTPLTGNAAT |
| | | | EEVRGNPRDLRVSDPTTSTMKLSWSGAPGK |
| | | | VKQYLVTYTPVAGGETQEVTVRGDTTNTVL |
| | | | QGLKEGTQYALSVTALYASGAGDALFGEGT |
| | | | TLEERGSPQDLVTKDITDTSIGAYWTSAPG |
| | | | MVRGYRVSWKSLYDDVDTGEKNLPEDAIHT |
| | | | MIENLQPETKYRISVFATYSSGEGEPLTGD |
| | | | ATTELSQDSKTLKVDEETENTMRVTWKPAP |
| | | | GKVVNYRVVYRPHGRGKQMVAKVPPTVTST |
| | | | VLKRLQPQTTYDITVLPIYKMGEGKLRQGS |
| | | | GTTASRFKSPRNLKTSDPTMSSFRVTWEPA |
| | | | PGEVKGYKVTFHPTGDDRRLGELVVGPYDN |
| | | | TVVLEELRAGTTYKVNVFGMFDGGESSPLV |
| | | | GQEMTTLSDTTVMPILSSGMECLTRAEADI |
| | | | VLLVDGSWSIGRANFRTVRSFISRIVEVFD |
| | | | IGPKRVQIALAQYSGDPRTEWQLNAHRDKK |
| | | | SLLQAVANLPYKGGNTLTGMALNFIRQQNF |
| | | | RTQAGMRPRARKIGVLITDGKSQDDVEAPS |
| | | | KKLKDEGVELFAIGIKNADEVELKMIATDP |
| | | | DDTHAYNVADFESLSRIVDDLTINLCNSVK |
| | | | GPGDLEAPSNLVISERTHRSFRVSWTPPSD |
| | | | SVDRYKVEYYPVSGGKRQEFYVSRMETSTV |
| | | | LKDLKPETEYVVNVYSVVEDEYSEPLKGTE |
| | | | KTLPVPVVSLNIYDVGPTTMHVQWQPVGGA |
| | | | TGYILSYKPVKDTEPTRPKEVRLGPTVNDM |
| | | | QLTDLVPNTEYAVTVQAVLHDLTSEPVTVR |
| | | | EVTLPLPRPQDLKLRDVTHSTMNVFWEPVP |
| | | | GKVRKYIVRYKTPEEDVKEVEVDRSETSTS |
| | | | LKDLFSQTLYTVSVSAVHDEGESPPVTAQE |
| | | | TTRPVPAPTNLKITEVTSEGFRGTWDHGAS |
| | | | DVSLYRITWAPFGSSDKMETILNGDENTLV |
| | | | FENLNPNTIYEVSITAIYPDESESDDLIGS |
| | | | ERTLPILTTQAPKSGPRNLQVYNATSNSLT |
| | | | VKWDPASGRVQKYRITYQPSTGEGNEQTTT |
| | | | IGGRQNSVVLQKLKPDTPYTITVSSLYPDG |
| | | | EGGRMTGRGKTKPLNTVRNLRVYDPSTSTL |
| | | | NVRWDHAEGNPRQYKLFYAPAAGGPEELVP |
| | | | IPGNTNYAILRNLQPDTSYTVTVVPVYTEG |
| | | | DGGRTSDTGRTLMRGLARNVQVYNPTPNSL |
| | | | DVRWDPAPGPVLQYRVVYSPVDGTRPSESI |
| | | | VVPGNTRMVHLERLIPDTLYSVNLVALYSD |
| | | | GEGNPSPAQGRTLPRSGPRNLRVFGETTNS |
| | | | LSVAWDHADGPVQQYRIIYSPTVGDPIDEY |
| | | | TTVPGRRNNVILQPLQPDTPYKITVIAVYE |
| | | | DGDGGHLTGNGRTVGLLPPQNIHISDEWYT |
| | | | RFRVSWDPSPSPVLGYKIVYKPVGSNEPME |
| | | | AFVGEMTSYTLHNLNPSTTYDVNVYAQYDS |
| | | | GLSVPLTDQGTTLYLNVTDLKTYQIGWDTF |
| | | | CVKWSPHRAATSYRLKLSPADGTRGQEITV |
| | | | RGSETSHCFTGLSPDTDYGVTVFVQTPNLE |
| | | | GPGVSVKEHTTVKPTEAPTEPPTPPPPPTI |
| | | | PPARDVCKGAKADIVFLTDASWSIGDDNFN |
| | | | KVVKFIFNTVGGFDEISPAGIQVSFVQYSD |
| | | | EVKSEFKLNTYNDKALALGALQNIRYRGGN |
| | | | TRTGKALTFIKEKVLTWESGMRKNVPKVLV |
| | | | VVTDGRSQDEVKKAALVIQQSGFSVFVVGV |
| | | | ADVDYNELANIASKPSERHVFIVDDFESFE |
| | | | KIEDNLITFVCETATSSCPLIYLDGYTSPG |
| | | | FKMLEAYNLTEKNFASVQGVSLESGSFPSY |
| | | | SAYRIQKNAFVNQPTADLHPNGLPPSYTII |
| | | | LLFRLLPETPSDPFAIWQITDRDYKPQVGV |
| | | | IADPSSKTLSFFNKDTRGEVQTVTFDTEEV |
| | | | KTLFYGSFHKVHIVVTSKSVKIYIDCYEII |
| | | | EKDIKEAGNITTDGYEILGKLLKGERKSAA |
| | | | FQIQSFDIVCSPVWTSRDRCCDIPSRRDEG |
| | | | KCPAFPNSCTCTQDSVGPPGPPGPAGGPGA |
| | | | KGPRGERGISGAIGPPGPRGDIGPPGPQGP |
| | | | PGPQGPNGLSIPGEQGRQGMKGDAGEPGLP |
| | | | GRTGTPGLPGPPGPMGPPGDRGFTGKDGAM |
| | | | GPRGPPGPPGSPGSPGVTGPSGKPGKPGDH |
| | | | GRPGPSGLKGEKGDRGDIASQNMMRAVARQ |
| | | | VCEQLISGQMNRFNQMLNQIPNDYQSSRNQ |
| | | | PGPPGPPGPPGSAGARGEPGPGGRPGFPGT |
| | | | PGMQGPPGERGLPGEKGERGTGSSGPRGLP |
| | | | GPPGPQGESRTGPPGSTGSRGPPGPPGRPG |
| | | | NSGIRGPPGPPGYCDSSQCASIPYNGQGYP |
| | | | GSG |
| Q99715 | 2 | 92 | MRSRLPPALAALGAALLLSSIEAEGMECLT |
| | | | RAEADIVLLVDGSWSIGRANFRTVRSFISR |
| | | | IVEVFDIGPKRVQIALAQYSGDPRTEWQLN |
| | | | AHRDKKSLLQAVANLPYKGGNTLTGMALNF |
| | | | IRQQNFRTQAGMRPRARKIGVLITDGKSQD |
| | | | DVEAPSKKLKDEGVELFAIGIKNADEVELK |
| | | | MIATDPDDTHAYNVADFESLSRIVDDLTIN |
| | | | LCNSVKGPGDLEAPSNLVISERTHRSFRVS |
| | | | WTPPSDSVDRYKVEYYPVSGGKRQEFYVSR |
| | | | METSTVLKDLKPETEYVVNVYSVVEDEYSE |
| | | | PLKGTEKTLPVPVVSLNIYDVGPTTMHVQW |
| | | | QPVGGATGYILSYKPVKDTEPTRPKEVRLG |
| | | | PTVNDMQLTDLVPNTEYAVTVQAVLHDLTS |
| | | | EPVTVREVTLPLPRPQDLKLRDVTHSTMNV |
| | | | FWEPVPGKVRKYIVRYKTPEEDVKEVEVDR |
| | | | SETSTSLKDLFSQTLYTVSVSAVHDEGESP |
| | | | PVTAQETTRPVPAPTNLKITEVTSEGFRGT |
| | | | WDHGASDVSLYRITWAPFGSSDKMETILNG |
| | | | DENTLVFENLNPNTIYEVSITAIYPDESES |
| | | | DDLIGSERTLPILTTQAPKSGPRNLQVYNA |
| | | | TSNSLTVKWDPASGRVQKYRITYQPSTGEG |
| | | | NEQTTTIGGRQNSVVLQKLKPDTPYTITVS |
| | | | SLYPDGEGGRMTGRGKTKPLNTVRNLRVYD |
| | | | PSTSTLNVRWDHAEGNPRQYKLFYAPAAGG |
| | | | PEELVPIPGNTNYAILRNLQPDTSYTVTVV |
| | | | PVYTEGDGGRTSDTGRTLMRGLARNVQVYN |
| | | | PTPNSLDVRWDPAPGPVLQYRVVYSPVDGT |
| | | | RPSESIVVPGNTRMVHLERLIPDTLYSVNL |
| | | | VALYSDGEGNPSPAQGRTLPRSGPRNLRVF |
| | | | GETTNSLSVAWDHADGPVQQYRITYSPTVG |
| | | | DPIDEYTTVPGRRNNVILQPLQPDTPYKIT |
| | | | VIAVYEDGDGGHLTGNGRTVGLLPPQNIHI |
| | | | SDEWYTRFRVSWDPSPSPVLGYKIVYKPVG |
| | | | SNEPMEAFVGEMTSYTLHNLNPSTTYDVNV |
| | | | YAQYDSGLSVPLTDQGTTLYLNVTDLKTYQ |
| | | | IGWDTFCVKWSPHRAATSYRLKLSPADGTR |
| | | | GQEITVRGSETSHCFTGLSPDTDYGVTVFV |
| | | | QTPNLEGPGVSVKEHTTVKPTEAPTEPPTP |
| | | | PPPPTIPPARDVCKGAKADIVFLTDASWSI |
| | | | GDDNFNKVVKFIFNTVGGFDEISPAGIQVS |
| | | | FVQYSDEVKSEFKLNTYNDKALALGALQNI |
| | | | RYRGGNTRTGKALTFIKEKVLTWESGMRKN |
| | | | VPKVLVVVTDGRSQDEVKKAALVIQQSGFS |
| | | | VFVVGVADVDYNELANIASKPSERHVFIVD |
| | | | DFESFEKIEDNLITFVCETATSSCPLIYLD |
| | | | GYTSPGFKMLEAYNLTEKNFASVQGVSLES |
| | | | GSFPSYSAYRIQKNAFVNQPTADLHPNGLP |
| | | | PSYTIILLFRLLPETPSDPFAIWQITDRDY |
| | | | KPQVGVIADPSSKTLSFFNKDTRGEVQTVT |
| | | | FDTEEVKTLFYGSFHKVHIVVTSKSVKIYI |
| | | | DCYEIIEKDIKEAGNITTDGYEILGKLLKG |
| | | | ERKSAAFQIQSFDIVCSPVWTSRDRCCDIP |
| | | | SRRDEGKCPAFPNSCTCTQDSVGPPGPPGP |
| | | | AGGPGAKGPRGERGISGAIGPPGPRGDIGP |
| | | | PGPQGPPGPQGPNGLSIPGEQGRQGMKGDA |
| | | | GEPGLPGRTGTPGLPGPPGPMGPPGDRGFT |
| | | | GKDGAMGPRGPPGPPGSPGSPGVTGPSGKP |
| | | | GKPGDHGRPGPSGLKGEKGDRGDIASQNMM |
| | | | RAVARQVCEQLISGQMNRFNQMLNQIPNDY |
| | | | QSSRNQPGPPGPPGPPGSAGARGEPGPGGR |
| | | | PGFPGTPGMQGPPGERGLPGEKGERGTGSS |
| | | | GPRGLPGPPGPQGESRTGPPGSTGSRGPPG |
| | | | PPGRPGNSGIRGPPGPPGYCDSSQCASIPY |
| | | | NGQGYPGSG |
| Q99715 | 4 | 93 | MRSRLPPALAALGAALLLSSIEAEVDPPSD |
| | | | LNFKIIDENTVHMSWAKPVDPIVGYRITVD |
| | | | PTTDGPTKEFTLSASTTETLLSELVPETEY |
| | | | VVTITSYDEVEESVPVIGQLTIQTGSSTKP |
| | | | VEKKPGKTEIQKCSVSAWTDLVFLVDGSWS |
| | | | VGRNNFKYILDFIAALVSAFDIGEEKTRVG |
| | | | VVQYSSDTRTEFNLNQYYQRDELLAAIKKI |
| | | | PYKGGNTMTGDAIDYLVKNTFTESAGARVG |
| | | | FPKVAIIITDGKSQDEVEIPARELRNVGVE |
| | | | VFSLGIKAADAKELKQIASTPSLNHVFNVA |
| | | | NFDAIVDIQNEIISQVCSGVDEQLGELVSG |
| | | | EEVVEPPSNLIAMEVSSKYVKLNWNPSPSP |
| | | | VTGYKVILTPMTAGSRQHALSVGPQTTTLS |
| | | | VRDLSADTEYQISVSAMKGMTSSEPISIME |
| | | | KTQPMKVQVECSRGVDIKADIVFLVDGSYS |
| | | | IGIANFVKVRAFLEVLVKSFEISPNRVQIS |
| | | | LVQYSRDPHTEFTLKKFTKVEDIIEAINTF |
| | | | PYRGGSTNTGKAMTYVREKIFVPSKGSRSN |
| | | | VPKVMILITDGKSSDAFRDPAIKLRNSDVE |
| | | | IFAVGVKDAVRSELEAIASPPAETHVFTVE |
| | | | DFDAFQRISFELTQSICLRIEQELAAIKKK |
| | | | AYVPPKDLSFSEVTSYGFKTNWSPAGENVF |
| | | | SYHITYKEAAGDDEVTVVEPASSTSVVLSS |
| | | | LKPETLYLVNVTAEYEDGFSIPLAGEETTE |
| | | | EVKGAPRNLKVTDETTDSFKITWTQAPGRV |
| | | | LRYRIIYRPVAGGESREVTTPPNQRRRTLE |
| | | | NLIPDTKYEVSVIPEYFSGPGTPLTGNAAT |
| | | | EEVRGNPRDLRVSDPTTSTMKLSWSGAPGK |
| | | | VKQYLVTYTPVAGGETQEVTVRGDTTNTVL |
| | | | QGLKEGTQYALSVTALYASGAGDALFGEGT |
| | | | TLEERGSPQDLVTKDITDTSIGAYWTSAPG |
| | | | MVRGYRVSWKSLYDDVDTGEKNLPEDAIHT |
| | | | MIENLQPETKYRISVFATYSSGEGEPLTGD |
| | | | ATTELSQDSKTLKVDEETENTMRVTWKPAP |
| | | | GKVVNYRVVYRPHGRGKQMVAKVPPTVTST |
| | | | VLKRLQPQTTYDITVLPIYKMGEGKLRQGS |
| | | | GTTASRFKSPRNLKTSDPTMSSFRVTWEPA |
| | | | PGEVKGYKVTFHPTGDDRRLGELVVGPYDN |
| | | | TVVLEELRAGTTYKVNVFGMFDGGESSPLV |
| | | | GQEMTTLSDTTVMPILSSGMECLTRAEADI |
| | | | VLLVDGSWSIGRANFRTVRSFISRIVEVFD |
| | | | IGPKRVQIALAQYSGDPRTEWQLNAHRDKK |
| | | | SLLQAVANLPYKGGNTLTGMALNFIRQQNF |
| | | | RTQAGMRPRARKIGVLITDGKSQDDVEAPS |
| | | | KKLKDEGVELFAIGIKNADEVELKMIATDP |
| | | | DDTHAYNVADFESLSRIVDDLTINLCNSVK |
| | | | GPGDLEAPSNLVISERTHRSFRVSWTPPSD |
| | | | SVDRYKVEYYPVSGGKRQEFYVSRMETSTV |
| | | | LKDLKPETEYVVNVYSVVEDEYSEPLKGTE |
| | | | KTLPVPVVSLNIYDVGPTTMHVQWQPVGGA |
| | | | TGYILSYKPVKDTEPTRPKEVRLGPTVNDM |
| | | | QLTDLVPNTEYAVTVQAVLHDLTSEPVTVR |
| | | | EVTLPLPRPQDLKLRDVTHSTMNVFWEPVP |
| | | | GKVRKYIVRYKTPEEDVKEVEVDRSETSTS |
| | | | LKDLFSQTLYTVSVSAVHDEGESPPVTAQE |
| | | | TTRPVPAPTNLKITEVTSEGFRGTWDHGAS |
| | | | DVSLYRITWAPFGSSDKMETILNGDENTLV |
| | | | FENLNPNTIYEVSITAIYPDESESDDLIGS |
| | | | ERTLPILTTQAPKSGPRNLQVYNATSNSLT |
| | | | VKWDPASGRVQKYRITYQPSTGEGNEQTTT |
| | | | IGGRQNSVVLQKLKPDTPYTITVSSLYPDG |
| | | | EGGRMTGRGKTKPLNTVRNLRVYDPSTSTL |
| | | | NVRWDHAEGNPRQYKLFYAPAAGGPEELVP |
| | | | IPGNTNYAILRNLQPDTSYTVTVVPVYTEG |
| | | | DGGRTSDTGRTLMRGLARNVQVYNPTPNSL |
| | | | DVRWDPAPGPVLQYRVVYSPVDGTRPSESI |
| | | | VVPGNTRMVHLERLIPDTLYSVNLVALYSD |
| | | | GEGNPSPAQGRTLPRSGPRNLRVFGETTNS |
| | | | LSVAWDHADGPVQQYRIIYSPTVGDPIDEY |
| | | | TTVPGRRNNVILQPLQPDTPYKITVIAVYE |
| | | | DGDGGHLTGNGRTVGLLPPQNIHISDEWYT |
| | | | RFRVSWDPSPSPVLGYKIVYKPVGSNEPME |
| | | | AFVGEMTSYTLHNLNPSTTYDVNVYAQYDS |
| | | | GLSVPLTDQGTTLYLNVTDLKTYQIGWDTF |
| | | | CVKWSPHRAATSYRLKLSPADGTRGQEITV |
| | | | RGSETSHCFTGLSPDTDYGVTVFVQTPNLE |
| | | | GPGVSVKEHTTVKPTEAPTEPPTPPPPPTI |
| | | | PPARDVCKGAKADIVFLTDASWSIGDDNFN |
| | | | KVVKFIFNTVGGFDEISPAGIQVSFVQYSD |
| | | | EVKSEFKLNTYNDKALALGALQNIRYRGGN |
| | | | TRTGKALTFIKEKVLTWESGMRKNVPKVLV |
| | | | VVTDGRSQDEVKKAALVIQQSGFSVFVVGV |
| | | | ADVDYNELANIASKPSERHVFIVDDFESFE |
| | | | KIEDNLITFVCETATSSCPLIYLDGYTSPG |
| | | | FKMLEAYNLTEKNFASVQGVSLESGSFPSY |
| | | | SAYRIQKNAFVNQPTADLHPNGLPPSYTII |
| | | | LLFRLLPETPSDPFAIWQITDRDYKPQVGV |
| | | | IADPSSKTLSFFNKDTRGEVQTVTFDTEEV |
| | | | KTLFYGSFHKRDEGKCPAFPNSCTCTQDSV |
| | | | GPPGPPGPAGGPGAKGPRGERGISGAIGPP |
| | | | GPRGDIGPPGPQGPPGPQGPNGLSIPGEQG |
| | | | RQGMKGDAGEPGLPGRTGTPGLPGPPGPMG |
| | | | PPGDRGFTGKDGAMGPRGPPGPPGSPGSPG |
| | | | VTGPSGKPGKPGDHGRPGPSGLKGEKGDRG |
| | | | DIASQNMMRAVARQVCEQLISGQMNRFNQM |
| | | | LNQIPNDYQSSRNQPGPPGPPGPPGSAGAR |
| | | | GEPGPGGRPGFPGTPGMQGPPGERGLPGEK |
| | | | GERGTGSSGPRGLPGPPGPQGESRTGPPGS |
| | | | TGSRGPPGPPGRPGNSGIRGPPGPPGYCDS |
| | | | SQCASIPYNGQGYPGSG |
| Q9H0B8 | 1 | 94 | MSCVLGGVIPLGLLFLVCGSQGYLLPNVTL |
| | | | LEELLSKYQHNESHSRVRRAIPREDKEEIL |
| | | | MLHNKLRGQVQPQASNMEYMTWDDELEKSA |
| | | | AAWASQCIWEHGPTSLLVSIGQNLGAHWGR |
| | | | YRSPGFHVQSWYDEVKDYTYPYPSECNPWC |
| | | | PERCSGPMCTHYTQIVWATTNKIGCAVNTC |
| | | | RKMTVWGEVWENAVYFVCNYSPKGNWIGEA |
| | | | PYKNGRPCSECPPSYGGSCRNNLCYREETY |
| | | | TPKPETDEMNEVETAPIPEENHVWLQPRVM |
| | | | RPTKPKKTSAVNYMTQVVRCDTKMKDRCKG |
| | | | STCNRYQCPAGCLNHKAKIFGTLFYESSSS |
| | | | ICRAAIHYGILDDKGGLVDITRNGKVPFFV |
| | | | KSERHGVQSLSKYKPSSSFMVSKVKVQDLD |
| | | | CYTTVAQLCPFEKPATHCPRIHCPAHCKDE |
| | | | PSYWAPVFGTNIYADTSSICKTAVHAGVIS |
| | | | NESGGDVDVMPVDKKKTYVGSLRNGVQSES |
| | | | LGTPRDGKAFRIFAVRQ |
| Q9H0B8 | 2 | 95 | MSCVLGGVIPLGLLFLVCGSQGYLLPNVTL |
| | | | LEELLSKYQHNESHSRVRRAIPREDKEEIL |
| | | | MLHNKLRGQVQPQASNMEYMTWDDELEKSA |
| | | | AAWASQCIWEHGPTSLLVSIGQNLGAHWGR |
| | | | YRSPGFHVQSWYDEVKDYTYPYPSECNPWC |
| | | | PERCSGPMCTHYTQIVWATTNKIGCAVNTC |
| | | | RKMTVWGEVWENAVYFVCNYSPKGNWIGEA |
| | | | PYKNGRPCSECPPSYGGSCRNNLCYREETY |
| | | | TPKPETDEMNEVETAPIPEENHVWLQPRVM |
| | | | RPTKPKKTSAVNYMTQVVRCDTKMKDRCKG |
| | | | STCNRYQCPAGCLNHKAKIFGTLFYESSSS |
| | | | ICRAAIHYGILDDKGGLVDITRNGKVPFFV |
| | | | KSERHGVQSLSKYKPSSSFMVSKVKVQDLD |
| | | | CYTTVAQLCPFEKPATHCPRIHCPAHCKDE |
| | | | PSYWAPVFGTNIYADVSRMHFQQLSRKAC |
| Q9H0B8 | 3 | 96 | MSCVLGGVIPLGLLFLVCGSQGYLLPNVTL |
| | | | LEELLSKYQHNESHSRVRRAIPREDKEEIL |
| | | | MLHNKLRGQVQPQASNMEYMTWDDELEKSA |
| | | | AAWASQCIWEHGPTSLLVSIGQNLGAHWGR |
| | | | YRSPGFHVQSWYDEVKDYTYPYPSECNPWC |
| | | | PERCSGPMCTHYTQIVWATTNKIGCAVNTC |
| | | | RKMTVWGEVWENAVYFVCNYSPKGNWIGEA |
| | | | PYKNGRPCSECPPSYGGSCRNNLCYREETY |
| | | | TPKPETDEMNEVETAPIPEENHVWLQPRVM |
| | | | RPTKPKKTSAVNYMTQVVRCDTKMKDRCKG |
| | | | STCNRYQCPAGCLNHKAKIFGTLFYESSSS |
| | | | ICRAAIHYGILDDKGGLVDITRNGKVPFFV |
| | | | KSERHGVQSLR |
| Q9H0B8 | 4 | 97 | MSCVLGGVIPLGLLFLVCGSQGYLLPNVTL |
| | | | LEELLSKYQHNESHSRVRRAIPREDKEEIL |
| | | | MLHNKLRGQVQPQASNMEYMVSAGSGRRGW |
| | | | HRGWGLGHQPALFPSQLCSPASACDGWLRV |
| | | | SSGRGGSRLCSVLFVCFETGSHSATDAGVQ |
| | | | WHNRHALKP |
| Q9H0B8 | 5 | 98 | MSCVLGGVIPLGLLFLVCGSQGYLLPNVTL |
| | | | LEELLSKYQHNESHSRVRRAIPREDKEEIL |
| | | | MLHNKLRGQVQPQASNMEYMTWDDELEKSA |
| | | | AAWASQCIWEHGPTSLLVSIGQNLGAHWGR |

### Example 3: Method for Searching pQTLs

A linear regression model with covariates was used to search pQTLs.

Phenotypes are represented by log 2 (Intensity) for each nanoparticle-protein group (NP-PG) combination.

A linear regression model is constructed with the form:

$$y = G {β}_{G} + X {β}_{X} + e$$

where y is NP-PG intensity, G is genotype, and X is covariate. β_{G} and β_{X} are learned coefficients from least-squares minimization. The model is evaluated to compute chi-squared goodness of fit statistics and p-value for 1 degree of freedom. Top PCs, sex, age, race, genotype array batch are used as covariates.

FDR is calculated by creating 20 shuffles of data, leading to 20 random intensities for each NP-PG in a sample. For a NP-PG, targets are SNP associations from the non-shuffled run and decoys are SNP associations from the 20 shuffles. The target/decoys are then ranked according to p-values to calculate FDR with ratio=20. The FDR has the form:

$$FDR = \frac{{N}_{decoy}}{{N}_{target} * ratio}$$

A pQTL is considered to be significant when the p-value is less than 1e-5 and the false discovery rate is less than 1e-2. A pQTL is considered to be a cis-pQTL if the SNP is within +/−1 megabase pairs (Mbp) of a transcription start site (TSS) stopping at TSS of another gene, with a minimum of 5 kb up and 1 kb downstream. Otherwise, a pQTL is considered to be a trans-pQTL.

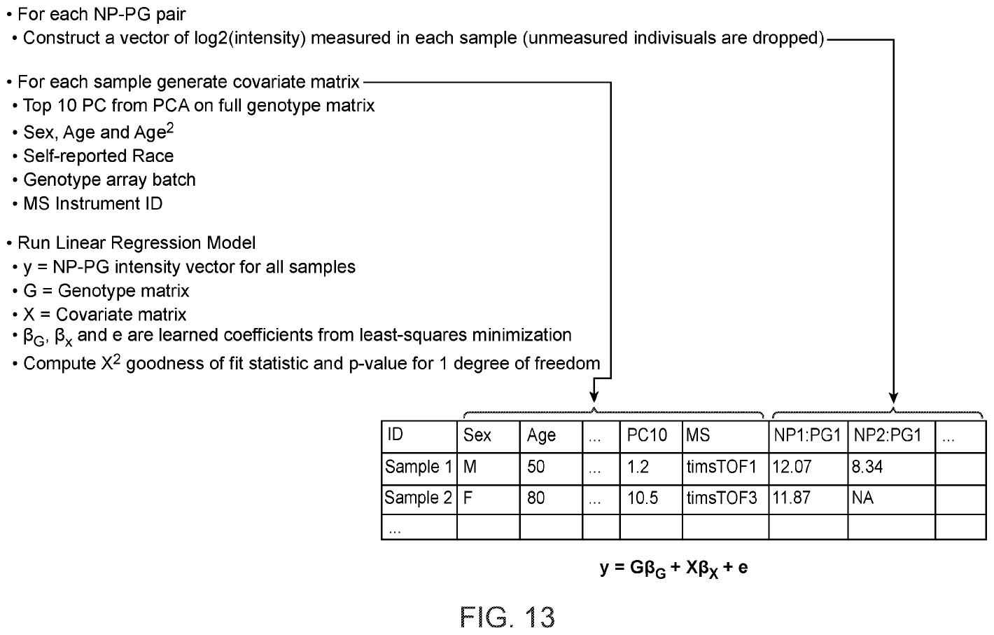

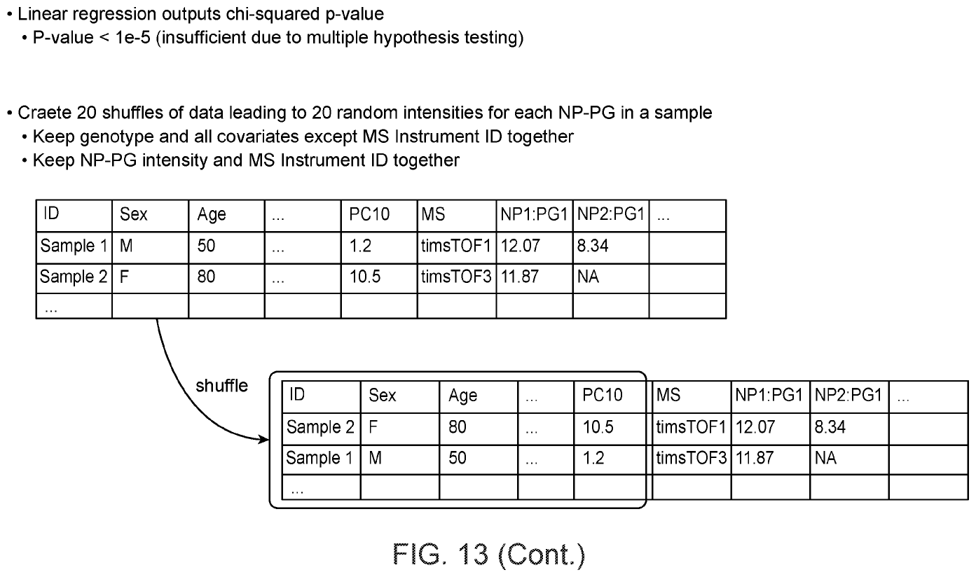

FIG. 13 schematically illustrates a calculation for pQTL association, in accordance with some embodiments.

### Example 4: Identification of QTL Biomarkers for Early-Stage Detection of Alzheimer's Disease

Proteogenomic analysis was conducted to identify pQTLs on a subset of the individuals (139) genotyped on the Global Screening Array platform. BOLT-LMM was used to apply a linear model with a χ² statistic with 1-degree of freedom to establish significance of association between every SNP and Protein: Nanoparticle intensity.

Phenotypes were represented by log 2 (intensity) per nanoparticle-protein group pairs. Linear regression was performed to create model that associates the phenotypes to genotypes (i.e., SNPs). The false discovery rate was calculated using shuffles, with the form:

$$FDR = \frac{{N}_{shuffle}}{{N}_{target} * ratio}$$

A pQTL was considered to be significant when the p-value was less than 1e-5 and the false discovery rate was less than 1e-2. A pQTL was considered to be a cis-pQTL if the SNP was within +/−1 megabase pairs (Mbp) of a transcription start site (TSS) stopping at TSS of another gene, with a minimum of 5 kb up and 1 kb downstream. Otherwise, a pQTL was considered to be a trans-pQTL.

The ratio of all possible trans-pQTL and cis-pQTL was 12109:1 for DDA, and 12107:1 for DIA. After FDR correction, the ratio of FDR corrected trans-pQTL to cis-pQTLs was 21:1 for DDA and 56:1 for DIA.

Overall, 73 cis-pQTLs associated with 20 protein groups and 41 SNPs, and 1,527 trans-pQTLs associated with 294 protein groups and 1269 SNPs using DDA data; 59 cis-pQTLs associated with 20 proteins and 38 SNPs, and 3,288 trans-pQTLs associated with 397 protein groups and 2,802 SNPs using DIA data.

A significant reduction in trans-pQTLs were observed with FDR correction, while the number of cis-pQTLs were preserved, indicating that the FDR strategy is robust at reducing false signals for trans-pQTLs.

TABLE 4
Statistics for trans-pQTLs and cis-QTLs associations.

| | DDA | DIA |
|---|---|---|
| Possible Associations (NP:PG) | 21,253 | 22,453 |
| Processed Associations | 10,612 | 14,092 |
| trans-pQTLs | 1,527 | 3,288 |
| PGs | 294 | 397 |
| SNPs | 1,269 | 2,802 |
| cis-pQTLs | 73 | 59 |
| PGs | 20 | 20 |
| SNPs | 41 | 38 |

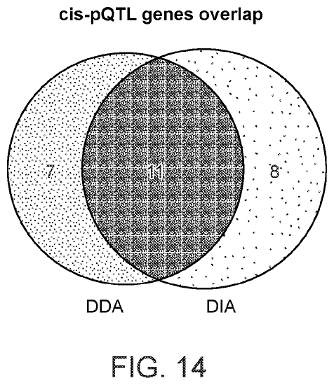

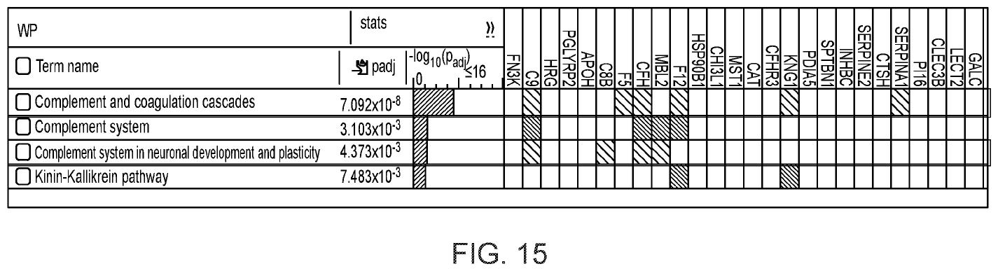

The cis-pQTLs identified from both DDA and DIA data, as shown in FIG. 14, were functionally annotated using G: PROFILER™. FIG. 15 shows functional annotations of proteins regulated by cis-pQTLs using the WIKIPATHWAYS™ (WP) database, in accordance with some embodiments.

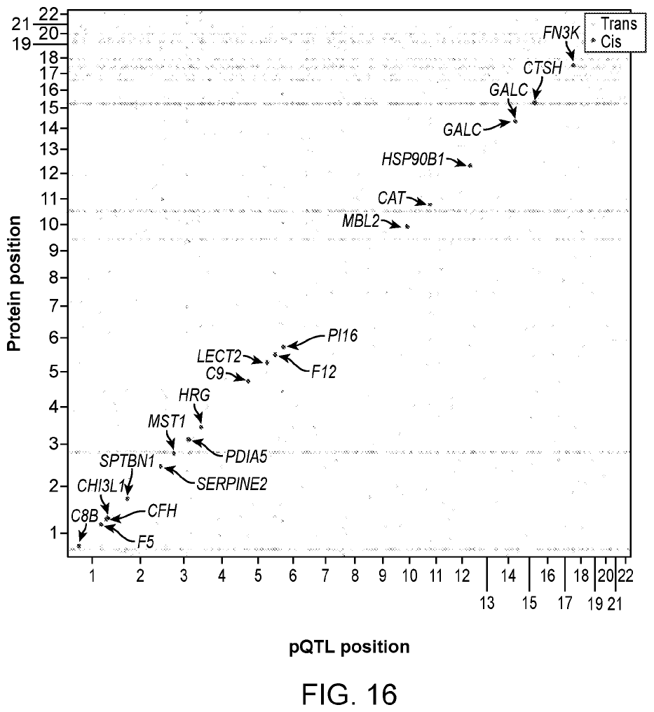

FIG. 16 shows proteins regulated by cis-pQTLs identified in association with Alzheimer's Disease, in accordance with some embodiments.

TABLE 5
Genes associated with DIA cis-pQTLs
and their biological functions.

| | |
|---|---|
| cis-pQTL | Biological Function Annotation (GENECARDS ™) |
| FN3K | phosphorylyses fructosamines |
| CTSH | degrades lysosomal proteins |
| GALC | hydrolyzes galactose ester bonds of glycolipids |
| HSP90B1 | ATP-metabolizing molecular chaperone |
| CAT | antioxidant enzyme |
| MBL2 | lectin involved in innate immune defense |
| PI16 | enables peptidase inhibitor activity |
| F12 | encodes coagulation factor XII |
| LECT2 | stimulates the growth of chondrocytes and osteoblasts |
| HRG | binds heme, dyes and divalent metal ions |
| PDIA5 | catalyzes protein folding and thiol-disulfide interchange |
| | reactions |
| MST1 | upon activation, stimulates ciliary motility of ciliated |
| | epithelial lung cells |
| SERPINE2 | inhibits serine proteases |
| SPTBN1 | determinescell shape; arranges transmembrane proteins and |
| | organelles |
| CHI3L1 | catalyzes hydrolysis of chitin |
| CFH | regulates complement activation |
| F5 | encodes an essential cofactor of the blood coagulation |
| | cascade |
| C9 | forms part of the Membrane Attack Complex (MAC) - |
| | immune response |
| C8B | another component of the MAC, which mediates cell lysis |

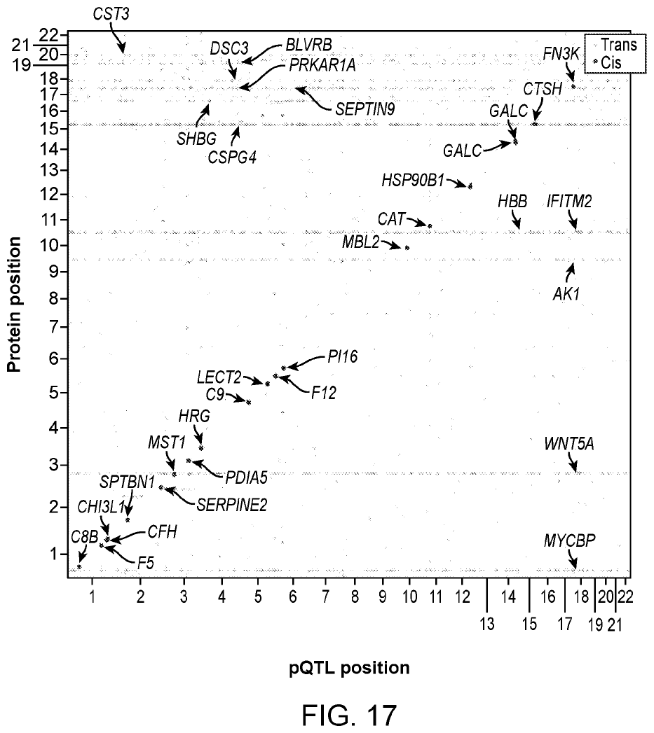

FIG. 17 shows proteins regulated by trans-pQTLs identified in association with Alzheimer's Disease, in accordance with some embodiments.

TABLE 6
Genes associated with DIA trans-pQTLs
and their biological functions.

| | |
|---|---|
| trans-pQTL | Biological Function Annotation (GENECARDS ™) |
| enriched gene | |
| CST3 | cysteine proteases; all over body |
| BLVRB | Biliverdin Reductase B |
| DSC3 | cell adhesion |
| PRKAR1A | cAMP signaling molecule |
| SEPTIN9 | cytokinesis and cell cycle control |
| SHBG | transports androgens/estrogens |
| CSPG4 | stabilizes cell-substratum interactions |
| HBB | Beta locus for hemoglobin chain |
| IFITM2 | interferon-induced antiviral protein |
| AK1 | metabolism + homeostasis |
| WNT5A | secreted signaling protein |
| MYCBP | transcription enhancer |

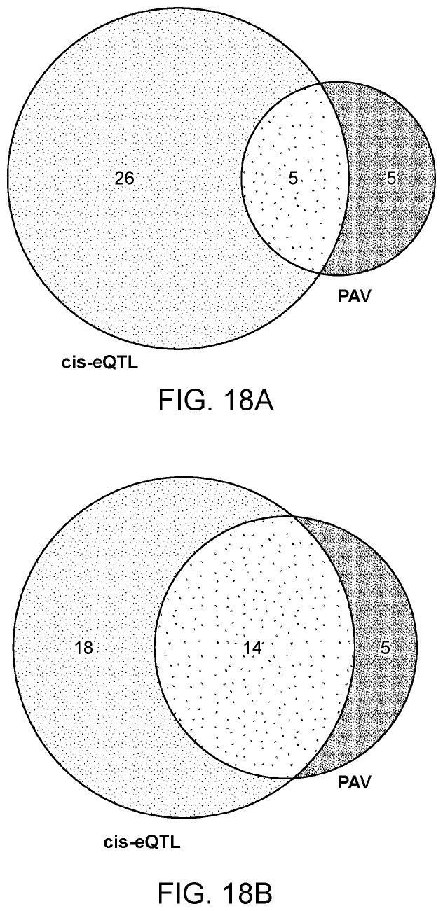

Majority of the discovered sentinel SNPs were found to be expression quantitative trait loci (eQTLs) for the same proteins, and in linkage disequilibrium with many protein altering variants. FIG. 18A shows the number of sentinel single nucleotide polymorphism (SNP) pQTLs identified as cis-expression quantitative trait loci (cis-eQTL) or protein altering variants, in accordance with some embodiments. FIG. 18B shows the number of sentinel SNP pQTLs identified to be in linkage disequilibrium with cis-eQTL or protein altering variants, in accordance with some embodiments. The 38 cis-pQTL SNPs correlate strongly with orthogonal support for function.

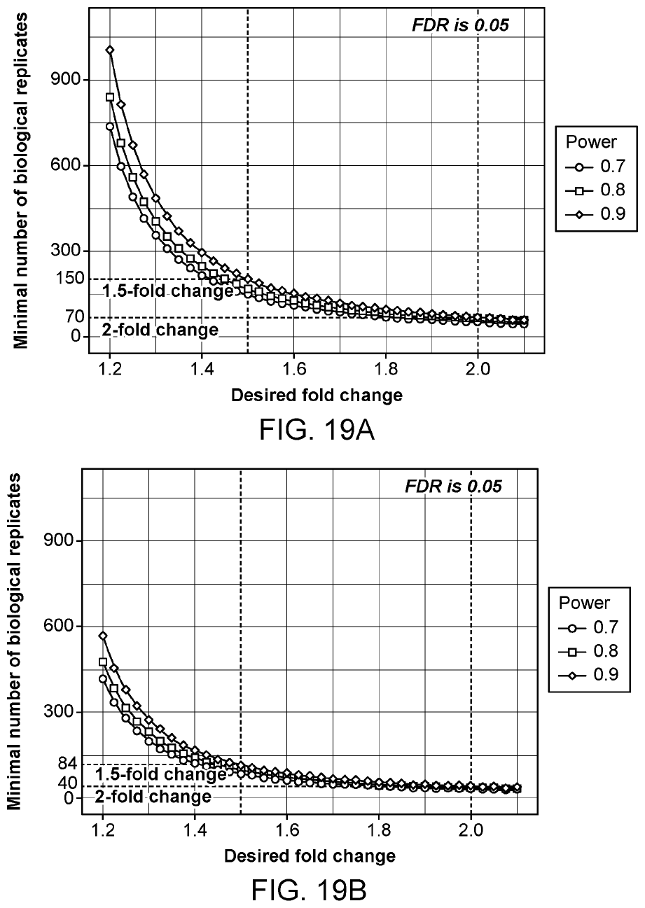

Statistical power of using DDA versus DIA with PROTEOGRAPH™ (maxLFQ) was evaluated. FIGS. 19A-19B show minimal number of biological replicates as a function of desired fold change in statistical confidence for identifying pQTLs using DDA and DIA, respectively, in accordance with some embodiments.

TABLE 7
Cis-pQTLs Association with Protein Groups from DDA and DIA.

| DDA | | DIA | |
|---|---|---|---|
| SNP | PG | SNP | PG |
| GSA-rs1046279 | Q6UXB8 | GSA-rs1046279 | Q6UXB8 |
| GSA-rs1131364 | P04196 | GSA-rs1165692 | P14625 |
| GSA-rs1165692 | P14625 | GSA-rs6030 | P12259 |
| GSA-rs6030 | P12259; | rs1001179 | P04040 |
| | A0A0A0MRJ7 | | |
| rs1061147 | Q02985 | rs1046875 | Q9H479 |
| rs10737680 | Q02985 | rs1046896 | Q9H479 |
| rs10801555 | Q02985 | rs1061147 | A0A0D9SG88; |
| | | | Q5TFM2 |
| rs1106766 | P55103 | rs10801555 | A0A0D9SG88; |
| | | | Q5TFM2 |
| rs11085954 | Q96PD5 | rs11003118 | P11226 |
| rs11613352 | P55103 | rs1165669 | P14625 |
| rs1165669 | P14625 | rs12067507 | P07358 |
| rs12067507 | F5GY80 | rs12148472 | P09668 |
| rs12067507 | P07358 | rs12493926 | P04196 |
| rs12493926 | P04196 | rs1329424 | A0A0D9SG88; |
| | | | Q5TFM2 |
| rs1329424 | Q02985 | rs17198 | P54803; |
| | | | P54803-3; |
| | | | P54803-4 |
| rs1329428 | Q02985 | rs1800450 | P11226 |
| rs1410996 | Q02985 | rs1801020 | P00748 |
| rs1520079 | P05452; | rs1831282 | A0A0D9SG88; |
| | E9PHK0 | | Q5TFM2 |
| rs17198 | P54803 | rs2289702 | P09668 |
| rs1801020 | P00748 | rs2545801 | P00748 |
| rs1801689 | P02749 | rs2731672 | P00748 |
| rs1831282 | Q02985 | rs3197999 | P26927 |
| rs2545801 | P00748 | rs34882957 | P02748 |
| rs2731672 | P00748 | rs3784539 | P09668 |
| rs2749534 | P01009 | rs3792366 | Q14554 |
| rs3197999 | P26927; | rs380390 | A0A0D9SG88; |
| | G3XAK1 | | Q5TFM2 |
| rs3741414 | P55103 | rs395544 | A0A0D9SG88; |
| | | | Q5TFM2 |
| rs3792366 | Q14554 | rs41268617 | P04196 |
| rs380390 | Q02985 | rs4524 | P12259 |
| rs395544 | Q02985 | rs62623707 | O14960 |
| rs41268617 | P04196 | rs6677604 | P08603 |
| rs5030062 | P01042 | rs6695321 | A0A0D9SG88; |
| | | | Q5TFM2 |
| rs5030062 | P01042-2 | rs68066031 | P07093-2; |
| | | | P07093-3 |
| rs62623707 | O14960 | rs707998 | Q6UXB8 |
| rs6647 | P01009 | rs7599241 | Q01082 |
| rs6677604 | P08603 | rs880633 | P36222 |
| rs6677604 | Q02985 | rs9820435 | Q14554 |
| rs6695321 | Q02985 | rs9898 | P04196 |
| rs68147365 | P55103 | | |
| rs698078 | P01042 | | |
| rs698078 | P01042-2 | | |
| rs710446 | P01042 | | |
| rs710446 | P01042-2 | | |
| rs8099840 | Q96PD5 | | |
| rs9820435 | Q14554 | | |
| rs9898 | P04196 | | |

TABLE 8
Genes associated with DIA or DDA cis-pQTLs.

| Gene | DDA | DIA | Gene | DDA | DIA |
|---|---|---|---|---|---|
| C9 | FALSE | TRUE | FN3K | FALSE | TRUE |
| GALC* | TRUE | TRUE | CHI3L1 | FALSE | TRUE |
| CFHR3 | TRUE | FALSE | HSP90B1* | TRUE | TRUE |
| F12* | TRUE | TRUE | SERPINE2 | FALSE | TRUE |
| SPTBN1 | FALSE | TRUE | MBL2 | FALSE | TRUE |
| SERPINA1 | TRUE | FALSE | PDIA5* | TRUE | TRUE |
| LECT2* | TRUE | TRUE | KNG1 | TRUE | FALSE |
| CFH* | TRUE | TRUE | PGLYRP2 | TRUE | FALSE |
| HRG* | TRUE | TRUE | INHBC | TRUE | FALSE |
| APOH | TRUE | FALSE | PI16* | TRUE | TRUE |
| F5* | TRUE | TRUE | MST1* | TRUE | TRUE |
| CTSH | FALSE | TRUE | CAT | FALSE | TRUE |
| CLEC3B | TRUE | FALSE | C8B* | TRUE | TRUE |

Asterisks (*) mark genes that are found with both DIA and DDA cis-pQTLs.

TABLE 9
Proteins associated with DIA or DDA cis-pQTLs.

| Protein | DDA | DIA | Protein | DDA | DIA |
|---|---|---|---|---|---|
| P14625* | TRUE | TRUE | Q14554* | TRUE | TRUE |
| O14960* | TRUE | TRUE | P00748* | TRUE | TRUE |
| P04040 | FALSE | TRUE | P54803; P54803-3; | FALSE | TRUE |
| | | | P54803-4 | | |
| P12259 | FALSE | TRUE | P02748 | FALSE | TRUE |
| P07358* | TRUE | TRUE | A0A0D9SG88; | FALSE | TRUE |
| | | | Q5TFM2 | | |
| P04196* | TRUE | TRUE | P02749 | TRUE | FALSE |
| P55103 | TRUE | FALSE | Q9H479 | FALSE | TRUE |
| P01009 | TRUE | FALSE | P36222 | FALSE | TRUE |
| F5GY80 | TRUE | FALSE | P54803 | TRUE | FALSE |
| Q96PD5 | TRUE | FALSE | Q02985 | TRUE | FALSE |
| P08603* | TRUE | TRUE | Q01082 | FALSE | TRUE |
| P07093-2; | FALSE | TRUE | P09668 | FALSE | TRUE |
| P07093-3 | | | | | |
| P05452; | TRUE | FALSE | P12259; | TRUE | FALSE |
| E9PHK0 | | | A0A0A0MRJ7 | | |
| Q6UXB8* | TRUE | TRUE | P26927; G3XAK1 | TRUE | FALSE |
| P01042-2 | TRUE | FALSE | P01042 | TRUE | FALSE |
| P26927 | FALSE | TRUE | P11226 | FALSE | TRUE |

Asterisks (*) mark proteins that are found with both DIA and DDA cis-pQTLs.

TABLE 10
SNPs in linkage disequilibrium (LD) with pQTL sentinel SNPS.

| | Acquisition | |
|---|---|---|
| Sentinel SNP | Mode | Protein Altering SNP Variants in LD with Sentinel SNP |
| GSA-rs1046279 | DDA, DIA | GSA-rs1046279, rs1063021, rs1405069 |
| GSA-rs1131364 | DDA | GSA-rs1131364, rs112001519, rs112673578, rs113183496, |
| | | rs11720187, rs11923752, rs1868154, rs1868155, rs34251945, |
| | | rs4438644, rs4440112, rs4645128, rs56262819, rs56373302, |
| | | rs58176562, rs59629826, rs61004104, rs62292571, rs6767451, |
| | | rs6773124, rs6791137, rs6797321, rs6803759, rs6807675, |
| | | rs6807764, rs6807860, rs7431676, rs7432647, rs74909364, |
| | | rs7615820, rs76880681, rs9819903, rs9820040, rs9823725, |
| | | rs9848081, rs9848086, rs9858037, rs9858058, rs9877577 |
| GSA-rs1165692 | DDA, DIA | GSA-rs1165692, rs1165693, rs1165694, rs1165695, rs1165696, |
| | | rs1177457, rs1179177, rs15805, rs2576975, rs2583254, |
| | | rs2583262, rs2583264, rs2700505, rs2700508, rs2700509, |
| | | rs2722190, rs2722191, rs2722192, rs703657, rs7645 |
| GSA-rs6030 | DDA, DIA | GSA-rs6030, rs10800454, rs1557570, rs1557572, rs1800594, |
| | | rs2157581, rs2239852, rs2301515, rs3820060, rs4656185, |
| | | rs4656687, rs6686805, rs6701330, rs7535409, rs916438, |
| | | rs9332599, rs9332665 |
| rs1001179 | DIA | rs1001179, rs61880810 |
| rs1046875 | DIA | rs1046875, rs1046896, rs1046917, rs113373052, rs12449739, |
| | | rs12947062, rs149836885, rs28485881, rs7208565, rs7211596, |
| | | rs7216058, rs72634341, rs9892459, rs9895455, rs9909940 |
| rs1046896 | DIA | rs1046875, rs1046896, rs1046917, rs113373052, rs12449739, |
| | | rs12947062, rs149836885, rs28485881, rs7208565, rs7211596, |
| | | rs7216058, rs72634341, rs9892459, rs9895455, rs9909940 |
| rs1061147 | DDA, DIA | rs1061147, rs1061170, rs10733086, rs10754199, rs10801553, |
| | | rs10801554, rs10801555, rs10801556, rs10801557, rs1089033, |
| | | rs10922092, rs10922094, rs10922097, rs10922098, rs10922099, |
| | | rs10922100, rs10922102, rs10922103, rs12029785, rs12033127, |
| | | rs12038333, rs12038674, rs12045503, rs1329421, rs1329422, |
| | | rs1329424, rs1410997, rs1831282, rs2019724, rs203672, |
| | | rs203674, rs203677, rs203679, rs203682, rs203683, rs203685, |
| | | rs203687, rs203688, rs2300430, rs2860102, rs28664709, |
| | | rs28853072, rs36137052, rs3645, rs368465, rs374896, rs375046, |
| | | rs379489, rs380390, rs381974, rs393955, rs395544, rs399469, |
| | | rs402056, rs402991, rs403846, rs412852, rs414539, rs4658046, |
| | | rs482934, rs485632, rs488380, rs506317, rs514943, rs528298, |
| | | rs529899, rs570618, rs572515, rs6428357, rs6664705, |
| | | rs7522681, rs7529589, rs7539005, rs7546015, rs76258507, |
| | | rs9970075, rs9970784 |
| rs10737680 | DDA | rs10465586, rs10737680, rs10801558, rs10801559, rs10922104, |
| | | rs10922105, rs10922106, rs10922108, rs10922109, rs1329427, |
| | | rs1329428, rs1410996, rs3753395, rs3766405, rs6677089, |
| | | rs6688272, rs7514261, rs7535263, rs7540032 |
| rs10801555 | DDA, DIA | rs1061147, rs1061170, rs10733086, rs10754199, rs10801553, |
| | | rs10801554, rs10801555, rs10801556, rs10801557, rs1089033, |
| | | rs10922092, rs10922094, rs10922097, rs10922098, rs10922099, |
| | | rs10922100, rs10922102, rs10922103, rs12029785, rs12033127, |
| | | rs12038333, rs12038674, rs12045503, rs1329421, rs1329422, |
| | | rs1329424, rs1410997, rs1831282, rs2019724, rs203672, |
| | | rs203674, rs203677, rs203679, rs203682, rs203683, rs203685, |
| | | rs203687, rs203688, rs2300430, rs2860102, rs28664709, |
| | | rs28853072, rs36137052, rs3645, rs368465, rs374896, rs375046, |
| | | rs379489, rs380390, rs381974, rs393955, rs395544, rs399469, |
| | | rs402056, rs402991, rs403846, rs412852, rs414539, rs4658046, |
| | | rs482934, rs485632, rs488380, rs506317, rs514943, rs528298, |
| | | rs529899, rs570618, rs572515, rs6428357, rs6664705, |
| | | rs7522681, rs7529589, rs7539005, rs7546015, rs76258507, |
| | | rs9970075, rs9970784 |
| rs11003118 | DIA | rs11003118 |
| rs1106766 | DDA | rs1106766, rs11172147, rs11172181, rs11609805, rs11613352, |
| | | rs11614506, rs12313306, rs12313762, rs12315434, rs2122982, |
| | | rs2229357, rs3741414, rs4760254, rs4760278, rs4760355, |
| | | rs540730, rs56205943, rs61352607, rs68147365, rs73119306, |
| | | rs7397189, rs7484541, rs79395356, rs7964492, rs7971133, |
| | | rs7974833 |
| rs11085954 | DDA | rs11085953, rs11085954 |
| rs11613352 | DDA | rs1106766, rs11172147, rs11172181, rs11609805, rs11613352, |
| | | rs11614506, rs12313306, rs12313762, rs12315434, rs2122982, |
| | | rs2229357, rs3741414, rs4760254, rs4760278, rs4760355, |
| | | rs540730, rs56205943, rs61352607, rs68147365, rs73119306, |
| | | rs7397189, rs7484541, rs79395356, rs7964492, rs7971133, |
| | | rs7974833 |
| rs1165669 | DDA, DIA | rs10778305, rs11111840, rs1165668, rs1165669, rs1165670, |
| | | rs1165674, rs1866071, rs2374293, rs2439624, rs2576954, |
| | | rs2576955, rs2583282, rs2583284 |
| rs12067507 | DDA, DIA | rs12067507, rs12085435, rs12092641, rs41452950, rs78415143 |
| rs12148472 | DIA | rs12148472, rs12592898, rs34843303, rs3784539 |
| rs12493926 | DDA, DIA | rs1042464, rs12493926, rs60531302, rs60693099, rs7617554, |
| | | rs7626301 |
| rs1329424 | DDA, DIA | rs1061147, rs1061170, rs10733086, rs10754199, rs10801553, |
| | | rs10801554, rs10801555, rs10801556, rs10801557, rs1089033, |
| | | rs10922092, rs10922094, rs10922097, rs10922098, rs10922099, |
| | | rs10922100, rs10922102, rs10922103, rs12029785, rs12033127, |
| | | rs12038333, rs12038674, rs12045503, rs1329421, rs1329422, |
| | | rs1329424, rs1410997, rs1831282, rs2019724, rs203672, |
| | | rs203674, rs203677, rs203679, rs203682, rs203683, rs203685, |
| | | rs203687, rs203688, rs2300430, rs2860102, rs28664709, |
| | | rs28853072, rs36137052, rs3645, rs368465, rs374896, rs375046, |
| | | rs379489, rs380390, rs381974, rs393955, rs395544, rs399469, |
| | | rs402056, rs402991, rs403846, rs412852, rs414539, rs4658046, |
| | | rs482934, rs485632, rs488380, rs506317, rs514943, rs528298, |
| | | rs529899, rs570618, rs572515, rs6428357, rs6664705, |
| | | rs7522681, rs7529589, rs7539005, rs7546015, rs76258507, |
| | | rs9970075, rs9970784 |
| rs1329428 | DDA | rs10465586, rs10737680, rs10801558, rs10801559, rs10922104, |
| | | rs10922105, rs10922106, rs10922108, rs10922109, rs1329427, |
| | | rs1329428, rs1410996, rs3753395, rs3766405, rs6677089, |
| | | rs6688272, rs7514261, rs7535263, rs7540032 |
| rs1410996 | DDA | rs10465586, rs10737680, rs10801558, rs10801559, rs10922104, |
| | | rs10922105, rs10922106, rs10922108, rs10922109, rs1329427, |
| | | rs1329428, rs1410996, rs3753395, rs3766405, rs6677089, |
| | | rs6688272, rs7514261, rs7535263, rs7540032 |
| rs1520079 | DDA | rs1004877, rs13320864, rs13321407, rs13963, rs1520079, |
| | | rs1520080, rs17570449, rs1850938, rs2139538, rs2280384, |
| | | rs2301282, rs2372850, rs2372851, rs2372862, rs3765173, |
| | | rs4323037, rs4683018, rs4683019, rs4683020, rs4683023, |
| | | rs4683024, rs4683025, rs4683026, rs4683027, rs4683029, |
| | | rs4683031, rs4683032, rs6441873, rs6441874, rs6441877, |
| | | rs6441881, rs66625825, rs66964756, rs6764088, rs6772855, |
| | | rs6772941, rs6790660, rs6792742, rs6807133, rs7612289, |
| | | rs7633604, rs8318, rs871085, rs883765, rs939310, rs939311, |
| | | rs9819447, rs9824101, rs9852384, rs9861505, rs9868885, |
| | | rs9872295, rs9873373, rs9877793, rs9880792, rs9880877 |
| rs17198 | DDA, DIA | rs10134254, rs10138271, rs10142611, rs10142621, rs1042029, |
| | | rs1042042, rs11159831, rs11624233, rs116873224, rs11848620, |
| | | rs12432149, rs12434297, rs12880526, rs12881008, rs12885948, |
| | | rs1661320, rs1661321, rs1661324, rs17198, rs1745632, |
| | | rs1745635, rs2285008, rs2285009, rs2285011, rs2301118, |
| | | rs2301120, rs2896066, rs2896069, rs2984825, rs3213917, |
| | | rs365448, rs366615, rs367636, rs368159, rs368585, rs376222, |
| | | rs378404, rs378816, rs380142, rs380557, rs381069, rs383994, |
| | | rs384128, rs3850377, rs386525, rs3910682, rs397480, rs397664, |
| | | rs398607, rs405567, rs406280, rs406294, rs408667, rs412915, |
| | | rs413420, rs415076, rs415217, rs416542, rs421749, rs422608, |
| | | rs424977, rs4254268, rs425598, rs429176, rs429923, rs431081, |
| | | rs432946, rs434541, rs442118, rs450976, rs452307, rs4537954, |
| | | rs4547273, rs45567441, rs45626938, rs4620899, rs4904408, |
| | | rs74845231, rs8014302 |
| rs1800450 | DIA | rs1031101, rs11003128, rs11003131, rs11003134, rs1800450 |
| rs1801020 | DDA, DIA | rs1801020, rs2545801, rs2731672, rs2731673, rs2731674 |
| rs1801689 | DDA | rs149394327, rs1801689, rs75003668, rs78357146, rs8178824 |
| rs1831282 | DDA, DIA | rs1061147, rs1061170, rs10733086, rs10754199, rs10801553, |
| | | rs10801554, rs10801555, rs10801556, rs10801557, rs1089033, |
| | | rs10922092, rs10922094, rs10922097, rs10922098, rs10922099, |
| | | rs10922100, rs10922102, rs10922103, rs10922140, rs12029785, |
| | | rs12033127, rs12038333, rs12038674, rs12045503, rs1329421, |
| | | rs1329422, rs1329424, rs1410997, rs1831282, rs2019724, |
| | | rs203672, rs203674, rs203677, rs203679, rs203682, rs203683, |
| | | rs203685, rs203687, rs203688, rs2300430, rs2860102, |
| | | rs28664709, rs28853072, rs36137052, rs3645, rs368465, |
| | | rs374896, rs375046, rs379489, rs380390, rs381974, rs393955, |
| | | rs395544, rs399469, rs402056, rs402991, rs403846, rs412852, |
| | | rs414539, rs4658046, rs482934, rs485632, rs488380, rs506317, |
| | | rs514943, rs528298, rs529899, rs570618, rs572515, rs6428357, |
| | | rs6664705, rs7522681, rs7529589, rs7539005, rs7546015, |
| | | rs76258507, rs9970075, rs9970784 |
| rs2289702 | DIA | rs2289702, rs34593439, rs34843303 |
| rs2545801 | DDA, DIA | rs1801020, rs2545801, rs2731672, rs2731673, rs2731674 |
| rs2731672 | DDA, DIA | rs1801020, rs2545801, rs2731672, rs2731673, rs2731674 |
| rs2749534 | DDA | rs1016686, rs11160172, rs1243167, rs12880218, rs1815228, |
| | | rs1956172, rs1956175, rs1987574, rs2003640, rs2250095, |
| | | rs2736885, rs2736888, rs2736895, rs2736896, rs2736897, |
| | | rs2736901, rs2749525, rs2749526, rs2749528, rs2749532, |
| | | rs2749533, rs2749534, rs4905192, rs4905193, rs6575422, |
| | | rs941597 |
| rs3197999 | DDA, DIA | rs10212296, rs1050450, rs11130203, rs11130213, rs111903592, |
| | | rs1131095, rs11706370, rs11708955, rs11709525, rs11710037, |
| | | rs11711536, rs11712569, rs11713474, rs11715915, rs11716974, |
| | | rs11718165, rs11719996, rs11720597, rs11917431, rs11921590, |
| | | rs11922013, rs11926781, rs12330269, rs13062429, rs13064576, |
| | | rs13079082, rs13079643, rs13085791, rs13086611, rs13087851, |
| | | rs13090388, rs13093385, rs13316695, rs148734725, rs17080528, |
| | | rs1800668, rs1801143, rs1873625, rs1987628, rs2172252, |
| | | rs3197999, rs34196454, rs34293138, rs34363169, rs34427167, |
| | | rs34588335, rs34762726, rs35115732, rs35169793, rs35261698, |
| | | rs35999162, rs3811697, rs3811699, rs3926569, rs4283605, |
| | | rs4625, rs6446264, rs6446272, rs6446277, rs67216675, |
| | | rs67485053, rs6766131, rs6766581, rs6767355, rs6770670, |
| | | rs6779524, rs6793308, rs6797664, rs6803222, rs6809216, |
| | | rs6997, rs71324962, rs71324979, rs7614725, rs7623659, |
| | | rs7630869, rs7633271, rs7646366, rs7648841, rs9811982, |
| | | rs9812791, rs9814873, rs9818590, rs9821311, rs9822268, |
| | | rs9823546, rs9824092, rs9827021, rs9827708, rs9833611, |
| | | rs9836291, rs9837027, rs9837341, rs9841110, rs9850072, |
| | | rs9852529, rs9853683, rs9858213, rs9858280, rs9858418, |
| | | rs9858542, rs9859556, rs9862080, rs9871380, rs9875617, |
| | | rs9878943, rs9882740 |
| rs34882957 | DIA | rs34882957, rs62358361 |
| rs3741414 | DDA | rs1106766, rs11172147, rs11172181, rs11609805, rs11613352, |
| | | rs11614506, rs12313306, rs12313762, rs12315434, rs2122982, |
| | | rs2229357, rs3741414, rs4760254, rs4760278, rs4760355, |
| | | rs540730, rs56205943, rs61352607, rs68147365, rs73119306, |
| | | rs7397189, rs7484541, rs79395356, rs7964492, rs7971133, |
| | | rs7974833 |
| rs3784539 | DIA | rs12148472, rs12592898, rs3784539 |
| rs3792366 | DDA, DIA | rs13314114, rs1530455, rs2278668, rs3792366, rs3792369, |
| | | rs3792376, rs3792378, rs3792382, rs3792386, rs3804749, |
| | | rs3804753, rs4677875, rs726681, rs920900, rs9820435, |
| | | rs9823527 |
| rs380390 | DDA, DIA | rs1061147, rs1061170, rs10733086, rs10754199, rs10801553, |
| | | rs10801554, rs10801555, rs10801556, rs10801557, rs1089033, |
| | | rs10922092, rs10922094, rs10922097, rs10922098, rs10922099, |
| | | rs10922100, rs10922102, rs10922103, rs10922140, rs12029785, |
| | | rs12033127, rs12038333, rs12038674, rs12045503, rs1329421, |
| | | rs1329422, rs1329424, rs1410997, rs1831282, rs2019724, |
| | | rs203672, rs203674, rs203677, rs203679, rs203682, rs203683, |
| | | rs203685, rs203687, rs203688, rs2300430, rs2860102, |
| | | rs28664709, rs28853072, rs36137052, rs3645, rs368465, |
| | | rs374896, rs375046, rs379489, rs380390, rs381974, rs393955, |
| | | rs395544, rs399469, rs402056, rs402991, rs403846, rs412852, |
| | | rs414539, rs4658046, rs482934, rs485632, rs488380, rs506317, |
| | | rs514943, rs528298, rs529899, rs570618, rs572515, rs6428357, |
| | | rs6664705, rs7522681, rs7529589, rs7539005, rs7546015, |
| | | rs76258507, rs9970075, rs9970784 |
| rs395544 | DDA, DIA | rs1061147, rs1061170, rs10733086, rs10754199, rs10801553, |
| | | rs10801554, rs10801555, rs10801556, rs10801557, rs1089033, |
| | | rs10922092, rs10922094, rs10922097, rs10922098, rs10922099, |
| | | rs10922100, rs10922102, rs10922103, rs10922140, rs12029785, |
| | | rs12033127, rs12038333, rs12038674, rs12045503, rs1329421, |
| | | rs1329422, rs1329424, rs1410997, rs1831282, rs2019724, |
| | | rs203672, rs203674, rs203677, rs203679, rs203682, rs203683, |
| | | rs203685, rs203687, rs203688, rs2300430, rs2860102, |
| | | rs28664709, rs28853072, rs36137052, rs3645, rs368465, |
| | | rs374896, rs375046, rs379489, rs380390, rs381974, rs393955, |
| | | rs395544, rs399469, rs402056, rs402991, rs403846, rs412852, |
| | | rs414539, rs4658046, rs482934, rs485632, rs488380, rs506317, |
| | | rs514943, rs528298, rs529899, rs570618, rs572515, rs6428357, |
| | | rs6664705, rs7522681, rs7529589, rs7539005, rs7546015, |
| | | rs76258507, rs9970075, rs9970784 |
| rs41268617 | DDA, DIA | rs3733159, rs41268617 |
| rs4524 | DIA | rs10800450, rs10800453, rs10919178, rs1121789, rs11579441, |
| | | rs12127364, rs2009814, rs2187952, rs2187953, rs2213866, |
| | | rs2213867, rs2227244, rs2239851, rs2420373, rs3766109, |
| | | rs4524, rs4525, rs4656187, rs4656684, rs4656685, rs6016, |
| | | rs6017, rs6021, rs6032, rs6662593, rs6662696, rs6670393, |
| | | rs6675244, rs9287090, rs9287092, rs9332600, rs9332619, |
| | | rs9332620, rs9332627, rs9332635, rs9332643, rs9332651, |
| | | rs9332655, rs9332667, rs970741, rs974793 |
| rs5030062 | DDA | rs5030062, rs5030068, rs5030072, rs5030073, rs5030074, |
| | | rs5030081, rs5030082, rs698078, rs710446 |
| rs62623707 | DDA, DIA | rs62623707 |
| rs6647 | DDA | rs1243162, rs1243164, rs1243165, rs6647 |
| rs6677604 | DDA, DIA | rs1092801, rs113632891, rs12134598, rs148235292, |
| | | rs149369377, rs16840522, rs16840639, rs182780270, |
| | | rs190577253, rs2019727, rs2064456, rs2300429, rs33915960, |
| | | rs34185185, rs35253683, rs35617250, rs370952977, |
| | | rs371960809, rs58175074, rs60642321, rs6664877, rs6677460, |
| | | rs6677604, rs6689009, rs67908756, rs71631868, rs731557, |
| | | rs74696321, rs7519758, rs7542235, rs75751943, rs79654026 |
| rs6695321 | DDA, DIA | rs6695321 |
| rs68066031 | DIA | rs13412535, rs68066031 |
| rs68147365 | DDA | rs1106766, rs11172147, rs11172181, rs11609805, rs11613352, |
| | | rs11614506, rs12313306, rs12313762, rs12315434, rs2122982, |
| | | rs2229357, rs3741414, rs4760254, rs4760278, rs4760355, |
| | | rs540730, rs56205943, rs61352607, rs68147365, rs73119306, |
| | | rs7397189, rs7484541, rs79395356, rs7964492, rs7971133, |
| | | rs7974833 |
| rs698078 | DDA | rs5030062, rs5030081, rs5030082, rs698078, rs710446 |
| rs707998 | DIA | rs12664695, rs13199343, rs13214150, rs139354998, rs16889316, |
| | | rs201629738, rs201903338, rs34165286, rs35310390, |
| | | rs35349900, rs35695974, rs35863861, rs4714025, rs56322497, |
| | | rs66570061, rs68028411, rs6919204, rs697747, rs697748, |
| | | rs707998, rs707999, rs831507, rs9470444 |
| rs710446 | DDA | rs5030062, rs5030081, rs5030082, rs698078, rs710446 |
| rs7599241 | DIA | rs10166844, rs10169954, rs10169975, rs10170066, rs10170355, |
| | | rs10176445, rs10182538, rs10182836, rs10183867, rs10184128, |
| | | rs10187338, rs10187447, rs10188545, rs10202030, rs10205410, |
| | | rs10206143, rs10208219, rs10208225, rs1137645, rs11892443, |
| | | rs11898032, rs11898033, rs11902987, rs12713264, rs12713265, |
| | | rs12713266, rs12713267, rs12713268, rs12713269, rs12713274, |
| | | rs13386146, rs13391522, rs13399656, rs13403284, rs13407214, |
| | | rs13423873, rs13432302, rs17046097, rs28581481, rs3287, |
| | | rs3796017, rs3796018, rs3796019, rs4346422, rs4347868, |
| | | rs4374410, rs4455200, rs4616509, rs4641979, rs6545430, |
| | | rs6706263, rs6739140, rs6740893, rs6749802, rs7340302, |
| | | rs7556752, rs7564719, rs7586311, rs7591204, rs7599241, |
| | | rs7608414, rs9309256, rs9677089 |
| rs8099840 | DDA | rs112620863, rs12609198, rs12610560, rs12971481, rs3813135, |
| | | rs4638719, rs733731, rs8099840, rs8105422, rs892145 |
| rs880633 | DIA | rs10920578, rs12128727, rs12141494, rs2071579, rs35405821, |
| | | rs55700740, rs880633, rs884209, rs903357, rs946259 |
| rs9820435 | DDA, DIA | rs13314114, rs1530455, rs2278668, rs3792366, rs3792369, |
| | | rs3792376, rs3792378, rs3792382, rs3792386, rs3804749, |
| | | rs3804753, rs4677875, rs726681, rs920900, rs9820435, |
| | | rs9823527 |
| rs9898 | DDA, DIA | rs114232169, rs59123177, rs9866126, rs9898 |

### Example 5: Identification of Additional AD Biomarkers

In another pQTL analysis workflow, 184 human plasma samples from a balanced Alzheimer's Disease (AD) cohort were genotyped on the GSA array. The same samples were processed using the PROTEOGRAPH workflow and a panel of five proprietary engineered nanoparticles. After processing, an LC-MS analysis was conducted on the digested peptides. These data were then interpreted by the DIA-NN data analysis pipeline using four spectral libraries: the in silico predicted library; the fractionated plasma (DDA) library; the DDA project-specific cohort plasma library; and the DIA project-specific cohort plasma library. We selected the optimal genetic association program as per its performance on the in silico predicted spectral library, then compared the number and spread of pQTLs across various libraries. Finally, we performed protein-based enrichment testing on the maximal set of pQTLs from these libraries to link the genetic etiology of the diseases to function.

We used PLINK to compute associations between the genotypes and protein quantifications (i.e., pQTLs). TABLE 11 lists 168 genome-wide significant associations based on Bonferroni-corrected p-value from this analysis. The results indicate that pQTLs found across the libraries are enriched for several complement, coagulation, and extracellular terms across several ontologies, with top terms: “GO: CC: extracellular region” (p=7.87×10-17), “KEGG: complement and coagulation cascades” (p=1.42×10-15), “GO: CC: extracellular space” (p=6.18×10-12), “GO: BP: response to external stimulus” (p=6.39×10-10), and “REAC: complement cascade” (p=1.17×10-9).

TABLE 11
Additional AD biomarkers.

| | | | P- | ASSOCIATION |
|---|---|---|---|---|
| ID | PHENO | LIBRARY | VALUE | TYPE |
| rs17198 | P54803 | Project-specific Cohort | 1.30E−36 | cis |
| | | Plasma - DDA | | |
| GSA- | P14625 | Fractionated Plasma - | 4.79E−34 | cis |
| rs1165692 | | DDA | | |
| rs1329424 | A0A0D9SG88_Q5TFM2 | Project-specific Cohort | 3.76E−30 | cis |
| | | Plasma - DDA | | |
| rs17198 | P54803_P54803_3_P54803_4 | Project-specific Cohort | 1.61E−29 | cis |
| | | Plasma - DIA | | |
| rs9898 | P04196 | In-silico Predicted | 3.92E−29 | cis |
| | | Library | | |
| rs4524 | O75093 | Project-specific Cohort | 3.32E−27 | trans |
| | | Plasma - DIA | | |
| rs6512033 | Q96PD5_2 | In-silico Predicted | 2.19E−26 | cis |
| | | Library | | |
| rs395544 | A0A0D9SG88_Q5TFM2 | Project-specific Cohort | 9.48E−26 | cis |
| | | Plasma - DDA | | |
| GSA- | P01892 | Project-specific Cohort | 2.67E−25 | trans |
| rs2975041 | | Plasma - DIA | | |
| rs2735097 | P01892 | Project-specific Cohort | 1.62E−23 | trans |
| | | Plasma - DIA | | |
| rs6677604 | Q03591 | Project-specific Cohort | 4.95E−23 | cis |
| | | Plasma - DDA | | |
| GSA- | O75093 | Project-specific Cohort | 2.06E−20 | trans |
| rs6030 | | Plasma - DIA | | |
| GSA- | Q15149_2 | Project-specific Cohort | 4.40E−20 | cis |
| rs55895668 | | Plasma - DIA | | |
| rs6512033 | Q96PD5 | Project-specific Cohort | 4.45E−20 | cis |
| | | Plasma - DDA | | |
| rs41268617 | P04196 | Project-specific Cohort | 7.74E−20 | cis |
| | | Plasma - DDA | | |
| rs380390 | A0A0D9SG88_Q5TFM2 | Project-specific Cohort | 2.87E−19 | cis |
| | | Plasma - DIA | | |
| rs272817 | Q92896_Q92896_2_Q92896_3 | Project-specific Cohort | 5.96E−19 | trans |
| | | Plasma - DDA | | |
| exm2265845 | Q92896_Q92896_2_Q92896_3 | Project-specific Cohort | 5.96E−19 | trans |
| | | Plasma - DDA | | |
| GSA- | Q92896_Q92896_2_Q92896_3 | Project-specific Cohort | 5.96E−19 | trans |
| rs36013739 | | Plasma - DDA | | |
| rs218396 | Q92896_Q92896_2_Q92896_3 | Project-specific Cohort | 5.96E−19 | trans |
| | | Plasma - DDA | | |
| exm2261761 | Q92896_Q92896_2_Q92896_3 | Project-specific Cohort | 5.96E−19 | trans |
| | | Plasma - DDA | | |
| rs73466148 | P01042 3 | Project-specific Cohort | 1.71E−18 | trans |
| | | Plasma - DIA | | |
| rs6677604 | P36980_P36980_2 | In-silico Predicted | 2.79E−18 | cis |
| | | Library | | |
| rs62623707 | O14960 | In-silico Predicted | 3.39E−18 | cis |
| | | Library | | |
| rs1801020 | P00748 | In-silico Predicted | 9.34E−18 | cis |
| | | Library | | |
| rs1056522 | P19827 | In-silico Predicted | 1.06E−17 | trans |
| | | Library | | |
| rs2735046 | P10316 | Fractionated Plasma - | 1.19E−17 | trans |
| | | DDA | | |
| rs9300510 | A0A0D9SG88_Q5TFM2 | In-silico Predicted | 2.03E−17 | trans |
| | | Library | | |
| rs2289702 | P09668 | Project-specific Cohort | 2.35E−17 | cis |
| | | Plasma - DIA | | |
| seq- | Q15149_2 | Project-specific Cohort | 3.77E−17 | cis |
| rs6993938 | | Plasma - DIA | | |
| exm2260367 | Q8N474 | Fractionated Plasma - | 4.14E−17 | trans |
| | | DDA | | |
| GSA- | A0A0D9SG88_Q5TFM2 | In-silico Predicted | 5.07E−17 | trans |
| rs7754077 | | Library | | |
| exm2260986 | A0A0D9SG88_Q5TFM2 | In-silico Predicted | 5.07E−17 | trans |
| | | Library | | |
| rs395544 | Q03591 | Project-specific Cohort | 6.08E−17 | cis |
| | | Plasma - DIA | | |
| rs2545801 | P00748 | In-silico Predicted | 9.58E−17 | cis |
| | | Library | | |
| rs1611715 | P01892 | Project-specific Cohort | 1.88E−16 | trans |
| | | Plasma - DIA | | |
| GSA- | P04196 | Project-specific Cohort | 2.03E−16 | cis |
| rs1131364 | | Plasma - DDA | | |
| GSA- | Q03591 | Project-specific Cohort | 3.34E−16 | trans |
| rs11915474 | | Plasma - DDA | | |
| GSA- | P36980_P36980_2 | In-silico Predicted | 3.79E−16 | trans |
| rs11915474 | | Library | | |
| rs1165669 | P14625 | Fractionated Plasma - | 3.86E−16 | cis |
| | | DDA | | |
| rs6783962 | P19827 | In-silico Predicted | 3.91E−16 | trans |
| | | Library | | |
| rs2523409 | P10316 | Fractionated Plasma - | 4.26E−16 | trans |
| | | DDA | | |
| rs2523409 | P01892 | Project-specific Cohort | 5.70E−16 | trans |
| | | Plasma - DIA | | |
| rs9357092 | P01892 | Project-specific Cohort | 5.85E−16 | trans |
| | | Plasma - DIA | | |
| rs11777239 | Q15149_2 | Project-specific Cohort | 6.26E−16 | cis |
| | | Plasma - DIA | | |
| rs1001179 | P04040 | Fractionated Plasma - | 6.79E−16 | cis |
| | | DDA | | |
| rs1046875 | Q9H479 | Project-specific Cohort | 7.01E−16 | cis |
| | | Plasma - DIA | | |
| rs3923387 | Q15149_2 | Project-specific Cohort | 9.05E−16 | cis |
| | | Plasma - DIA | | |
| rs4305381 | P19827 | In-silico Predicted | 9.86E−16 | trans |
| | | Library | | |
| exm2260367 | P21741 | Project-specific Cohort | 1.17E−15 | trans |
| | | Plasma - DIA | | |
| rs2523946 | P10316 | Fractionated Plasma - | 1.33E−15 | trans |
| | | DDA | | |
| rs73466148 | P40937_P40937_2 | Fractionated Plasma - | 1.44E−15 | trans |
| | | DDA | | |
| rs12148472 | P09668 | Project-specific Cohort | 1.65E−15 | cis |
| | | Plasma - DIA | | |
| rs1884989 | P04180 | Project-specific Cohort | 1.78E−15 | trans |
| | | Plasma - DDA | | |
| exm2271590 | A0A0D9SG88_Q5TFM2 | In-silico Predicted | 2.60E−15 | trans |
| | | Library | | |
| rs12493926 | P04196 | Project-specific Cohort | 2.75E−15 | cis |
| | | Plasma - DDA | | |
| rs7464572 | Q15149_2 | Project-specific Cohort | 2.85E−15 | cis |
| | | Plasma - DIA | | |
| rs9898 | Q709C8_Q709C8_3 | Project-specific Cohort | 3.10E−15 | trans |
| | | Plasma - DDA | | |
| rs7014582 | Q15149_2 | Project-specific Cohort | 3.27E−15 | cis |
| | | Plasma - DIA | | |
| GSA- | P08833 | Fractionated Plasma - | 3.30E−15 | trans |
| rs7793026 | | DDA | | |
| rs6695321 | A0A0D9SG88_Q5TFM2 | Project-specific Cohort | 3.82E−15 | cis |
| | | Plasma - DDA | | |
| rs1329424 | Q03591 | Project-specific Cohort | 4.42E−15 | cis |
| | | Plasma - DIA | | |
| rs1045922 | Q93091 | Project-specific Cohort | 6.83E−15 | cis |
| | | Plasma - DDA | | |
| rs2975034 | P01892 | Project-specific Cohort | 8.08E−15 | trans |
| | | Plasma - DIA | | |
| rs6003 | Q03591 | Project-specific Cohort | 9.43E−15 | cis |
| | | Plasma - DDA | | |
| rs11635626 | H3BPZ1 | Project-specific Cohort | 1.09E−14 | cis |
| | | Plasma - DIA | | |
| exm2270485 | P21741 | Fractionated Plasma - | 1.34E−14 | trans |
| | | DDA | | |
| rs6890853 | P16871_P16871_2_P16871_{—} | Project-specific Cohort | 1.39E−14 | cis |
| | 3_P16871_4 | Plasma - DIA | | |
| rs11783655 | Q15149_2 | Project-specific Cohort | 1.62E−14 | cis |
| | | Plasma - DIA | | |
| rs3856650 | P19827 | Project-specific Cohort | 1.66E−14 | trans |
| | | Plasma - DDA | | |
| GSA- | Q15149_Q15149_2_Q15149_{—} | Fractionated Plasma - | 1.89E−14 | cis |
| rs55895668 | 4_Q15149_5_Q15149_6_{—} | DDA | | |
| | Q15149_7_Q15149_8_{—} | | | |
| | Q15149_9 | | | |
| chr6: | P10316 | Fractionated Plasma - | 2.01E−14 | trans |
| 29939240 | | DDA | | |
| rs4344876 | Q9UQN3_Q9UQN3_2 | Project-specific Cohort | 2.47E−14 | trans |
| | | Plasma - DIA | | |
| rs4404487 | Q9ULI3 | Fractionated Plasma - | 3.24E−14 | cis |
| | | DDA | | |
| rs9380141 | P01892 | Project-specific Cohort | 3.81E−14 | trans |
| | | Plasma - DIA | | |
| rs4740 | Q14213 | Fractionated Plasma - | 3.82E−14 | cis |
| | | DDA | | |
| GSA- | Q15149_4 | Project-specific Cohort | 5.01E−14 | cis |
| rs55895668 | | Plasma - DDA | | |
| rs6677604 | P36980 | Project-specific Cohort | 5.49E−14 | cis |
| | | Plasma - DIA | | |
| rs2372813 | O75900_2 | Fractionated Plasma - | 6.26E−14 | trans |
| | | DDA | | |
| exm2264447 | P00915 | Project-specific Cohort | 8.70E−14 | trans |
| | | Plasma - DDA | | |
| rs1426654 | P00915 | Project-specific Cohort | 8.70E−14 | trans |
| | | Plasma - DDA | | |
| rs6457110 | P01892 | Project-specific Cohort | 1.04E−13 | trans |
| | | Plasma - DIA | | |
| rs11718493 | P19827 | Project-specific Cohort | 1.09E−13 | trans |
| | | Plasma - DDA | | |
| rs9836291 | P26927 | In-silico Predicted | 1.10E−13 | trans |
| | | Library | | |
| GSA- | P10321_2 | Project-specific Cohort | 1.28E−13 | trans |
| rs1611704 | | Plasma - DDA | | |
| rs2735097 | P10316 | Fractionated Plasma - | 1.29E−13 | trans |
| | | DDA | | |
| rs812498 | P14625 | Fractionated Plasma - | 1.44E−13 | cis |
| | | DDA | | |
| rs11635626 | Q9P035 | Project-specific Cohort | 1.46E−13 | cis |
| | | Plasma - DDA | | |
| rs11136344 | Q15149_2 | Project-specific Cohort | 1.86E−13 | cis |
| | | Plasma - DIA | | |
| GSA- | P36980 | Fractionated Plasma - | 2.05E−13 | trans |
| rs11915474 | | DDA | | |
| GSA- | P21741 | Project-specific Cohort | 2.34E−13 | trans |
| rs11901661 | | Plasma - DDA | | |
| rs2735046 | P01892 | Project-specific Cohort | 2.65E−13 | trans |
| | | Plasma - DIA | | |
| rs12493410 | P19827 | Project-specific Cohort | 2.97E−13 | trans |
| | | Plasma - DDA | | |
| exm2264486 | Q9NUQ9 | Fractionated Plasma - | 3.55E−13 | trans |
| | | DDA | | |
| rs9824398 | P04196 | Project-specific Cohort | 3.96E−13 | cis |
| | | Plasma - DDA | | |
| rs1426654 | P02042 | Project-specific Cohort | 4.41E−13 | trans |
| | | Plasma - DIA | | |
| exm2264447 | P02042 | Project-specific Cohort | 4.41E−13 | trans |
| | | Plasma - DIA | | |
| rs7533936 | P10643 | Project-specific Cohort | 4.69E−13 | trans |
| | | Plasma - DDA | | |
| rs11764079 | Q15165_Q15165_1_Q15165_3 | Project-specific Cohort | 4.96E−13 | cis |
| | | Plasma - DDA | | |
| rs1736982 | P10316 | Fractionated Plasma - | 5.16E−13 | trans |
| | | DDA | | |
| GSA- | O75900_2 | Fractionated Plasma - | 6.36E−13 | trans |
| rs7872425 | | DDA | | |
| rs3784539 | P09668 | Project-specific Cohort | 7.02E−13 | cis |
| | | Plasma - DDA | | |
| rs4807563 | Q14213 | Project-specific Cohort | 7.36E−13 | cis |
| | | Plasma - DDA | | |
| exm2264447 | A5YKK6 | Project-specific Cohort | 8.88E−13 | trans |
| | | Plasma - DDA | | |
| rs1426654 | A5YKK6 | Project-specific Cohort | 8.88E−13 | trans |
| | | Plasma - DDA | | |
| rs8099840 | Q96PD5_2 | In-silico Predicted | 9.02E−13 | cis |
| | | Library | | |
| rs3115627 | P10316 | Fractionated Plasma - | 9.41E−13 | trans |
| | | DDA | | |
| GSA- | A0A0D9SG88_Q5TFM2 | In-silico Predicted | 9.55E−13 | trans |
| rs17706123 | | Library | | |
| rs9357092 | P10321_2 | Project-specific Cohort | 1.15E−12 | trans |
| | | Plasma - DDA | | |
| rs1040425 | Q16678 | Project-specific Cohort | 1.35E−12 | trans |
| | | Plasma - DDA | | |
| seq- | A0A3B3ISR2_B4DPQ0_{—} | Project-specific Cohort | 1.49E−12 | cis |
| rs1126605 | F5H2D0 | Plasma - DDA | | |
| chr6: | P01892 | Project-specific Cohort | 1.53E−12 | trans |
| 29928649 | | Plasma - DIA | | |
| rs2345436 | Q08257_3 | Fractionated Plasma - | 1.54E−12 | cis |
| | | DDA | | |
| rs1056522 | Q9NR34 | Project-specific Cohort | 1.59E−12 | trans |
| | | Plasma - DIA | | |
| rs68066031 | P07093_3 | Fractionated Plasma - | 1.61E−12 | cis |
| | | DDA | | |
| rs9380141 | P10316 | Fractionated Plasma - | 1.64E−12 | trans |
| | | DDA | | |
| rs2523946 | P01892 | Project-specific Cohort | 1.72E−12 | trans |
| | | Plasma - DIA | | |
| rs1056522 | P19823 | In-silico Predicted | 1.75E−12 | trans |
| | | Library | | |
| rs2975034 | P10321 2 | Project-specific Cohort | 1.79E−12 | trans |
| | | Plasma - DDA | | |
| rs7493 | Q15165_Q15165_1_Q15165_3 | Project-specific Cohort | 1.79E−12 | cis |
| | | Plasma - DDA | | |
| rs6457110 | P10316 | Fractionated Plasma - | 1.84E−12 | trans |
| | | DDA | | |
| rs68066031 | P07093_2_P07093_3 | Project-specific Cohort | 1.92E−12 | cis |
| | | Plasma - DIA | | |
| rs6003 | P36980_P36980_2 | In-silico Predicted | 2.09E−12 | cis |
| | | Library | | |
| rs3845960 | P19827 | In-silico Predicted | 2.55E−12 | trans |
| | | Library | | |
| GSA- | Q15517 | In-silico Predicted | 3.01E−12 | trans |
| rs76221975 | | Library | | |
| rs73466148 | P28799 | In-silico Predicted | 3.28E−12 | trans |
| | | Library | | |
| rs1800450 | P11226 | In-silico Predicted | 3.29E−12 | cis |
| | | Library | | |
| exm2266941 | Q13131_Q13131_2 | Project-specific Cohort | 3.36E−12 | trans |
| | | Plasma - DIA | | |
| GSA- | Q9P035 | Project-specific Cohort | 3.55E−12 | cis |
| rs78374057 | | Plasma - DDA | | |
| rs8099840 | Q96PD5 | Project-specific Cohort | 3.57E−12 | cis |
| | | Plasma - DDA | | |
| GSA- | Q9Y450_Q9Y450_4 | Fractionated Plasma - | 3.64E−12 | trans |
| rs7088203 | | DDA | | |
| rs508406 | Q8N6R0_Q8N6R0_1_Q8N6R0_3 | Project-specific Cohort | 3.64E−12 | trans |
| | | Plasma - DDA | | |
| rs1611715 | P10321 2 | Project-specific Cohort | 3.72E−12 | trans |
| | | Plasma - DDA | | |
| rs612766 | A0A0D9SG88_Q5TFM2 | In-silico Predicted | 4.11E−12 | trans |
| | | Library | | |
| GSA- | O75093 | In-silico Predicted | 4.13E−12 | trans |
| rs1651025 | | Library | | |
| rs440770 | P54803 | Project-specific Cohort | 4.13E−12 | cis |
| | | Plasma - DDA | | |
| seq- | Q15149_4 | Project-specific Cohort | 4.16E−12 | cis |
| rs6993938 | | Plasma - DDA | | |
| rs35620248 | P26927 | Fractionated Plasma - | 4.28E−12 | trans |
| | | DDA | | |
| GSA- | O14980 | Project-specific Cohort | 4.50E−12 | trans |
| rs7088203 | | Plasma - DDA | | |
| GSA- | A0A0B4J1U7 | Fractionated Plasma - | 5.42E−12 | cis |
| rs4280141 | | DDA | | |
| rs11779776 | Q15149_2 | Project-specific Cohort | 5.55E−12 | cis |
| | | Plasma - DIA | | |
| rs12258356 | Q03591 | Project-specific Cohort | 5.61E−12 | trans |
| | | Plasma - DDA | | |
| rs387608 | P00751 | In-silico Predicted | 5.75E−12 | cis |
| | | Library | | |
| GSA- | H3BPZ1 | Project-specific Cohort | 5.99E−12 | cis |
| rs78374057 | | Plasma - DIA | | |
| GSA- | A8TX70_A8TX70_2 | Fractionated Plasma - | 6.33E−12 | trans |
| rs13355990 | | DDA | | |
| rs10051419 | P26572 | In-silico Predicted | 6.73E−12 | trans |
| | | Library | | |
| rs11635626 | Q9P035_Q9P035_2 | Project-specific Cohort | 7.26E−12 | cis |
| | | Plasma - DIA | | |
| rs6049301 | Q9Y263 | In-silico Predicted | 7.28E−12 | trans |
| | | Library | | |
| rs399419 | Q9Y263 | In-silico Predicted | 7.53E−12 | trans |
| | | Library | | |
| rs10048970 | Q9Y263 | In-silico Predicted | 7.53E−12 | trans |
| | | Library | | |
| GSA- | Q9Y263 | In-silico Predicted | 7.53E−12 | trans |
| rs12353327 | | Library | | |
| GSA- | Q9Y263 | In-silico Predicted | 7.53E−12 | trans |
| rs113453449 | | Library | | |
| GSA- | P10321_2 | Project-specific Cohort | 7.65E−12 | trans |
| rs2975041 | | Plasma - DDA | | |
| rs7464572 | Q15149_Q15149_2_Q15149_{—} | Fractionated Plasma - | 7.77E−12 | cis |
| | 4_Q15149_5_Q15149_6_{—} | DDA | | |
| | Q15149_7_Q15149_8_{—} | | | |
| | Q15149_9 | | | |
| rs6003 | P36980 | Project-specific Cohort | 7.89E−12 | cis |
| | | Plasma - DIA | | |
| GSA- | P08833 | Fractionated Plasma - | 8.29E−12 | trans |
| rs9932711 | | DDA | | |
| GSA- | P78539_P78539_2_P78539_5 | In-silico Predicted | 9.29E−12 | trans |
| rs12085181 | | Library | | |
| exm2260367 | Q9H4F8_Q9H4F8_2 | Project-specific Cohort | 9.75E−12 | trans |
| | | Plasma - DDA | | |
| rs11783655 | Q15149_4 | Project-specific Cohort | 9.76E−12 | cis |
| | | Plasma - DDA | | |
| rs2508037 | P10316 | Fractionated Plasma - | 1.02E−11 | trans |
| | | DDA | | |
| rs1056522 | Q06033_Q06033_2 | In-silico Predicted | 1.03E−11 | trans |
| | | Library | | |
| exm2261461 | P02042 | In-silico Predicted | 1.05E−11 | trans |
| | | Library | | |
| rs203850 | P40937_P40937_2 | Fractionated Plasma - | 1.17E−11 | trans |
| | | DDA | | |
| rs11003118 | P11226 | Project-specific Cohort | 1.17E−11 | cis |
| | | Plasma - DIA | | |
| exm2264447 | P13747 | Fractionated Plasma - | 1.21E−11 | trans |
| | | DDA | | |
| rs1426654 | P13747 | Fractionated Plasma - | 1.21E−11 | trans |
| | | DDA | | |
| rs2743941 | P10316 | Fractionated Plasma - | 1.40E−11 | trans |
| | | DDA | | |
| rs3823374 | P10316 | Fractionated Plasma - | 1.71E−11 | trans |
| | | DDA | | |

### LIST OF EMBODIMENTS

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment 1. A method for determining a risk or state of a neurodegenerative disease of a subject comprising: (a) detecting a presence of a biomarker in a biological sample from the subject, wherein the biomarker comprises at least one of: E7EUF1, O94812, P02549, P02730, P05019, P05154, P05546, P13497, P16157, P16452, P17936, P24593, P27918, P35858, P41218, Q12797, Q13214, Q13822, Q8NI99, Q96IY4, Q99715, Q9BXN1, Q9H0B8, or a proteoform thereof; and (b) determining the risk or state of the neurodegenerative disease of the subject based on the presence of the biomarker in the biological sample.

Embodiment 2. The method of Embodiment 1, wherein the biomarker further comprises at least one of: P04114, P30043, P00742, or a proteoform thereof.

Embodiment 3. The method of Embodiment 1 or 2, wherein O94812 proteoform is a proteoform selected from the group consisting of: O94812-2, O94812-3, O94812-5, O94812-6, and O94812-7.

Embodiment 4. The method of any one of Embodiments 1-3, wherein P02549 proteoform is a P02549-2 proteoform.

Embodiment 5. The method of any one of Embodiments 1-4, wherein P35858 proteoform is a P35858-2 proteoform.

Embodiment 6. The method of any one of Embodiments 1-5, wherein Q13214 proteoform is a Q13214-2 proteoform.

Embodiment 7. The method of any one of Embodiments 1-6, wherein Q13822 proteoform is a Q13822-3 proteoform.

Embodiment 8. The method of any one of Embodiments 1-7, wherein Q9H0B8 proteoform is a Q9H0B8-3 proteoform.

Embodiment 9. The method of any one of Embodiments 1-8, wherein P16157 proteoform is a P16157-12 proteoform.

Embodiment 10. The method of any one of Embodiments 1-9, wherein P16452 proteoform is a P16452-2 proteoform.

Embodiment 11. A method for determining a risk or state of a neurodegenerative disease of a subject comprising: (a) detecting a presence of a biomarker in a biological sample from the subject, wherein the biomarker comprises at least one of: 0A0B4J1U7, A0A0D9SG88; Q5TFM2, A0A3B3ISR2; B4DPQ0; F5H2D0, A5YKK6, A8TX70; A8TX70-2, H3BPZ1, O14960, O14980, O75093, O75900-2, P00748, P00751, P00915, P01042-3, P01892, P02042, P04040, P04180, P04196, P07093-2; P07093-3, P070933, P08833, P09668, P10316, P10321-2, P10643, P11226, P13747, P14625, P16871; P16871-2; P16871-3; P16871-4, P19823, P19827, P21741, P26572, P26927, P28799, P36980, P36980; P36980-2, P40937; P40937-2, P54803, P54803; P54803-3; P54803-4, P78539; P78539-2; P78539-5, Q03591, Q06033; Q06033-2, Q08257-3, Q13131; Q13131-2, Q14213, Q15149-2, Q15149-4, Q15149; Q15149-2; Q15149-4; Q15149-5; Q15149-6; Q15149-7; Q15149-8; Q15149-9, Q15165; Q15165-1; Q15165-3, Q15517, Q16678, Q709C8; Q709C8-3, Q8N474, Q8N6R0; Q8N6R0-1; Q8N6R0-3, Q92896; Q92896-2; Q92896-3, Q93091, Q96PD5, Q96PD5-2, Q9H479, Q9H4F8; Q9H4F8-2, Q9NR34, Q9NUQ9, Q9P035, Q9ULI3, Q9UQN3; Q9UQN3-2, Q9Y263, Q9Y450; Q9Y450-4, or a proteoform thereof; and (b) determining the risk or state of the neurodegenerative disease of the subject based on the presence of the biomarker in the biological sample.

Embodiment 12. The method of Embodiment 11, wherein the biomarker further comprises at least one of: P54803, P14625, P30043, P00742, A0A0D9SG88, Q5TFM2, P54803, P54803-3, P54803-4, P04196, or a proteoform thereof.

Embodiment 13. The method of any one of Embodiments 1-12, wherein the state is a diagnosis or a prognosis of the neurodegenerative disease in the subject.

Embodiment 14. The method of any one of Embodiments 1-10, wherein the state is a stratification of the neurodegenerative disease in the subject.

Embodiment 15. The method of any one of Embodiments 1-10, wherein the state is a monitoring of the neurodegenerative disease in the subject.

Embodiment 16. The method of any one of Embodiments 1-10, wherein the risk is an expected probability that the subject develops the neurodegenerative disease later in life.

Embodiment 17. The method of any one of Embodiments 1-16, wherein the neurodegenerative disease is Alzheimer's disease.

Embodiment 18. The method of any one of Embodiments 1-17, wherein the biological sample comprises plasma, serum, urine, cerebrospinal fluid, synovial fluid, tears, saliva, whole blood, a white blood cell, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

Embodiment 19. The method of Embodiment 18, wherein the biological sample is a blood, serum, or plasma.

Embodiment 20. The method of Embodiment 19, wherein the biological sample is plasma.

Embodiment 21. The method of any one of Embodiments 1-20, wherein the presence of the biomarker comprises a level of the biomarker.

Embodiment 22. The method of Embodiment 21, wherein the level of the biomarker is indicative for the presence of the neurodegenerative disease in the subject when the level is higher in the biological sample compared to a reference value.

Embodiment 23. The method of Embodiment 21, wherein the level of the biomarker is indicative for the absence of the neurodegenerative disease in the subject when the level is lower in the biological sample compared to a reference value.

Embodiment 24. The method of any one of Embodiments 1-23, wherein the biomarker comprises a plurality of biomarkers comprising at least two of: E7EUF1, O94812, P02549, P02730, P05019, P05154, P05546, P13497, P16157, P16452, P17936, P24593, P27918, P35858, P41218, Q12797, Q13214, Q13822, Q8NI99, Q96IY4, Q99715, Q9BXN1, Q9H0B8, or a proteoform thereof.

Embodiment 25. The method of Embodiment 24, wherein the presence of the biomarker comprises a pattern of levels of the plurality biomarkers.

Embodiment 26. The method of Embodiment 25, wherein the pattern of levels of the plurality of biomarkers is indicative for the presence of the neurodegenerative disease in the subject when the pattern of levels is substantially similar in the biological sample compared to a reference pattern of levels.

Embodiment 27. The method of Embodiment 25, wherein the pattern of levels of the plurality of biomarkers is indicative for the presence of the neurodegenerative disease in the subject when the pattern of levels is substantially different in the biological sample compared to a reference pattern of levels.

Embodiment 28. The method of Embodiment 26 or 27, wherein the determining comprises using a machine learning algorithm to analyze the pattern of levels.

Embodiment 29. The method of Embodiment 28, wherein the machine learning algorithm is configured to determine if the pattern of levels of the plurality of biomolecules of the biomarker is substantially similar or substantially different in the biological sample compared to the reference pattern of levels.

Embodiment 30. The method of any one of Embodiments 1-29, wherein the detecting a presence of a biomarker in the biological sample comprises selectively enriching the one or more biomolecules.

Embodiment 31. The method of any one of Embodiments 1-30, wherein the detecting a presence of a biomarker in the biological sample comprises performing mass spectrometry with the one or more biomolecules.

Embodiment 32. The method of any one of Embodiments 1-31, wherein the subject is suspected of having early-stage Alzheimer's Disease.

Embodiment 33. The method of any one of Embodiments 1-31, wherein the subject is asymptomatic for the neurodegenerative disease.

Embodiment 34. The method of any one of Embodiments 1-31, wherein the subject exhibits or reports one or more symptoms of Alzheimer's Disease comprising: impairment in cognition, impairment in memory, impairment in language, or impairment in perception.

Embodiment 35. The method of any one of Embodiments 1-31, wherein the subject has or is suspected of having Alzheimer's Disease based on an expert evaluation.

Embodiment 36. The method any one of Embodiments 1-31, wherein the subject has or is suspected of having Alzheimer's Disease based on a positive Alzheimer's Disease diagnosis from another diagnostic.

Embodiment 37. A method for determining a risk or state of a neurodegenerative disease of a subject comprising: (a) detecting a presence of a biomarker in a biological sample from the subject, wherein the biomarker is involved in a fibrin clot formation cascade; and (b) determining the risk or state of the neurodegenerative disease of the subject based on the presence of the biomarker in the biological sample.

Embodiment 38. The method of Embodiment 37, wherein the biomarker is involved in an extrinsic pathway of the fibrin clot formation cascade.

Embodiment 39. The method of Embodiment 37 or 38, wherein the biomarker is involved in an intrinsic pathway of the fibrin clot formation cascade.

Embodiment 40. The method of any one of Embodiments 37-39, wherein the biomarker is involved in a common pathway of the fibrin clot formation cascade.

Embodiment 41. The method of any one of Embodiments 37-40, wherein the biomarker comprises at least one of: P00451, P00734, P00740, P00742, P00748, P01008, P01023, P01042, P02671, P02675, P02679, P02776, P03951, P03952, P04070, P04275, P05154, P05155, P05160, P05546, P07093, P07204, P07225, P07359, P08709, P10646, P10720, P12259, P13224, P13726, P14770, P24158, P25116, P40197, P42785, Q07021, Q8N6Q3, Q9UNN8, or a proteoform thereof.

Embodiment 42. The method of any one of Embodiments 37-41, wherein the biomarker is involved in a complement and coagulation cascade, a IGF1-IGFBP3-ALS complex, or a regulation of insulin-like growth factor (IGF) transport and uptake by Insulin-like Growth Factor Binding Proteins (IGFBPs).

Embodiment 43. The method of Embodiment 42, wherein the biomarker is involved in the complement and coagulation cascades.

Embodiment 44. The method of Embodiment 43, wherein the biomarker comprises at least one of: K03901, K01320, K01314, K03902, K01313, K01328, K01323, K01321, K03900, K03899, K03907, K06557, K01344, K03914, K04235, K04236, K03917, K03906, K01300, K03903, K03904, K03905, K01324, K03898, K03915, K03916, K01315, K03909, K03911, K03912, K03913, K03908, K03982, K19821, K01343, K01348, K03985, K03984, K03983, K03910, K01335, K01334, K03990, K03994, K03995, K03996, K03997, K03998, K03999, K04000, K03986, K03987, K03988, K01330, K01331, K03991, K03992, K03993, K01332, K03989, K04009, K19822, K04011, K04012, K06461, K06464, K06462, K04010, K04004, K01333, K04001, K04006, K04007, K04002, K04003, K04008, K17252, K06251, or a proteoform thereof.

Embodiment 45. The method of Embodiment 42, wherein the biomarker is involved in the IGF1-IGFBP3-ALS complex.

Embodiment 46. The method of Embodiment 45, wherein biomarker comprises at least one of: P05019, P17936, P35858, or a proteoform thereof.

Embodiment 47. The method of Embodiment 42, wherein biomarker is involved in the regulation of insulin-like growth factor (IGF) transport and uptake by Insulin-like Growth Factor Binding Proteins (IGFBPs).

Embodiment 48. The method of Embodiment 47, wherein biomarker comprises at least one of: O00255, O00391, O00622, O14672, O14791, O15232, O15240, O43493, O43852, O76024, O76061, O95084, O95633, O95972, P00450, P00734, P00747, P01008, P01009, P01024, P01033, P01034, P01042, P01210, P01344, P02647, P02649, P02652, P02671, P02679, P02751, P02765, P02768, P02771, P02787, P03956, P04070, P04114, P05019, P05060, P05067, P05231, P05546, P06870, P07237, P07288, P07942, P08253, P08311, P08582, P08833, P09382, P09603, POCOL4, P10451, P11047, P12259, P12644, P13521, P13611, P14314, P14625, P17936, P18065, P19022, P19823, P20151, P20718, P22692, P23327, P24592, P24593, P24821, P34741, P35555, P35858, P51654, P55268, Q02818, Q06481, Q07065, Q08431, Q12841, Q13103, Q13217, Q13219, Q13316, Q13421, Q14393, Q14515, Q14703, Q14766, Q15084, Q15293, Q16270, Q24JP5, Q5JRA6, Q6P988, Q6PCB0, Q6Q788, Q6UX39, Q86UP2, Q8IXL6, Q8N114, Q8N4F0, Q8NBJ4, Q8NBP7, Q8WXD2, Q96AD5, Q96MK3, Q99217, Q9BRK3, Q9BTY2, Q9BU40, Q9BXP8, Q9GZV9, Q9H8M9, Q9HCE9, Q9NP70, Q9NQ76, Q9NRM1, Q9UK55, Q9UKR3, Q9UM21, or a proteoform thereof.

Embodiment 49. The method of any one of Embodiments 37-48, wherein the subject is suspected of having early-stage Alzheimer's Disease.

Embodiment 50. The method of any one of Embodiments 37-48, wherein the subject is asymptomatic for the neurodegenerative disease.

Embodiment 51. The method of any one of Embodiments 37-48, wherein the subject exhibits or reports one or more symptoms of Alzheimer's Disease comprising: impairment in cognition, impairment in memory, impairment in language, or impairment in perception.

Embodiment 52. The method of any one of Embodiments 37-48, wherein the subject has or is suspected of having Alzheimer's Disease based on an expert evaluation.

Embodiment 53. The method of any one of Embodiments 37-48, wherein the subject has or is suspected of having Alzheimer's Disease based on a positive Alzheimer's Disease diagnosis from another diagnostic.

Embodiment 54. A method for determining a risk or state of a disease of a subject comprising: (a) detecting a presence of one or more single nucleotide polymorphisms (SNPs) in a biological sample from the subject, wherein the one or more SNPs comprise at least one of: GSA-rs1046279, GSA-rs1131364, GSA-rs1165692, GSA-rs6030, rs1001179, rs1046875, rs1046896, rs1061147, rs1061147, rs10737680, rs10801555, rs11003118, rs1106766, rs11085954, rs11613352, rs1165669, rs12067507, rs12148472, rs12493926, rs1329424, rs1329428, rs1410996, rs1520079, rs17198, rs1800450, rs1801020, rs1801689, rs1831282, rs2289702, rs2545801, rs2731672, rs2749534, rs3197999, rs34882957, rs3741414, rs3784539, rs3792366, rs380390, rs395544, rs41268617, rs4524, rs5030062, rs62623707, rs6647, rs6677604, rs6695321, rs68066031, rs68147365, rs698078, rs707998, rs710446, rs7599241, rs8099840, rs880633, rs9820435, rs9898, or a SNP in linkage disequilibrium thereof; and (b) determining the risk or state of the disease of the subject based on the presence of the one or more SNPs in the biological sample.

Embodiment 55. A method for determining a risk or state of a disease of a subject comprising: (a) detecting a presence of one or more single nucleotide polymorphisms (SNPs) in a biological sample from the subject, wherein the one or more SNPs comprise at least one of: chr6:29928649, chr6:29939240, exm2260367, exm2260986, exm2261461, exm2261761, exm2264447, exm2264486, exm2265845, exm2270485, exm2271590, GSA-rs1131364, GSA-rs11901661, GSA-rs11915474, GSA-rs12085181, GSA-rs13355990, GSA-rs1611704, GSA-rs1651025, GSA-rs17706123, GSA-rs2975041, GSA-rs36013739, GSA-rs4280141, GSA-rs55895668, GSA-rs6030, GSA-rs7088203, GSA-rs76221975, GSA-rs78374057, GSA-rs7872425, GSA-rs7793026, GSA-rs7754077, GSA-rs7088203, rs3923387, rs11718493, rs11764079, rs11777239, rs11783655, rs11915474, rs12085181, rs12148472, rs12258356, rs12493410, rs12493926, rs1426654, rs1611715, rs1736982, rs1800450, rs1801020, rs203850, rs218396, rs2289702, rs2345436, rs2372813, rs2523409, rs2523946, rs2545801, rs272817, rs2735046, rs2735097, rs2743941, rs2975034, rs3115627, rs35620248, rs380390, rs3856650, rs387608, rs395544, rs399419, rs41268617, rs4305381, rs4344876, rs4404487, rs440770, rs4524, rs4740, rs508406, rs6003, rs6049301, rs6512033, rs6677604, rs6783962, rs68066031, rs6890853, rs7014582, rs73466148, rs7464572, rs7493, rs7533936, rs8099840, rs812498, rs9357092, rs9380141, rs9824398, rs9898, seq-rs6993938, seq-rs1126605, or a SNP in linkage disequilibrium thereof; and (b) determining the risk or state of the disease of the subject based on the presence of the one or more SNPs in the biological sample.

Embodiment 56. The method of Embodiments 54 or 55, wherein the disease is a neurodegenerative disease.

Embodiment 57. The method of Embodiment 56, wherein the state is a diagnosis or a prognosis of the neurodegenerative disease in the subject.

Embodiment 58. The method of Embodiment 56, wherein the state is a stratification of the neurodegenerative disease in the subject.

Embodiment 59. The method of Embodiment 56, wherein the state is a monitoring of the neurodegenerative disease in the subject.

Embodiment 60. The method of Embodiment 56, wherein the risk is an expected probability that the subject develops the neurodegenerative disease later in life.

Embodiment 61. The method of any one of Embodiments 54-60, wherein the neurodegenerative disease is Alzheimer's disease.

Embodiment 62. The method of any one of Embodiments 54-61, wherein the biological sample comprises plasma, serum, urine, cerebrospinal fluid, synovial fluid, tears, saliva, whole blood, a white blood cell, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof.

Embodiment 63. The method of Embodiment 62, wherein the detecting comprises nucleic acid sequencing.

Embodiment 64. The method of Embodiment 63, wherein the nucleic acid sequencing comprises DNA sequencing.

Embodiment 65. The method of any one of Embodiments 56-64, wherein the subject is suspected of having early-stage Alzheimer's Disease.

Embodiment 66. The method of any one of Embodiments 56-64, wherein the subject is asymptomatic for the neurodegenerative disease.

Embodiment 67. The method of any one of Embodiments 56-64, wherein the subject exhibits or reports one or more symptoms of Alzheimer's Disease comprising: impairment in cognition, impairment in memory, impairment in language, or impairment in perception.

Embodiment 68. The method of any one of Embodiments 56-64, wherein the subject has or is suspected of having Alzheimer's Disease based on an expert evaluation.

Embodiment 69. The method of any one of Embodiments 56-64, wherein the subject has or is suspected of having Alzheimer's Disease based on a positive Alzheimer's Disease diagnosis from another diagnostic.

Embodiment 70. A method for determining a risk or state of a neurodegenerative disease of the subject comprising: (a) providing a biological sample from a subject; (b) detecting a presence of one or more protein quantitative trait loci (pQTLs) in the biological sample, wherein the one or more pQTLs is associated with a protein level in vivo of one or more proteins comprising at least one of: C9, C8B, F5, CFH, MBL2, F12, KNG1, and SERPINA1; and (c) determining the state of the neurodegenerative disease of the subject based on the presence of the one or more pQTLs in the biological sample.

Embodiment 71. A method for determining a risk or state of a neurodegenerative disease of a subject comprising: (a) detecting a presence of one or more single nucleotide polymorphisms (SNPs) in a biological sample from the subject, wherein the one or more SNPs is associated with a protein level in vivo of one or more proteins that are involved in (i) a complement and coagulation cascade, (ii) a complement system, (iii) a complement system in neuronal development and plasticity, (iv) a Kinin-Kallikrein pathway, or (v) any combination thereof; and (b) determining the risk or state of the neurodegenerative disease of the subject based on the presence of the one or more protein quantitative trait loci in the biological sample.

Embodiment 72. The method of Embodiment 71, wherein the one or more proteins are involved in the complement and coagulation cascade.

Embodiment 73. The method of Embodiment 72, wherein the one or more proteins comprise at least one of: 109821, A2M, ADN, BDKRB1, Bradykinin, C1QA, C1QB, C1QG, C1R, C1S, C2, C3, C3AR1, C4, C5R1, C6, C7, C8G, C9, CD59A, CFH, CFI, CLU, CPB2, CR1, CR2, CRRY, Daf1, Daf2, F10, F12, F13B, F2, F2R, F3, F5, F7, F8, F9, FGB, Fibrin monomer, H2-BF, Hc, KLKB1, KNG1, MASP1, MASP2, MBL1, MCP, PLAT, PLAU, PLAUR, PLG, PROC, PROS1, SERPINA1, SERPINA5, SERPINC1, SERPIND1, SERPINE1, SERPINF2, SERPING1, TFPI, THBD, VWF, or a proteoform thereof.

Embodiment 74. The method of Embodiment 71, wherein the one or more proteins are involved in the complement system.

Embodiment 75. The method of Embodiment 74, wherein the one or more proteins comprise at least one of: Leishmanial protein kinase-1, A2M, ADIPOQ, ADM, ALB, APOA1, ARRB2, C1INH, C1q, C1qRp, C1r, C1s, C2, C3 (H2O), C3, C3a-desArg, C3a, C3aR1, C3bB3b, C3bB3bP, C3bBb, C3bBbP, C3b, C3c, C3d, C3f, C4-A, C4BP, C4a-desArg, C4a, C4b2b, C4b2b3b, C4b, C5, C5L2, C5a-desArg, C5a, C5aR1, C5b, C6, C7, C8, C9, CD16a, CD19, CD23, CD40, CD59, CFB, CFD, CFH, CFHR1, CFHR4, CFI, CFP, CHIPS, CK, CLEC4M, CPB2, CPN, CR1, CR2, CR3, CR4, CRIg, CRP, CbpA, ClfA, ClfB, DAF, DCN, E-LDL, ELANE, Efb, Ehp, FCN1, FCN2, FGA, FGA, FGB, FGB, FGG, FKBP13, FN, FPR1, FX, FXIII, FXIIa, FXIa, FnBPA, FnBPB, GNA15, GNA1870, GNAI2, GNAI3, Gpm1p, Gpm1p, HIV-gp41, HS, HSV-gC, Heparin, IBSP, ICAM1, ICAM2, ITGA2B, ITGA2, ITGB3, KLKB1, LAMA5, LAMB1, LAMC1, LRP2, LTA, LfhA, MASP1, MAp19, MAp44, MBL2, MCP, NS1, OMCI, OPN, OmpK36, PKA, PKC, PLAUR, PLG, PTX3, Por1A, Por1B, PrP, Protein M, Protein-A, Protein-S, RPS19, RgpA, SAP, SCIN, SCP, SELE, SELL, SELP, SELPLG, SFTPA1, SFTPA2, SIC, SPICE, SSL10, SSL11, SSL5, SSL7, Sbi, Sfb, Skp, StcE, TFECP, THBS, TLR2, TXN, VCP, VTN, WAS, beta-Glucan, cC1qR, fMLP, gC1qR, gp350, iC3b, or a proteoform thereof.

Embodiment 76. The method of Embodiment 71, wherein the one or more proteins are involved in the complement system in neuronal development and plasticity.

Embodiment 77. The method of Embodiment 76, wherein the one or more proteins comprise at least one of: ADP, APAF1, ATP10A, ATP11A, ATP11C, ATP8B1, ATP8B2, ATP8B3, ATP, AXL, BAK1, BAX, BID, C1QA, C1QB, C1QC, C1R, C1S, C2, C2a, C2b, C3, C3AR1, C3a, C3b, C3bi, C3c, C3dg, C3f, C4A, C4B, C4BPA, C4BPB, C4a, C4b, C5, C5AR1, C5AR2, C5a, C5b, C6, C7, C8A, C8B, C8G, C9, CAP1, CASP10, CASP3, CASP7, CASP8, CASP9, CD46, CD55, CD59, CFB, CFBa, CFBb, CFD, CFH, CFI, CFP, CLU, COLEC10, COLEC11, COLEC12, CR1, CR2, CRB1, CRB2, CSMD1, CX3CL1, CX3CR1, CYCS, Ca2+, DEDD, DIABLO, DLGAP5, FAS, FASLG, FCN1, FCN2, FCN3, GAS6, H2O, HTRA2, IFNG, ITGAM, ITGAV, ITGAX, ITGB2, ITGB3, LLGL2, MARK1, MARK2, MARK3, MASP1, MASP2, MBLIP, MBL2, MBP, MERTK, MFGE8, MPP5, PARD3, PARD6A, PARD6B, PARD6G, PATJ, PLSCR1, PLSCR3, PLSCR4, PRKCI, PRKCZ, PROS1, SCRIB, SERPING1, SUSD4, TGFB1, TGFB2, TGFB3, TYRO3, VTN, XIAP, tBID, or a proteoform thereof.

Embodiment 78. The method of Embodiment 71, wherein the one or more proteins are involved in the Kinin-Kallikrein pathway.

Embodiment 79. The method of Embodiment 78, wherein the one or more proteins comprise at least one of: BDKRB1, BDKRB2, Bradykinin, CPN1, Coagulation factor XII, KLKB1, Kininogen-1, SERPING1, des-arg (9) bradykinin, or a proteoform thereof.

Embodiment 80. The method of any one of Embodiments 71-79, wherein the detecting comprises nucleic acid sequencing.

Embodiment 81. The method of any one of Embodiments 71-80, wherein the subject is suspected of having early-stage Alzheimer's Disease.

Embodiment 82. The method of any one of Embodiments 71-80, wherein the subject is asymptomatic for the neurodegenerative disease.

Embodiment 83. The method of any one of Embodiments 71-80, wherein the subject exhibits or reports one or more symptoms of Alzheimer's Disease comprising: impairment in cognition, impairment in memory, impairment in language, or impairment in perception.

Embodiment 84. The method of any one of Embodiments 71-80, wherein the subject has or is suspected of having Alzheimer's Disease based on an expert evaluation.

Embodiment 85. The method of any one of Embodiments 71-80, wherein the subject has or is suspected of having Alzheimer's Disease based on a positive Alzheimer's Disease diagnosis from another diagnostic.

Embodiment 86. A kit for use in determining a risk or state of a neurodegenerative disease of the subject comprising: one or more surfaces capable of binding to a biomarker comprising at least one of: E7EUF1, O94812, P02549, P02730, P05019, P05154, P05546, P13497, P16157, P16452, P17936, P24593, P27918, P35858, P41218, Q12797, Q13214, Q13822, Q8NI99, Q96IY4, Q99715, Q9BXN1, Q9H0B8, or a proteoform thereof, wherein binding of the biomarker to the surface is indicative of the state of the neurodegenerative disease of the subject.

Embodiment 87. A kit for use in determining a risk or state of a neurodegenerative disease of the subject comprising: one or more surfaces capable of binding to a biomarker comprising at least one of: 0A0B4J1U7, A0A0D9SG88; Q5TFM2, A0A3B3ISR2; B4DPQ0; F5H2D0, A5YKK6, A8TX70; A8TX70-2, H3BPZ1, O14960, O14980, O75093, O75900-2, P00748, P00751, P00915, P01042-3, P01892, P02042, P04040, P04180, P04196, P07093-2; P07093-3, P070933, P08833, P09668, P10316, P10321-2, P10643, P11226, P13747, P14625, P16871; P16871-2; P16871-3; P16871-4, P19823, P19827, P21741, P26572, P26927, P28799, P36980, P36980; P36980-2, P40937; P40937-2, P54803, P54803; P54803-3; P54803-4, P78539; P78539-2; P78539-5, Q03591, Q06033; Q06033-2, Q08257-3, Q13131; Q13131-2, Q14213, Q15149-2, Q15149-4, Q15149; Q15149-2; Q15149-4; Q15149-5; Q15149-6; Q15149-7; Q15149-8; Q15149-9, Q15165; Q15165-1; Q15165-3, Q15517, Q16678, Q709C8; Q709C8-3, Q8N474, Q8N6R0; Q8N6R0-1; Q8N6R0-3, Q92896; Q92896-2; Q92896-3, Q93091, Q96PD5, Q96PD5-2, Q9H479, Q9H4F8; Q9H4F8-2, Q9NR34, Q9NUQ9, Q9P035, Q9ULI3, Q9UQN3; Q9UQN3-2, Q9Y263, Q9Y450; Q9Y450-4, or a proteoform thereof, wherein binding of the biomarker to the surface is indicative of the state of the neurodegenerative disease of the subject.

Embodiment 88. The kit of Embodiments 86 or 87, wherein the surface is capable of specifically binding to the biomarker.

Embodiment 89. The kit of Embodiments 86 or 87, wherein the surface is capable of non-specifically binding to the biomarker.

Embodiment 90. The kit of any one of Embodiments 86-89, wherein the surface is capable of releasing the biomarker after the binding.

Embodiment 91. The kit of any one of Embodiments 86-90, wherein the kit comprises a plurality of surfaces capable of binding to the biomarker.

Embodiment 92. The kit of Embodiment 91, wherein the plurality of surfaces comprises at least 2, 3, 4, or 5 surfaces.

Embodiment 93. The kit of Embodiment 92, wherein the plurality of surfaces is capable of binding to a plurality of biomarkers.

Embodiment 94. The kit of Embodiment 93, wherein each surface in the plurality of surfaces are capable of binding to a different biomarker in the plurality of biomarkers.

Embodiment 95. The kit of any one of Embodiments 86-94, wherein the one or more surfaces comprise one or more antibodies capable of binding to the biomarker.

Embodiment 96. The kit of Embodiment 95, wherein a pair of antibodies in the one or more antibodies are capable of binding to the biomarker, wherein the pair of antibodies comprises complementary single-stranded nucleic acid sequences attached thereto, such that when the pair of antibodies bind to the biomarker, the complementary nucleic acids hybridize to form a double stranded nucleic acid.

Embodiment 97. The kit of Embodiment 96, wherein the double stranded nucleic acid is configured to form a binding complex with a polymerase and a plurality of nucleotides, nucleosides, nucleotide analogs, and/or nucleoside analogs to perform an amplification reaction to produce a detectable signal.

Embodiment 98. The kit of any one of Embodiments 86-91, wherein the one or more surfaces comprise one or more aptamers capable of binding to the biomarker.

Embodiment 99. The kit of 98, wherein the one or more aptamers comprise are coupled to a surface via a cleavable linker.

Embodiment 100. The kit of 99, wherein the surface is a particle surface.

Embodiment 101. The kit of 99, wherein the cleavable linker is photocleavable.

Embodiment 102. The kit of 99, further comprising a macromolecular competitor configured to, in a fluid composition, reduce dissociation of a complex comprising the one or more aptamers and the biomarker.

Embodiment 103. The kit of 102, wherein the macromolecular competitor is further configured to bind to a biomolecule that is different from the biomarker.

Embodiment 104. The kit of 102, wherein the macromolecular competitor is a polyanionic macromolecule.

Embodiment 105. A kit for use in determining a risk or state of a neurodegenerative disease of the subject comprising: one or more surfaces capable of binding to a biomarker comprising at least one of: P00451, P00734, P00740, P00742, P00748, P01008, P01023, P01042, P02671, P02675, P02679, P02776, P03951, P03952, P04070, P04275, P05154, P05155, P05160, P05546, P07093, P07204, P07225, P07359, P08709, P10646, P10720, P12259, P13224, P13726, P14770, P24158, P25116, P40197, P42785, Q07021, Q8N6Q3, Q9UNN8, K03901, K01320, K01314, K03902, K01313, K01328, K01323, K01321, K03900, K03899, K03907, K06557, K01344, K03914, K04235, K04236, K03917, K03906, K01300, K03903, K03904, K03905, K01324, K03898, K03915, K03916, K01315, K03909, K03911, K03912, K03913, K03908, K03982, K19821, K01343, K01348, K03985, K03984, K03983, K03910, K01335, K01334, K03990, K03994, K03995, K03996, K03997, K03998, K03999, K04000, K03986, K03987, K03988, K01330, K01331, K03991, K03992, K03993, K01332, K03989, K04009, K19822, K04011, K04012, K06461, K06464, K06462, K04010, K04004, K01333, K04001, K04006, K04007, K04002, K04003, K04008, K17252, K06251, O00255, O00391, O00622, O14672, O14791, O15232, O15240, O43493, O43852, O76024, O76061, O95084, O95633, O95972, P00450, P00734, P00747, P01008, P01009, P01024, P01033, P01034, P01042, P01210, P01344, P02647, P02649, P02652, P02671, P02679, P02751, P02765, P02768, P02771, P02787, P03956, P04070, P04114, P05019, P05060, P05067, P05231, P05546, P06870, P07237, P07288, P07942, P08253, P08311, P08582, P08833, P09382, P09603, P0C0L4, P10451, P11047, P12259, P12644, P13521, P13611, P14314, P14625, P17936, P18065, P19022, P19823, P20151, P20718, P22692, P23327, P24592, P24593, P24821, P34741, P35555, P35858, P51654, P55268, Q02818, Q06481, Q07065, Q08431, Q12841, Q13103, Q13217, Q13219, Q13316, Q13421, Q14393, Q14515, Q14703, Q14766, Q15084, Q15293, Q16270, Q24JP5, Q5JRA6, Q6P988, Q6PCB0, Q6Q788, Q6UX39, Q86UP2, Q8IXL6, Q8N114, Q8N4F0, Q8NBJ4, Q8NBP7, Q8WXD2, Q96AD5, Q96MK3, Q99217, Q9BRK3, Q9BTY2, Q9BU40, Q9BXP8, Q9GZV9, Q9H8M9, Q9HCE9, Q9NP70, Q9NQ76, Q9NRM1, Q9UK55, Q9UKR3, Q9UM21, and a proteoform thereof, wherein the binding of the biomarker to the one or more surfaces are indicative of the state of the neurodegenerative disease of the subject.

Embodiment 106. The kit of Embodiment 105, wherein the one or more surfaces are capable of specifically binding to the biomarker.

Embodiment 107. The kit of Embodiment 105, wherein the one or more surfaces are capable of non-specifically binding to the biomarker.

Embodiment 108. The kit of any one of Embodiments 105-107, wherein the one or more surfaces are capable of releasing the biomarker after the binding.

Embodiment 109. The kit of any one of Embodiments 105-108, wherein the one or more surfaces comprise a plurality of surfaces capable of binding to the biomarker.

Embodiment 110. The kit of Embodiment 109, wherein the plurality of surfaces comprises at least 2, 3, 4, or 5 surfaces.

Embodiment 111. The kit of Embodiment 110, wherein the plurality of surfaces is capable of binding to a plurality of biomarkers.

Embodiment 112. The kit of Embodiment 111, wherein each surface in the plurality of surfaces are capable of binding to a different biomarker in the plurality of biomarkers.

Embodiment 113. A kit for use in determining a risk or state of a neurodegenerative disease of the subject comprising: one or more antibodies capable of binding to a biomarker comprising at least one of: E7EUF1, O94812, P02549, P02730, P05019, P05154, P05546, P13497, P16157, P16452, P17936, P24593, P27918, P35858, P41218, Q12797, Q13214, Q13822, Q8NI99, Q96IY4, Q99715, Q9BXN1, Q9H0B8, or a proteoform thereof, wherein binding of the biomarker to the one or more antibodies is indicative of the state of the neurodegenerative disease of the subject.

Embodiment 114. A kit for use in determining a risk or state of a neurodegenerative disease of the subject comprising: one or more antibodies capable of binding to a biomarker comprising at least one of: 0A0B4J1U7, A0A0D9SG88; Q5TFM2, A0A3B3ISR2; B4DPQ0; F5H2D0, A5YKK6, A8TX70; A8TX70-2, H3BPZ1, O14960, O14980, O75093, O75900-2, P00748, P00751, P00915, P01042-3, P01892, P02042, P04040, P04180, P04196, P07093-2; P07093-3, P070933, P08833, P09668, P10316, P10321-2, P10643, P11226, P13747, P14625, P16871; P16871-2; P16871-3; P16871-4, P19823, P19827, P21741, P26572, P26927, P28799, P36980, P36980; P36980-2, P40937; P40937-2, P54803, P54803; P54803-3; P54803-4, P78539; P78539-2; P78539-5, Q03591, Q06033; Q06033-2, Q08257-3, Q13131; Q13131-2, Q14213, Q15149-2, Q15149-4, Q15149; Q15149-2; Q15149-4; Q15149-5; Q15149-6; Q15149-7; Q15149-8; Q15149-9, Q15165; Q15165-1; Q15165-3, Q15517, Q16678, Q709C8; Q709C8-3, Q8N474, Q8N6R0; Q8N6R0-1; Q8N6R0-3, Q92896; Q92896-2; Q92896-3, Q93091, Q96PD5, Q96PD5-2, Q9H479, Q9H4F8; Q9H4F8-2, Q9NR34, Q9NUQ9, Q9P035, Q9ULI3, Q9UQN3; Q9UQN3-2, Q9Y263, Q9Y450; Q9Y450-4, or a proteoform thereof, wherein binding of the biomarker to the one or more antibodies is indicative of the state of the neurodegenerative disease of the subject.

Embodiment 115. The kit of Embodiments 113 or 114, wherein a pair of antibodies in the one or more antibodies are capable of binding to the biomarker, wherein the pair of antibodies comprises complementary single-stranded nucleic acid sequences attached thereto, such that when the pair of antibodies bind to the biomarker, the complementary nucleic acids hybridize to form a double stranded nucleic acid.

Embodiment 116. The kit of Embodiment 114, wherein the double stranded nucleic acid is configured to form a binding complex with a polymerase and a plurality of nucleotides, nucleosides, nucleotide analogs, and/or nucleoside analogs to perform an amplification reaction to produce a detectable signal.

Embodiment 117. The kit of Embodiment 116, further comprising the polymerase and/or the plurality of nucleotides.

Embodiment 118. A kit for use in determining a risk or state of a neurodegenerative disease of the subject comprising: one or more aptamers capable of binding to a biomarker comprising at least one of: E7EUF1, O94812, P02549, P02730, P05019, P05154, P05546, P13497, P16157, P16452, P17936, P24593, P27918, P35858, P41218, Q12797, Q13214, Q13822, Q8NI99, Q96IY4, Q99715, Q9BXN1, Q9H0B8, or a proteoform thereof, wherein binding of the biomarker to the one or more antibodies is indicative of the state of the neurodegenerative disease of the subject.

Embodiment 119. A kit for use in determining a risk or state of a neurodegenerative disease of the subject comprising: one or more aptamers capable of binding to a biomarker comprising at least one of: 0A0B4J1U7, A0A0D9SG88; Q5TFM2, A0A3B3ISR2; B4DPQ0; F5H2D0, A5YKK6, A8TX70; A8TX70-2, H3BPZ1, O14960, O14980, O75093, O75900-2, P00748, P00751, P00915, P01042-3, P01892, P02042, P04040, P04180, P04196, P07093-2; P07093-3, P070933, P08833, P09668, P10316, P10321-2, P10643, P11226, P13747, P14625, P16871; P16871-2; P16871-3; P16871-4, P19823, P19827, P21741, P26572, P26927, P28799, P36980, P36980; P36980-2, P40937; P40937-2, P54803, P54803; P54803-3; P54803-4, P78539; P78539-2; P78539-5, Q03591, Q06033; Q06033-2, Q08257-3, Q13131; Q13131-2, Q14213, Q15149-2, Q15149-4, Q15149; Q15149-2; Q15149-4; Q15149-5; Q15149-6; Q15149-7; Q15149-8; Q15149-9, Q15165; Q15165-1; Q15165-3, Q15517, Q16678, Q709C8; Q709C8-3, Q8N474, Q8N6R0; Q8N6R0-1; Q8N6R0-3, Q92896; Q92896-2; Q92896-3, Q93091, Q96PD5, Q96PD5-2, Q9H479, Q9H4F8; Q9H4F8-2, Q9NR34, Q9NUQ9, Q9P035, Q9ULI3, Q9UQN3; Q9UQN3-2, Q9Y263, Q9Y450; Q9Y450-4, or a proteoform thereof, wherein binding of the biomarker to the one or more antibodies is indicative of the state of the neurodegenerative disease of the subject.

Embodiment 120. The kit of Embodiments 118 or 119, wherein the one or more aptamers are coupled to a surface via a cleavable linker.

Embodiment 121. The kit of Embodiment 119, wherein the surface is a particle surface.

Embodiment 122. The kit of Embodiment 121, wherein the cleavable linker is photocleavable.

Embodiment 123. The kit of Embodiment 122, further comprising a macromolecular competitor configured to, in a fluid composition, reduce dissociation of a complex comprising the one or more aptamers and the biomarker.

Embodiment 124. The kit of Embodiment 123, wherein the macromolecular competitor is further configured to bind to a biomolecule that is different from the biomarker.

Embodiment 125. The kit of Embodiment 123, wherein the macromolecular competitor is a polyanionic macromolecule.

Embodiment 126. The kit of any one of Embodiments 118-125, wherein the one or more aptamers are coupled to a plurality of surfaces.

Embodiment 127. The kit of Embodiment 126, wherein the plurality of surfaces comprises at least 2, 3, 4, or 5 surfaces.

Embodiment 128. The kit of Embodiment 127, wherein the plurality of surfaces each comprises a different aptamer in the one or more aptamers, wherein each aptamer in the one or more aptamers is capable of binding to a different biomarker.

Embodiment 129. A method for treating a subject afflicted with or at a risk of being afflicted with a neurodegenerative disease, comprising: administering to the subject one or more drugs to modulate a level or an activity of at least one of: E7EUF1, O94812, P02549, P02730, P05019, P05154, P05546, P13497, P16157, P16452, P17936, P24593, P27918, P35858, P41218, Q12797, Q13214, Q13822, Q8NI99, Q96IY4, Q99715, Q9BXN1, Q9H0B8, or a proteoform thereof.

Embodiment 130. The method of Embodiment 129, wherein the biomarker further comprises at least one of: P04114, P30043, P00742, or a proteoform thereof.

Embodiment 131. The method of Embodiment 129 or 130, wherein O94812 proteoform is selected from the group consisting of: O94812-2, O94812-3, O94812-5, O94812-6, and O94812-7.

Embodiment 132. The method of any one of Embodiments 129-131, wherein P02549 is a P02549-2 proteoform.

Embodiment 133. The method of any one of Embodiments 129-132, wherein P35858 is a P35858-2 proteoform.

Embodiment 134. The method of any one of Embodiments 129-133, wherein Q13214 is a Q13214-2 proteoform.

Embodiment 135. The method of any one of Embodiments 129-134, wherein Q13822 is a Q13822-3 proteoform.

Embodiment 136. The method of any one of Embodiments 129-135, wherein Q9H0B8 is a Q9H0B8-3 proteoform.

Embodiment 137. The method of any one of Embodiments 129-136, wherein P16157 is a P16157-12 proteoform.

Embodiment 138. The method of any one of Embodiments 129-137, wherein P16452 is a P16452-2 proteoform.

Embodiment 139. A method for treating a subject afflicted with or at a risk of being afflicted with a neurodegenerative disease, comprising: administering to the subject one or more drugs to modulate a level or an activity of at least one of: GSA-rs1046279, GSA-rs1131364, GSA-rs1165692, GSA-rs6030, rs1001179, rs1046875, rs1046896, rs1061147, rs1061147, rs10737680, rs10801555, rs11003118, rs1106766, rs11085954, rs11613352, rs1165669, rs12067507, rs12148472, rs12493926, rs1329424, rs1329428, rs1410996, rs1520079, rs17198, rs1800450, rs1801020, rs1801689, rs1831282, rs2289702, rs2545801, rs2731672, rs2749534, rs3197999, rs34882957, rs3741414, rs3784539, rs3792366, rs380390, rs395544, rs41268617, rs4524, rs5030062, rs62623707, rs6647, rs6677604, rs6695321, rs68066031, rs68147365, rs698078, rs707998, rs710446, rs7599241, rs8099840, rs880633, rs9820435, and rs9898.

Embodiment 140. A method for determining a risk or state of a neurodegenerative disease of a subject comprising: (a) detecting a presence of a biomarker in a biological sample from the subject, wherein the biomarker comprises at least one of the biomarkers in Table 11; and (b) determining the risk or state of the neurodegenerative disease of the subject based on the presence of the biomarker in the biological sample.

Embodiment 141. The method of any one of Embodiments 129-140, wherein the subject has or is suspected of having Alzheimer's Disease based on a positive Alzheimer's Disease diagnosis from another diagnostic.

Embodiment 142. A device comprising a computer-readable medium having computer-executable code encoded therein, the computer-executable code adapted to be executed to implement any one of the methods of Embodiments 1-85 and 129-141.

Embodiment 143. A computer program product comprising a computer-readable medium having computer-executable code encoded therein, the computer-executable code adapted to be executed to implement any one of the methods of Embodiments 1-85 and 129-141.

Embodiment 144. A non-transitory computer-readable storage media encoded with a computer program including instructions executable by one or more processors to implement any one of the methods of Embodiments 1-85 and 129-141.

Embodiment 145. A computer-implemented system comprising: a digital processing device comprising: at least one processor, an operating system configured to perform executable instructions, a memory, and a computer program including instructions executable by the digital processing device to perform any one of the methods of Embodiments 1-85 and 129-141.

Embodiment 146. A method for determining a risk or state of a neurodegenerative disease of a subject comprising: a. detecting a presence of a biomarker in a biological sample from the subject, wherein the biomarker comprises at least one of: P10451, P00748, Q06481, P02748, Q6Q788, P05060, P0C0L4, or a proteoform thereof; and b. determining the risk or state of the neurodegenerative disease of the subject based on the presence of the biomarker in the biological sample.

Embodiment 147. A kit for use in determining a risk or state of a neurodegenerative disease of the subject comprising: one or more surfaces capable of binding to a biomarker comprising at least one of: P10451, P00748, Q06481, P02748, Q6Q788, P05060, P0C0L4, or a proteoform thereof, wherein binding of the biomarker to the surface is indicative of the state of the neurodegenerative disease of the subject.

Embodiment 148. A kit for use in determining a risk or state of a neurodegenerative disease of the subject comprising: one or more antibodies capable of binding to a biomarker comprising at least one of: P10451, P00748, Q06481, P02748, Q6Q788, P05060, P0C0L4, or a proteoform thereof, wherein binding of the biomarker to the one or more antibodies is indicative of the state of the neurodegenerative disease of the subject.

Embodiment 149. A kit for use in determining a risk or state of a neurodegenerative disease of the subject comprising: one or more aptamers capable of binding to a biomarker comprising at least one of: P10451, P00748, Q06481, P02748, Q6Q788, P05060, P0C0L4, or a proteoform thereof, wherein binding of the biomarker to the one or more antibodies is indicative of the state of the neurodegenerative disease of the subject.

Embodiment 150. A method for treating a subject afflicted with or at a risk of being afflicted with a neurodegenerative disease, comprising: administering to the subject one or more drugs to modulate a level or an activity of at least one of: P10451, P00748, Q06481, P02748, Q6Q788, P05060, P0C0L4, or a proteoform thereof.

Embodiment 151. The method or the kit of any one of Embodiments 146-151, wherein the biomarker comprises at least one of: P00748, Q6Q788, P05060, or a proteoform thereof.

Embodiment 152. A method for determining a risk or state of a neurodegenerative disease of a subject comprising: a. detecting a presence of a biomarker in a biological sample from the subject, wherein the biomarker is involved at least one of degradation of the extracellular matrix; signaling by Platelet-Derived Growth Factor (PDGF); integrin cell surface interactions; regulation of Insulin-like Growth Factor (IGF) transport and uptake by Insulin-like Growth Factor Binding Proteins (IGFBPs); runt-domain transcription factor (RUNX3) which regulates immune response and cell migration; post-translational protein phosphorylation; intrinsic pathway of fibrin clot formation; defective factor XII causing hereditary angioedema; defective SERPING1 causing hereditary angioedema; platelet degranulation; terminal pathway of complement; regulation of Complement cascade; peroxisome proliferator-activated receptor alpha (PPARA) which activates gene expression; assembly of active LPL and LIPC lipase complexes; chylomicron remodeling; initial triggering of complement; and activation of C3 and C5; and b. determining the risk or state of the neurodegenerative disease of the subject based on the presence of the biomarker in the biological sample.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. A method for determining a risk or state of a neurodegenerative disease of a subject comprising:
(a) detecting a presence of a biomarker in a biological sample from the subject, wherein the biomarker comprises at least one of: E7EUF1, O94812, P02549, P02730, P05019, P05154, P05546, P13497, P16157, P16452, P17936, P24593, P27918, P35858, P41218, Q12797, Q13214, Q13822, Q8NI99, Q96IY4, Q99715, Q9BXN1, Q9H0B8, or a proteoform thereof; and
(b) determining the risk or state of the neurodegenerative disease of the subject based on the presence of the biomarker in the biological sample.

2. The method of claim 1, wherein the biomarker further comprises at least one of: P04114, P30043, P00742, or a proteoform thereof.

3. The method of claim 1, wherein the biomarker comprises a O94812 proteoform selected from the group consisting of: O94812-2, O94812-3, O94812-5, O94812-6, and O94812-7.

4. A method for determining a risk or state of a neurodegenerative disease of a subject comprising:
(a) detecting a presence of a biomarker in a biological sample from the subject, wherein the biomarker comprises at least one of: 0A0B4J1U7, A0A0D9SG88; Q5TFM2, A0A3B3ISR2; B4DPQ0; FSH2D0, ASYKK6, A8TX70; ASTX70-2, H3BPZ1, O14960, O14980, O75093, O75900-2, P00748, P00751, P00915, P01042-3, P01892, P02042, P04040, P04180, P04196, P07093-2; P07093-3, P070933, P08833, P09668, P10316, P10321-2, P10643, P11226, P13747, P14625, P16871; P16871-2; P16871-3; P16871-4, P19823, P19827, P21741, P26572, P26927, P28799, P36980, P36980; P36980-2, P40937; P40937-2, P54803, P54803; P54803-3; P54803-4, P78539; P78539-2; P78539-5, Q03591, Q06033; Q06033-2, Q08257-3, Q13131; Q13131-2, Q14213, Q15149-2, Q15149-4, Q15149; Q15149-2; Q15149-4; Q15149-5; Q15149-6; Q15149-7; Q15149-8; Q15149-9, Q15165; Q15165-1; Q15165-3, Q15517, Q16678, Q709C8; Q709C8-3, Q8N474, Q8N6R0; Q8N6R0-1; Q8N6R0-3, Q92896; Q92896-2; Q92896-3, Q93091, Q96PD5, Q96PD5-2, Q9H479, Q9H4F8; Q9H4F8-2, Q9NR34, Q9NUQ9, Q9P035, Q9ULI3, Q9UQN3; Q9UQN3-2, Q9Y263, Q9Y450; Q9Y450-4, or a proteoform thereof; and
(b) determining the risk or state of the neurodegenerative disease of the subject based on the presence of the biomarker in the biological sample.

5. The method of claim 4, wherein the neurodegenerative disease is Alzheimer's disease.

6. The method of claim 4, wherein the biological sample is plasma.

7. The method of claim 4, wherein the presence of the biomarker comprises a pattern of levels of the plurality biomarkers.

8. The method of claim 4, wherein the subject is suspected of having early-stage Alzheimer's Disease.

9. A method for determining a risk or state of a neurodegenerative disease of a subject comprising:
(a) detecting a presence of a biomarker in a biological sample from the subject, wherein the biomarker is involved in a fibrin clot formation cascade; and
(b) determining the risk or state of the neurodegenerative disease of the subject based on the presence of the biomarker in the biological sample.

10. The method of claim 9, wherein the biomarker is involved in an extrinsic pathway of the fibrin clot formation cascade.

11. The method of claim 9, wherein the biomarker is involved in an intrinsic pathway of the fibrin clot formation cascade.

12. The method of claim 9, wherein the biomarker is involved in a common pathway of the fibrin clot formation cascade.

13. A method for determining a risk or state of a disease of a subject comprising:
(a) detecting a presence of one or more single nucleotide polymorphisms (SNPs) in a biological sample from the subject, wherein the one or more SNPs comprise at least one of: GSA-rs1046279, GSA-rs1131364, GSA-ms1165692, GSA-rs6030, rs1001179, rs1046875, rs1046896, rs1061147, rs1061147, rs10737680, rs10801555, rs11003118, rs1106766, rs11085954, rs11613352, rs1165669, rs12067507, rs12148472, rs12493926, rs1329424, rs1329428, rs1410996, rs1520079, rs17198, rs1800450, rs1801020, rs1801689, rs1831282, rs2289702, rs2545801, rs2731672, rs2749534, rs3197999, rs34882957, rs3741414, rs3784539, rs3792366, rs380390, rs395544, rs41268617, rs4524, rs5030062, rs62623707, rs6647, rs6677604, rs6695321, rs68066031, rs68147365, rs698078, rs707998, rs710446, rs7599241, rs8099840, rs880633, rs9820435, rs9898, or a SNP in linkage disequilibrium thereof; and
(b) determining the risk or state of the disease of the subject based on the presence of the one or more SNPs in the biological sample.

14. A method for determining a risk or state of a disease of a subject comprising:
(a) detecting a presence of one or more single nucleotide polymorphisms (SNPs) in a biological sample from the subject, wherein the one or more SNPs comprise at least one of: chr6:29928649, chr6:29939240, exm2260367, exm260986, exm2261461, exm2261761, exm2264447, exm2264486, exm2265845, exm2270485, exm2271590, GSA-rs1131364, GSA-rs11901661, GSA-rs11915474, GSA-rs12085181, GSA-rs13355990, GSA-rs1611704, GSA-rs1651025, GSA-rs17706123, GSA-rs2975041, GSA-rs36013739, GSA-rs4280141, GSA-rs55895668, GSA-rs6030, GSA-rs7088203, GSA-rs76221975, GSA-rs78374057, GSA-rs7872425, GS A-rs7793026, GSA-rs7754077, GSA-rs7088203, rs3923387, rs11718493, rs11764079, rs11777239, rs11783655, rs11915474, rs12085181, rs12148472, rs12258356, rs12493410, rs12493926, rs1426654, rs1611715, rs1736982, rs1800450, rs1801020, rs203850, rs218396, rs2289702, rs2345436, rs2372813, rs2523409, rs2523946, rs2545801, rs272817, rs2735046, rs2735097, rs2743941, rs2975034, rs3115627, rs35620248, rs380390, rs3856650, rs387608, rs395544, rs399419, rs41268617, rs4305381, rs4344876, rs4404487, rs440770, rs4524, rs4740, rs508406, rs6003, rs6049301, rs6512033, rs6677604, rs6783962, rs68066031, rs6890853, rs7014582, rs73466148, rs7464572, rs7493, rs7533936, rs8099840, rs812498, rs9357092, rs9380141, rs9824398, rs9898, seq-rs6993938, seq-rs1126605, or a SNP in linkage disequilibrium thereof; and
(b) determining the risk or state of the disease of the subject based on the presence of the one or more SNPs in the biological sample.

15. A method for determining a risk or state of a neurodegenerative disease of the subject comprising:
(a) providing a biological sample from a subject;
(b) detecting a presence of one or more protein quantitative trait loci (pQTLs) in the biological sample, wherein the one or more pQTLs is associated with a protein level in vivo of one or more proteins comprising at least one of: C9, C8B, F5, CFH, MBL2, F12, KNG1, and SERPINA1; and
(c) determining the state of the neurodegenerative disease of the subject based on the presence of the one or more pQTLs in the biological sample.

16. A method for determining a risk or state of a neurodegenerative disease of a subject comprising:
(a) detecting a presence of one or more single nucleotide polymorphisms (SNPs) in a biological sample from the subject, wherein the one or more SNPs is associated with a protein level in vivo of one or more proteins that are involved in (i) a complement and coagulation cascade, (ii) a complement system, (iii) a complement system in neuronal development and plasticity, (iv) a Kinin-Kallikrein pathway, or (v) any combination thereof; and
(b) determining the risk or state of the neurodegenerative disease of the subject based on the presence of the one or more protein quantitative trait loci in the biological sample.

17. A kit for use in determining a risk or state of a neurodegenerative disease of the subject comprising:
one or more surfaces capable of binding to a biomarker comprising at least one of: E7EUF1, O94812, P02549, P02730, P05019, P05154, P05546, P13497, P16157, P16452, P17936, P24593, P27918, P35858, P41218, Q12797, Q13214, Q13822, Q8NI99, Q96IY4, Q99715, Q9BXN1, Q9H0B8, or a proteoform thereof,
wherein binding of the biomarker to the surface is indicative of the state of the neurodegenerative disease of the subject.

18. A kit for use in determining a risk or state of a neurodegenerative disease of the subject comprising:
one or more surfaces capable of binding to a biomarker comprising at least one of: 0A0B4JIU7, A0A0D9SG88; Q5TFM2, A0A3B3ISR2; B4DPQ0; F5H2D0, A5YKK6, A8TX70; A8TX70-2, H3BPZ1, O14960, O14980, O75093, O75900-2, P00748, P00751, P00915, P01042-3, P01892, P02042, P04040, P04180, P04196, P07093-2; P07093-3, P070933, P08833, P09668, P10316, P10321-2, P10643, P11226, P13747, P14625, P16871; P16871-2; P16871-3; P16871-4, P19823, P19827, P21741, P26572, P26927, P28799, P36980, P36980; P36980-2, P40937; P40937-2, P54803, P54803; P54803-3; P54803-4, P78539; P78539-2; P78539-5, Q03591, Q06033; Q06033-2, Q08257-3, Q13131; Q13131-2, Q14213, Q15149-2, Q15149-4, Q15149; Q15149-2; Q15149-4; Q15149-5; Q15149-6, Q15149-7; Q15149-8; Q15149-9, Q15165; Q15165-1; Q15165-3, Q15517, Q16678, Q709C8; Q709C8-3, Q8N474, Q8N6R0; Q8N6R0-1; Q8N6R0-3, Q92896; Q92896-2; Q92896-3, Q93091, Q96PD5, Q96PD5-2, Q9H479, Q9H4F8; Q9H4F8-2, Q9NR34, Q9NUQ9, Q9P035, Q9ULI3, Q9UQN3; Q9UQN3-2, Q9Y263, Q9Y450; Q9Y450-4, or a proteoform thereof,
wherein binding of the biomarker to the surface is indicative of the state of the neurodegenerative disease of the subject.

19. A kit for use in determining a risk or state of a neurodegenerative disease of the subject comprising:
one or more surfaces capable of binding to a biomarker comprising at least one of: P00451, P00734, P00740, P00742, P00748, P01008, P01023, P01042, P02671, P02675, P02679, P02776, P03951, P03952, P04070, P04275, P05154, P05155, P05160, P05546, P07093, P07204, P07225, P07359, P08709, P10646, P10720, P12259, P13224, P13726, P14770, P24158, P25116, P40197, P42785, Q07021, Q8N6Q3, Q9UNN8, K03901, K01320, K01314, K03902, K01313, K01328, K01323, K01321, K03900, K03899, K03907, K06557, K01344, K03914, K04235, K04236, K03917, K03906, K01300 K03903, K03904, K03905, K01324, K03898, K03915, K03916, K01315, K03909, K03911, K03912, K03913, K03908, K03982, K19821, K01343, K01348, K03985, K03984, K03983, K03910, K01335, K01334, K03990, K03994, K0399S, K03996, K03997, K03998, K03999, K04000, K03986, K03987, K03988, K01330, K01331, K03991, K03992, K03993, K01332, K03989, K04009, K19822, K04011, K04012, K06461, K06464, K06462, K04010, K04004, K01333, K04001, K04006, K04007, K04002, K04003, K04008, K17252, K06251, O00255, O00391, O00622, O14672, O14791, O15232, O15240, O43493, O43852, O76024, O76061, O95084, O95633, O95972, P00450, P00734, P00747, P01008, P01009, P01024, P01033, P01034, P01042, P01210, P01344, P02647, P02649, P02652, P02671, P02679, P02751, P02765, P02768, P02771, P02787, P03956, P04070, P04114, P05019, P05060, P05067, P05231, P05546, P06870, P07237, P07288, P07942, P08253, P08311, P08582, P08833, P09382, P09603, P0C0L4, P10451, P11047, P12259, P12644, P13521, P13611, P14314, P14625, P17936, P18065, P19022, P19823, P20151, P20718, P22692, P23327, P24592, P24593, P24821, P34741, P35555, P35858, P51654, P55268, Q02818, Q06481, Q07065, Q08431, Q12841, Q13103, Q13217, Q13219, Q13316, Q13421, Q14393, Q14515, Q14703, Q14766, Q15084, Q15293, Q16270, Q24JP5, Q5JRA6, Q6P988, Q6PCB0, Q6Q788, Q6UX39, Q86UP2, Q8IXL6, Q8N114, Q8N4F0, Q8NBJ4, Q8NBP7, Q8WXD2, Q96AD5, Q96MK3, Q99217, Q9BRK3, Q9BTY2, Q9BU40, Q9BXP8, Q9GZV9, Q9H8M9, Q9HCE9, Q9NP70, Q9NQ76, Q9NRM1, Q9UK55, Q9UKR3, Q9UM21, and a proteoform thereof,
wherein the binding of the biomarker to the one or more surfaces are indicative of the state of the neurodegenerative disease of the subject.

20. A kit for use in determining a risk or state of a neurodegenerative disease of the subject comprising:
one or more antibodies capable of binding to a biomarker comprising at least one of: E7EUF1, O94812, P02549, P02730, P05019, P05154, P05546, P13497, P16157, P16452, P17936, P24593, P27918, P35858, P41218, Q12797, Q13214, Q13822, Q8NI99, Q96IY4, Q99715, Q9BXN1, Q9H0B8, or a proteoform thereof,
wherein binding of the biomarker to the one or more antibodies is indicative of the state of the neurodegenerative disease of the subject.

21. A kit for use in determining a risk or state of a neurodegenerative disease of the subject comprising:
one or more antibodies capable of binding to a biomarker comprising at least one of: 0A0B4JIU7, A0A0D9SG88; Q5TFM2, A0A3B3ISR2; B4DPQ0; FSH2DO, A5YKK6, A8TX70; A8TX70-2, H3BPZ1, O14960, O14980, O75093, O75900-2, P00748, P00751, P00915, P01042-3, P01892, P02042, P04040, P04180, P04196, P07093-2, P07093-3, P070933, P08833, P09668, P10316, P10321-2, P10643, P11226, P13747, P14625, P16871; P16871-2; P16871-3; P16871-4, P19823, P19827, P21741, P26572, P26927, P28799, P36980, P36980; P36980-2, P40937; P40937-2, P54803, P54803; P54803-3; P54803-4, P78539; P78539-2; P78539-5, Q03591, Q06033; Q06033-2, Q08257-3, Q13131; Q13131-2, Q14213, Q15149-2, Q15149-4, Q15149; Q15149-2; Q15149-4; Q15149-5; Q15149-6; Q15149-7; Q15149-8; Q15149-9, Q15165; Q15165-1; Q15165-3, Q15517, Q16678, Q709C8; Q709C8-3, Q8N474, Q8N6R0; Q8N6R0-1; Q8N6R0-3, Q92896; Q92896-2; Q92896-3, Q93091, Q96PD5, Q96PD5-2, Q9H479, Q9H4F8; Q9H4F8-2, Q9NR34, Q9NUQ9, Q9P035, Q9ULI3, Q9UQN3; Q9UQN3-2, Q9Y263, Q9Y450; Q9Y450-4, or a proteoform thereof,
wherein binding of the biomarker to the one or more antibodies is indicative of the state of the neurodegenerative disease of the subject.

22. A kit for use in determining a risk or state of a neurodegenerative disease of the subject comprising:
one or more aptamers capable of binding to a biomarker comprising at least one of: E7EUF1, O94812, P02549, P02730, P05019, P05154, P05546, P13497, P16157, P16452, P17936, P24593, P27918, P35858, P41218, Q12797, Q13214, Q13822, Q8N199, Q961Y4, Q99715, Q9BXN1, Q9H0B8, or a proteoform thereof,
wherein binding of the biomarker to the one or more antibodies is indicative of the state of the neurodegenerative disease of the subject.

23. A kit for use in determining a risk or state of a neurodegenerative disease of the subject comprising:
one or more aptamers capable of binding to a biomarker comprising at least one of: 0A0B4JIU7, A0A0D9SG88; Q5TFM2, A0A3B3ISR2; B4DPQ0; FSH2D0, A5YKK6, A8TX70; A8TX70-2, H3BPZ1, 014960, 014980, 075093, 075900-2, P00748, P00751, P0091S, P01042-3, P01892, P02042, P04040, P04180, P04196, P07093-2; P07093-3, P070933, P08833, P09668, P10316, P10321-2, P10643, P11226, P13747, P14625, P16871; P16871-2; P16871-3; P16871-4, P19823, P19827, P21741, P26572, P26927, P28799, P36980, P36980; P36980-2, P40937; P40937-2, P54803, P54803; P54803-3; P54803-4, P78539; P78539-2; P78539-5, Q03591, Q06033; Q06033-2, Q08257-3, Q13131; Q13131-2, Q14213, Q15149-2, Q15149-4, Q15149; Q15149-2; Q15149-4; Q15149-5; Q15149-6; Q15149-7; Q15149-8; Q15149-9, Q15165; Q15165-1; Q15165-3, Q15517, Q16678, Q709C8; Q709C8-3, Q8N474, Q8N6R0; Q8N6R0-1; Q8N6R0-3, Q92896; Q92896-2; Q92896-3, Q93091, Q96PD5, Q96PD5-2, Q9H479, Q9H4F8; Q9H4F8-2, Q9NR34, Q9NUQ9, Q9P035, Q9ULI3, Q9UQN3; Q9UQN3-2, Q9Y263, Q9Y450; Q9Y450-4, or a proteoform thereof,
wherein binding of the biomarker to the one or more antibodies is indicative of the state of the neurodegenerative disease of the subject.

24. A method for treating a subject afflicted with or at a risk of being afflicted with a neurodegenerative disease, comprising:
administering to the subject one or more drugs to modulate a level or an activity of at least one of: E7EUF1, O94812, P02549, P02730, P05019, P05154, P05546, P13497, P16157, P16452, P17936, P24593, P27918, P35858, P41218, Q12797, Q13214, Q13822, Q8NI99, Q96IY4, Q99715, Q9BXN1, Q9H0B8, or a proteoform thereof.

25. A method for treating a subject afflicted with or at a risk of being afflicted with a neurodegenerative disease, comprising:
administering to the subject one or more drugs to modulate a level or an activity of at least one of: GSA-rs1046279, GS A-rs1131364, GSA-rs1165692, GSA-rs6030, rs1001179, rs1046875, rs1046896, rs1061147, rs1061147, rs10737680, rs10801555, rs11003118, rs1106766, rs11085954, rs11613352, rs1165669, rs12067507, rs12148472, rs12493926, rs1329424, rs1329428, rs1410996, rs1520079, rs17198, rs1800450, rs1801020, rs1801689, rs1831282, rs2289702, rs2545801, rs2731672, rs2749534, rs3197999, rs34882957, rs3741414, rs3784539, rs3792366, rs380390, rs395544, rs41268617, rs4524, rs5030062, rs62623707, rs6647, rs6677604, rs6695321, rs68066031, rs68147365, rs698078, rs707998, rs710446, rs7599241, rs8099840, rs880633, rs9820435, and rs9898.

26. A method for determining a risk or state of a neurodegenerative disease of a subject comprising:
(a) detecting a presence of a biomarker in a biological sample from the subject, wherein the biomarker comprises at least one of the biomarkers in Table 11; and
(b) determining the risk or state of the neurodegenerative disease of the subject based on the presence of the biomarker in the biological sample.

27. A method for determining a risk or state of a neurodegenerative disease of a subject comprising:
(a) detecting a presence of a biomarker in a biological sample from the subject, wherein the biomarker comprises at least one of: P10451, P00748, Q06481, P02748, Q6Q788, P05060, P0C0L4, or a proteoform thereof; and
(b) determining the risk or state of the neurodegenerative disease of the subject based on the presence of the biomarker in the biological sample.

28. A kit for use in determining a risk or state of a neurodegenerative disease of the subject comprising:
one or more surfaces capable of binding to a biomarker comprising at least one of: P10451, P00748, Q06481, P02748, Q6Q788, P05060, P0C0L4, or a proteoform thereof,
wherein binding of the biomarker to the surface is indicative of the state of the neurodegenerative disease of the subject.

29. A kit for use in determining a risk or state of a neurodegenerative disease of the subject comprising:
one or more antibodies capable of binding to a biomarker comprising at least one of: P10451, P00748, Q06481, P02748, Q6Q788, P05060, POCOLA, or a proteoform thereof,
wherein binding of the biomarker to the one or more anti bodies is indicative of the state of the neurodegenerative disease of the subject.

30. A kit for use in determining a risk or state of a neurodegenerative disease of the subject comprising:
one or more aptamers capable of binding to a biomarker comprising at least one of: P10451, P00748, Q06481, P02748, Q6Q788, P05060, P0C0L4, or a proteoform thereof,
wherein binding of the biomarker to the one or more antibodies is indicative of the state of the neurodegenerative disease of the subject.

31. A method for treating a subject afflicted with or at a risk of being afflicted with a neurodegenerative disease, comprising:
administering to the subject one or more drugs to modulate a level or an activity of at least one of: P10451, P00748, Q06481, P02748, Q6Q788, P05060, P0C0L4, or a proteoform thereof.

32. The method or the kit of any one of claims 27-31, wherein the biomarker comprises at least one of: P00748, Q6Q788, P05060, or a proteoform thereof.

33. A method for determining a risk or state of a neurodegenerative disease of a subject comprising:
(a) detecting a presence of a biomarker in a biological sample from the subject, wherein the biomarker is involved at least one of:
i. degradation of the extracellular matrix;
ii. signaling by Platelet-Derived Growth Factor (PDGF);
iii. integrin cell surface interactions;
iv. regulation of Insulin-like Growth Factor (IGF) transport and uptake by Insulin-like Growth Factor Binding Proteins (IGFBPs);
v. runt-domain transcription factor (RUNX3) which regulates immune response and cell migration;
vi. post-translational protein phosphorylation;
vii. intrinsic pathway of fibrin clot formation;
viii. defective factor XII causing hereditary angioedema;
ix. defective SERPING1 causing hereditary angioedema;
x. platelet degranulation;
xi. terminal pathway of complement;
xii. regulation of Complement cascade;
xiii. peroxisome proliferator-activated receptor alpha (PPARA) which activates gene expression;
xiv. assembly of active LPL and LIPC lipase complexes;
xv. chylomicron remodeling;
xvi. initial triggering of complement; and
xvii. activation of C3 and C5; and
(b) determining the risk or state of the neurodegenerative disease of the subject based on the presence of the biomarker in the biological sample.


SEQUENCE LISTING (98 sequences)

SEQ ID NO: 1 - protein, 488 aa, Homo sapiens
MGRPLHLVLLSASLAGLLLLGESLFIRREQANNILARVTRANSFLEEMKKGHLERECMEETCSYEEAREVFEDSDKTNEFWNKYKDGDQCETSPCQNQGKCKDGLGEYTCTCLEGFEGKNCELFTRKLCSLDNGDCDQFCHEEQNSVVCSCARGYTLADNGKACIPTGPYPCGKQTLERRKRSVAQATSSSGEAPDSITWKPYDAADLDPTENPFDLLDFNQTQPERGDNNLTRIVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPLK

SEQ ID NO: 2 - protein, 911 aa, Homo sapiens
MEELQDDYEDMMEENLEQEEYEDPDIPESQMEEPAAHDTEATATDYHTTSHPGTHKVYVELQELVMDEKNQELRWMEAARWVQLEENLGENGAWGRPHLSHLTFWSLLELRRVFTKGTVLLDLQETSLAGVANQLLDRFIFEDQIRPQDREELLRALLLKHSHAGELEALGGVKPAVLTRSGDPSQPLLPQHSSLETQLFCEQGDGGTEGHSPSGILEKIPPDSEATLVLVGRADFLEQPVLGFVRLQEAAELEAVELPVPIRFLFVLLGPEAPHIDYTQLGRAAATLMSERVFRIDAYMAQSRGELLHSLEGFLDCSLVLPPTDAPSEQALLSLVPVQRELLRRRYQSSPAKPDSSFYKGLDLNGGPDDPLQQTGQLFGGLVRDIRRRYPYYLSDITDAFSPQVLAAVIFIYFAALSPAITFGGLLGEKTRNQMGVSELLISTAVQGILFALLGAQPLLVVGFSGPLLVFEEAFFSFCETNGLEYIVGRVWIGFWLILLVVLVVAFEGSFLVRFISRYTQEIFSFLISLIFIYETFSKLIKIFQDHPLQKTYNYNVLMVPKPQGPLPNTALLSLVLMAGTFFFAMMLRKFKNSSYFPGKLRRVIGDFGVPISILIMVLVDFFIQDTYTQKLSVPDGFKVSNSSARGWVIHPLGLRSEFPIWMMFASALPALLVFILIFLESQITTLIVSKPERKMVKGSGFHLDLLLVVGMGGVAALFGMPWLSATTVRSVTHANALTVMGKASTPGAAAQIQEVKEQRISGLLVAVLVGLSILMEPILSRIPLAVLFGIFLYMGVTSLSGIQLFDRILLLFKPPKYHPDVPYVKRVKTWRMHLFTGIQIICLAVLWVVKSTPASLALPFVLILTVPLRRVLLPLIFRNVELQCLDADDAKATFDEEEGRDEYDEVAMPV

SEQ ID NO: 3 - protein, 846 aa, Homo sapiens
MDEKNQELRWMEAARWVQLEENLGENGAWGRPHLSHLTFWSLLELRRVFTKGTVLLDLQETSLAGVANQLLDRFIFEDQIRPQDREELLRALLLKHSHAGELEALGGVKPAVLTRSGDPSQPLLPQHSSLETQLFCEQGDGGTEGHSPSGILEKIPPDSEATLVLVGRADFLEQPVLGFVRLQEAAELEAVELPVPIRFLFVLLGPEAPHIDYTQLGRAAATLMSERVFRIDAYMAQSRGELLHSLEGFLDCSLVLPPTDAPSEQALLSLVPVQRELLRRRYQSSPAKPDSSFYKGLDLNGGPDDPLQQTGQLFGGLVRDIRRRYPYYLSDITDAFSPQVLAAVIFIYFAALSPAITFGGLLGEKTRNQMGVSELLISTAVQGILFALLGAQPLLVVGFSGPLLVFEEAFFSFCETNGLEYIVGRVWIGFWLILLVVLVVAFEGSFLVRFISRYTQEIFSFLISLIFIYETFSKLIKIFQDHPLQKTYNYNVLMVPKPQGPLPNTALLSLVLMAGTFFFAMMLRKFKNSSYFPGKLRRVIGDFGVPISILIMVLVDFFIQDTYTQKLSVPDGFKVSNSSARGWVIHPLGLRSEFPIWMMFASALPALLVFILIFLESQITTLIVSKPERKMVKGSGFHLDLLLVVGMGGVAALFGMPWLSATTVRSVTHANALTVMGKASTPGAAAQIQEVKEQRISGLLVAVLVGLSILMEPILSRIPLAVLFGIFLYMGVTSLSGIQLFDRILLLFKPPKYHPDVPYVKRVKTWRMHLFTGIQIICLAVLWVVKSTPASLALPFVLILTVPLRRVLLPLIFRNVELQCLDADDAKATFDEEEGRDEYDEVAMPV

SEQ ID NO: 4 - protein, 4563 aa, Homo sapiens
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYKNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPQTDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSYDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVLENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDILEHVKHFVINLIGDFEVAEKINAFRAKVHELIERYEVDQQIQVLMDKLVELAHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMLNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPDFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVATTTKAQIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFIIPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTFAHRDFSAEYEEDGKYEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQEFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFSDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL

SEQ ID NO: 5 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

SEQ ID NO: 6 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 7 - protein, 137 aa, Homo sapiens
MITPTVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 8 - protein, 158 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGSTFEERK

SEQ ID NO: 9 - protein, 406 aa, Homo sapiens
MQLFLLLCLVLLSPQGASLHRHHPREMKKRVEDLHVGATVAPSSRRDFTFDLYRALASAAPSQSIFFSPVSISMSLAMLSLGAGSSTKMQILEGLGLNLQKSSEKELHRGFQQLLQELNQPRDGFQLSLGNALFTDLVVDLQDTFVSAMKTLYLADTFPTNFRDSAGAMKQINDYVAKQTKGKIVDLLKNLDSNAVVIMVNYIFFKAKWETSFNHKGTQEQDFYVTSETVVRVPMMSREDQYHYLLDRNLSCRVVGVPYQGNATALFILPSEGKMQQVENGLSEKTLRKWLKMFKKRQLELYLPKFSIEGSYQLEKVLPSLGISNVFTSHADLSGISNHSNIQVSEMVHKAVVEVDESGTRAAAATGTIFTFRSARLNSQRLVFNRPFLMFIVDNNILFLGKVNRP

SEQ ID NO: 10 - protein, 986 aa, Homo sapiens
MPGVARLPLLLGLLLLPRPGRPLDLADYTYDLAEEDDSEPLNYKDPCKAAAFLGDIALDEEDLRAFQVQQAVDLRRHTARKSSIKAAVPGNTSTPSCQSTNGQPQRGACGRWRGRSRSRRAATSRPERVWPDGVIPFVIGGNFTGSQRAVFRQAMRHWEKHTCVTFLERTDEDSYIVFTYRPCGCCSYVGRRGGGPQAISIGKNCDKFGIVVHELGHVVGFWHEHTRPDRDRHVSIVRENIQPGQEYNFLKMEPQEVESLGETYDFDSIMHYARNTFSRGIFLDTIVPKYEVNGVKPPIGQRTRLSKGDIAQARKLYKCPACGETLQDSTGNFSSPEYPNGYSAHMHCVWRISVTPGEKIILNFTSLDLYRSRLCWYDYVEVRDGFWRKAPLRGRFCGSKLPEPIVSTDSRLWVEFRSSSNWVGKGFFAVYEAICGGDVKKDYGHIQSPNYPDDYRPSKVCIWRIQVSEGFHVGLTFQSFEIERHDSCAYDYLEVRDGHSESSTLIGRYCGYEKPDDIKSTSSRLWLKFVSDGSINKAGFAVNFFKEVDECSRPNRGGCEQRCLNTLGSYKCSCDPGYELAPDKRRCEAACGGFLTKLNGSITSPGWPKEYPPNKNCIWQLVAPTQYRISLQFDFFETEGNDVCKYDFVEVRSGLTADSKLHGKFCGSEKPEVITSQYNNMRVEFKSDNTVSKKGFKAHFFSDKDECSKDNGGCQQDCVNTFGSYECQCRSGFVLHDNKHDCKEAGCDHKVTSTSGTITSPNWPDKYPSKKECTWAISSTPGHRVKLTFMEMDIESQPECAYDHLEVFDGRDAKAPVLGRFCGSKKPEPVLATGSRMFLRFYSDNSVQRKGFQASHATECGGQVRADVKTKDLYSHAQFGDNNYPGGVDCEWVIVAEEGYGVELVFQTFEVEEETDCGYDYMELFDGYDSTAPRLGRYCGSGPPEEVYSAGDSVLVKFHSDDTITKKGFHLRYTSTKFQDTLHSRK

SEQ ID NO: 11 - protein, 730 aa, Homo sapiens
MPGVARLPLLLGLLLLPRPGRPLDLADYTYDLAEEDDSEPLNYKDPCKAAAFLGDIALDEEDLRAFQVQQAVDLRRHTARKSSIKAAVPGNTSTPSCQSTNGQPQRGACGRWRGRSRSRRAATSRPERVWPDGVIPFVIGGNFTGSQRAVFRQAMRHWEKHTCVTFLERTDEDSYIVFTYRPCGCCSYVGRRGGGPQAISIGKNCDKFGIVVHELGHVVGFWHEHTRPDRDRHVSIVRENIQPGQEYNFLKMEPQEVESLGETYDFDSIMHYARNTFSRGIFLDTIVPKYEVNGVKPPIGQRTRLSKGDIAQARKLYKCPACGETLQDSTGNFSSPEYPNGYSAHMHCVWRISVTPGEKIILNFTSLDLYRSRLCWYDYVEVRDGFWRKAPLRGRFCGSKLPEPIVSTDSRLWVEFRSSSNWVGKGFFAVYEAICGGDVKKDYGHIQSPNYPDDYRPSKVCIWRIQVSEGFHVGLTFQSFEIERHDSCAYDYLEVRDGHSESSTLIGRYCGYEKPDDIKSTSSRLWLKFVSDGSINKAGFAVNFFKEVDECSRPNRGGCEQRCLNTLGSYKCSCDPGYELAPDKRRCEAACGGFLTKLNGSITSPGWPKEYPPNKNCIWQLVAPTQYRISLQFDFFETEGNDVCKYDFVEVRSGLTADSKLHGKFCGSEKPEVITSQYNNMRVEFKSDNTVSKKGFKAHFFSEKRPALQPPRGRPHQLKFRVQKRNRTPQ

SEQ ID NO: 12 - protein, 302 aa, Homo sapiens
MPGVARLPLLLGLLLLPRPGRPLDLADYTYDLAEEDDSEPLNYKDPCKAAAFLGDIALDEEDLRAFQVQQAVDLRRHTARKSSIKAAVPGNTSTPSCQSTNGQPQRGACGRWRGRSRSRRAATSRPERVWPDGVIPFVIGGNFTGSQRAVFRQAMRHWEKHTCVTFLERTDEDSYIVFTYRPCGCCSYVGRRGGGPQAISIGKNCDKFGIVVHELGHVVGFWHEHTRPDRDRHVSIVRENIQPGVLHSSLLLLSCGSRNGASFPCSLESSTHQALCWTGLFLRPSPFPRLPLAAPRTLRAGV

SEQ ID NO: 13 - protein, 622 aa, Homo sapiens
MPGVARLPLLLGLLLLPRPGRPLDLADYTYDLAEEDDSEPLNYKDPCKAAAFLGDIALDEEDLRAFQVQQAVDLRRHTARKSSIKAAVPGNTSTPSCQSTNGQPQRGACGRWRGRSRSRRAATSRPERVWPDGVIPFVIGGNFTGSQRAVFRQAMRHWEKHTCVTFLERTDEDSYIVFTYRPCGCCSYVGRRGGGPQAISIGKNCDKFGIVVHELGHVVGFWHEHTRPDRDRHVSIVRENIQPGQEYNFLKMEPQEVESLGETYDFDSIMHYARNTFSRGIFLDTIVPKYEVNGVKPPIGQRTRLSKGDIAQARKLYKCPACGETLQDSTGNFSSPEYPNGYSAHMHCVWRISVTPGEKIILNFTSLDLYRSRLCWYDYVEVRDGFWRKAPLRGRFCGSKLPEPIVSTDSRLWVEFRSSSNWVGKGFFAVYEAICGGDVKKDYGHIQSPNYPDDYRPSKVCIWRIQVSEGFHVGLTFQSFEIERHDSCAYDYLEVRDGHSESSTLIGRYCGYEKPDDIKSTSSRLWLKFVSDGSINKAGFAVNFFKEVDECSRPNRGGCEQRCLNTLGSYKCSCDPGYELAPDKRRCEGCYDLQVGKPLLWDRHCFRLSTHGPEMLGTALRG

SEQ ID NO: 14 - protein, 717 aa, Homo sapiens
MPGVARLPLLLGLLLLPRPGRPLDLADYTYDLAEEDDSEPLNYKDPCKAAAFLGDIALDEEDLRAFQVQQAVDLRRHTARKSSIKAAVPGNTSTPSCQSTNGQPQRGACGRWRGRSRSRRAATSRPERVWPDGVIPFVIGGNFTGSQRAVFRQAMRHWEKHTCVTFLERTDEDSYIVFTYRPCGCCSYVGRRGGGPQAISIGKNCDKFGIVVHELGHVVGFWHEHTRPDRDRHVSIVRENIQPGQEYNFLKMEPQEVESLGETYDFDSIMHYARNTFSRGIFLDTIVPKYEVNGVKPPIGQRTRLSKGDIAQARKLYKCPACGETLQDSTGNFSSPEYPNGYSAHMHCVWRISVTPGEKIILNFTSLDLYRSRLCWYDYVEVRDGFWRKAPLRGRFCGSKLPEPIVSTDSRLWVEFRSSSNWVGKGFFAVYEAICGGDVKKDYGHIQSPNYPDDYRPSKVCIWRIQVSEGFHVGLTFQSFEIERHDSCAYDYLEVRDGHSESSTLIGRYCGYEKPDDIKSTSSRLWLKFVSDGSINKAGFAVNFFKEVDECSRPNRGGCEQRCLNTLGSYKCSCDPGYELAPDKRRCEAACGGFLTKLNGSITSPGWPKEYPPNKNCIWQLVAPTQYRISLQFDFFETEGNDVCKYDFVEVRSGLTADSKLHGKFCGSEKPEVITSQYNNMRVEFKSDNTVSKKGFKAHFFSGGELFGLLGHPPRRP

SEQ ID NO: 15 - protein, 823 aa, Homo sapiens
MPGVARLPLLLGLLLLPRPGRPLDLADYTYDLAEEDDSEPLNYKDPCKAAAFLGDIALDEEDLRAFQVQQAVDLRRHTARKSSIKAAVPGNTSTPSCQSTNGQPQRGACGRWRGRSRSRRAATSRPERVWPDGVIPFVIGGNFTGSQRAVFRQAMRHWEKHTCVTFLERTDEDSYIVFTYRPCGCCSYVGRRGGGPQAISIGKNCDKFGIVVHELGHVVGFWHEHTRPDRDRHVSIVRENIQPGQEYNFLKMEPQEVESLGETYDFDSIMHYARNTFSRGIFLDTIVPKYEVNGVKPPIGQRTRLSKGDIAQARKLYKCPACGETLQDSTGNFSSPEYPNGYSAHMHCVWRISVTPGEKIILNFTSLDLYRSRLCWYDYVEVRDGFWRKAPLRGRFCGSKLPEPIVSTDSRLWVEFRSSSNWVGKGFFAVYEAICGGDVKKDYGHIQSPNYPDDYRPSKVCIWRIQVSEGFHVGLTFQSFEIERHDSCAYDYLEVRDGHSESSTLIGRYCGYEKPDDIKSTSSRLWLKFVSDGSINKAGFAVNFFKEVDECSRPNRGGCEQRCLNTLGSYKCSCDPGYELAPDKRRCEAACGGFLTKLNGSITSPGWPKEYPPNKNCIWQLVAPTQYRISLQFDFFETEGNDVCKYDFVEVRSGLTADSKLHGKFCGSEKPEVITSQYNNMRVEFKSDNTVSKKGFKAHFFSVLEGAGDRHSHLSGLELLLCPHALVDTVPAPPSALHGDTHAHTHTHVHTHCPIAQETCRGPPLGASRLSPQGPGHLTLAPQEGSYLDFWDTHRGDPKPRRRRKSLKTFSLTPATFRGIWAL

SEQ ID NO: 16 - skipped

SEQ ID NO: 17 - protein, 1881 aa, Homo sapiens
MPYSVGFREADAATSFLRAARSGNLDKALDHLRNGVDINTCNQNGLNGLHLASKEGHVKMVVELLHKEIILETTTKKGNTALHIAALAGQDEVVRELVNYGANVNAQSQKGFTPLYMAAQENHLEVVKFLLENGANQNVATEDGFTPLAVALQQGHENVVAHLINYGTKGKVRLPALHIAARNDDTRTAAVLLQNDPNPDVLSKTGFTPLHIAAHYENLNVAQLLLNRGASVNFTPQNGITPLHIASRRGNVIMVRLLLDRGAQIETKTKDELTPLHCAARNGHVRISEILLDHGAPIQAKTKNGLSPIHMAAQGDHLDCVRLLLQYDAEIDDITLDHLTPLHVAAHCGHHRVAKVLLDKGAKPNSRALNGFTPLHIACKKNHVRVMELLLKTGASIDAVTESGLTPLHVASFMGHLPIVKNLLQRGASPNVSNVKVETPLHMAARAGHTEVAKYLLQNKAKVNAKAKDDQTPLHCAARIGHTNMVKLLLENNANPNLATTAGHTPLHIAAREGHVETVLALLEKEASQACMTKKGFTPLHVAAKYGKVRVAELLLERDAHPNAAGKNGLTPLHVAVHHNNLDIVKLLLPRGGSPHSPAWNGYTPLHIAAKQNQVEVARSLLQYGGSANAESVQGVTPLHLAAQEGHAEMVALLLSKQANGNLGNKSGLTPLHLVAQEGHVPVADVLIKHGVMVDATTRMGYTPLHVASHYGNIKLVKFLLQHQADVNAKTKLGYSPLHQAAQQGHTDIVTLLLKNGASPNEVSSDGTTPLAIAKRLGYISVTDVLKVVTDETSFVLVSDKHRMSFPETVDEILDVSEDEGEELISFKAERRDSRDVDEEKELLDFVPKLDQVVESPAIPRIPCAMPETVVIRSEEQEQASKEYDEDSLIPSSPATETSDNISPVASPVHTGFLVSFMVDARGGSMRGSRHNGLRVVIPPRTCAAPTRITCRLVKPQKLSTPPPLAEEEGLASRIIALGPTGAQFLSPVIVEIPHFASHGRGDRELVVLRSENGSVWKEHRSRYGESYLDQILNGMDEELGSLEELEKKRVCRIITTDFPLYFVIMSRLCQDYDTIGPEGGSLKSKLVPLVQATFPENAVTKRVKLALQAQPVPDELVTKLLGNQATFSPIVTVEPRRRKFHRPIGLRIPLPPSWTDNPRDSGEGDTTSLRLLCSVIGGTDQAQWEDITGTTKLVYANECANFTTNVSARFWLSDCPRTAEAVNFATLLYKELTAVPYMAKFVIFAKMNDPREGRLRCYCMTDDKVDKTLEQHENFVEVARSRDIEVLEGMSLFAELSGNLVPVKKAAQQRSFHFQSFRENRLAMPVKVRDSSREPGGSLSFLRKAMKYEDTQHILCHLNITMPPCAKGSGAEDRRRTPTPLALRYSILSESTPGSLSGTEQAEMKMAVISEHLGLSWAELARELQFSVEDINRIRVENPNSLLEQSVALLNLWVIREGQNANMENLYTALQSIDRGEIVNMLEGSGRQSRNLKPDRRHTDRDYSLSPSQMNGYSSLQDELLSPASLGCALSSPLRADQYWNEVAVLDAIPLAATEHDTMLEMSDMQVWSAGLTPSLVTAEDSSLECSKAEDSDATGHEWKLEGALSEEPRGPELGSLELVEDDTVDSDATNGLIDLLEQEEGQRSEEKLPGSKRQDDATGAGQDSENEVSLVSGHQRGQARITHSPTVSQVTERSQDRLQDWDADGSIVSYLQDAAQGSWQEEVTQGPHSFQGTSTMTEGLEPGGSQEYEKVLVSVSEHTWTEQPEAESSQADRDRRQQGQEEQVQEAKNTFTQVVQGNEFQNIPGEQVTEEQFTDEQGNIVTKKIIRKVVRQIDLSSADAAQEHEEVTVEGPLEDPSELEVDIDYFMKHSKDHTSTPNP

SEQ ID NO: 18 - protein, 1519 aa, Homo sapiens
MPYSVGFREADAATSFLRAARSGNLDKALDHLRNGVDINTCNQNGLNGLHLASKEGHVKMVVELLHKEIILETTTKKGNTALHIAALAGQDEVVRELVNYGANVNAQSQKGFTPLYMAAQENHLEVVKFLLENGANQNVATEDGFTPLAVALQQGHENVVAHLINYGTKGKVRLPALHIAARNDDTRTAAVLLQNDPNPDVLSKTGFTPLHIAAHYENLNVAQLLLNRGASVNFTPQNGITPLHIASRRGNVIMVRLLLDRGAQIETKTKDELTPLHCAARNGHVRISEILLDHGAPIQAKTKNGLSPIHMAAQGDHLDCVRLLLQYDAEIDDITLDHLTPLHVAAHCGHHRVAKVLLDKGAKPNSRALNGFTPLHIACKKNHVRVMELLLKTGASIDAVTESGLTPLHVASFMGHLPIVKNLLQRGASPNVSNVKVETPLHMAARAGHTEVAKYLLQNKAKVNAKAKDDQTPLHCAARIGHTNMVKLLLENNANPNLATTAGHTPLHIAAREGHVETVLALLEKEASQACMTKKGFTPLHVAAKYGKVRVAELLLERDAHPNAAGKNGLTPLHVAVHHNNLDIVKLLLPRGGSPHSPAWNGYTPLHIAAKQNQVEVARSLLQYGGSANAESVQGVTPLHLAAQEGHAEMVALLLSKQANGNLGNKSGLTPLHLVAQEGHVPVADVLIKHGVMVDATTRMGYTPLHVASHYGNIKLVKFLLQHQADVNAKTKLGYSPLHQAAQQGHTDIVTLLLKNGASPNEVSSDGTTPLAIAKRLGYISVTDVLKVVTDETSFVLVSDKHRMSFPETVDEILDVSEDEGEELISFKAERRDSRDVDEEKELLDFVPKLDQVVESPAIPRIPCAMPETVVIRSEEQEQASKEYDEDSLIPSSPATETSDNISPVASPVHTGFLVSFMVDARGGSMRGSRHNGLRVVIPPRTCAAPTRITCRLVKPQKLSTPPPLAEEEGLASRIIALGPTGAQFLSPVIVEIPHFASHGRGDRELVVLRSENGSVWKEHRSRYGESYLDQILNGMDEELGSLEELEKKRVCRIITTDFPLYFVIMSRLCQDYDTIGPEGGSLKSKLVPLVQATFPENAVTKRVKLALQAQPVPDELVTKLLGNQATFSPIVTVEPRRRKFHRPIGLRIPLPPSWTDNPRDSGEGDTTSLRLLCSVIGGTDQAQWEDITGTTKLVYANECANFTTNVSARFWLSDCPRTAEAVNFATLLYKELTAVPYMAKFVIFAKMNDPREGRLRCYCMTDDKVDKTLEQHENFVEVARSRDIEVLEGMSLFAELSGNLVPVKKAAQQRSFHFQSFRENRLAMPVKVRDSSREPGGSLSFLRKAMKYEDTQHILCHLNITMPPCAKGSGAEDRRRTPTPLALRYSILSESTPGSLSGTEQAEMKMAVISEHLGLSWAELARELQFSVEDINRIRVENPNSLLEQSVALLNLWVIREGQNANMENLYTALQSIDRGEIVNMLEGSGRQSRNLKPDRRHTDRDYSLSPSQMNDTSTPNP

SEQ ID NO: 19 - protein, 1880 aa, Homo sapiens
MPYSVGFREADAATSFLRAARSGNLDKALDHLRNGVDINTCNQNGLNGLHLASKEGHVKMVVELLHKEIILETTTKKGNTALHIAALAGQDEVVRELVNYGANVNAQSQKGFTPLYMAAQENHLEVVKFLLENGANQNVATEDGFTPLAVALQQGHENVVAHLINYGTKGKVRLPALHIAARNDDTRTAAVLLQNDPNPDVLSKTGFTPLHIAAHYENLNVAQLLLNRGASVNFTPQNGITPLHIASRRGNVIMVRLLLDRGAQIETKTKDELTPLHCAARNGHVRISEILLDHGAPIQAKTKNGLSPIHMAAQGDHLDCVRLLLQYDAEIDDITLDHLTPLHVAAHCGHHRVAKVLLDKGAKPNSRALNGFTPLHIACKKNHVRVMELLLKTGASIDAVTESGLTPLHVASFMGHLPIVKNLLQRGASPNVSNVKVETPLHMAARAGHTEVAKYLLQNKAKVNAKAKDDQTPLHCAARIGHTNMVKLLLENNANPNLATTAGHTPLHIAAREGHVETVLALLEKEASQACMTKKGFTPLHVAAKYGKVRVAELLLERDAHPNAAGKNGLTPLHVAVHHNNLDIVKLLLPRGGSPHSPAWNGYTPLHIAAKQNQVEVARSLLQYGGSANAESVQGVTPLHLAAQEGHAEMVALLLSKQANGNLGNKSGLTPLHLVAQEGHVPVADVLIKHGVMVDATTRMGYTPLHVASHYGNIKLVKFLLQHQADVNAKTKLGYSPLHQAAQQGHTDIVTLLLKNGASPNEVSSDGTTPLAIAKRLGYISVTDVLKVVTDETSFVLVSDKHRMSFPETVDEILDVSEDEGEELISFKAERRDSRDVDEEKELLDFVPKLDQVVESPAIPRIPCAMPETVVIRSEEQEQASKEYDEDSLIPSSPATETSDNISPVASPVHTGFLVSFMVDARGGSMRGSRHNGLRVVIPPRTCAAPTRITCRLVKPQKLSTPPPLAEEEGLASRIIALGPTGAQFLSPVIVEIPHFASHGRGDRELVVLRSENGSVWKEHRSRYGESYLDQILNGMDEELGSLEELEKKRVCRIITTDFPLYFVIMSRLCQDYDTIGPEGGSLKSKLVPLVQATFPENAVTKRVKLALQAQPVPDELVTKLLGNQATFSPIVTVEPRRRKFHRPIGLRIPLPPSWTDNPRDSGEGDTTSLRLLCSVIGGTDQAQWEDITGTTKLVYANECANFTTNVSARFWLSDCPRTAEAVNFATLLYKELTAVPYMAKFVIFAKMNDPREGRLRCYCMTDDKVDKTLEQHENFVEVARSRDIEVLEGMSLFAELSGNLVPVKKAAQQRSFHFQSFRENRLAMPVKVRDSSREPGGSLSFLRKAMKYEDTQHILCHLNITMPPCAKGSGAEDRRRTPTPLALRYSILSESTPGSLSGTEQAEMKMAVISEHLGLSWAELARELQFSVEDINRIRVENPNSLLEQSVALLNLWVIREGQNANMENLYTALQSIDRGEIVNMLEGSGRQSRNLKPDRRHTDRDYSLSPSQMNGYSSLQDELLSPASLGCALSSPLRADQYWNEVAVLDAIPLAATEHDTMLEMSDMQVWSAGLTPSLVTAEDSSLECSKAEDSDATGHEWKLEGALSEEPRGPELGSLELVEDDTVDSDATNGLIDLLEQEEGQRSEEKLPGSKRQDDATGAGQDSENEVSLVSGHQRGQARITHSPTVSQVTERSQDRLQDWDADGSIVSYLQDAAQGSWQEEVTQGPHSFQGTSTMTEGLEPGGSQEYEKVLVSVSEHTWTEQPEAESSQADRDRRQQGQEEQVQEAKNTFTQVVQGNEFQNIPGEQVTEEQFTDEQGNIVTKKIIRKVVRQIDLSSADAAQEHEEVELRGSGLQPDLIEGRKGAQIVKRASLKRGKQ

SEQ ID NO: 20 - protein, 1719 aa, Homo sapiens
MPYSVGFREADAATSFLRAARSGNLDKALDHLRNGVDINTCNQNGLNGLHLASKEGHVKMVVELLHKEIILETTTKKGNTALHIAALAGQDEVVRELVNYGANVNAQSQKGFTPLYMAAQENHLEVVKFLLENGANQNVATEDGFTPLAVALQQGHENVVAHLINYGTKGKVRLPALHIAARNDDTRTAAVLLQNDPNPDVLSKTGFTPLHIAAHYENLNVAQLLLNRGASVNFTPQNGITPLHIASRRGNVIMVRLLLDRGAQIETKTKDELTPLHCAARNGHVRISEILLDHGAPIQAKTKNGLSPIHMAAQGDHLDCVRLLLQYDAEIDDITLDHLTPLHVAAHCGHHRVAKVLLDKGAKPNSRALNGFTPLHIACKKNHVRVMELLLKTGASIDAVTESGLTPLHVASFMGHLPIVKNLLQRGASPNVSNVKVETPLHMAARAGHTEVAKYLLQNKAKVNAKAKDDQTPLHCAARIGHTNMVKLLLENNANPNLATTAGHTPLHIAAREGHVETVLALLEKEASQACMTKKGFTPLHVAAKYGKVRVAELLLERDAHPNAAGKNGLTPLHVAVHHNNLDIVKLLLPRGGSPHSPAWNGYTPLHIAAKQNQVEVARSLLQYGGSANAESVQGVTPLHLAAQEGHAEMVALLLSKQANGNLGNKSGLTPLHLVAQEGHVPVADVLIKHGVMVDATTRMGYTPLHVASHYGNIKLVKFLLQHQADVNAKTKLGYSPLHQAAQQGHTDIVTLLLKNGASPNEVSSDGTTPLAIAKRLGYISVTDVLKVVTDETSFVLVSDKHRMSFPETVDEILDVSEDEGEELISFKAERRDSRDVDEEKELLDFVPKLDQVVESPAIPRIPCAMPETVVIRSEEQEQASKEYDEDSLIPSSPATETSDNISPVASPVHTGFLVSFMVDARGGSMRGSRHNGLRVVIPPRTCAAPTRITCRLVKPQKLSTPPPLAEEEGLASRIIALGPTGAQFLSPVIVEIPHFASHGRGDRELVVLRSENGSVWKEHRSRYGESYLDQILNGMDEELGSLEELEKKRVCRIITTDFPLYFVIMSRLCQDYDTIGPEGGSLKSKLVPLVQATFPENAVTKRVKLALQAQPVPDELVTKLLGNQATFSPIVTVEPRRRKFHRPIGLRIPLPPSWTDNPRDSGEGDTTSLRLLCSVIGGTDQAQWEDITGTTKLVYANECANFTTNVSARFWLSDCPRTAEAVNFATLLYKELTAVPYMAKFVIFAKMNDPREGRLRCYCMTDDKVDKTLEQHENFVEVARSRDIEVLEGMSLFAELSGNLVPVKKAAQQRSFHFQSFRENRLAMPVKVRDSSREPGGSLSFLRKAMKYEDTQHILCHLNITMPPCAKGSGAEDRRRTPTPLALRYSILSESTPGSLSGTEQAEMKMAVISEHLGLSWAELARELQFSVEDINRIRVENPNSLLEQSVALLNLWVIREGQNANMENLYTALQSIDRGEIVNMLEGSGRQSRNLKPDRRHTDRDYSLSPSQMNGHQRGQARITHSPTVSQVTERSQDRLQDWDADGSIVSYLQDAAQGSWQEEVTQGPHSFQGTSTMTEGLEPGGSQEYEKVLVSVSEHTWTEQPEAESSQADRDRRQQGQEEQVQEAKNTFTQVVQGNEFQNIPGEQVTEEQFTDEQGNIVTKKIIRKVVRQIDLSSADAAQEHEEVTVEGPLEDPSELEVDIDYFMKHSKDHTSTPNP

SEQ ID NO: 21 - protein, 1856 aa, Homo sapiens
MPYSVGFREADAATSFLRAARSGNLDKALDHLRNGVDINTCNQNGLNGLHLASKEGHVKMVVELLHKEIILETTTKKGNTALHIAALAGQDEVVRELVNYGANVNAQSQKGFTPLYMAAQENHLEVVKFLLENGANQNVATEDGFTPLAVALQQGHENVVAHLINYGTKGKVRLPALHIAARNDDTRTAAVLLQNDPNPDVLSKTGFTPLHIAAHYENLNVAQLLLNRGASVNFTPQNGITPLHIASRRGNVIMVRLLLDRGAQIETKTKDELTPLHCAARNGHVRISEILLDHGAPIQAKTKNGLSPIHMAAQGDHLDCVRLLLQYDAEIDDITLDHLTPLHVAAHCGHHRVAKVLLDKGAKPNSRALNGFTPLHIACKKNHVRVMELLLKTGASIDAVTESGLTPLHVASFMGHLPIVKNLLQRGASPNVSNVKVETPLHMAARAGHTEVAKYLLQNKAKVNAKAKDDQTPLHCAARIGHTNMVKLLLENNANPNLATTAGHTPLHIAAREGHVETVLALLEKEASQACMTKKGFTPLHVAAKYGKVRVAELLLERDAHPNAAGKNGLTPLHVAVHHNNLDIVKLLLPRGGSPHSPAWNGYTPLHIAAKQNQVEVARSLLQYGGSANAESVQGVTPLHLAAQEGHAEMVALLLSKQANGNLGNKSGLTPLHLVAQEGHVPVADVLIKHGVMVDATTRMGYTPLHVASHYGNIKLVKFLLQHQADVNAKTKLGYSPLHQAAQQGHTDIVTLLLKNGASPNEVSSDGTTPLAIAKRLGYISVTDVLKVVTDETSFVLVSDKHRMSFPETVDEILDVSEDEGEELISFKAERRDSRDVDEEKELLDFVPKLDQVVESPAIPRIPCAMPETVVIRSEEQEQASKEYDEDSLIPSSPATETSDNISPVASPVHTGFLVSFMVDARGGSMRGSRHNGLRVVIPPRTCAAPTRITCRLVKPQKLSTPPPLAEEEGLASRIIALGPTGAQFLSPVIVEIPHFASHGRGDRELVVLRSENGSVWKEHRSRYGESYLDQILNGMDEELGSLEELEKKRVCRIITTDFPLYFVIMSRLCQDYDTIGPEGGSLKSKLVPLVQATFPENAVTKRVKLALQAQPVPDELVTKLLGNQATFSPIVTVEPRRRKFHRPIGLRIPLPPSWTDNPRDSGEGDTTSLRLLCSVIGGTDQAQWEDITGTTKLVYANECANFTTNVSARFWLSDCPRTAEAVNFATLLYKELTAVPYMAKFVIFAKMNDPREGRLRCYCMTDDKVDKTLEQHENFVEVARSRDIEVLEGMSLFAELSGNLVPVKKAAQQRSFHFQSFRENRLAMPVKVRDSSREPGGSLSFLRKAMKYEDTQHILCHLNITMPPCAKGSGAEDRRRTPTPLALRYSILSESTPGSLSGTEQAEMKMAVISEHLGLSWAELARELQFSVEDINRIRVENPNSLLEQSVALLNLWVIREGQNANMENLYTALQSIDRGEIVNMLEGSGRQSRNLKPDRRHTDRDYSLSPSQMNGYSSLQDELLSPASLGCALSSPLRADQYWNEVAVLDAIPLAATEHDTMLEMSDMQVWSAGLTPSLVTAEDSSLECSKAEDSDATGHEWKLEGALSEEPRGPELGSLELVEDDTVDSDATNGLIDLLEQEEGQRSEEKLPGSKRQDDATGAGQDSENEVSLVSGHQRGQARITHSPTVSQVTERSQDRLQDWDADGSIVSYLQDAAQGSWQEEVTQGPHSFQGTSTMTEGLEPGGSQEYEKVLVSVSEHTWTEQPEAESSQADRDRRQQGQEEQVQEAKNTFTQVVQGNEFQNIPGEQVTEEQFTDEQGNIVTKKIIRKVVRQIDLSSADAAQEHEEDHTSTPNP

SEQ ID NO: 22 - protein, 1694 aa, Homo sapiens
MPYSVGFREADAATSFLRAARSGNLDKALDHLRNGVDINTCNQNGLNGLHLASKEGHVKMVVELLHKEIILETTTKKGNTALHIAALAGQDEVVRELVNYGANVNAQSQKGFTPLYMAAQENHLEVVKFLLENGANQNVATEDGFTPLAVALQQGHENVVAHLINYGTKGKVRLPALHIAARNDDTRTAAVLLQNDPNPDVLSKTGFTPLHIAAHYENLNVAQLLLNRGASVNFTPQNGITPLHIASRRGNVIMVRLLLDRGAQIETKTKDELTPLHCAARNGHVRISEILLDHGAPIQAKTKNGLSPIHMAAQGDHLDCVRLLLQYDAEIDDITLDHLTPLHVAAHCGHHRVAKVLLDKGAKPNSRALNGFTPLHIACKKNHVRVMELLLKTGASIDAVTESGLTPLHVASFMGHLPIVKNLLQRGASPNVSNVKVETPLHMAARAGHTEVAKYLLQNKAKVNAKAKDDQTPLHCAARIGHTNMVKLLLENNANPNLATTAGHTPLHIAAREGHVETVLALLEKEASQACMTKKGFTPLHVAAKYGKVRVAELLLERDAHPNAAGKNGLTPLHVAVHHNNLDIVKLLLPRGGSPHSPAWNGYTPLHIAAKQNQVEVARSLLQYGGSANAESVQGVTPLHLAAQEGHAEMVALLLSKQANGNLGNKSGLTPLHLVAQEGHVPVADVLIKHGVMVDATTRMGYTPLHVASHYGNIKLVKFLLQHQADVNAKTKLGYSPLHQAAQQGHTDIVTLLLKNGASPNEVSSDGTTPLAIAKRLGYISVTDVLKVVTDETSFVLVSDKHRMSFPETVDEILDVSEDEGEELISFKAERRDSRDVDEEKELLDFVPKLDQVVESPAIPRIPCAMPETVVIRSEEQEQASKEYDEDSLIPSSPATETSDNISPVASPVHTGFLVSFMVDARGGSMRGSRHNGLRVVIPPRTCAAPTRITCRLVKPQKLSTPPPLAEEEGLASRIIALGPTGAQFLSPVIVEIPHFASHGRGDRELVVLRSENGSVWKEHRSRYGESYLDQILNGMDEELGSLEELEKKRVCRIITTDFPLYFVIMSRLCQDYDTIGPEGGSLKSKLVPLVQATFPENAVTKRVKLALQAQPVPDELVTKLLGNQATFSPIVTVEPRRRKFHRPIGLRIPLPPSWTDNPRDSGEGDTTSLRLLCSVIGGTDQAQWEDITGTTKLVYANECANFTTNVSARFWLSDCPRTAEAVNFATLLYKELTAVPYMAKFVIFAKMNDPREGRLRCYCMTDDKVDKTLEQHENFVEVARSRDIEVLEGMSLFAELSGNLVPVKKAAQQRSFHFQSFRENRLAMPVKVRDSSREPGGSLSFLRKAMKYEDTQHILCHLNITMPPCAKGSGAEDRRRTPTPLALRYSILSESTPGSLSGTEQAEMKMAVISEHLGLSWAELARELQFSVEDINRIRVENPNSLLEQSVALLNLWVIREGQNANMENLYTALQSIDRGEIVNMLEGSGRQSRNLKPDRRHTDRDYSLSPSQMNGHQRGQARITHSPTVSQVTERSQDRLQDWDADGSIVSYLQDAAQGSWQEEVTQGPHSFQGTSTMTEGLEPGGSQEYEKVLVSVSEHTWTEQPEAESSQADRDRRQQGQEEQVQEAKNTFTQVVQGNEFQNIPGEQVTEEQFTDEQGNIVTKKIIRKVVRQIDLSSADAAQEHEEDHTSTPNP

SEQ ID NO: 23 - protein, 1718 aa, Homo sapiens
MPYSVGFREADAATSFLRAARSGNLDKALDHLRNGVDINTCNQNGLNGLHLASKEGHVKMVVELLHKEIILETTTKKGNTALHIAALAGQDEVVRELVNYGANVNAQSQKGFTPLYMAAQENHLEVVKFLLENGANQNVATEDGFTPLAVALQQGHENVVAHLINYGTKGKVRLPALHIAARNDDTRTAAVLLQNDPNPDVLSKTGFTPLHIAAHYENLNVAQLLLNRGASVNFTPQNGITPLHIASRRGNVIMVRLLLDRGAQIETKTKDELTPLHCAARNGHVRISEILLDHGAPIQAKTKNGLSPIHMAAQGDHLDCVRLLLQYDAEIDDITLDHLTPLHVAAHCGHHRVAKVLLDKGAKPNSRALNGFTPLHIACKKNHVRVMELLLKTGASIDAVTESGLTPLHVASFMGHLPIVKNLLQRGASPNVSNVKVETPLHMAARAGHTEVAKYLLQNKAKVNAKAKDDQTPLHCAARIGHTNMVKLLLENNANPNLATTAGHTPLHIAAREGHVETVLALLEKEASQACMTKKGFTPLHVAAKYGKVRVAELLLERDAHPNAAGKNGLTPLHVAVHHNNLDIVKLLLPRGGSPHSPAWNGYTPLHIAAKQNQVEVARSLLQYGGSANAESVQGVTPLHLAAQEGHAEMVALLLSKQANGNLGNKSGLTPLHLVAQEGHVPVADVLIKHGVMVDATTRMGYTPLHVASHYGNIKLVKFLLQHQADVNAKTKLGYSPLHQAAQQGHTDIVTLLLKNGASPNEVSSDGTTPLAIAKRLGYISVTDVLKVVTDETSFVLVSDKHRMSFPETVDEILDVSEDEGEELISFKAERRDSRDVDEEKELLDFVPKLDQVVESPAIPRIPCAMPETVVIRSEEQEQASKEYDEDSLIPSSPATETSDNISPVASPVHTGFLVSFMVDARGGSMRGSRHNGLRVVIPPRTCAAPTRITCRLVKPQKLSTPPPLAEEEGLASRIIALGPTGAQFLSPVIVEIPHFASHGRGDRELVVLRSENGSVWKEHRSRYGESYLDQILNGMDEELGSLEELEKKRVCRIITTDFPLYFVIMSRLCQDYDTIGPEGGSLKSKLVPLVQATFPENAVTKRVKLALQAQPVPDELVTKLLGNQATFSPIVTVEPRRRKFHRPIGLRIPLPPSWTDNPRDSGEGDTTSLRLLCSVIGGTDQAQWEDITGTTKLVYANECANFTTNVSARFWLSDCPRTAEAVNFATLLYKELTAVPYMAKFVIFAKMNDPREGRLRCYCMTDDKVDKTLEQHENFVEVARSRDIEVLEGMSLFAELSGNLVPVKKAAQQRSFHFQSFRENRLAMPVKVRDSSREPGGSLSFLRKAMKYEDTQHILCHLNITMPPCAKGSGAEDRRRTPTPLALRYSILSESTPGSLSGTEQAEMKMAVISEHLGLSWAELARELQFSVEDINRIRVENPNSLLEQSVALLNLWVIREGQNANMENLYTALQSIDRGEIVNMLEGSGRQSRNLKPDRRHTDRDYSLSPSQMNGHQRGQARITHSPTVSQVTERSQDRLQDWDADGSIVSYLQDAAQGSWQEEVTQGPHSFQGTSTMTEGLEPGGSQEYEKVLVSVSEHTWTEQPEAESSQADRDRRQQGQEEQVQEAKNTFTQVVQGNEFQNIPGEQVTEEQFTDEQGNIVTKKIIRKVVRQIDLSSADAAQEHEEVELRGSGLQPDLIEGRKGAQIVKRASLKRGKQ

SEQ ID NO: 24 - protein, 1834 aa, Homo sapiens
MPYSVGFREADAATSFLRAARSGNLDKALDHLRNGVDINTCNQNGLNGLHLASKEGHVKMVVELLHKEIILETTTKKGNTALHIAALAGQDEVVRELVNYGANVNAQSQKGFTPLYMAAQENHLEVVKFLLENGANQNVATEDGFTPLAVALQQGHENVVAHLINYGTKGKVRLPALHIAARNDDTRTAAVLLQNDPNPDVLSKTGFTPLHIAAHYENLNVAQLLLNRGASVNFTPQNGITPLHIASRRGNVIMVRLLLDRGAQIETKTKDELTPLHCAARNGHVRISEILLDHGAPIQAKTKNGLSPIHMAAQGDHLDCVRLLLQYDAEIDDITLDHLTPLHVAAHCGHHRVAKVLLDKGAKPNSRALNGFTPLHIACKKNHVRVMELLLKTGASIDAVTESGLTPLHVASFMGHLPIVKNLLQRGASPNVSNVKVETPLHMAARAGHTEVAKYLLQNKAKVNAKAKDDQTPLHCAARIGHTNMVKLLLENNANPNLATTAGHTPLHIAAREGHVETVLALLEKEASQACMTKKGFTPLHVAAKYGKVRVAELLLERDAHPNAAGKNGLTPLHVAVHHNNLDIVKLLLPRGGSPHSPAWNGYTPLHIAAKQNQVEVARSLLQYGGSANAESVQGVTPLHLAAQEGHAEMVALLLSKQANGNLGNKSGLTPLHLVAQEGHVPVADVLIKHGVMVDATTRMGYTPLHVASHYGNIKLVKFLLQHQADVNAKTKLGYSPLHQAAQQGHTDIVTLLLKNGASPNEVSSDGTTPLAIAKRLGYISVTDVLKVVTDETSFVLVSDKHRMSFPETVDEILDVSEDEGEELISFKAERRDSRDVDEEKELLDFVPKLDQVVESPAIPRIPCAMPETVVIRSEEQEQASKEYDEDSLIPSSPATETSDNISPVASPVHTGFLVSFMVDARGGSMRGSRHNGLRVVIPPRTCAAPTRITCRLVKPQKLSTPPPLAEEEGLASRIIALGPTGAQFLSPVIVEIPHFASHGRGDRELVVLRSENGSVWKEHRSRYGESYLDQILNGMDEELGSLEELEKKRVCRIITTDFPLYFVIMSRLCQDYDTIGPEGGSLKSKLVPLVQATFPENAVTKRVKLALQAQPVPDELVTKLLGNQATFSPIVTVEPRRRKFHRPIGLRIPLPPSWTDNPRDSGEGDTTSLRLLCSVIGGTDQAQWEDITGTTKLVYANECANFTTNVSARFWLSDCPRTAEAVNFATLLYKELTAVPYMAKFVIFAKMNDPREGRLRCYCMTDDKVDKTLEQHENFVEVARSRDIEVLEGMSLFAELSGNLVPVKKAAQQRSFHFQSFRENRLAMPVKVRDSSREPGGSLSFLRKAMKYEDTQHILCHLNITMPPCAKGSGAEDRRRTPTPLALRYSILSESTPGSLSGTEQAEMKMAVISEHLGLSWAELARELQFSVEDINRIRVENPNSLLEQSVALLNLWVIREGQNANMENLYTALQSIDRGEIVNMLEGSGRQSRNLKPDRRHTDRDYSLSPSQMNGYSSLQDELLSPASLGCALSSPLRADQYWNEVAVLDAIPLAATEHDTMLEMSDMQVWSAGLTPSLVTAEDSSLECSKAEDSDATGHEWKLEGALSEEPRGPELGSLELVEDDTVDSDATNGLIDLLEQEEGQRSEEKLPGSKRQDDATGAGQDSENEVSLVSGHQRGQARITHSPTVSQVTERSQDRLQDWDADGSIVSYLQDAAQGSWQEEVTQGPHSFQGTSTMTEGLEPGGSQEYEKVLVSVSEHTWTEQPEAESSQADRDRRQQGQEEQVQEAKNTFTQVVQGNEFQNIPGEQVTEEQFTDEQGNIVTKKDHTSTPNP

SEQ ID NO: 25 - protein, 1672 aa, Homo sapiens
MPYSVGFREADAATSFLRAARSGNLDKALDHLRNGVDINTCNQNGLNGLHLASKEGHVKMVVELLHKEIILETTTKKGNTALHIAALAGQDEVVRELVNYGANVNAQSQKGFTPLYMAAQENHLEVVKFLLENGANQNVATEDGFTPLAVALQQGHENVVAHLINYGTKGKVRLPALHIAARNDDTRTAAVLLQNDPNPDVLSKTGFTPLHIAAHYENLNVAQLLLNRGASVNFTPQNGITPLHIASRRGNVIMVRLLLDRGAQIETKTKDELTPLHCAARNGHVRISEILLDHGAPIQAKTKNGLSPIHMAAQGDHLDCVRLLLQYDAEIDDITLDHLTPLHVAAHCGHHRVAKVLLDKGAKPNSRALNGFTPLHIACKKNHVRVMELLLKTGASIDAVTESGLTPLHVASFMGHLPIVKNLLQRGASPNVSNVKVETPLHMAARAGHTEVAKYLLQNKAKVNAKAKDDQTPLHCAARIGHTNMVKLLLENNANPNLATTAGHTPLHIAAREGHVETVLALLEKEASQACMTKKGFTPLHVAAKYGKVRVAELLLERDAHPNAAGKNGLTPLHVAVHHNNLDIVKLLLPRGGSPHSPAWNGYTPLHIAAKQNQVEVARSLLQYGGSANAESVQGVTPLHLAAQEGHAEMVALLLSKQANGNLGNKSGLTPLHLVAQEGHVPVADVLIKHGVMVDATTRMGYTPLHVASHYGNIKLVKFLLQHQADVNAKTKLGYSPLHQAAQQGHTDIVTLLLKNGASPNEVSSDGTTPLAIAKRLGYISVTDVLKVVTDETSFVLVSDKHRMSFPETVDEILDVSEDEGEELISFKAERRDSRDVDEEKELLDFVPKLDQVVESPAIPRIPCAMPETVVIRSEEQEQASKEYDEDSLIPSSPATETSDNISPVASPVHTGFLVSFMVDARGGSMRGSRHNGLRVVIPPRTCAAPTRITCRLVKPQKLSTPPPLAEEEGLASRIIALGPTGAQFLSPVIVEIPHFASHGRGDRELVVLRSENGSVWKEHRSRYGESYLDQILNGMDEELGSLEELEKKRVCRIITTDFPLYFVIMSRLCQDYDTIGPEGGSLKSKLVPLVQATFPENAVTKRVKLALQAQPVPDELVTKLLGNQATFSPIVTVEPRRRKFHRPIGLRIPLPPSWTDNPRDSGEGDTTSLRLLCSVIGGTDQAQWEDITGTTKLVYANECANFTTNVSARFWLSDCPRTAEAVNFATLLYKELTAVPYMAKFVIFAKMNDPREGRLRCYCMTDDKVDKTLEQHENFVEVARSRDIEVLEGMSLFAELSGNLVPVKKAAQQRSFHFQSFRENRLAMPVKVRDSSREPGGSLSFLRKAMKYEDTQHILCHLNITMPPCAKGSGAEDRRRTPTPLALRYSILSESTPGSLSGTEQAEMKMAVISEHLGLSWAELARELQFSVEDINRIRVENPNSLLEQSVALLNLWVIREGQNANMENLYTALQSIDRGEIVNMLEGSGRQSRNLKPDRRHTDRDYSLSPSQMNGHQRGQARITHSPTVSQVTERSQDRLQDWDADGSIVSYLQDAAQGSWQEEVTQGPHSFQGTSTMTEGLEPGGSQEYEKVLVSVSEHTWTEQPEAESSQADRDRRQQGQEEQVQEAKNTFTQVVQGNEFQNIPGEQVTEEQFTDEQGNIVTKKDHTSTPNP

SEQ ID NO: 26 - protein, 1806 aa, Homo sapiens
MPYSVGFREADAATSFLRAARSGNLDKALDHLRNGVDINTCNQNGLNGLHLASKEGHVKMVVELLHKEIILETTTKKGNTALHIAALAGQDEVVRELVNYGANVNAQSQKGFTPLYMAAQENHLEVVKFLLENGANQNVATEDGFTPLAVALQQGHENVVAHLINYGTKGKVRLPALHIAARNDDTRTAAVLLQNDPNPDVLSKTGFTPLHIAAHYENLNVAQLLLNRGASVNFTPQNGITPLHIASRRGNVIMVRLLLDRGAQIETKTKDELTPLHCAARNGHVRISEILLDHGAPIQAKTKNGLSPIHMAAQGDHLDCVRLLLQYDAEIDDITLDHLTPLHVAAHCGHHRVAKVLLDKGAKPNSRALNGFTPLHIACKKNHVRVMELLLKTGASIDAVTESGLTPLHVASFMGHLPIVKNLLQRGASPNVSNVKVETPLHMAARAGHTEVAKYLLQNKAKVNAKAKDDQTPLHCAARIGHTNMVKLLLENNANPNLATTAGHTPLHIAAREGHVETVLALLEKEASQACMTKKGFTPLHVAAKYGKVRVAELLLERDAHPNAAGKNGLTPLHVAVHHNNLDIVKLLLPRGGSPHSPAWNGYTPLHIAAKQNQVEVARSLLQYGGSANAESVQGVTPLHLAAQEGHAEMVALLLSKQANGNLGNKSGLTPLHLVAQEGHVPVADVLIKHGVMVDATTRMGYTPLHVASHYGNIKLVKFLLQHQADVNAKTKLGYSPLHQAAQQGHTDIVTLLLKNGASPNEVSSDGTTPLAIAKRLGYISVTDVLKVVTDETSFVLVSDKHRMSFPETVDEILDVSEDEGEELISFKAERRDSRDVDEEKELLDFVPKLDQVVESPAIPRIPCAMPETVVIRSEEQEQASKEYDEDSLIPSSPATETSDNISPVASPVHTGFLVSFMVDARGGSMRGSRHNGLRVVIPPRTCAAPTRITCRLVKPQKLSTPPPLAEEEGLASRIIALGPTGAQFLSPVIVEIPHFASHGRGDRELVVLRSENGSVWKEHRSRYGESYLDQILNGMDEELGSLEELEKKRVCRIITTDFPLYFVIMSRLCQDYDTIGPEGGSLKSKLVPLVQATFPENAVTKRVKLALQAQPVPDELVTKLLGNQATFSPIVTVEPRRRKFHRPIGLRIPLPPSWTDNPRDSGEGDTTSLRLLCSVIGGTDQAQWEDITGTTKLVYANECANFTTNVSARFWLSDCPRTAEAVNFATLLYKELTAVPYMAKFVIFAKMNDPREGRLRCYCMTDDKVDKTLEQHENFVEVARSRDIEVLEGMSLFAELSGNLVPVKKAAQQRSFHFQSFRENRLAMPVKVRDSSREPGGSLSFLRKAMKYEDTQHILCHLNITMPPCAKGSGAEDRRRTPTPLALRYSILSESTPGSLSGTEQAEMKMAVISEHLGLSWAELARELQFSVEDINRIRVENPNSLLEQSVALLNLWVIREGQNANMENLYTALQSIDRGEIVNMLEGSGRQSRNLKPDRRHTDRDYSLSPSQMNGYSSLQDELLSPASLGCALSSPLRADQYWNEVAVLDAIPLAATEHDTMLEMSDMQVWSAGLTPSLVTAEDSSLECSKAEDSDATGHEWKLEGALSEEPRGPELGSLELVEDDTVDSDATNGLIDLLEQEEGQRSEEKLPGSKRQDDATGAGQDSENEVSLVSGHQRGQARITHSPTVSQVTERSQDRLQDWDADGSIVSYLQDAAQGSWQEEVTQGPHSFQGTSTMTEGLEPGGSQEYEKVLVSVSEHTWTEQPEAESSQADRDRRQQGQEEQVQEAKNTFTQVVQDHTSTPNP

SEQ ID NO: 27 - protein, 1644 aa, Homo sapiens
MPYSVGFREADAATSFLRAARSGNLDKALDHLRNGVDINTCNQNGLNGLHLASKEGHVKMVVELLHKEIILETTTKKGNTALHIAALAGQDEVVRELVNYGANVNAQSQKGFTPLYMAAQENHLEVVKFLLENGANQNVATEDGFTPLAVALQQGHENVVAHLINYGTKGKVRLPALHIAARNDDTRTAAVLLQNDPNPDVLSKTGFTPLHIAAHYENLNVAQLLLNRGASVNFTPQNGITPLHIASRRGNVIMVRLLLDRGAQIETKTKDELTPLHCAARNGHVRISEILLDHGAPIQAKTKNGLSPIHMAAQGDHLDCVRLLLQYDAEIDDITLDHLTPLHVAAHCGHHRVAKVLLDKGAKPNSRALNGFTPLHIACKKNHVRVMELLLKTGASIDAVTESGLTPLHVASFMGHLPIVKNLLQRGASPNVSNVKVETPLHMAARAGHTEVAKYLLQNKAKVNAKAKDDQTPLHCAARIGHTNMVKLLLENNANPNLATTAGHTPLHIAAREGHVETVLALLEKEASQACMTKKGFTPLHVAAKYGKVRVAELLLERDAHPNAAGKNGLTPLHVAVHHNNLDIVKLLLPRGGSPHSPAWNGYTPLHIAAKQNQVEVARSLLQYGGSANAESVQGVTPLHLAAQEGHAEMVALLLSKQANGNLGNKSGLTPLHLVAQEGHVPVADVLIKHGVMVDATTRMGYTPLHVASHYGNIKLVKFLLQHQADVNAKTKLGYSPLHQAAQQGHTDIVTLLLKNGASPNEVSSDGTTPLAIAKRLGYISVTDVLKVVTDETSFVLVSDKHRMSFPETVDEILDVSEDEGEELISFKAERRDSRDVDEEKELLDFVPKLDQVVESPAIPRIPCAMPETVVIRSEEQEQASKEYDEDSLIPSSPATETSDNISPVASPVHTGFLVSFMVDARGGSMRGSRHNGLRVVIPPRTCAAPTRITCRLVKPQKLSTPPPLAEEEGLASRIIALGPTGAQFLSPVIVEIPHFASHGRGDRELVVLRSENGSVWKEHRSRYGESYLDQILNGMDEELGSLEELEKKRVCRIITTDFPLYFVIMSRLCQDYDTIGPEGGSLKSKLVPLVQATFPENAVTKRVKLALQAQPVPDELVTKLLGNQATFSPIVTVEPRRRKFHRPIGLRIPLPPSWTDNPRDSGEGDTTSLRLLCSVIGGTDQAQWEDITGTTKLVYANECANFTTNVSARFWLSDCPRTAEAVNFATLLYKELTAVPYMAKFVIFAKMNDPREGRLRCYCMTDDKVDKTLEQHENFVEVARSRDIEVLEGMSLFAELSGNLVPVKKAAQQRSFHFQSFRENRLAMPVKVRDSSREPGGSLSFLRKAMKYEDTQHILCHLNITMPPCAKGSGAEDRRRTPTPLALRYSILSESTPGSLSGTEQAEMKMAVISEHLGLSWAELARELQFSVEDINRIRVENPNSLLEQSVALLNLWVIREGQNANMENLYTALQSIDRGEIVNMLEGSGRQSRNLKPDRRHTDRDYSLSPSQMNGHQRGQARITHSPTVSQVTERSQDRLQDWDADGSIVSYLQDAAQGSWQEEVTQGPHSFQGTSTMTEGLEPGGSQEYEKVLVSVSEHTWTEQPEAESSQADRDRRQQGQEEQVQEAKNTFTQVVQDHTSTPNP

SEQ ID NO: 28 - protein, 1905 aa, Homo sapiens
MPYSVGFREADAATSFLRAARSGNLDKALDHLRNGVDINTCNQNGLNGLHLASKEGHVKMVVELLHKEIILETTTKKGNTALHIAALAGQDEVVRELVNYGANVNAQSQKGFTPLYMAAQENHLEVVKFLLENGANQNVATEDGFTPLAVALQQGHENVVAHLINYGTKGKVRLPALHIAARNDDTRTAAVLLQNDPNPDVLSKTGFTPLHIAAHYENLNVAQLLLNRGASVNFTPQNGITPLHIASRRGNVIMVRLLLDRGAQIETKTKDELTPLHCAARNGHVRISEILLDHGAPIQAKTKNGLSPIHMAAQGDHLDCVRLLLQYDAEIDDITLDHLTPLHVAAHCGHHRVAKVLLDKGAKPNSRALNGFTPLHIACKKNHVRVMELLLKTGASIDAVTESGLTPLHVASFMGHLPIVKNLLQRGASPNVSNVKVETPLHMAARAGHTEVAKYLLQNKAKVNAKAKDDQTPLHCAARIGHTNMVKLLLENNANPNLATTAGHTPLHIAAREGHVETVLALLEKEASQACMTKKGFTPLHVAAKYGKVRVAELLLERDAHPNAAGKNGLTPLHVAVHHNNLDIVKLLLPRGGSPHSPAWNGYTPLHIAAKQNQVEVARSLLQYGGSANAESVQGVTPLHLAAQEGHAEMVALLLSKQANGNLGNKSGLTPLHLVAQEGHVPVADVLIKHGVMVDATTRMGYTPLHVASHYGNIKLVKFLLQHQADVNAKTKLGYSPLHQAAQQGHTDIVTLLLKNGASPNEVSSDGTTPLAIAKRLGYISVTDVLKVVTDETSFVLVSDKHRMSFPETVDEILDVSEDEGEELISFKAERRDSRDVDEEKELLDFVPKLDQVVESPAIPRIPCAMPETVVIRSEEQEQASKEYDEDSLIPSSPATETSDNISPVASPVHTGFLVSFMVDARGGSMRGSRHNGLRVVIPPRTCAAPTRITCRLVKPQKLSTPPPLAEEEGLASRIIALGPTGAQFLSPVIVEIPHFASHGRGDRELVVLRSENGSVWKEHRSRYGESYLDQILNGMDEELGSLEELEKKRVCRIITTDFPLYFVIMSRLCQDYDTIGPEGGSLKSKLVPLVQATFPENAVTKRVKLALQAQPVPDELVTKLLGNQATFSPIVTVEPRRRKFHRPIGLRIPLPPSWTDNPRDSGEGDTTSLRLLCSVIGGTDQAQWEDITGTTKLVYANECANFTTNVSARFWLSDCPRTAEAVNFATLLYKELTAVPYMAKFVIFAKMNDPREGRLRCYCMTDDKVDKTLEQHENFVEVARSRDIEVLEGMSLFAELSGNLVPVKKAAQQRSFHFQSFRENRLAMPVKVRDSSREPGGSLSFLRKAMKYEDTQHILCHLNITMPPCAKGSGAEDRRRTPTPLALRYSILSESTPGSLSGTEQAEMKMAVISEHLGLSWAELARELQFSVEDINRIRVENPNSLLEQSVALLNLWVIREGQNANMENLYTALQSIDRGEIVNMLEGSGRQSRNLKPDRRHTDRDYSLSPSQMNGYSSLQDELLSPASLGCALSSPLRADQYWNEVAVLDAIPLAATEHDTMLEMSDMQVWSAGLTPSLVTAEDSSLECSKAEDSDATGHEWKLEGALSEEPRGPELGSLELVEDDTVDSDATNGLIDLLEQEEGQRSEEKLPGSKRQDDATGAGQDSENEVSLVSGHQRGQARITHSPTVSQVTERSQDRLQDWDADGSIVSYLQDAAQGSWQEEVTQGPHSFQGTSTMTEGLEPGGSQEYEKVLVSVSEHTWTEQPEAESSQADRDRRQQGQEEQVQEAKNTFTQVVQGNEFQNIPGEQVTEEQFTDEQGNIVTKKIIRKVVRQIDLSSADAAQEHEEVTVEGPLEDPSELEVDIDYFMKHSKVELRGSGLQPDLIEGRKGAQIVKRASLKRGKQ

SEQ ID NO: 29 - protein, 1743 aa, Homo sapiens
MPYSVGFREADAATSFLRAARSGNLDKALDHLRNGVDINTCNQNGLNGLHLASKEGHVKMVVELLHKEIILETTTKKGNTALHIAALAGQDEVVRELVNYGANVNAQSQKGFTPLYMAAQENHLEVVKFLLENGANQNVATEDGFTPLAVALQQGHENVVAHLINYGTKGKVRLPALHIAARNDDTRTAAVLLQNDPNPDVLSKTGFTPLHIAAHYENLNVAQLLLNRGASVNFTPQNGITPLHIASRRGNVIMVRLLLDRGAQIETKTKDELTPLHCAARNGHVRISEILLDHGAPIQAKTKNGLSPIHMAAQGDHLDCVRLLLQYDAEIDDITLDHLTPLHVAAHCGHHRVAKVLLDKGAKPNSRALNGFTPLHIACKKNHVRVMELLLKTGASIDAVTESGLTPLHVASFMGHLPIVKNLLQRGASPNVSNVKVETPLHMAARAGHTEVAKYLLQNKAKVNAKAKDDQTPLHCAARIGHTNMVKLLLENNANPNLATTAGHTPLHIAAREGHVETVLALLEKEASQACMTKKGFTPLHVAAKYGKVRVAELLLERDAHPNAAGKNGLTPLHVAVHHNNLDIVKLLLPRGGSPHSPAWNGYTPLHIAAKQNQVEVARSLLQYGGSANAESVQGVTPLHLAAQEGHAEMVALLLSKQANGNLGNKSGLTPLHLVAQEGHVPVADVLIKHGVMVDATTRMGYTPLHVASHYGNIKLVKFLLQHQADVNAKTKLGYSPLHQAAQQGHTDIVTLLLKNGASPNEVSSDGTTPLAIAKRLGYISVTDVLKVVTDETSFVLVSDKHRMSFPETVDEILDVSEDEGEELISFKAERRDSRDVDEEKELLDFVPKLDQVVESPAIPRIPCAMPETVVIRSEEQEQASKEYDEDSLIPSSPATETSDNISPVASPVHTGFLVSFMVDARGGSMRGSRHNGLRVVIPPRTCAAPTRITCRLVKPQKLSTPPPLAEEEGLASRIIALGPTGAQFLSPVIVEIPHFASHGRGDRELVVLRSENGSVWKEHRSRYGESYLDQILNGMDEELGSLEELEKKRVCRIITTDFPLYFVIMSRLCQDYDTIGPEGGSLKSKLVPLVQATFPENAVTKRVKLALQAQPVPDELVTKLLGNQATFSPIVTVEPRRRKFHRPIGLRIPLPPSWTDNPRDSGEGDTTSLRLLCSVIGGTDQAQWEDITGTTKLVYANECANFTTNVSARFWLSDCPRTAEAVNFATLLYKELTAVPYMAKFVIFAKMNDPREGRLRCYCMTDDKVDKTLEQHENFVEVARSRDIEVLEGMSLFAELSGNLVPVKKAAQQRSFHFQSFRENRLAMPVKVRDSSREPGGSLSFLRKAMKYEDTQHILCHLNITMPPCAKGSGAEDRRRTPTPLALRYSILSESTPGSLSGTEQAEMKMAVISEHLGLSWAELARELQFSVEDINRIRVENPNSLLEQSVALLNLWVIREGQNANMENLYTALQSIDRGEIVNMLEGSGRQSRNLKPDRRHTDRDYSLSPSQMNGHQRGQARITHSPTVSQVTERSQDRLQDWDADGSIVSYLQDAAQGSWQEEVTQGPHSFQGTSTMTEGLEPGGSQEYEKVLVSVSEHTWTEQPEAESSQADRDRRQQGQEEQVQEAKNTFTQVVQGNEFQNIPGEQVTEEQFTDEQGNIVTKKIIRKVVRQIDLSSADAAQEHEEVTVEGPLEDPSELEVDIDYFMKHSKVELRGSGLQPDLIEGRKGAQIVKRASLKRGKQ

SEQ ID NO: 30 - protein, 1983 aa, Homo sapiens
MPYSVGFREADAATSFLRAARSGNLDKALDHLRNGVDINTCNQNGLNGLHLASKEGHVKMVVELLHKEIILETTTKKGNTALHIAALAGQDEVVRELVNYGANVNAQSQKGFTPLYMAAQENHLEVVKFLLENGANQNVATEDGFTPLAVALQQGHENVVAHLINYGTKGKVRLPALHIAARNDDTRTAAVLLQNDPNPDVLSKTGFTPLHIAAHYENLNVAQLLLNRGASVNFTPQNGITPLHIASRRGNVIMVRLLLDRGAQIETKTKDELTPLHCAARNGHVRISEILLDHGAPIQAKTKNGLSPIHMAAQGDHLDCVRLLLQYDAEIDDITLDHLTPLHVAAHCGHHRVAKVLLDKGAKPNSRALNGFTPLHIACKKNHVRVMELLLKTGASIDAVTESGLTPLHVASFMGHLPIVKNLLQRGASPNVSNVKVETPLHMAARAGHTEVAKYLLQNKAKVNAKAKDDQTPLHCAARIGHTNMVKLLLENNANPNLATTAGHTPLHIAAREGHVETVLALLEKEASQACMTKKGFTPLHVAAKYGKVRVAELLLERDAHPNAAGKNGLTPLHVAVHHNNLDIVKLLLPRGGSPHSPAWNGYTPLHIAAKQNQVEVARSLLQYGGSANAESVQGVTPLHLAAQEGHAEMVALLLSKQANGNLGNKSGLTPLHLVAQEGHVPVADVLIKHGVMVDATTRMGYTPLHVASHYGNIKLVKFLLQHQADVNAKTKLGYSPLHQAAQQGHTDIVTLLLKNGASPNEVSSDGTTPLAIAKRLGYISVTDVLKVVTDETSFVLVSDKHRMSFPETVDEILDVSEDEGEELISFKAERRDSRDVDEEKELLDFVPKLDQVVESPAIPRIPCAMPETVVIRSEEQEQASKEYDEDSLIPSSPATETSDNISPVASPVHTGFLVSFMVDARGGSMRGSRHNGLRVVIPPRTCAAPTRITCRLVKPQKLSTPPPLAEEEGLASRIIALGPTGAQFLSPVIVEIPHFASHGRGDRELVVLRSENGSVWKEHRSRYGESYLDQILNGMDEELGSLEELEKKRVCRIITTDFPLYFVIMSRLCQDYDTIGPEGGSLKSKLVPLVQATFPENAVTKRVKLALQAQPVPDELVTKLLGNQATFSPIVTVEPRRRKFHRPIGLRIPLPPSWTDNPRDSGEGDTTSLRLLCSVIGGTDQAQWEDITGTTKLVYANECANFTTNVSARFWLSDCPRTAEAVNFATLLYKELTAVPYMAKFVIFAKMNDPREGRLRCYCMTDDKVDKTLEQHENFVEVARSRDIEVLEGMSLFAELSGNLVPVKKAAQQRSFHFQSFRENRLAMPVKVRDSSREPGGSLSFLRKAMKYEDTQHILCHLNITMPPCAKGSGAEDRRRTPTPLALRYSILSESTPGSLSGTEQAEMKMAVISEHLGLSWAELARELQFSVEDINRIRVENPNSLLEQSVALLNLWVIREGQNANMENLYTALQSIDRGEIVNMLEGSGRQSRNLKPDRRHTDRDYSLSPSQMNGYSSLQDELLSPASLGCALSSPLRADQYWNEVAVLDAIPLAATEHDTMLEMSDMQVWSAGLTPSLVTAEDSSLECSKAEDSDATGHEWKLEGALSEEPRGPELGSLELVEDDTVDSDATNGLIDLLEQEEGQRSEEKLPGSKRQDDATGAGQDSENEVSLVSGHQRGQARITHSPTVSQVTERSQDRLQDWDADGSIVSYLQDAAQGSWQEEVTQGPHSFQGTSTMTEGLEPGGSQEYEKVLVSVSEHTWTEQPEAESSQADRDRRQQGQEEQVQEAKNTFTQVVQGNEFQNIPGEQVTEEQFTDEQGNIVTKKIIRKVVRQIDLSSADAAQEHEEVTVEGPLEDPSELEVDIDYFMKHSKVLRRPRPWGTQRHHCCLALPGRLHDTSLHSPLYELSLQSLFSLVGSVSAPPCRSFRSSACVLPVFAICPAFCLCCCLQVELRGSGLQPDLIEGRKGAQIVKRASLKRGKQ

SEQ ID NO: 31 - protein, 1821 aa, Homo sapiens
MPYSVGFREADAATSFLRAARSGNLDKALDHLRNGVDINTCNQNGLNGLHLASKEGHVKMVVELLHKEIILETTTKKGNTALHIAALAGQDEVVRELVNYGANVNAQSQKGFTPLYMAAQENHLEVVKFLLENGANQNVATEDGFTPLAVALQQGHENVVAHLINYGTKGKVRLPALHIAARNDDTRTAAVLLQNDPNPDVLSKTGFTPLHIAAHYENLNVAQLLLNRGASVNFTPQNGITPLHIASRRGNVIMVRLLLDRGAQIETKTKDELTPLHCAARNGHVRISEILLDHGAPIQAKTKNGLSPIHMAAQGDHLDCVRLLLQYDAEIDDITLDHLTPLHVAAHCGHHRVAKVLLDKGAKPNSRALNGFTPLHIACKKNHVRVMELLLKTGASIDAVTESGLTPLHVASFMGHLPIVKNLLQRGASPNVSNVKVETPLHMAARAGHTEVAKYLLQNKAKVNAKAKDDQTPLHCAARIGHTNMVKLLLENNANPNLATTAGHTPLHIAAREGHVETVLALLEKEASQACMTKKGFTPLHVAAKYGKVRVAELLLERDAHPNAAGKNGLTPLHVAVHHNNLDIVKLLLPRGGSPHSPAWNGYTPLHIAAKQNQVEVARSLLQYGGSANAESVQGVTPLHLAAQEGHAEMVALLLSKQANGNLGNKSGLTPLHLVAQEGHVPVADVLIKHGVMVDATTRMGYTPLHVASHYGNIKLVKFLLQHQADVNAKTKLGYSPLHQAAQQGHTDIVTLLLKNGASPNEVSSDGTTPLAIAKRLGYISVTDVLKVVTDETSFVLVSDKHRMSFPETVDEILDVSEDEGEELISFKAERRDSRDVDEEKELLDFVPKLDQVVESPAIPRIPCAMPETVVIRSEEQEQASKEYDEDSLIPSSPATETSDNISPVASPVHTGFLVSFMVDARGGSMRGSRHNGLRVVIPPRTCAAPTRITCRLVKPQKLSTPPPLAEEEGLASRIIALGPTGAQFLSPVIVEIPHFASHGRGDRELVVLRSENGSVWKEHRSRYGESYLDQILNGMDEELGSLEELEKKRVCRIITTDFPLYFVIMSRLCQDYDTIGPEGGSLKSKLVPLVQATFPENAVTKRVKLALQAQPVPDELVTKLLGNQATFSPIVTVEPRRRKFHRPIGLRIPLPPSWTDNPRDSGEGDTTSLRLLCSVIGGTDQAQWEDITGTTKLVYANECANFTTNVSARFWLSDCPRTAEAVNFATLLYKELTAVPYMAKFVIFAKMNDPREGRLRCYCMTDDKVDKTLEQHENFVEVARSRDIEVLEGMSLFAELSGNLVPVKKAAQQRSFHFQSFRENRLAMPVKVRDSSREPGGSLSFLRKAMKYEDTQHILCHLNITMPPCAKGSGAEDRRRTPTPLALRYSILSESTPGSLSGTEQAEMKMAVISEHLGLSWAELARELQFSVEDINRIRVENPNSLLEQSVALLNLWVIREGQNANMENLYTALQSIDRGEIVNMLEGSGRQSRNLKPDRRHTDRDYSLSPSQMNGHQRGQARITHSPTVSQVTERSQDRLQDWDADGSIVSYLQDAAQGSWQEEVTQGPHSFQGTSTMTEGLEPGGSQEYEKVLVSVSEHTWTEQPEAESSQADRDRRQQGQEEQVQEAKNTFTQVVQGNEFQNIPGEQVTEEQFTDEQGNIVTKKIIRKVVRQIDLSSADAAQEHEEVTVEGPLEDPSELEVDIDYFMKHSKVLRRPRPWGTQRHHCCLALPGRLHDTSLHSPLYELSLQSLFSLVGSVSAPPCRSFRSSACVLPVFAICPAFCLCCCLQVELRGSGLQPDLIEGRKGAQIVKRASLKRGKQ

SEQ ID NO: 32 - protein, 1858 aa, Homo sapiens
MPYSVGFREADAATSFLRAARSGNLDKALDHLRNGVDINTCNQNGLNGLHLASKEGHVKMVVELLHKEIILETTTKKGNTALHIAALAGQDEVVRELVNYGANVNAQSQKGFTPLYMAAQENHLEVVKFLLENGANQNVATEDGFTPLAVALQQGHENVVAHLINYGTKGKVRLPALHIAARNDDTRTAAVLLQNDPNPDVLSKTGFTPLHIAAHYENLNVAQLLLNRGASVNFTPQNGITPLHIASRRGNVIMVRLLLDRGAQIETKTKDELTPLHCAARNGHVRISEILLDHGAPIQAKTKNGLSPIHMAAQGDHLDCVRLLLQYDAEIDDITLDHLTPLHVAAHCGHHRVAKVLLDKGAKPNSRALNGFTPLHIACKKNHVRVMELLLKTGASIDAVTESGLTPLHVASFMGHLPIVKNLLQRGASPNVSNVKVETPLHMAARAGHTEVAKYLLQNKAKVNAKAKDDQTPLHCAARIGHTNMVKLLLENNANPNLATTAGHTPLHIAAREGHVETVLALLEKEASQACMTKKGFTPLHVAAKYGKVRVAELLLERDAHPNAAGKNGLTPLHVAVHHNNLDIVKLLLPRGGSPHSPAWNGYTPLHIAAKQNQVEVARSLLQYGGSANAESVQGVTPLHLAAQEGHAEMVALLLSKQANGNLGNKSGLTPLHLVAQEGHVPVADVLIKHGVMVDATTRMGYTPLHVASHYGNIKLVKFLLQHQADVNAKTKLGYSPLHQAAQQGHTDIVTLLLKNGASPNEVSSDGTTPLAIAKRLGYISVTDVLKVVTDETSFVLVSDKHRMSFPETVDEILDVSEDEGEELISFKAERRDSRDVDEEKELLDFVPKLDQVVESPAIPRIPCAMPETVVIRSEEQEQASKEYDEDSLIPSSPATETSDNISPVASPVHTGFLVSFMVDARGGSMRGSRHNGLRVVIPPRTCAAPTRITCRLVKPQKLSTPPPLAEEEGLASRIIALGPTGAQFLSPVIVEIPHFASHGRGDRELVVLRSENGSVWKEHRSRYGESYLDQILNGMDEELGSLEELEKKRVCRIITTDFPLYFVIMSRLCQDYDTIGPEGGSLKSKLVPLVQATFPENAVTKRVKLALQAQPVPDELVTKLLGNQATFSPIVTVEPRRRKFHRPIGLRIPLPPSWTDNPRDSGEGDTTSLRLLCSVIGGTDQAQWEDITGTTKLVYANECANFTTNVSARFWLSDCPRTAEAVNFATLLYKELTAVPYMAKFVIFAKMNDPREGRLRCYCMTDDKVDKTLEQHENFVEVARSRDIEVLEGMSLFAELSGNLVPVKKAAQQRSFHFQSFRENRLAMPVKVRDSSREPGGSLSFLRKAMKYEDTQHILCHLNITMPPCAKGSGAEDRRRTPTPLALRYSILSESTPGSLSGTEQAEMKMAVISEHLGLSWAELARELQFSVEDINRIRVENPNSLLEQSVALLNLWVIREGQNANMENLYTALQSIDRGEIVNMLEGSGRQSRNLKPDRRHTDRDYSLSPSQMNGYSSLQDELLSPASLGCALSSPLRADQYWNEVAVLDAIPLAATEHDTMLEMSDMQVWSAGLTPSLVTAEDSSLECSKAEDSDATGHEWKLEGALSEEPRGPELGSLELVEDDTVDSDATNGLIDLLEQEEGQRSEEKLPGSKRQDDATGAGQDSENEVSLVSGHQRGQARITHSPTVSQVTERSQDRLQDWDADGSIVSYLQDAAQGSWQEEVTQGPHSFQGTSTMTEGLEPGGSQEYEKVLVSVSEHTWTEQPEAESSQADRDRRQQGQEEQVQEAKNTFTQVVQGNEFQNIPGEQVTEEQFTDEQGNIVTKKVELRGSGLQPDLIEGRKGAQIVKRASLKRGKQ

SEQ ID NO: 33 - protein, 155 aa, Homo sapiens
MWTFVTQLLVTLVLLSFFLVSCQNVMHIVRGSLCFVLKHIHQELDKELGESEGLSDDEETISTRVVRRRVFLKGNEFQNIPGEQVTEEQFTDEQGNIVTKKIIRKVVRQIDLSSADAAQEHEEVELRGSGLQPDLIEGRKGAQIVKRASLKRGKQ

SEQ ID NO: 34 - protein, 133 aa, Homo sapiens
MWTFVTQLLVTLVLLSFFLVSCQNVMHIVRGSLCFVLKHIHQELDKELGESEGLSDDEETISTRVVRRRVFLKGNEFQNIPGEQVTEEQFTDEQGNIVTKKVELRGSGLQPDLIEGRKGAQIVKRASLKRGKQ

SEQ ID NO: 35 - protein, 81 aa, Homo sapiens
MWTFVTQLLVTLVLLSFFLVSCQNVMHIVRGSLCFVLKHIHQELDKELGESEGLSDDEETISTRVVRRRVFLKDHTSTPNP

SEQ ID NO: 36 - protein, 74 aa, Homo sapiens
MWTFVTQLLVTLVLLSFFLVSCQNVMHIVRGSLCFVLKHIHQVELRGSGLQPDLIEGRKGAQIVKRASLKRGKQ

SEQ ID NO: 37 - protein, 1897 aa, Homo sapiens
MAQAAKQLKKIKDIEAQALQEQKEKEESNRKRRNRSRDRKKKADAATSFLRAARSGNLDKALDHLRNGVDINTCNQNGLNGLHLASKEGHVKMVVELLHKEIILETTTKKGNTALHIAALAGQDEVVRELVNYGANVNAQSQKGFTPLYMAAQENHLEVVKFLLENGANQNVATEDGFTPLAVALQQGHENVVAHLINYGTKGKVRLPALHIAARNDDTRTAAVLLQNDPNPDVLSKTGFTPLHIAAHYENLNVAQLLLNRGASVNFTPQNGITPLHIASRRGNVIMVRLLLDRGAQIETKTKDELTPLHCAARNGHVRISEILLDHGAPIQAKTKNGLSPIHMAAQGDHLDCVRLLLQYDAEIDDITLDHLTPLHVAAHCGHHRVAKVLLDKGAKPNSRALNGFTPLHIACKKNHVRVMELLLKTGASIDAVTESGLTPLHVASFMGHLPIVKNLLQRGASPNVSNVKVETPLHMAARAGHTEVAKYLLQNKAKVNAKAKDDQTPLHCAARIGHTNMVKLLLENNANPNLATTAGHTPLHIAAREGHVETVLALLEKEASQACMTKKGFTPLHVAAKYGKVRVAELLLERDAHPNAAGKNGLTPLHVAVHHNNLDIVKLLLPRGGSPHSPAWNGYTPLHIAAKQNQVEVARSLLQYGGSANAESVQGVTPLHLAAQEGHAEMVALLLSKQANGNLGNKSGLTPLHLVAQEGHVPVADVLIKHGVMVDATTRMGYTPLHVASHYGNIKLVKFLLQHQADVNAKTKLGYSPLHQAAQQGHTDIVTLLLKNGASPNEVSSDGTTPLAIAKRLGYISVTDVLKVVTDETSFVLVSDKHRMSFPETVDEILDVSEDEGTAHITIMGEELISFKAERRDSRDVDEEKELLDFVPKLDQVVESPAIPRIPCAMPETVVIRSEEQEQASKEYDEDSLIPSSPATETSDNISPVASPVHTGFLVSFMVDARGGSMRGSRHNGLRVVIPPRTCAAPTRITCRLVKPQKLSTPPPLAEEEGLASRIIALGPTGAQFLSPVIVEIPHFASHGRGDRELVVLRSENGSVWKEHRSRYGESYLDQILNGMDEELGSLEELEKKRVCRIITTDFPLYFVIMSRLCQDYDTIGPEGGSLKSKLVPLVQATFPENAVTKRVKLALQAQPVPDELVTKLLGNQATFSPIVTVEPRRRKFHRPIGLRIPLPPSWTDNPRDSGEGDTTSLRLLCSVIGGTDQAQWEDITGTTKLVYANECANFTTNVSARFWLSDCPRTAEAVNFATLLYKELTAVPYMAKFVIFAKMNDPREGRLRCYCMTDDKVDKTLEQHENFVEVARSRDIEVLEGMSLFAELSGNLVPVKKAAQQRSFHFQSFRENRLAMPVKVRDSSREPGGSLSFLRKAMKYEDTQHILCHLNITMPPCAKGSGAEDRRRTPTPLALRYSILSESTPGSLSGTEQAEMKMAVISEHLGLSWAELARELQFSVEDINRIRVENPNSLLEQSVALLNLWVIREGQNANMENLYTALQSIDRGEIVNMLEGSGRQSRNLKPDRRHTDRDYSLSPSQMNGYSSLQDELLSPASLGCALSSPLRADQYWNEVAVLDAIPLAATEHDTMLEMSDMQVWSAGLTPSLVTAEDSSLECSKAEDSDATGHEWKLEGALSEEPRGPELGSLELVEDDTVDSDATNGLIDLLEQEEGQRSEEKLPGSKRQDDATGAGQDSENEVSLVSGHQRGQARITHSPTVSQVTERSQDRLQDWDADGSIVSYLQDAAQGSWQEEVTQGPHSFQGTSTMTEGLEPGGSQEYEKVLVSVSEHTWTEQPEAESSQADRDRRQQGQEEQVQEAKNTFTQVVQGNEFQNIPGEQVTEEQFTDEQGNIVTKKIIRKVVRQIDLSSADAAQEHEEDHTSTPNP

SEQ ID NO: 38 - protein, 109 aa, Homo sapiens
MWTFVTQLLVTLVLLSFFLVSCQNVMHIVRGSLCFVLKHIHQELDKELGESEGLSDDEETISTRVVRRRVFLKGNEFQNIPGEQVTEEQFTDEQGNIVTKKDHTSTPNP

SEQ ID NO: 39 - protein, 156 aa, Homo sapiens
MWTFVTQLLVTLVLLSFFLVSCQNVMHIVRGSLCFVLKHIHQELDKELGESEGLSDDEETISTRVVRRRVFLKGNEFQNIPGEQVTEEQFTDEQGNIVTKKIIRKVVRQIDLSSADAAQEHEEVTVEGPLEDPSELEVDIDYFMKHSKDHTSTPNP

SEQ ID NO: 40 - protein, 691 aa, Homo sapiens
MGQALGIKSCDFQAARNNEEHHTKALSSRRLFVRRGQPFTIILYFRAPVRAFLPALKKVALTAQTGEQPSKINRTQATFPISSLGDRKWWSAVVEERDAQSWTISVTTPADAVIGHYSLLLQVSGRKQLLLGQFTLLFNPWNREDAVFLKNEAQRMEYLLNQNGLIYLGTADCIQAESWDFGQFEGDVIDLSLRLLSKDKQVEKWSQPVHVARVLGALLHFLKEQRVLPTPQTQATQEGALLNKRRGSVPILRQWLTGRGRPVYDGQAWVLAAVACTVLRCLGIPARVVTTFASAQGTGGRLLIDEYYNEEGLQNGEGQRGRIWIFQTSTECWMTRPALPQGYDGWQILHPSAPNGGGVLGSCDLVPVRAVKEGTLGLTPAVSDLFAAINASCVVWKCCEDGTLELTDSNTKYVGNNISTKGVGSDRCEDITQNYKYPEGSLQEKEVLERVEKEKMEREKDNGIRPPSLETASPLYLLLKAPSSLPLRGDAQISVTLVNHSEQEKAVQLAIGVQAVHYNGVLAAKLWRKKLHLTLSANLEKIITIGLFFSNFERNPPENTFLRLTAMATHSESNLSCFAQEDIAICRPHLAIKMPEKAEQYQPLTASVSLQNSLDAPMEDCVISILGRGLIHRERSYRFRSVWPENTMCAKFQFTPTHVGLQRLTVEVDCNMFQNLTNYKSVTVVAPELSA

SEQ ID NO: 41 - protein, 721 aa, Homo sapiens
MGQGEPSQRSTGLAGLYAAPAASPVFIKGSGMDALGIKSCDFQAARNNEEHHTKALSSRRLFVRRGQPFTIILYFRAPVRAFLPALKKVALTAQTGEQPSKINRTQATFPISSLGDRKWWSAVVEERDAQSWTISVTTPADAVIGHYSLLLQVSGRKQLLLGQFTLLFNPWNREDAVFLKNEAQRMEYLLNQNGLIYLGTADCIQAESWDFGQFEGDVIDLSLRLLSKDKQVEKWSQPVHVARVLGALLHFLKEQRVLPTPQTQATQEGALLNKRRGSVPILRQWLTGRGRPVYDGQAWVLAAVACTVLRCLGIPARVVTTFASAQGTGGRLLIDEYYNEEGLQNGEGQRGRIWIFQTSTECWMTRPALPQGYDGWQILHPSAPNGGGVLGSCDLVPVRAVKEGTLGLTPAVSDLFAAINASCVVWKCCEDGTLELTDSNTKYVGNNISTKGVGSDRCEDITQNYKYPEGSLQEKEVLERVEKEKMEREKDNGIRPPSLETASPLYLLLKAPSSLPLRGDAQISVTLVNHSEQEKAVQLAIGVQAVHYNGVLAAKLWRKKLHLTLSANLEKIITIGLFFSNFERNPPENTFLRLTAMATHSESNLSCFAQEDIAICRPHLAIKMPEKAEQYQPLTASVSLQNSLDAPMEDCVISILGRGLIHRERSYRFRSVWPENTMCAKFQFTPTHVGLQRLTVEVDCNMFQNLTNYKSVTVVAPELSA

SEQ ID NO: 42 - protein, 619 aa, Homo sapiens
MGQALGIKSCDFQAARNNEEHHTKALSSRRLFVRRGQPFTIILYFRAPVRAFLPALKKVALTAQTGEQPSKINRTQATFPISSLGDRKWWSAVVEERDAQSWTISVTTPADAVIGHYSLLLQVSGRKQLLLGQFTLLFNPWNREDAVFLKNEAQRMEYLLNQNGLIYLGTADCIQAESWDFGQFEGDVIDLSLRLLSKDKQVEKWSQPVHVARVLGALLHFLKEQRVLPTPQTQATQEGALLNKRRGSVPILRQWLTGRGRPVYDGQAWVLAAVACTVLRCLGIPARVVTTFASAQGTGGRLLIDEYYNEEGLQNGEGQRGRIWKCCEDGTLELTDSNTKYVGNNISTKGVGSDRCEDITQNYKYPEGSLQEKEVLERVEKEKMEREKDNGIRPPSLETASPLYLLLKAPSSLPLRGDAQISVTLVNHSEQEKAVQLAIGVQAVHYNGVLAAKLWRKKLHLTLSANLEKIITIGLFFSNFERNPPENTFLRLTAMATHSESNLSCFAQEDIAICRPHLAIKMPEKAEQYQPLTASVSLQNSLDAPMEDCVISILGRGLIHRERSYRFRSVWPENTMCAKFQFTPTHVGLQRLTVEVDCNMFQNLTNYKSVTVVAPELSA

SEQ ID NO: 43 - protein, 291 aa, Homo sapiens
MQRARPTLWAAALTLLVLLRGPPVARAGASSAGLGPVVRCEPCDARALAQCAPPPAVCAELVREPGCGCCLTCALSEGQPCGIYTERCGSGLRCQPSPDEARPLQALLDGRGLCVNASAVSRLRAYLLPAPPAPGNASESEEDRSAGSVESPSVSSTHRVSDPKFHPLHSKIIIIKKGHAKDSQRYKVDYESQSTDTQNFSSESKRETEYGPCRREMEDTLNHLKFLNVLSPRGVHIPNCDKKGFYKKKQCRPSKGRKRGFCWCVDKYGQPLPGYTTKGKEDVHCYSMQSK

SEQ ID NO: 44 - protein, 297 aa, Homo sapiens
MQRARPTLWAAALTLLVLLRGPPVARAGASSAGLGPVVRCEPCDARALAQCAPPPAVCAELVREPGCGCCLTCALSEGQPCGIYTERCGSGLRCQPSPDEARPLQALLDGRGLCVNASAVSRLRAYLLPAPPAPGEPPAPGNASESEEDRSAGSVESPSVSSTHRVSDPKFHPLHSKIIIIKKGHAKDSQRYKVDYESQSTDTQNFSSESKRETEYGPCRREMEDTLNHLKFLNVLSPRGVHIPNCDKKGFYKKKQCRPSKGRKRGFCWCVDKYGQPLPGYTTKGKEDVHCYSMQSK

SEQ ID NO: 45 - protein, 469 aa, Homo sapiens
MITEGAQAPRLLLPPLLLLLTLPATGSDPVLCFTQYEESSGKCKGLLGGGVSVEDCCLNTAFAYQKRSGGLCQPCRSPRWSLWSTWAPCSVTCSEGSQLRYRRCVGWNGQCSGKVAPGTLEWQLQACEDQQCCPEMGGWSGWGPWEPCSVTCSKGTRTRRRACNHPAPKCGGHCPGQAQESEACDTQQVCPTHGAWATWGPWTPCSASCHGGPHEPKETRSRKCSAPEPSQKPPGKPCPGLAYEQRRCTGLPPCPVAGGWGPWGPVSPCPVTCGLGQTMEQRTCNHPVPQHGGPFCAGDATRTHICNTAVPCPVDGEWDSWGEWSPCIRRNMKSISCQEIPGQQSRGRTCRGRKFDGHRCAGQQQDIRHCYSIQHCPLKGSWSEWSTWGLCMPPCGPNPTRARQRLCTPLLPKYPPTVSMVEGQGEKNVTFWGRPLPRCEELQGQKLVVEEKRPCLHVPACKDPEEEEL

SEQ ID NO: 46 - protein, 206 aa, Homo sapiens
MAVKKIAIFGATGQTGLTTLAQAVQAGYEVTVLVRDSSRLPSEGPRPAHVVVGDVLQAADVDKTVAGQDAVIVLLGTRNDLSPTTVMSEGARNIVAAMKAHGVDKVVACTSAFLLWDPTKVPPRLQAVTDDHIRMHKVLRESGLKYVAVMPPHIGDQPLTGAYTVTLDGRGPSRVISKHDLGHFMLRCLTTDEYDGHSTYPSHQYQ

SEQ ID NO: 47 - protein, 758 aa, Homo sapiens
MAQRKNAKSSGNSSSSGSGSGSTSAGSSSPGARRETKHGGHKNGRKGGLSGTSFFTWFMVIALLGVWTSVAVVWFDLVDYEEVLGKLGIYDADGDGDFDVDDAKVLLGLKERSTSEPAVPPEEAEPHTEPEEQVPVEAEPQNIEDEAKEQIQSLLHEMVHAEHVEGEDLQQEDGPTGEPQQEDDEFLMATDVDDRFETLEPEVSHEETEHSYHVEETVSQDCNQDMEEMMSEQENPDSSEPVVEDERLHHDTDDVTYQVYEEQAVYEPLENEGIEITEVTAPPEDNPVEDSQVIVEEVSIFPVEEQQEVPPETNRKTDDPEQKAKVKKKKPKLLNKFDKTIKAELDAAEKLRKRGKIEEAVNAFKELVRKYPQSPRARYGKAQCEDDLAEKRRSNEVLRGAIETYQEVASLPDVPADLLKLSLKRRSDRQQFLGHMRGSLLTLQRLVQLFPNDTSLKNDLGVGYLLIGDNDNAKKVYEEVLSVTPNDGFAKVHYGFILKAQNKIAESIPYLKEGIESGDPGTDDGRFYFHLGDAMQRVGNKEAYKWYELGHKRGHFASVWQRSLYNVNGLKAQPWWTPKETGYTELVKSLERNWKLIRDEGLAVMDKAKGLFLPEDENLREKGDWSQFTLWQQGRRNENACKGAPKTCTLLEKFPETTGCRRGQIKYSIMHPGTHVWPHTGPTNCRLRMHLGLVIPKEGCKIRCANETKTWEEGKVLIFDDSFEHEVWQDASSFRLIFIVDVWHPELTPQQRRSLPAI

SEQ ID NO: 48 - protein, 313 aa, Homo sapiens
MAQRKNAKSSGNSSSSGSGSGSTSAGSSSPGARRETKHGGHKNGRKGGLSGTSFFTWFMVIALLGVWTSVAVVWFDLVDYEEVLGKLGIYDADGDGDFDVDDAKVLLGLKERSTSEPAVPPEEAEPHTEPEEQVPVEAEPQNIEDEAKEQIQSLLHEMVHAEHVEGEDLQQEDGPTGEPQQEDDEFLMATDVDDRFETLEPEVSHEETEHSYHVEETVSQDCNQDMEEMMSEQENPDSSEPVVEDERLHHDTDDVTYQVYEEQAVYEPLENEGIEITEVTAPPEDNPVEDSQVIVEEVSIFPVEEQQEVPPDT

SEQ ID NO: 49 - protein, 225 aa, Homo sapiens
MAEDKETKHGGHKNGRKGGLSGTSFFTWFMVIALLGVWTSVAVVWFDLVDYEEVLAKAKDFRYNLSEVLQGKLGIYDADGDGDFDVDDAKVLLEGPSGVAKRKTKAKVKELTKEELKKEKEKPESRKESKNEERKKGKKEDVRKDKKIADADLSRKESPKGKKDREKEKVDLEKSAKTKENRKKSTNMKDVSSKMASRDKDDRKESRSSTRYAHLTKGNTQKRNG

SEQ ID NO: 50 - protein, 210 aa, Homo sapiens
MAEDKETKHGGHKNGRKGGLSGTSFFTWFMVIALLGVWTSVAVVWFDLVDYEEVLGKLGIYDADGDGDFDVDDAKVLLEGPSGVAKRKTKAKVKELTKEELKKEKEKPESRKESKNEERKKGKKEDVRKDKKIADADLSRKESPKGKKDREKEKVDLEKSAKTKENRKKSTNMKDVSSKMASRDKDDRKESRSSTRYAHLTKGNTQKRNG

SEQ ID NO: 51 - protein, 298 aa, Homo sapiens
MAEDKETKHGGHKNGRKGGLSGTSFFTWFMVIALLGVWTSVAVVWFDLVDYEEVLAKAKDFRYNLSEVLQGKLGIYDADGDGDFDVDDAKVLLGLKERSTSEPAVPPEEAEPHTEPEEQVPVEAEPQNIEDEAKEQIQSLLHEMVHAEHVEGEDLQQEDGPTGEPQQEDDEFLMATDVDDRFETLEPEVSHEETEHSYHVEETVSQDCNQDMEEMMSEQENPDSSEPVVEDERLHHDTDDVTYQVYEEQVYEPLENEGIEITEVTAPPEDNPVEDSQVIVEEVSIFPVEEQQEVPPDT

SEQ ID NO: 52 - protein, 270 aa, Homo sapiens
MAQRKNAKSSGNSSSSGSGSGSTSAGSSSPGARRETKHGGHKNGRKGGLSGTSFFTWFMVIALLGVWTSVAVVWFDLVDYEEVLGKLGIYDADGDGDFDVDDAKVLLGLKERSTSEPAVPPEEAEPHTEPEEQVPVEAEPQNIEDEAKEQIQSLLHEMVHAEHETEHSYHVEETVSQDCNQDMEEMMSEQENPDSSEPVVEDERLHHDTDDVTYQVYEEQAVYEPLENEGIEITEVTAPPEDNPVEDSQVIVEEVSIFPVEEQQEVPPDT

SEQ ID NO: 53 - protein, 203 aa, Homo sapiens
MAQRKNAKSSGNSSSSGSGSGSTSAGSSSPGARRETKHGGHKNGRKGGLSGTSFFTWFMVIALLGVWTSVAVVWFDLVDYEEVLAKAKDFRYNLSEVLQGKLGIYDADGDGDFDVDDAKVLLGLTKDGSNENIDSLEEVLNILAEESSDWFYGFLSFLYDIMTPFEMLEEEEEESETADGVDGTSQNEGVQGKTCVILDLHNQ

SEQ ID NO: 54 - protein, 299 aa, Homo sapiens
MAEDKETKHGGHKNGRKGGLSGTSFFTWFMVIALLGVWTSVAVVWFDLVDYEEVLAKAKDFRYNLSEVLQGKLGIYDADGDGDFDVDDAKVLLGLKERSTSEPAVPPEEAEPHTEPEEQVPVEAEPQNIEDEAKEQIQSLLHEMVHAEHVEGEDLQQEDGPTGEPQQEDDEFLMATDVDDRFETLEPEVSHEETEHSYHVEETVSQDCNQDMEEMMSEQENPDSSEPVVEDERLHHDTDDVTYQVYEEQAVYEPLENEGIEITEVTAPPEDNPVEDSQVIVEEVSIFPVEEQQEVPPDT

SEQ ID NO: 55 - protein, 256 aa, Homo sapiens
MAEDKETKHGGHKNGRKGGLSGTSFFTWFMVIALLGVWTSVAVVWFDLVDYEEVLAKAKDFRYNLSEVLQGKLGIYDADGDGDFDVDDAKVLLGLKERSTSEPAVPPEEAEPHTEPEEQVPVEAEPQNIEDEAKEQIQSLLHEMVHAEHETEHSYHVEETVSQDCNQDMEEMMSEQENPDSSEPVVEDERLHHDTDDVTYQVYEEQAVYEPLENEGIEITEVTAPPEDNPVEDSQVIVEEVSIFPVEEQQEVPPDT

SEQ ID NO: 56 - protein, 729 aa, Homo sapiens
MAEDKETKHGGHKNGRKGGLSGTSFFTWFMVIALLGVWTSVAVVWFDLVDYEEVLGKLGIYDADGDGDFDVDDAKVLLGLKERSTSEPAVPPEEAEPHTEPEEQVPVEAEPQNIEDEAKEQIQSLLHEMVHAEHVEGEDLQQEDGPTGEPQQEDDEFLMATDVDDRFETLEPEVSHEETEHSYHVEETVSQDCNQDMEEMMSEQENPDSSEPVVEDERLHHDTDDVTYQVYEEQAVYEPLENEGIEITEVTAPPEDNPVEDSQVIVEEVSIFPVEEQQEVPPETNRKTDDPEQKAKVKKKKPKLLNKFDKTIKAELDAAEKLRKRGKIEEAVNAFKELVRKYPQSPRARYGKAQCEDDLAEKRRSNEVLRGAIETYQEVASLPDVPADLLKLSLKRRSDRQQFLGHMRGSLLTLQRLVQLFPNDTSLKNDLGVGYLLIGDNDNAKKVYEEVLSVTPNDGFAKVHYGFILKAQNKIAESIPYLKEGIESGDPGTDDGRFYFHLGDAMQRVGNKEAYKWYELGHKRGHFASVWQRSLYNVNGLKAQPWWTPKETGYTELVKSLERNWKLIRDEGLAVMDKAKGLFLPEDENLREKGDWSQFTLWQQGRRNENACKGAPKTCTLLEKFPETTGCRRGQIKYSIMHPGTHVWPHTGPTNCRLRMHLGLVIPKEGCKIRCANETKTWEEGKVLIFDDSFEHEVWQDASSFRLIFIVDVWHPELTPQQRRSLPAI

SEQ ID NO: 57 - protein, 294 aa, Homo sapiens
MAQRKNAKSSGNSSSSGSGSGSTSAGSSSPGARRETKHGGHKNGRKGGLSGTSFFTWFMVIALLGVWTSVAVVWFDLVDYEEVLGKLGIYDADGDGDFDVDDAKVLLGLKERSTSEPAVPPEEAEPHTEPEEQVPVEAEPQNIEDEAKEQIQSLLHEMVHAEHVEGEDLQQEDGPTGEPQQEDDEFLMATDVDDRFETLEPEVSHEETEHSYHVEETDSSEPVVEDERLHHDTDDVTYQVYEEQAVYEPLENEGIEITEVTAPPEDNPVEDSQVIVEEVSIFPVEEQQEVPPDT

SEQ ID NO: 58 - protein, 863 aa, Homo sapiens
MARRSSFQSCQIISLFTFAVGVNICLGFTAHRIKRAEGWEEGPPTVLSDSPWTNISGSCKGRCFELQEAGPPDCRCDNLCKSYTSCCHDFDELCLKTARGWECTKDRCGEVRNEENACHCSEDCLARGDCCTNYQVVCKGESHWVDDDCEEIKAAECPAGFVRPPLIIFSVDGFRASYMKKGSKVMPNIEKLRSCGTHSPYMRPVYPTKTFPNLYTLATGLYPESHGIVGNSMYDPVFDATFHLRGREKFNHRWWGGQPLWITATKQGVKAGTFFWSVVIPHERRILTILQWLTLPDHERPSVYAFYSEQPDFSGHKYGPFGPEMTNPLREIDKIVGQLMDGLKQLKLHRCVNVIFVGDHGMEDVTCDRTEFLSNYLTNVDDITLVPGTLGRIRSKFSNNAKYDPKAIIANLTCKKPDQHFKPYLKQHLPKRLHYANNRRIEDIHLLVERRWHVARKPLDVYKKPSGKCFFQGDHGFDNKVNSMQTVFVGYGSTFKYKTKVPPFENIELYNVMCDLLGLKPAPNNGTHGSLNHLLRTNTFRPTMPEEVTRPNYPGIMYLQSDFDLGCTCDDKVEPKNKLDELNKRLHTKGSTEERHLLYGRPAVLYRTRYDILYHTDFESGYSEIFLMPLWTSYTVSKQAEVSSVPDHLTSCVRPDVRVSPSFSQNCLAYKNDKQMSYGFLFPPYLSSSPEAKYDAFLVTNMVPMYPAFKRVWNYFQRVLVKKYASERNGVNVISGPIFDYDYDGLHDTEDKIKQYVEGSSIPVPTHYYSIITSCLDFTQPADKCDGPLSVSSFILPHRPDNEESCNSSEDESKWVEELMKMHTARVRDIEHLTSLDFFRKTSRSYPEILTLKTYLHTYESEI

SEQ ID NO: 59 - protein, 915 aa, Homo sapiens
MARRSSFQSCQIISLFTFAVGVNICLGFTAHRIKRAEGWEEGPPTVLSDSPWTNISGSCKGRCFELQEAGPPDCRCDNLCKSYTSCCHDFDELCLKTARGWECTKDRCGEVRNEENACHCSEDCLARGDCCTNYQVVCKGESHWVDDDCEEIKAAECPAGFVRPPLIIFSVDGFRASYMKKGSKVMPNIEKLRSCGTHSPYMRPVYPTKTFPNLYTLATGLYPESHGIVGNSMYDPVFDATFHLRGREKFNHRWWGGQPLWITATKQGVKAGTFFWSVVIPHERRILTILQWLTLPDHERPSVYAFYSEQPDFSGHKYGPFGPEESSYGSPFTPAKRPKRKVAPKRRQERPVAPPKKRRRKIHRMDHYAAETRQDKMTNPLREIDKIVGQLMDGLKQLKLHRCVNVIFVGDHGMEDVTCDRTEFLSNYLTNVDDITLVPGTLGRIRSKFSNNAKYDPKAIIANLTCKKPDQHFKPYLKQHLPKRLHYANNRRIEDIHLLVERRWHVARKPLDVYKKPSGKCFFQGDHGFDNKVNSMQTVFVGYGSTFKYKTKVPPFENIELYNVMCDLLGLKPAPNNGTHGSLNHLLRTNTFRPTMPEEVTRPNYPGIMYLQSDFDLGCTCDDKVEPKNKLDELNKRLHTKGSTEERHLLYGRPAVLYRTRYDILYHTDFESGYSEIFLMPLWTSYTVSKQAEVSSVPDHLTSCVRPDVRVSPSFSQNCLAYKNDKQMSYGFLFPPYLSSSPEAKYDAFLVTNMVPMYPAFKRVWNYFQRVLVKKYASERNGVNVISGPIFDYDYDGLHDTEDKIKQYVEGSSIPVPTHYYSIITSCLDFTQPADKCDGPLSVSSFILPHRPDNEESCNSSEDESKWVEELMKMHTARVRDIEHLTSLDFFRKTSRSYPEILTLKTYLHTYESEI

SEQ ID NO: 60 - protein, 888 aa, Homo sapiens
MARRSSFQSCQIISLFTFAVGVNICLGFTAHRIKRAEGWEEGPPTVLSDSPWTNISGSCKGRCFELQEAGPPDCRCDNLCKSYTSCCHDFDELCLKTARGWECTKDRCGEVRNEENACHCSEDCLARGDCCTNYQVVCKGESHWVDDDCEEIKAAECPAGFVRPPLIIFSVDGFRASYMKKGSKVMPNIEKLRSCGTHSPYMRPVYPTKTFPNLYTLATGLYPESHGIVGNSMYDPVFDATFHLRGREKFNHRWWGGQPLWITATKQGVKAGTFFWSVVIPHERRILTILQWLTLPDHERPSVYAFYSEQPDFSGHKYGPFGPEMTNPLREIDKIVGQLMDGLKQLKLHRCVNVIFVGDHGMEDVTCDRTEFLSNYLTNVDDITLVPGTLGRIRSKFSNNAKYDPKAIIANLTCKKPDQHFKPYLKQHLPKRLHYANNRRIEDIHLLVERRWHVARKPLDVYKKPSGKCFFQGDHGFDNKVNSMQTVFVGYGSTFKYKTKVPPFENIELYNVMCDLLGLKPAPNNGTHGSLNHLLRTNTFRPTMPEEVTRPNYPGIMYLQSDFDLGCTCDDKVEPKNKLDELNKRLHTKGSTEAETRKFRGSRNENKENINGNFEPRKERHLLYGRPAVLYRTRYDILYHTDFESGYSEIFLMPLWTSYTVSKQAEVSSVPDHLTSCVRPDVRVSPSFSQNCLAYKNDKQMSYGFLFPPYLSSSPEAKYDAFLVTNMVPMYPAFKRVWNYFQRVLVKKYASERNGVNVISGPIFDYDYDGLHDTEDKIKQYVEGSSIPVPTHYYSIITSCLDFTQPADKCDGPLSVSSFILPHRPDNEESCNSSEDESKWVEELMKMHTARVRDIEHLTSLDFFRKTSRSYPEILTLKTYLHTYESEI

SEQ ID NO: 61 - protein, 884 aa, Homo sapiens
MTRHADRIISLFTFAVGVNICLGFTAHRIKRAEGWEEGPPTVLSDSPWTNISGSCKGRCFELQEAGPPDCRCDNLCKSYTSCCHDFDELCLKTARGWECTKDRCGEVRNEENACHCSEDCLARGDCCTNYQVVCKGESHWVDDDCEEIKAAECPAGFVRPPLIIFSVDGFRASYMKKGSKVMPNIEKLRSCGTHSPYMRPVYPTKTFPNLYTLATGLYPESHGIVGNSMYDPVFDATFHLRGREKFNHRWWGGQPLWITATKQGVKAGTFFWSVVIPHERRILTILQWLTLPDHERPSVYAFYSEQPDFSGHKYGPFGPEMTNPLREIDKIVGQLMDGLKQLKLHRCVNVIFVGDHGMEDVTCDRTEFLSNYLTNVDDITLVPGTLGRIRSKFSNNAKYDPKAIIANLTCKKPDQHFKPYLKQHLPKRLHYANNRRIEDIHLLVERRWHVARKPLDVYKKPSGKCFFQGDHGFDNKVNSMQTVFVGYGSTFKYKTKVPPFENIELYNVMCDLLGLKPAPNNGTHGSLNHLLRTNTFRPTMPEEVTRPNYPGIMYLQSDFDLGCTCDDKVEPKNKLDELNKRLHTKGSTEAETRKFRGSRNENKENINGNFEPRKERHLLYGRPAVLYRTRYDILYHTDFESGYSEIFLMPLWTSYTVSKQAEVSSVPDHLTSCVRPDVRVSPSFSQNCLAYKNDKQMSYGFLFPPYLSSSPEAKYDAFLVTNMVPMYPAFKRVWNYFQRVLVKKYASERNGVNVISGPIFDYDYDGLHDTEDKIKQYVEGSSIPVPTHYYSIITSCLDFTQPADKCDGPLSVSSFILPHRPDNEESCNSSEDESKWVEELMKMHTARVRDIEHLTSLDFFRKTSRSYPEILTLKTYLHTYESEI

SEQ ID NO: 62 - protein, 470 aa, Homo sapiens
MGKPWLRALQLLLLLGASWARAGAPRCTYTFVLPPQKFTGAVCWSGPASTRATPEAANASELAALRMRVGRHEELLRELQRLAAADGAVAGEVRALRKESRGLSARLGQLRAQLQHEAGPGAGPGADLGAEPAAALALLGERVLNASAEAQRAAARFHQLDVKFRELAQLVTQQSSLIARLERLCPGGAGGQQQVLPPPPLVPVVPVRLVGSTSDTSRMLDPAPEPQRDQTQRQQEPMASPMPAGHPAVPTKPVGPWQDCAEARQAGHEQSGVYELRVGRHVVSVWCEQQLEGGGWTVIQRRQDGSVNFFTTWQHYKAGFGRPDGEYWLGLEPVYQLTSRGDHELLVLLEDWGGRGARAHYDGFSLEPESDHYRLRLGQYHGDAGDSLSWHNDKPFSTVDRDRDSYSGNCALYQRGGWWYHACAHSNLNGVWHHGGHYRSRYQDGVYWAEFRGGAYSLRKAAMLIRPLKL

SEQ ID NO: 63 - protein, 423 aa, Homo sapiens
MKLCSLAVLVPIVLFCEQHVFAFQSGQVLAALPRTSRQVQVLQNLTTTYEIVLWQPVTADLIVKKKQVHFFVNASDVDNVKAHLNVSGIPCSVLLADVEDLIQQQISNDTVSPRASASYYEQYHSLNEIYSWIEFITERHPDMLTKIHIGSSFEKYPLYVLKVSGKEQAAKNAIWIDCGIHAREWISPAFCLWFIGHITQFYGIIGQYTNLLRLVDFYVMPVVNVDGYDYSWKKNRMWRKNRSFYANNHCIGTDLNRNFASKHWCEEGASSSSCSETYCGLYPESEPEVKAVASFLRRNINQIKAYISMHSYSQHIVFPYSYTRSKSKDHEELSLVASEAVRAIEKISKNTRYTHGHGSETLYLAPGGGDDWIYDLGIKYSFTIELRDTGTYGFLLPERYIKPTCREAFAAVSKIAWHVIRNV

SEQ ID NO: 64 - protein, 360 aa, Homo sapiens
MKLCSLAVLVPIVLFCEQHVFAFQSGQVLAALPRTSRQVQVLQNLTTTYEIVLWQPVTADLIVKKKQVHFFVNASDVDNVKAHLNVSGIPCSVLLADVEDLIQQQISNDTVSPRASASYYEQYHSLNEIYSWIEFITERHPDMLTKIHIGSSFEKYPLYVLKVSGKEQAAKNAIWIDCGIHAREWISPAFCLWFIGHNRMWRKNRSFYANNHCIGTDLNRNFASKHWCEEGASSSSCSETYCGLYPESEPEVKAVASFLRRNINQIKAYISMHSYSQHIVFPYSYTRSKSKDHEELSLVASEAVRAIEKISKNTRYTHGHGSETLYLAPGGGDDWIYDLGIKYSFTSNPPVEKLLPLSLK

SEQ ID NO: 65 - protein, 380 aa, Homo sapiens
MKEYVLLLFLALCSAKPFFSPSHIALKNMMLKDMEDTDDDDDDDDDDDDDDEDNSLFPTREPRSHFFPFDLFPMCPFGCQCYSRVVHCSDLGLTSVPTNIPFDTRMLDLQNNKIKEIKENDFKGLTSLYGLILNNNKLTKIHPKAFLTTKKLRRLYLSHNQLSEIPLNLPKSLAELRIHENKVKKIQKDTFKGMNALHVLEMSANPLDNNGIEPGAFEGVTVFHIRIAEAKLTSVPKGLPPTLLELHLDYNKISTVELEDFKRYKELQRLGLGNNKITDIENGSLANIPRVREIHLENNKLKKIPSGLPELKYLQIIFLHSNSIARVGVNDFCPTVPKMKKSLYSAISLFNNPVKYWEMQPATFRCVLSRMSVQLGNFGM

SEQ ID NO: 66 - protein, 1187 aa, Homo sapiens
MRPRGAAFAAGPPGDLHLGTAIGFAGAIWRSRSPAMSTLLDIKSSVLRQVQVCPSFRRRTEQDPGSASADPQEPATGAWKPGDGVEFFAHMRLMLKKGEGRQGLPCLEVPLRSGSPAPPEPVDPSLGLRALAPEEVEMLYEEALYTVLYRAGTMGPDQVDDEEALLSYLQQVFGTSLEEHTEAIERVRKAKAPTYALKVSVMRAKNLLAKDPNGFSDPYCMLGILPASDATREPRAQKEQRFGFRKGSKRGGPLPAKCIQVTEVKSSTLNPVWKEHFLFEIEDVSTDQLHLDIWDHDDDVSLVEACRKLNEVIGLKGMGRYFKQIVKSARANGTAGPTEDHTDDFLGCLNIPVREVPVAGVDRWFKLEPRSSASRVQGHCHLVLKLITTQRDTAMSQRGRSGFLSHLLLLSHLLRLEHSAEEPNSSSWRGELSTPAATILCLHGAQSNLSPLQLAVLHWQVSSRHHQTCTLDYSYLLGLLEDMQAHWEEAPSLPQEQEESLADSLSAFSEFGLQLLRQLRDYFPATNSTAVHRLELLLKCLGKLQLFQPSFEICPFESELNMDIAAALKRGNREWYDRILNDKSPREQPGPQRLPGLVVLADAVYDDLQFCYSVYASLFHSILNVDVFTLTFRQLERLVAEEAWVLTEELSPKMTLEVASGLFELYLTLADLQRFWDSIPGRDSRSLALAGIHAPFLPAVKLWFQVLRDQAKWRLQGAVDMDTLEPVDASSRHSSSAATAGLCLSHIQELWVRLAWPDPAQAQGLGTQLGQDVCEATLFYTELLRKKVDTQPGAAGEAVSEALCVVLNNVELVRKAAGQALKGLAWPEGATGPEGVLPRPLLSCTQALDDDLQREAHTVTAHLTSKMVGDIRKYVQHISLSPDSIQNDEAVAPLMKYLDEKLALLNASLVKGNLSRVLEALWELLLQAILQALGANRDVSADFYSRFHFTLEALVSFFHAEGQGLPLESLRDGSYKRLKEELRLHKCSTRECIEQFYLDKLKQRTLEQNRFGRLSVRCHYEAAEQRLAVEVLHAADLLPLDANGLSDPFVIVELGPPHLFPLVRSQRTQVKTRTLHPVYDELFYFSVPAEACRRRAACVLFTVMDHDWLSTNDFAGEAALGLGGVTGVARPQVGGGARAGQPVTLHLCRPRAQVRSALRRLEGRTSKEAQEFVKKLKELEKCMEADP

SEQ ID NO: 67 - protein, 1169 aa, Homo sapiens
MSTLLDIKSSVLRQVQVCPSFRRRTEQDPGSASADPQEPATGAWKPGDGVEFFAHMRLMLKKGEGRQGLPCLEAWGSPCRQSPHPGPHTQVPLRSGSPAPPEPVDPSLGLRALAPEEVEMLYEEALYTVLYRAGTMGPDQVDDEEALLSYLQQVFGTSLEEHTEAIERVRKAKAPTYALKVSVMRAKNLLAKDPNGFSDPYCMLGILPASDATREPRAQKEQRFGFRKGSKRGGPLPAKCIQVTEVKSSTLNPVWKEHFLFEIEDVSTDQLHLDIWDHDDDVSLVEACRKLNEVIGLKGMGRYFKQIVKSARANGTAGPTEDHTDDFLGCLNIPVREVPVAGVDRWFKLEPRSSASRVQGHCHLVLKLITTQRDTAMSQRGRSGFLSHLLLLSHLLRLEHSAEEPNSSSWRGELSTPAATILCLHGAQSNLSPLQLAVLHWQVSSRHHQTCTLDYSYLLGLLEDMQAHWEEAPSLPQEQEESLADSLSAFSEFGLQLLRQLRDYFPATNSTAVHRLELLLKCLGKLQLFQPSFEICPFESELNMDIAAALKRGNREWYDRILNDKSPREQPGPQRLPGLVVLADAVYDDLQFCYSVYASLFHSILNVDVFTLTFRQLERLVAEEAWVLTEELSPKMTLEVASGLFELYLTLADLQRFWDSIPGRDSRSLALAGIHAPFLPAVKLWFQVLRDQAKWRLQGAVDMDTLEPVDASSRHSSSAATAGLCLSHIQELWVRLAWPDPAQAQGLGTQLGQDVCEATLFYTELLRKKVDTQPGAAGEAVSEALCVVLNNVELVRKAAGQALKGLAWPEGATGPEGVLPRPLLSCTQALDDDLQREAHTVTAHLTSKMVGDIRKYVQHISLSPDSIQNDEAVAPLMKYLDEKLALLNASLVKGNLSRVLEALWELLLQAILQALGANRDVSADFYSRFHFTLEALVSFFHAEGQGLPLESLRDGSYKRLKEELRLHKCSTRECIEQFYLDKLKQRTLEQNRFGRLSVRCHYEAAEQRLAVEVLHAADLLPLDANGLSDPFVIVELGPPHLFPLVRSQRTQVKTRTLHPVYDELFYFSVPAEACRRRAACVLFTVMDHDWLSTNDFAGEAALGLGGVTGVARPQVGGGARAGQPVTLHLCRPRAQVRSALRRLEGRTSKEAQEFVKKLKELEKCMEADP

SEQ ID NO: 68 - protein, 1129 aa, Homo sapiens
MSTLLDIKSSVLRQVQVCPSFRRRTEQDPGSASADPQEPATGAWKPGDGVEFFAHMRLMLKKGEGRQGLPCLEVPLRSGSPAPPEPVDPSLGLRALAPEEVDDEEALLSYLQQVFGTSLEEHTEAIERVRKAKAPTYALKVSVMRAKNLLAKDPNGFSDPYCMLGILPASDATREPRAQKEQRFGFRKGSKRGGPLPAKCIQVTEVKSSTLNPVWKEHFLFEIEDVSTDQLHLDIWDHDDDVSLVEACRKLNEVIGLKGMGRYFKQIVKSARANGTAGPTEDHTDDFLGCLNIPVREVPVAGVDRWFKLEPRSSASRVQGHCHLVLKLITTQRDTAMSQRGRSGFLSHLLLLSHLLRLEHSAEEPNSSSWRGELSTPAATILCLHGAQSNLSPLQLAVLHWQVSSRHHQTCTLDYSYLLGLLEDMQAHWEEAPSLPQEQEESLADSLSAFSEFGLQLLRQLRDYFPATNSTAVHRLELLLKCLGKLQLFQPSFEICPFESELNMDIAAALKRGNREWYDRILNDKSPREQPGPQRLPGLVVLADAVYDDLQFCYSVYASLFHSILNVDVFTLTFRQLERLVAEEAWVLTEELSPKMTLEVASGLFELYLTLADLQRFWDSIPGRDSRSLALAGIHAPFLPAVKLWFQVLRDQAKWRLQGAVDMDTLEPVDASSRHSSSAATAGLCLSHIQELWVRLAWPDPAQAQGLGTQLGQDVCEATLFYTELLRKKVDTQPGAAGEAVSEALCVVLNNVELVRKAAGQALKGLAWPEGATGPEGVLPRPLLSCTQALDDDLQREAHTVTAHLTSKMVGDIRKYVQHISLSPDSIQNDEAVAPLMKYLDEKLALLNASLVKGNLSRVLEALWELLLQAILQALGANRDVSADFYSRFHFTLEALVSFFHAEGQGLPLESLRDGSYKRLKEELRLHKCSTRECIEQFYLDKLKQRTLEQNRFGRLSVRCHYEAAEQRLAVEVLHAADLLPLDANGLSDPFVIVELGPPHLFPLVRSQRTQVKTRTLHPVYDELFYFSVPAEACRRRAACVLFTVMDHDWLSTNDFAGEAALGLGGVTGVARPQVGGGARAGQPVTLHLCRPRAQVRSALRRLEGRTSKEAQEFVKKLKELEKCMEADP

SEQ ID NO: 69 - protein, 1124 aa, Homo sapiens
MSTLLDIKSSVLRQVQVCPSFRRRTEQDPGSASADPQEPATGAWKPGDGVEFFAHMRLMLKKGEGRQGLPCLEVPLRSGSPAPPEPVDPSLGLRALAPEEALLSYLQQVFGTSLEEHTEAIERVRKAKAPTYALKVSVMRAKNLLAKDPNGFSDPYCMLGILPASDATREPRAQKEQRFGFRKGSKRGGPLPAKCIQVTEVKSSTLNPVWKEHFLFEIEDVSTDQLHLDIWDHDDDVSLVEACRKLNEVIGLKGMGRYFKQIVKSARANGTAGPTEDHTDDFLGCLNIPVREVPVAGVDRWFKLEPRSSASRVQGHCHLVLKLITTQRDTAMSQRGRSGFLSHLLLLSHLLRLEHSAEEPNSSSWRGELSTPAATILCLHGAQSNLSPLQLAVLHWQVSSRHHQTCTLDYSYLLGLLEDMQAHWEEAPSLPQEQEESLADSLSAFSEFGLQLLRQLRDYFPATNSTAVHRLELLLKCLGKLQLFQPSFEICPFESELNMDIAAALKRGNREWYDRILNDKSPREQPGPQRLPGLVVLADAVYDDLQFCYSVYASLFHSILNVDVFTLTFRQLERLVAEEAWVLTEELSPKMTLEVASGLFELYLTLADLQRFWDSIPGRDSRSLALAGIHAPFLPAVKLWFQVLRDQAKWRLQGAVDMDTLEPVDASSRHSSSAATAGLCLSHIQELWVRLAWPDPAQAQGLGTQLGQDVCEATLFYTELLRKKVDTQPGAAGEAVSEALCVVLNNVELVRKAAGQALKGLAWPEGATGPEGVLPRPLLSCTQALDDDLQREAHTVTAHLTSKMVGDIRKYVQHISLSPDSIQNDEAVAPLMKYLDEKLALLNASLVKGNLSRVLEALWELLLQAILQALGANRDVSADFYSRFHFTLEALVSFFHAEGQGLPLESLRDGSYKRLKEELRLHKCSTRECIEQFYLDKLKQRTLEQNRFGRLSVRCHYEAAEQRLAVEVLHAADLLPLDANGLSDPFVIVELGPPHLFPLVRSQRTQVKTRTLHPVYDELFYFSVPAEACRRRAACVLFTVMDHDWLSTNDFAGEAALGLGGVTGVARPQVGGGARAGQPVTLHLCRPRAQVRSALRRLEGRTSKEAQEFVKKLKELEKCMEADP

SEQ ID NO: 70 - protein, 1152 aa, Homo sapiens
MSTLLDIKSSVLRQVQVCPSFRRRTEQDPGSASADPQEPATGAWKPGDGVEFFAHMRLMLKKGEGRQGLPCLEVPLRSGSPAPPEPVDPSLGLRALAPEEVEMLYEEALYTVLYRAGTMGPDQVDDEEALLSYLQQVFGTSLEEHTEAIERVRKAKAPTYALKVSVMRAKNLLAKDPNGFSDPYCMLGILPASDATREPRAQKEQRFGFRKGSKRGGPLPAKCIQVTEVKSSTLNPVWKEHFLFEIEDVSTDQLHLDIWDHDDDVSLVEACRKLNEVIGLKGMGRYFKQIVKSARANGTAGPTEDHTDDFLGCLNIPVREVPVAGVDRWFKLEPRSSASRVQGHCHLVLKLITTQRDTAMSQRGRSGFLSHLLLLSHLLRLEHSAEEPNSSSWRGELSTPAATILCLHGAQSNLSPLQLAVLHWQVSSRHHQTCTLDYSYLLGLLEDMQAHWEEAPSLPQEQEESLADSLSAFSEFGLQLLRQLRDYFPATNSTAVHRLELLLKCLGKLQLFQPSFEICPFESELNMDIAAALKRGNREWYDRILNDKSPREQPGPQRLPGLVVLADAVYDDLQFCYSVYASLFHSILNVDVFTLTFRQLERLVAEEAWVLTEELSPKMTLEVASGLFELYLTLADLQRFWDSIPGRDSRSLALAGIHAPFLPAVKLWFQVLRDQAKWRLQGAVDMDTLEPVDASSRHSSSAATAGLCLSHIQELWVRLAWPDPAQAQGLGTQLGQDVCEATLFYTELLRKKVDTQPGAAGEAVSEALCVVLNNVELVRKAAGQALKGLAWPEGATGPEGVLPRPLLSCTQALDDDLQREAHTVTAHLTSKMVGDIRKYVQHISLSPDSIQNDEAVAPLMKYLDEKLALLNASLVKGNLSRVLEALWELLLQAILQALGANRDVSADFYSRFHFTLEALVSFFHAEGQGLPLESLRDGSYKRLKEELRLHKCSTRECIEQFYLDKLKQRTLEQNRFGRLSVRCHYEAAEQRLAVEVLHAADLLPLDANGLSDPFVIVELGPPHLFPLVRSQRTQVKTRTLHPVYDELFYFSVPAEACRRRAACVLFTVMDHDWLSTNDFAGEAALGLGGVTGVARPQVGGGARAGQPVTLHLCRPRAQVRSALRRLEGRTSKEAQEFVKKLKELEKCMEADP

SEQ ID NO: 71 - protein, 1116 aa, Homo sapiens
MSTLLDIKSSVLRQVQVCPSFRRRTEQDPGSASADPQEPATGAWKPGDGVEFFAHMRLMLKKGEGRQGLPCLEVPLRSGSPAPPEPVDPSLGLRALAPEEVEMLYEEALYTVLYRAGTMGPDQVDDEEALLSYLQQVFGTSLEEHTEAIERVRKAKAPTYALKVSVMRAKNLLAKDPNGFSDPYCMLGILPASDATREPRAQKEQRFGFRKGSKRGGPLPAKCIQVTEVKSSTLNPVWKEHFLFEIEDVSTDQLHLDIWDHDDDVSLVEACRKLNEVIGLKGMGRYFKQIVKSARANGTAGPTEDHTDDFLGCLNIPVRRDTAMSQRGRSGFLSHLLLLSHLLRLEHSAEEPNSSSWRGELSTPAATILCLHGAQSNLSPLQLAVLHWQVSSRHHQTCTLDYSYLLGLLEDMQAHWEEAPSLPQEQEESLADSLSAFSEFGLQLLRQLRDYFPATNSTAVHRLELLLKCLGKLQLFQPSFEICPFESELNMDIAAALKRGNREWYDRILNDKSPREQPGPQRLPGLVVLADAVYDDLQFCYSVYASLFHSILNVDVFTLTFRQLERLVAEEAWVLTEELSPKMTLEVASGLFELYLTLADLQRFWDSIPGRDSRSLALAGIHAPFLPAVKLWFQVLRDQAKWRLQGAVDMDTLEPVDASSRHSSSAATAGLCLSHIQELWVRLAWPDPAQAQGLGTQLGQDVCEATLFYTELLRKKVDTQPGAAGEAVSEALCVVLNNVELVRKAAGQALKGLAWPEGATGPEGVLPRPLLSCTQALDDDLQREAHTVTAHLTSKMVGDIRKYVQHISLSPDSIQNDEAVAPLMKYLDEKLALLNASLVKGNLSRVLEALWELLLQAILQALGANRDVSADFYSRFHFTLEALVSFFHAEGQGLPLESLRDGSYKRLKEELRLHKCSTRECIEQFYLDKLKQRTLEQNRFGRLSVRCHYEAAEQRLAVEVLHAADLLPLDANGLSDPFVIVELGPPHLFPLVRSQRTQVKTRTLHPVYDELFYFSVPAEACRRRAACVLFTVMDHDWLSTNDFAGEAALGLGGVTGVARPQVGGGARAGQPVTLHLCRPRAQVRSALRRLEGRTSKEAQEFVKKLKELEKCMEADP

SEQ ID NO: 72 - protein, 488 aa, Homo sapiens; identical to SEQ ID NO: 1

SEQ ID NO: 73 - protein, 2419 aa, Homo sapiens
MEQFPKETVVESSGPKVLETAEEIQERRQEVLTRYQSFKERVAERGQKLEDSYHLQVFKRDADDLGKWIMEKVNILTDKSYEDPTNIQGKYQKHQSLEAEVQTKSRLMSELEKTREERFTMGHSAHEETKAHIEELRHLWDLLLELTLEKGDQLLRALKFQQYVQECADILEWIGDKEAIATSVELGEDWERTEVLHKKFEDFQVELVAKEGRVVEVNQYANECAEENHPDLPLIQSKQNEVNAAWERLRGLALQRQKALSNAANLQRFKRDVTEAIQWIKEKEPVLTSEDYGKDLVASEGLFHSHKGLERNLAVMSDKVKELCAKAEKLTLSHPSDAPQIQEMKEDLVSSWEHIRALATSRYEKLQATYWYHRFSSDFDELSGWMNEKTAAINADELPTDVAGGEVLLDRHQQHKHEIDSYDDRFQSADETGQDLVNANHEASDEVREKMEILDNNWTALLELWDERHRQYEQCLDFHLFYRDSEQVDSWMSRQEAFLENEDLGNSLGSAEALLQKHEDFEEAFTAQEEKIITVDKTATKLIGDDHYDSENIKAIRDGLLARRDALREKAATRRRLLKESLLLQKLYEDSDDLKNWINKKKKLADDEDYKDIQNLKSRVQKQQVFEKELAVNKTQLENIQKTGQEMIEGGHYASDNVTTRLSEVASLWEELLEATKQKGTQLHEANQQLQFENNAEDLQRWLEDVEWQVTSEDYGKGLAEVQNRLRKHGLLESAVAARQDQVDILTDLAAYFEEIGHPDSKDIRARQESLVCRFEALKEPLATRKKKLLDLLHLQLICRDTEDEEAWIQETEPSATSTYLGKDLIASKKLLNRHRVILENIASHEPRIQEITERGNKMVEEGHFAAEDVASRVKSLNQNMESLRARAARRQNDLEANVQFQQYLADLHEAETWIREKEPIVDNTNYGADEEAAGALLKKHEAFLLDLNSFGDSMKALRNQANACQQQQAAPVEGVAGEQRVMALYDFQARSPREVTMKKGDVLTLLSSINKDWWKVEAADHQGIVPAVYVRRLAHDEFPMLPQRRREEPGNITQRQEQIENQYRSLLDRAEERRRRLLQRYNEFLLAYEAGDMLEWIQEKKAENTGVELDDVWELQKKFDEFQKDLNTNEPRLRDINKVADDLLFEGLLTPEGAQIRQELNSRWGSLQRLADEQRQLLGSAHAVEVFHREADDTKEQIEKKCQALSAADPGSDLFSVQALQRRHEGFERDLVPLGDKVTILGETAERLSESHPDATEDLQRQKMELNEAWEDLQGRTKDRKESLNEAQKFYLFLSKARDLQNWISSIGGMVSSQELAEDLTGIEILLERHQEHRADMEAEAPTFQALEDFSAELIDSGHHASPEIEKKLQAVKLERDDLEKAWEKRKKILDQCLELQMFQGNCDQVESWMVARENSLRSDDKSSLDSLEALMKKRDDLDKAITAQEGKITDLEHFAESLIADEHYAKEEIATRLQRVLDRWKALKAQLIDERTKLGDYANLKQFYRDLEELEEWISEMLPTACDESYKDATNIQRKYLKHQTFAHEVDGRSEQVHGVINLGNSLIECSACDGNEEAMKEQLEQLKEHWDHLLERTNDKGKKLNEASRQQRFNTSIRDFEFWLSEAETLLAMKDQARDLASAGNLLKKHQLLEREMLAREDALKDLNTLAEDLLSSGTFNVDQIVKKKDNVNKRFLNVQELAAAHHEKLKEAYALFQFFQDLDDEESWIEEKLIRVSSQDYGRDLQGVQNLLKKHKRLEGELVAHEPAIQNVLDMAEKLKDKAAVGQEEIQLRLAQFVEHWEKLKELAKARGLKLEESLEYLQFMQNAEEEEAWINEKNALAVRGDCGDTLAATQSLLMKHEALENDFAVHETRVQNVCAQGEDILNKVLQEESQNKEISSKIEALNEKTPSLAKAIAAWKLQLEDDYAFQEFNWKADVVEAWIADKETSLKTNGNGADLGDFLTLLAKQDTLDASLQSFQQERLPEITDLKDKLISAQHNQSKAIEERYAALLKRWEQLLEASAVHRQKLLEKQLPLQKAEDLFVEFAHKASALNNWCEKMEENLSEPVHCVSLNEIRQLQKDHEDFLASLARAQADFKCLLELDQQIKALGVPSSPYTWLTVEVLERTWKHLSDIIEEREQELQKEEARQVKNFEMCQEFEQNASTFLQWILETRAYFLDGSLLKETGTLESQLEANKRKQKEIQAMKRQLTKIVDLGDNLEDALILDIKYSTIGLAQQWDQLYQLGLRMQHNLEQQIQAKDIKGVSEETLKEFSTIYKHFDENLTGRLTHKEFRSCLRGLNYYLPMVEEDEHEPKFEKFLDAVDPGRKGYVSLEDYTAFLIDKESENIKSSDEIENAFQALAEGKSYITKEDMKQALTPEQVSFCATHMQQYMDPRGRSHLSGYDYVGFTNSYFGN

SEQ ID NO: 74 - protein, 2416 aa, Homo sapiens
MEQFPKETVVESSGPKVLETAEEIQERRQEVLTRYQSFKERVAERGQKLEDSYHLQVFKRDADDLGKWIMEKVNILTDKSYEDPTNIQGKYQKHQSLEAEVQTKSRLMSELEKTREERFTMGHSAHEETKAHIEELRHLWDLLLELTLEKGDQLLRALKFQQYVQECADILEWIGDKEAIATSVELGEDWERTEVLHKKFEDFQVELVAKEGRVVEVNQYANECAEENHPDLPLIQSKQNEVNAAWERLRGLALQRQKALSNAANLQRFKRDVTEAIQWIKEKEPVLTSEDYGKDLVASEGLFHSHKGLERNLAVMSDKVKELCAKAEKLTLSHPSDAPQIQEMKEDLVSSWEHIRALATSRYEKLQATYWYHRFSSDFDELSGWMNEKTAAINADELPTDVAGGEVLLDRHQQHKHEIDSYDDRFQSADETGQDLVNANHEASDEVREKMEILDNNWTALLELWDERHRQYEQCLDFHLFYRDSEQVDSWMSRQEAFLENEDLGNSLGSAEALLQKHEDFEEAFTAQEEKIITVDKTATKLIGDDHYDSENIKAIRDGLLARRDALREKAATRRRLLKESLLLQKLYEDSDDLKNWINKKKKLADDEDYKDIQNLKSRVQKQQVFEKELAVNKTQLENIQKTGQEMIEGGHYASDNVTTRLSEVASLWEELLEATKQKGTQLHEANQQLQFENNAEDLQRWLEDVEWQVTSEDYGKGLAEVQNRLRKHGLLESAVAARQDQVDILTDLAAYFEEIGHPDSKDIRARQESLVCRFEALKEPLATRKKKLLDLLHLQLICRDTEDEEAWIQETEPSATSTYLGKDLIASKKLLNRHRVILENIASHEPRIQEITERGNKMVEEGHFAAEDVASRVKSLNQNMESLRARAARRQNDLEANVQFQQYLADLHEAETWIREKEPIVDNTNYGADEEAAGALLKKHEAFLLDLNSFGDSMKALRNQANACQQQQAAPVEGVAGEQRVMALYDFQARSPREVTMKKGDVLTLLSSINKDWWKVEAADHQGIVPAVYVRRLAHDEFPMLPQRRREEPGNITQRQEQIENQYRSLLDRAEERRRRLLQRYNEFLLAYEAGDMLEWIQEKKAENTGVELDDVWELQKKFDEFQKDLNTNEPRLRDINKVADDLLFEGLLTPEGAQIRQELNSRWGSLQRLADEQRQLLGSAHAVEVFHREADDTKEQIEKKCQALSAADPGSDLFSVQALQRRHEGFERDLVPLGDKVTILGETAERLSESHPDATEDLQRQKMELNEAWEDLQGRTKDRKESLNEAQKFYLFLSKARDLQNWISSIGGMVSSQELAEDLTGIEILLERHQEHRADMEAEAPTFQALEDFSAELIDSGHHASPEIEKKLQAVKLERDDLEKAWEKRKKILDQCLELQMFQGNCDQVESWMVARENSLRSDDKSSLDSLEALMKKRDDLDKAITAQEGKITDLEHFAESLIADEHYAKEEIATRLQRVLDRWKALKAQLIDERTKLGDYANLKQFYRDLEELEEWISEMLPTACDESYKDATNIQRKYLKHQTFAHEVDGRSEQVHGVINLGNSLIECSACDGNEEAMKEQLEQLKEHWDHLLERTNDKGKKLNEASRQQRFNTSIRDFEFWLSEAETLLAMKDQARDLASAGNLLKKHQLLEREMLAREDALKDLNTLAEDLLSSGTFNVDQIVKKKDNVNKRFLNVQELAAAHHEKLKEAYALFQFFQDLDDEESWIEEKLIRVSSQDYGRDLQGVQNLLKKHKRLEGELVAHEPAIQNVLDMAEKLKDKAAVGQEEIQLRLAQFVEHWEKLKELAKARGLKLEESLEYLQFMQNAEEEEAWINEKNALAVRGDCGDTLAATQSLLMKHEALENDFAVHETRVQNVCAQGEDILNKEESQNKEISSKIEALNEKTPSLAKAIAAWKLQLEDDYAFQEFNWKADVVEAWIADKETSLKTNGNGADLGDFLTLLAKQDTLDASLQSFQQERLPEITDLKDKLISAQHNQSKAIEERYAALLKRWEQLLEASAVHRQKLLEKQLPLQKAEDLFVEFAHKASALNNWCEKMEENLSEPVHCVSLNEIRQLQKDHEDFLASLARAQADFKCLLELDQQIKALGVPSSPYTWLTVEVLERTWKHLSDIIEEREQELQKEEARQVKNFEMCQEFEQNASTFLQWILETRAYFLDGSLLKETGTLESQLEANKRKQKEIQAMKRQLTKIVDLGDNLEDALILDIKYSTIGLAQQWDQLYQLGLRMQHNLEQQIQAKDIKGVSEETLKEFSTIYKHFDENLTGRLTHKEFRSCLRGLNYYLPMVEEDEHEPKFEKFLDAVDPGRKGYVSLEDYTAFLIDKESENIKSSDEIENAFQALAEGKSYITKEDMKQALTPEQVSFCATHMQQYMDPRGRSHLSGYDYVGFTNSYFGN

SEQ ID NO: 75 - protein, 4563 aa, Homo sapiens; identical to SEQ ID NO: 4

SEQ ID NO: 76 - protein, 406 aa, Homo sapiens; identical to SEQ ID NO: 9

SEQ ID NO: 77 - protein, 499 aa, Homo sapiens
MKHSLNALLIFLIITSAWGGSKGPLDQLEKGGETAQSADPQWEQLNNKNLSMPLLPADFHKENTVTNDWIPEGEEDDDYLDLEKIFSEDDDYIDIVDSLSVSPTDSDVSAGNILQLFHGKSRIQRLNILNAKFAFNLYRVLKDQVNTFDNIFIAPVGISTAMGMISLGLKGETHEQVHSILHFKDFVNASSKYEITTIHNLFRKLTHRLFRRNFGYTLRSVNDLYIQKQFPILLDFKTKVREYYFAEAQIADFSDPAFISKTNNHIMKLTKGLIKDALENIDPATQMMILNCIYFKGSWVNKFPVEMTHNHNFRLNEREVVKVSMMQTKGNFLAANDQELDCDILQLEYVGGISMLIVVPHKMSGMKTLEAQLTPRVVERWQKSMTNRTREVLLPKFKLEKNYNLVESLKLMGIRMLFDKNGNMAGISDQRIAIDLFKHQGTITVNEEGTQATTVTTVGFMPLSTQVRFTVDRPFLFLIYEHRTSCLLFMGRVANPSRS

SEQ ID NO: 78 - protein, 272 aa, Homo sapiens
MVLLTAVLLLLAAYAGPAQSLGSFVHCEPCDEKALSMCPPSPLGCELVKEPGCGCCMTCALAEGQSCGVYTERCAQGLRCLPRQDEEKPLHALLHGRGVCLNEKSYREQVKIERDSREHEEPTTSEMAEETYSPKIFRPKHTRISELKAEAVKKDRRKKLTQSKFVGGAENTAHPRIISAPEMRQESEQGPCRRHMEASLQELKASPRMVPRAVYLPNCDRKGFYKRKQCKPSRGRKRGICWCVDKYGMKLPGMEYVDGDFQCHTFDSSNVE

SEQ ID NO: 79 - protein, 206 aa, Homo sapiens; identical to SEQ ID NO: 46

SEQ ID NO: 80 - protein, 605 aa, Homo sapiens
MALRKGGLALALLLLSWVALGPRSLEGADPGTPGEAEGPACPAACVCSYDDDADELSVFCSSRNLTRLPDGVPGGTQALWLDGNNLSSVPPAAFQNLSSLGFLNLQGGQLGSLEPQALLGLENLCHLHLERNQLRSLALGTFAHTPALASLGLSNNRLSRLEDGLFEGLGSLWDLNLGWNSLAVLPDAAFRGLGSLRELVLAGNRLAYLQPALFSGLAELRELDLSRNALRAIKANVFVQLPRLQKLYLDRNLIAAVAPGAFLGLKALRWLDLSHNRVAGLLEDTFPGLLGLRVLRLSHNAIASLRPRTFKDLHFLEELQLGHNRIRQLAERSFEGLGQLEVLTLDHNQLQEVKAGAFLGLTNVAVMNLSGNCLRNLPEQVFRGLGKLHSLHLEGSCLGRIRPHTFTGLSGLRRLFLKDNGLVGIEEQSLWGLAELLELDLTSNQLTHLPHRLFQGLGKLEYLLLSRNRLAELPADALGPLQRAFWLDVSHNRLEALPNSLLAPLGRLRYLSLRNNSLRTFTPQPPGLERLWLEGNPWDCGCPLKALRDFALQNPSAVPRFVQAICEGDDCQPPAYTYNNITCASPPEVVGLDLRDLSEAHFAPC

SEQ ID NO: 81 - protein, 643 aa, Homo sapiens
MALRKAGDLEPQFTPERRFRLCWYQAHSGRALLGPPPQASPPAGGLALALLLLSWVALGPRSLEGADPGTPGEAEGPACPAACVCSYDDDADELSVFCSSRNLTRLPDGVPGGTQALWLDGNNLSSVPPAAFQNLSSLGFLNLQGGQLGSLEPQALLGLENLCHLHLERNQLRSLALGTFAHTPALASLGLSNNRLSRLEDGLFEGLGSLWDLNLGWNSLAVLPDAAFRGLGSLRELVLAGNRLAYLQPALFSGLAELRELDLSRNALRAIKANVFVQLPRLQKLYLDRNLIAAVAPGAFLGLKALRWLDLSHNRVAGLLEDTFPGLLGLRVLRLSHNAIASLRPRTFKDLHFLEELQLGHNRIRQLAERSFEGLGQLEVLTLDHNQLQEVKAGAFLGLTNVAVMNLSGNCLRNLPEQVFRGLGKLHSLHLEGSCLGRIRPHTFTGLSGLRRLFLKDNGLVGIEEQSLWGLAELLELDLTSNQLTHLPHRLFQGLGKLEYLLLSRNRLAELPADALGPLQRAFWLDVSHNRLEALPNSLLAPLGRLRYLSLRNNSLRTFTPQPPGLERLWLEGNPWDCGCPLKALRDFALQNPSAVPRFVQAICEGDDCQPPAYTYNNITCASPPEVVGLDLRDLSEAHFAPC

SEQ ID NO: 82 - protein, 407 aa, Homo sapiens
MVNEYKKILLLKGFELMDDYHFTSIKSLLAYDLGLTTKMQEEYNRIKITDLMEKKFQGVACLDKLIELAKDMPSLKNLVNNLRKEKSKVAKKIKTQEKAPVKKINQEEVGLAAPAPTARNKLTSEARGRIPVAQKRKTPNKEKTEAKRNKVSQEQSKPPGPSGASTSAAVDHPPLPQTSSSTPSNTSFTPNQETQAQRQVDARRNVPQNDPVTVVVLKATAPFKYESPENGKSTMFHATVASKTQYFHVKVFDINLKEKFVRKKVITISDYSECKGVMEIKEASSVSDFNQNFEVPNRIIEIANKTPKISQLYKQASGTMVYGLFMLQKKSVHKKNTIYEIQDNTGSMDVVGSGKWHNIKCEKGDKLRLFCLQLRTVDRKLKLVCGSHSFIKVIKAKKNKEGPMNVN

SEQ ID NO: 83 - protein, 749 aa, Homo sapiens
MGRAGAAAVIPGLALLWAVGLGSAAPSPPRLRLSFQELQAWHGLQTFSLERTCCYQALLVDEERGRLFVGAENHVASLNLDNISKRAKKLAWPAPVEWREECNWAGKDIGTECMNFVKLLHAYNRTHLLACGTGAFHPTCAFVEVGHRAEEPVLRLDPGRIEDGKGKSPYDPRHRAASVLVGEELYSGVAADLMGRDFTIFRSLGQRPSLRTEPHDSRWLNEPKFVKVFWIPESENPDDDKIYFFFRETAVEAAPALGRLSVSRVGQICRNDVGGQRSLVNKWTTFLKARLVCSVPGVEGDTHFDQLQDVFLLSSRDHRTPLLYAVFSTSSSIFQGSAVCVYSMNDVRRAFLGPFAHKEGPMHQWVSYQGRVPYPRPGMCPSKTFGTFSSTKDFPDDVIQFARNHPLMYNSVLPTGGRPLFLQVGANYTFTQIAADRVAAADGHYDVLFIGTDVGTVLKVISVPKGSRPSAEGLLLEELHVFEDSAAVTSMQISSKRHQLYVASRSAVAQIALHRCAAHGRVCTECCLARDPYCAWDGVACTRFQPSAKRRFRRQDVRNGDPSTLCSGDSSRPALLEHKVFGVEGSSAFLECEPRSLQARVEWTFQRAGVTAHTQVLAEERTERTARGLLLRRLRRRDSGVYLCAAVEQGFTQPLRRLSLHVLSATQAERLARAEEAAPAAPPGPKLWYRDFLQLVEPGGGGSANSLRMCRPQPALQSLPLESRRKGRNRRTHAPEPRAERGPRSATHW

SEQ ID NO: 84 - protein, 748 aa, Homo sapiens
MGRAGAAAVIPGLALLWAVGLGSAAPSPPRLRLSFQELQAWHGLQTFSLERTCCYQALLVDEERGRLFVGAENHVASLNLDNISKRAKKLAWPAPVEWREECNWAGKDIGTECMNFVKLLHAYNRTHLLACGTGAFHPTCAFVEVGHRAEEPVLRLDPGRIEDGKGKSPYDPRHRAASVLVGEELYSGVAADLMGRDFTIFRSLGQRPSLRTEPHDSRWLNEPKFVKVFWIPESENPDDDKIYFFFRETAVEAAPALGRLSVSRVGQICRNDVGGQRSLVNKWTTFLKARLVCSVPGVEGDTHFDQLQDVFLLSSRDHRTPLLYAVFSTSSIFQGSAVCVYSMNDVRRAFLGPFAHKEGPMHQWVSYQGRVPYPRPGMCPSKTFGTFSSTKDFPDDVIQFARNHPLMYNSVLPTGGRPLFLQVGANYTFTQIAADRVAAADGHYDVLFIGTDVGTVLKVISVPKGSRPSAEGLLLEELHVFEDSAAVTSMQISSKRHQLYVASRSAVAQIALHRCAAHGRVCTECCLARDPYCAWDGVACTRFQPSAKRRFRRQDVRNGDPSTLCSGDSSRPALLEHKVFGVEGSSAFLECEPRSLQARVEWTFQRAGVTAHTQVLAEERTERTARGLLLRRLRRRDSGVYLCAAVEQGFTQPLRRLSLHVLSATQAERLARAEEAAPAAPPGPKLWYRDFLQLVEPGGGGSANSLRMCRPQPALQSLPLESRRKGRNRRTHAPEPRAERGPRSATHW

SEQ ID NO: 85 - protein, 863 aa, Homo sapiens; identical to SEQ ID NO: 58

SEQ ID NO: 86 - protein, 915 aa, Homo sapiens; identical to SEQ ID NO: 59

SEQ ID NO: 87 - protein, 888 aa, Homo sapiens; identical to SEQ ID NO: 60

SEQ ID NO: 88 - protein, 470 aa, Homo sapiens; identical to SEQ ID NO: 62

SEQ ID NO: 89 - protein, 423 aa, Homo sapiens; identical to SEQ ID NO: 63

SEQ ID NO: 90 - protein, 360 aa, Homo sapiens; identical to SEQ ID NO: 64

SEQ ID NO: 91 - protein, 3063 aa, Homo sapiens
MRSRLPPALAALGAALLLSSIEAEVDPPSDLNFKIIDENTVHMSWAKPVDPIVGYRITVDPTTDGPTKEFTLSASTTETLLSELVPETEYVVTITSYDEVEESVPVIGQLTIQTGSSTKPVEKKPGKTEIQKCSVSAWTDLVFLVDGSWSVGRNNFKYILDFIAALVSAFDIGEEKTRVGVVQYSSDTRTEFNLNQYYQRDELLAAIKKIPYKGGNTMTGDAIDYLVKNTFTESAGARVGFPKVAIIITDGKSQDEVEIPARELRNVGVEVFSLGIKAADAKELKQIASTPSLNHVFNVANFDAIVDIQNEIISQVCSGVDEQLGELVSGEEVVEPPSNLIAMEVSSKYVKLNWNPSPSPVTGYKVILTPMTAGSRQHALSVGPQTTTLSVRDLSADTEYQISVSAMKGMTSSEPISIMEKTQPMKVQVECSRGVDIKADIVFLVDGSYSIGIANFVKVRAFLEVLVKSFEISPNRVQISLVQYSRDPHTEFTLKKFTKVEDIIEAINTFPYRGGSTNTGKAMTYVREKIFVPSKGSRSNVPKVMILITDGKSSDAFRDPAIKLRNSDVEIFAVGVKDAVRSELEAIASPPAETHVFTVEDFDAFQRISFELTQSICLRIEQELAAIKKKAYVPPKDLSFSEVTSYGFKTNWSPAGENVFSYHITYKEAAGDDEVTVVEPASSTSVVLSSLKPETLYLVNVTAEYEDGFSIPLAGEETTEEVKGAPRNLKVTDETTDSFKITWTQAPGRVLRYRIIYRPVAGGESREVTTPPNQRRRTLENLIPDTKYEVSVIPEYFSGPGTPLTGNAATEEVRGNPRDLRVSDPTTSTMKLSWSGAPGKVKQYLVTYTPVAGGETQEVTVRGDTTNTVLQGLKEGTQYALSVTALYASGAGDALFGEGTTLEERGSPQDLVTKDITDTSIGAYWTSAPGMVRGYRVSWKSLYDDVDTGEKNLPEDAIHTMIENLQPETKYRISVFATYSSGEGEPLTGDATTELSQDSKTLKVDEETENTMRVTWKPAPGKVVNYRVVYRPHGRGKQMVAKVPPTVTSTVLKRLQPQTTYDITVLPIYKMGEGKLRQGSGTTASRFKSPRNLKTSDPTMSSFRVTWEPAPGEVKGYKVTFHPTGDDRRLGELVVGPYDNTVVLEELRAGTTYKVNVFGMFDGGESSPLVGQEMTTLSDTTVMPILSSGMECLTRAEADIVLLVDGSWSIGRANFRTVRSFISRIVEVFDIGPKRVQIALAQYSGDPRTEWQLNAHRDKKSLLQAVANLPYKGGNTLTGMALNFIRQQNFRTQAGMRPRARKIGVLITDGKSQDDVEAPSKKLKDEGVELFAIGIKNADEVELKMIATDPDDTHAYNVADFESLSRIVDDLTINLCNSVKGPGDLEAPSNLVISERTHRSFRVSWTPPSDSVDRYKVEYYPVSGGKRQEFYVSRMETSTVLKDLKPETEYVVNVYSVVEDEYSEPLKGTEKTLPVPVVSLNIYDVGPTTMHVQWQPVGGATGYILSYKPVKDTEPTRPKEVRLGPTVNDMQLTDLVPNTEYAVTVQAVLHDLTSEPVTVREVTLPLPRPQDLKLRDVTHSTMNVFWEPVPGKVRKYIVRYKTPEEDVKEVEVDRSETSTSLKDLFSQTLYTVSVSAVHDEGESPPVTAQETTRPVPAPTNLKITEVTSEGFRGTWDHGASDVSLYRITWAPFGSSDKMETILNGDENTLVFENLNPNTIYEVSITAIYPDESESDDLIGSERTLPILTTQAPKSGPRNLQVYNATSNSLTVKWDPASGRVQKYRITYQPSTGEGNEQTTTIGGRQNSVVLQKLKPDTPYTITVSSLYPDGEGGRMTGRGKTKPLNTVRNLRVYDPSTSTLNVRWDHAEGNPRQYKLFYAPAAGGPEELVPIPGNTNYAILRNLQPDTSYTVTVVPVYTEGDGGRTSDTGRTLMRGLARNVQVYNPTPNSLDVRWDPAPGPVLQYRVVYSPVDGTRPSESIVVPGNTRMVHLERLIPDTLYSVNLVALYSDGEGNPSPAQGRTLPRSGPRNLRVFGETTNSLSVAWDHADGPVQQYRIIYSPTVGDPIDEYTTVPGRRNNVILQPLQPDTPYKITVIAVYEDGDGGHLTGNGRTVGLLPPQNIHISDEWYTRFRVSWDPSPSPVLGYKIVYKPVGSNEPMEAFVGEMTSYTLHNLNPSTTYDVNVYAQYDSGLSVPLTDQGTTLYLNVTDLKTYQIGWDTFCVKWSPHRAATSYRLKLSPADGTRGQEITVRGSETSHCFTGLSPDTDYGVTVFVQTPNLEGPGVSVKEHTTVKPTEAPTEPPTPPPPPTIPPARDVCKGAKADIVFLTDASWSIGDDNFNKVVKFIFNTVGGFDEISPAGIQVSFVQYSDEVKSEFKLNTYNDKALALGALQNIRYRGGNTRTGKALTFIKEKVLTWESGMRKNVPKVLVVVTDGRSQDEVKKAALVIQQSGFSVFVVGVADVDYNELANIASKPSERHVFIVDDFESFEKIEDNLITFVCETATSSCPLIYLDGYTSPGFKMLEAYNLTEKNFASVQGVSLESGSFPSYSAYRIQKNAFVNQPTADLHPNGLPPSYTIILLFRLLPETPSDPFAIWQITDRDYKPQVGVIADPSSKTLSFFNKDTRGEVQTVTFDTEEVKTLFYGSFHKVHIVVTSKSVKIYIDCYEIIEKDIKEAGNITTDGYEILGKLLKGERKSAAFQIQSFDIVCSPVWTSRDRCCDIPSRRDEGKCPAFPNSCTCTQDSVGPPGPPGPAGGPGAKGPRGERGISGAIGPPGPRGDIGPPGPQGPPGPQGPNGLSIPGEQGRQGMKGDAGEPGLPGRTGTPGLPGPPGPMGPPGDRGFTGKDGAMGPRGPPGPPGSPGSPGVTGPSGKPGKPGDHGRPGPSGLKGEKGDRGDIASQNMMRAVARQVCEQLISGQMNRFNQMLNQIPNDYQSSRNQPGPPGPPGPPGSAGARGEPGPGGRPGFPGTPGMQGPPGERGLPGEKGERGTGSSGPRGLPGPPGPQGESRTGPPGSTGSRGPPGPPGRPGNSGIRGPPGPPGYCDSSQCASIPYNGQGYPGSG

SEQ ID NO: 92 - protein, 1899 aa, Homo sapiens
MRSRLPPALAALGAALLLSSIEAEGMECLTRAEADIVLLVDGSWSIGRANFRTVRSFISRIVEVFDIGPKRVQIALAQYSGDPRTEWQLNAHRDKKSLLQAVANLPYKGGNTLTGMALNFIRQQNFRTQAGMRPRARKIGVLITDGKSQDDVEAPSKKLKDEGVELFAIGIKNADEVELKMIATDPDDTHAYNVADFESLSRIVDDLTINLCNSVKGPGDLEAPSNLVISERTHRSFRVSWTPPSDSVDRYKVEYYPVSGGKRQEFYVSRMETSTVLKDLKPETEYVVNVYSVVEDEYSEPLKGTEKTLPVPVVSLNIYDVGPTTMHVQWQPVGGATGYILSYKPVKDTEPTRPKEVRLGPTVNDMQLTDLVPNTEYAVTVQAVLHDLTSEPVTVREVTLPLPRPQDLKLRDVTHSTMNVFWEPVPGKVRKYIVRYKTPEEDVKEVEVDRSETSTSLKDLFSQTLYTVSVSAVHDEGESPPVTAQETTRPVPAPTNLKITEVTSEGFRGTWDHGASDVSLYRITWAPFGSSDKMETILNGDENTLVFENLNPNTIYEVSITAIYPDESESDDLIGSERTLPILTTQAPKSGPRNLQVYNATSNSLTVKWDPASGRVQKYRITYQPSTGEGNEQTTTIGGRQNSVVLQKLKPDTPYTITVSSLYPDGEGGRMTGRGKTKPLNTVRNLRVYDPSTSTLNVRWDHAEGNPRQYKLFYAPAAGGPEELVPIPGNTNYAILRNLQPDTSYTVTVVPVYTEGDGGRTSDTGRTLMRGLARNVQVYNPTPNSLDVRWDPAPGPVLQYRVVYSPVDGTRPSESIVVPGNTRMVHLERLIPDTLYSVNLVALYSDGEGNPSPAQGRTLPRSGPRNLRVFGETTNSLSVAWDHADGPVQQYRIIYSPTVGDPIDEYTTVPGRRNNVILQPLQPDTPYKITVIAVYEDGDGGHLTGNGRTVGLLPPQNIHISDEWYTRFRVSWDPSPSPVLGYKIVYKPVGSNEPMEAFVGEMTSYTLHNLNPSTTYDVNVYAQYDSGLSVPLTDQGTTLYLNVTDLKTYQIGWDTFCVKWSPHRAATSYRLKLSPADGTRGQEITVRGSETSHCFTGLSPDTDYGVTVFVQTPNLEGPGVSVKEHTTVKPTEAPTEPPTPPPPPTIPPARDVCKGAKADIVFLTDASWSIGDDNFNKVVKFIFNTVGGFDEISPAGIQVSFVQYSDEVKSEFKLNTYNDKALALGALQNIRYRGGNTRTGKALTFIKEKVLTWESGMRKNVPKVLVVVTDGRSQDEVKKAALVIQQSGFSVFVVGVADVDYNELANIASKPSERHVFIVDDFESFEKIEDNLITFVCETATSSCPLIYLDGYTSPGFKMLEAYNLTEKNFASVQGVSLESGSFPSYSAYRIQKNAFVNQPTADLHPNGLPPSYTIILLFRLLPETPSDPFAIWQITDRDYKPQVGVIADPSSKTLSFFNKDTRGEVQTVTFDTEEVKTLFYGSFHKVHIVVTSKSVKIYIDCYEIIEKDIKEAGNITTDGYEILGKLLKGERKSAAFQIQSFDIVCSPVWTSRDRCCDIPSRRDEGKCPAFPNSCTCTQDSVGPPGPPGPAGGPGAKGPRGERGISGAIGPPGPRGDIGPPGPQGPPGPQGPNGLSIPGEQGRQGMKGDAGEPGLPGRTGTPGLPGPPGPMGPPGDRGFTGKDGAMGPRGPPGPPGSPGSPGVTGPSGKPGKPGDHGRPGPSGLKGEKGDRGDIASQNMMRAVARQVCEQLISGQMNRFNQMLNQIPNDYQSSRNQPGPPGPPGPPGSAGARGEPGPGGRPGFPGTPGMQGPPGERGLPGEKGERGTGSSGPRGLPGPPGPQGESRTGPPGSTGSRGPPGPPGRPGNSGIRGPPGPPGYCDSSQCASIPYNGQGYPGSG

SEQ ID NO: 93 - protein, 2987 aa, Homo sapiens
MRSRLPPALAALGAALLLSSIEAEVDPPSDLNFKIIDENTVHMSWAKPVDPIVGYRITVDPTTDGPTKEFTLSASTTETLLSELVPETEYVVTITSYDEVEESVPVIGQLTIQTGSSTKPVEKKPGKTEIQKCSVSAWTDLVFLVDGSWSVGRNNFKYILDFIAALVSAFDIGEEKTRVGVVQYSSDTRTEFNLNQYYQRDELLAAIKKIPYKGGNTMTGDAIDYLVKNTFTESAGARVGFPKVAIIITDGKSQDEVEIPARELRNVGVEVFSLGIKAADAKELKQIASTPSLNHVFNVANFDAIVDIQNEIISQVCSGVDEQLGELVSGEEVVEPPSNLIAMEVSSKYVKLNWNPSPSPVTGYKVILTPMTAGSRQHALSVGPQTTTLSVRDLSADTEYQISVSAMKGMTSSEPISIMEKTQPMKVQVECSRGVDIKADIVFLVDGSYSIGIANFVKVRAFLEVLVKSFEISPNRVQISLVQYSRDPHTEFTLKKFTKVEDIIEAINTFPYRGGSTNTGKAMTYVREKIFVPSKGSRSNVPKVMILITDGKSSDAFRDPAIKLRNSDVEIFAVGVKDAVRSELEAIASPPAETHVFTVEDFDAFQRISFELTQSICLRIEQELAAIKKKAYVPPKDLSFSEVTSYGFKTNWSPAGENVFSYHITYKEAAGDDEVTVVEPASSTSVVLSSLKPETLYLVNVTAEYEDGFSIPLAGEETTEEVKGAPRNLKVTDETTDSFKITWTQAPGRVLRYRIIYRPVAGGESREVTTPPNQRRRTLENLIPDTKYEVSVIPEYFSGPGTPLTGNAATEEVRGNPRDLRVSDPTTSTMKLSWSGAPGKVKQYLVTYTPVAGGETQEVTVRGDTTNTVLQGLKEGTQYALSVTALYASGAGDALFGEGTTLEERGSPQDLVTKDITDTSIGAYWTSAPGMVRGYRVSWKSLYDDVDTGEKNLPEDAIHTMIENLQPETKYRISVFATYSSGEGEPLTGDATTELSQDSKTLKVDEETENTMRVTWKPAPGKVVNYRVVYRPHGRGKQMVAKVPPTVTSTVLKRLQPQTTYDITVLPIYKMGEGKLRQGSGTTASRFKSPRNLKTSDPTMSSFRVTWEPAPGEVKGYKVTFHPTGDDRRLGELVVGPYDNTVVLEELRAGTTYKVNVFGMFDGGESSPLVGQEMTTLSDTTVMPILSSGMECLTRAEADIVLLVDGSWSIGRANFRTVRSFISRIVEVFDIGPKRVQIALAQYSGDPRTEWQLNAHRDKKSLLQAVANLPYKGGNTLTGMALNFIRQQNFRTQAGMRPRARKIGVLITDGKSQDDVEAPSKKLKDEGVELFAIGIKNADEVELKMIATDPDDTHAYNVADFESLSRIVDDLTINLCNSVKGPGDLEAPSNLVISERTHRSFRVSWTPPSDSVDRYKVEYYPVSGGKRQEFYVSRMETSTVLKDLKPETEYVVNVYSVVEDEYSEPLKGTEKTLPVPVVSLNIYDVGPTTMHVQWQPVGGATGYILSYKPVKDTEPTRPKEVRLGPTVNDMQLTDLVPNTEYAVTVQAVLHDLTSEPVTVREVTLPLPRPQDLKLRDVTHSTMNVFWEPVPGKVRKYIVRYKTPEEDVKEVEVDRSETSTSLKDLFSQTLYTVSVSAVHDEGESPPVTAQETTRPVPAPTNLKITEVTSEGFRGTWDHGASDVSLYRITWAPFGSSDKMETILNGDENTLVFENLNPNTIYEVSITAIYPDESESDDLIGSERTLPILTTQAPKSGPRNLQVYNATSNSLTVKWDPASGRVQKYRITYQPSTGEGNEQTTTIGGRQNSVVLQKLKPDTPYTITVSSLYPDGEGGRMTGRGKTKPLNTVRNLRVYDPSTSTLNVRWDHAEGNPRQYKLFYAPAAGGPEELVPIPGNTNYAILRNLQPDTSYTVTVVPVYTEGDGGRTSDTGRTLMRGLARNVQVYNPTPNSLDVRWDPAPGPVLQYRVVYSPVDGTRPSESIVVPGNTRMVHLERLIPDTLYSVNLVALYSDGEGNPSPAQGRTLPRSGPRNLRVFGETTNSLSVAWDHADGPVQQYRIIYSPTVGDPIDEYTTVPGRRNNVILQPLQPDTPYKITVIAVYEDGDGGHLTGNGRTVGLLPPQNIHISDEWYTRFRVSWDPSPSPVLGYKIVYKPVGSNEPMEAFVGEMTSYTLHNLNPSTTYDVNVYAQYDSGLSVPLTDQGTTLYLNVTDLKTYQIGWDTFCVKWSPHRAATSYRLKLSPADGTRGQEITVRGSETSHCFTGLSPDTDYGVTVFVQTPNLEGPGVSVKEHTTVKPTEAPTEPPTPPPPPTIPPARDVCKGAKADIVFLTDASWSIGDDNFNKVVKFIFNTVGGFDEISPAGIQVSFVQYSDEVKSEFKLNTYNDKALALGALQNIRYRGGNTRTGKALTFIKEKVLTWESGMRKNVPKVLVVVTDGRSQDEVKKAALVIQQSGFSVFVVGVADVDYNELANIASKPSERHVFIVDDFESFEKIEDNLITFVCETATSSCPLIYLDGYTSPGFKMLEAYNLTEKNFASVQGVSLESGSFPSYSAYRIQKNAFVNQPTADLHPNGLPPSYTIILLFRLLPETPSDPFAIWQITDRDYKPQVGVIADPSSKTLSFFNKDTRGEVQTVTFDTEEVKTLFYGSFHKRDEGKCPAFPNSCTCTQDSVGPPGPPGPAGGPGAKGPRGERGISGAIGPPGPRGDIGPPGPQGPPGPQGPNGLSIPGEQGRQGMKGDAGEPGLPGRTGTPGLPGPPGPMGPPGDRGFTGKDGAMGPRGPPGPPGSPGSPGVTGPSGKPGKPGDHGRPGPSGLKGEKGDRGDIASQNMMRAVARQVCEQLISGQMNRFNQMLNQIPNDYQSSRNQPGPPGPPGPPGSAGARGEPGPGGRPGFPGTPGMQGPPGERGLPGEKGERGTGSSGPRGLPGPPGPQGESRTGPPGSTGSRGPPGPPGRPGNSGIRGPPGPPGYCDSSQCASIPYNGQGYPGSG

SEQ ID NO: 94 - protein, 497 aa, Homo sapiens
MSCVLGGVIPLGLLFLVCGSQGYLLPNVTLLEELLSKYQHNESHSRVRRAIPREDKEEILMLHNKLRGQVQPQASNMEYMTWDDELEKSAAAWASQCIWEHGPTSLLVSIGQNLGAHWGRYRSPGFHVQSWYDEVKDYTYPYPSECNPWCPERCSGPMCTHYTQIVWATTNKIGCAVNTCRKMTVWGEVWENAVYFVCNYSPKGNWIGEAPYKNGRPCSECPPSYGGSCRNNLCYREETYTPKPETDEMNEVETAPIPEENHVWLQPRVMRPTKPKKTSAVNYMTQVVRCDTKMKDRCKGSTCNRYQCPAGCLNHKAKIFGTLFYESSSSICRAAIHYGILDDKGGLVDITRNGKVPFFVKSERHGVQSLSKYKPSSSFMVSKVKVQDLDCYTTVAQLCPFEKPATHCPRIHCPAHCKDEPSYWAPVFGTNIYADTSSICKTAVHAGVISNESGGDVDVMPVDKKKTYVGSLRNGVQSESLGTPRDGKAFRIFAVRQ

SEQ ID NO: 95 - protein, 449 aa, Homo sapiens
MSCVLGGVIPLGLLFLVCGSQGYLLPNVTLLEELLSKYQHNESHSRVRRAIPREDKEEILMLHNKLRGQVQPQASNMEYMTWDDELEKSAAAWASQCIWEHGPTSLLVSIGQNLGAHWGRYRSPGFHVQSWYDEVKDYTYPYPSECNPWCPERCSGPMCTHYTQIVWATTNKIGCAVNTCRKMTVWGEVWENAVYFVCNYSPKGNWIGEAPYKNGRPCSECPPSYGGSCRNNLCYREETYTPKPETDEMNEVETAPIPEENHVWLQPRVMRPTKPKKTSAVNYMTQVVRCDTKMKDRCKGSTCNRYQCPAGCLNHKAKIFGTLFYESSSSICRAAIHYGILDDKGGLVDITRNGKVPFFVKSERHGVQSLSKYKPSSSFMVSKVKVQDLDCYTTVAQLCPFEKPATHCPRIHCPAHCKDEPSYWAPVFGTNIYADVSRMHFQQLSRKAC

SEQ ID NO: 96 - protein, 371 aa, Homo sapiens
MSCVLGGVIPLGLLFLVCGSQGYLLPNVTLLEELLSKYQHNESHSRVRRAIPREDKEEILMLHNKLRGQVQPQASNMEYMTWDDELEKSAAAWASQCIWEHGPTSLLVSIGQNLGAHWGRYRSPGFHVQSWYDEVKDYTYPYPSECNPWCPERCSGPMCTHYTQIVWATTNKIGCAVNTCRKMTVWGEVWENAVYFVCNYSPKGNWIGEAPYKNGRPCSECPPSYGGSCRNNLCYREETYTPKPETDEMNEVETAPIPEENHVWLQPRVMRPTKPKKTSAVNYMTQVVRCDTKMKDRCKGSTCNRYQCPAGCLNHKAKIFGTLFYESSSSICRAAIHYGILDDKGGLVDITRNGKVPFFVKSERHGVQSLR

SEQ ID NO: 97 - protein, 159 aa, Homo sapiens
MSCVLGGVIPLGLLFLVCGSQGYLLPNVTLLEELLSKYQHNESHSRVRRAIPREDKEEILMLHNKLRGQVQPQASNMEYMVSAGSGRRGWHRGWGLGHQPALFPSQLCSPASACDGWLRVSSGRGGSRLCSVLFVCFETGSHSATDAGVQWHNRHALKP

SEQ ID NO: 98 - protein, 120 aa, Homo sapiens
MSCVLGGVIPLGLLFLVCGSQGYLLPNVTLLEELLSKYQHNESHSRVRRAIPREDKEEILMLHNKLRGQVQPQASNMEYMTWDDELEKSAAAWASQCIWEHGPTSLLVSIGQNLGAHWGR